(19) United States
(12) Patent Application Publication
(10) Pub. No.: US 2025/0250606 A1
(43) Pub. Date: Aug. 7, 2025

(54) BIOELECTROCHEMICAL DETERMINATION OF LACTATE

(71) Applicant: DIRECTSENS GMBH, Klosterneuburg (AT)

(72) Inventors: Leander SÜTZL, Wien (AT); Lidiia TSVIK, Wien (AT); Christopher SCHULZ, Wien (AT); Peter HERZOG, Obersdorf (AT); Beate STEINER, Korneuburg (AT); Alfons FELICE, Wien (AT)

(73) Assignee: DIRECTSENS GMBH, Klosterneuburg (AT)

(21) Appl. No.: 18/856,695

(22) PCT Filed: Apr. 13, 2023
(86) PCT No.: PCT/EP2023/059656

§ 371 Date: Oct. 14, 2024

(30) Foreign Application Priority Data
Apr. 14, 2022 (EP) 22168488.9

(51) Int. Cl. C12Q 1/32; C12N 9/04; C12N 11/14; G01N 27/327

(52) U.S. Cl. CPC C12Q 1/32; C12N 9/0006; C12N 11/14; G01N 27/3277; C12Y 101/01027

(57) ABSTRACT

The present invention relates to methods and means for detecting and/or quantifying lactate. Specifically, the present invention is directed to a method for detecting and/or quantifying lactate in a sample comprising the steps of: a) providing an electrode comprising a lactate dehydrogenase (LDH) comprising a flavin mononucleotide (FMN) and an amino acid sequence of SEQ ID NO: 2, or an amino acid sequence having at least 50% sequence identity to SEQ ID NO: 2, comprising the following sequence T-(Xₓ)ₙ₁-F-(Xₓ)ₙ₂-X₁-X₂-(Xₓ)ₙ₃-X₃-X₄ (SEQ ID NO: 1), wherein T is T122 of SEQ ID NO: 2, or corresponding to T122, F is F152 of SEQ ID NO: 2, or corresponding to F152, Xₓ is any amino acid, n1 is an integer of 25 to 35, n2 is an integer of 40 to 45, n3 is an integer of 5 to 20, X₁ is N or F, X₂ is L or F, X₃ is G, T, or S, and X₄ is I or V; b) contacting the sample with the electrode; and c) detecting the oxidation of lactate by the LDH, wherein the oxidation of lactate is performed in the presence of a redox mediator.

### FIELD OF THE INVENTION

The present invention relates to the field of detection and quantification of analytes, in particular to methods and means for the detection and/or quantification of lactate by providing an electrode comprising a lactate dehydrogenase.

### BACKGROUND OF THE INVENTION

In the food and beverage industries, as well as clinical diagnostics, there is a need for highly selective, sensitive, rapid, and reliable methods of determining the presence of key ingredients or metabolites which determine the quality of the product, serve as markers for diseases or as markers for the physiological state of humans. L-lactate is one of these metabolites. Analysis of lactate levels is also important in blood for the clinical diagnosis of hypoxia, lactic acidosis, for drug toxicity tests, hyperlactemia in diabetic and liver diseases, sepsis, and thiamine deficit. Lactate is also measured when monitoring the performance of athletes and developing optimum training regimens.

In pharmaceutical industry, the presence of the metabolite lactate has to be frequently monitored. For example, during the production of recombinant proteins in Chinese hamster ovary (CHO) cell cultures, lactate is one of the critical parameters because of its toxic and growth inhibiting effect on the cells.

Thereby, the relevant concentration ranges for L-lactate vary among the specific sample types and aims of the measurement. For example, the physiological relevant range of L-lactate in blood is usually between 1 and 25 mM (Goodwin et al., 2007) but the physiological relevant range of L-lactate can also be up to >100 mM in sweat after exhaustive exercise (Mitsubayashi et al., 1994).

Currently, the determination of lactate is based on an enzymatic test system measuring the oxidation of L-lactate to pyruvate. Traditionally, these enzymatic methods are based on NAD+-dependent lactate dehydrogenases (LDH) isolated from animal muscles or heart, or on bacterial lactate oxidase (LOx). Thereby, the lactate content is determined by the spectrophotometric detection of NADH or a colorimetric assay of H₂O₂. Many other methods have been proposed as well; for example, spectrophotometry, fluorimetry, and pH potentiometric measurements. Also amperometric biosensors have been proposed based on O₂ consumption and H₂O₂ formation which is detected on electrodes.

Biosensors would be the preferred method to monitor L-lactate levels. Rathee et al. (2016) describes basic principles of lactate biosensors. In general, a biosensor consists of a biological recognition element e.g., an enzyme, which is able to specifically interact with a target molecule and a transducer able to convert this interaction into a measurable signal. In biosensors, enzymes are connected to electrodes and electric currents are measured that are proportional to the enzymes' substrate concentration in a sample. Biosensors are easy to operate, highly specific and do not need expensive and heavy equipment like it is the case for HPLC or NMR.

The currently preferred enzyme used for the construction of L-lactate biosensors is L-lactate oxidase (LOx), especially LOx from Aerococcus viridans (AvLOx). LOx belongs to the family of FMN-dependent α-hydroxy acid oxidoreductases (HAOx; EC: 1.1.3.15; Maeda-Yorita et al., 1995) and as such oxidizes α-hydroxy acids to their corresponding α-keto acids via a ping-pong reaction mechanism (Maeda-Yorita et al., 1995). L-lactate, the preferred substrate of LOx, is oxidized to pyruvate in the first (reductive) half-reaction while FMN is reduced. In the second (oxidative) half-reaction, oxygen is used as an electron acceptor to re-oxidize FMN and is itself reduced to hydrogen peroxide (H₂O₂). Therefore, a biosensor based on an oxidase such as LOx is dependent on oxygen and is a first-generation biosensor.

In second-generation biosensors, redox mediators other than the O₂/H₂O₂ redox mediator are applied to transfer electrons from the enzyme to the electrode. Such a setup enables the use of low potentials, to avoid oxygen dependence and also to avoid the impact of interfering molecules. However, using oxidases as recognition element in such second-generation biosensors leads to oxygen interference in the presence of oxygen since oxygen is usually still present and competes with the alternative electron acceptor at the active site of the oxidase i.e., LOx. Thereby oxygen “steals” electrons away from the detectable electron flow, leading to a reduced sensor signal.

Oxygen interference is usually avoided by applying dehydrogenases instead of oxidases for second-generation biosensors. In contrast to oxidases, dehydrogenases do not use oxygen as primary and preferred electron acceptor. The application of a lactate dehydrogenase for the bio-electrochemical detection and/or quantification of lactate could prevent oxygen interference even in the presence of oxygen.

Unfortunately, up until now, the only known L-lactate dehydrogenases (LDHs) are either membrane bound or NAD+ dependent, both of which are unsuitable for the application in biosensors, e.g., for continuous lactate measurement in vivo, since production, purification and application of membrane-bound proteins comes with various challenges and NAD+ would have to be resupplied during measurement as it is continuously lost due to diffusion or degradation (Cardosi and Liu 2012; Kucherenko et al. 2019).

WO 2022/054044 A1 discloses a L-lactate biosensor with L-LDH from baker's yeast (ScLDH). ScLDH is a fungal flavocytochrome b2 enzyme located in the mitochondrion intermembrane space. This membrane associated enzyme shows impracticable yields upon recombinant expression.

Thus, there is an urgent need in the field for methods and means enabling lactate detection and/or quantification based on oxygen independent second-generation biosensors for application in industry.

### SUMMARY OF THE INVENTION

It is the objective of the present invention to provide a method and means for bio-electrochemically detecting and/or quantifying lactate in a sample which is independent of oxygen.

The objective is solved by the subject matter of the present invention.

The inventors of the present invention surprisingly found a distinct class of flavin mononucleotide (FMN) dependent lactate dehydrogenases capable of oxidizing lactate and transferring the gained electrons to a redox mediator with a limited capability to transferring electrons to oxygen. Specifically, the inventors found a specific sequence with distinct conserved amino acid residues showing this preferable dehydrogenase activity pattern of lactate oxidation for the application on electrodes for second-generation biosensors. These electrodes do not have or have a very low oxygen interference and enable the application of lactate biosensors in industry for applications such as continuous lactate measurement. An advantage of the capability of transferring the gained electrons from the lactate oxidation to a redox mediator of the herein described enzymes is that this transfer to a redox mediator is occurring even if oxygen is present during the oxidation of lactate. Since the enzymes described herein do not have or have a very low activity with oxygen as electron acceptor, the presence of oxygen does not disturb in the methods and means described herein. Another advantage of the capability of transferring the gained electrons from the lactate oxidation to a redox mediator instead of to oxygen is that several different redox mediators may be used in the methods and means described herein. It is generally known and described e.g., by Rathee, et al., (2016) that if an enzyme capable of oxidizing a molecule, e.g., lactate, does not accept oxygen as electron acceptor, i.e., is not a lactate oxidase, and if this enzyme accepts redox mediators as electron acceptors, i.e, the enzyme is a lactate dehydrogenase, such an enzyme accepts several different redox mediators which are generally known to the skilled person.

According to the invention there is provided a method for detecting and/or quantifying lactate in a sample comprising the steps of:
a) providing an electrode comprising a lactate dehydrogenase (LDH), said LDH comprising a flavin mononucleotide (FMN) and an amino acid sequence of SEQ ID NO: 2, or an amino acid sequence having at least 50% sequence identity to SEQ ID NO: 2, comprising the following sequence
   T-(Xₓ)ₙ₁-F-(Xₓ)ₙ₂-X₁-X₂-(Xₓ)ₙ₃-X₃-X₄ (SEQ ID NO: 1), wherein
   T is T122 of SEQ ID NO: 2, or corresponding to T122,
   F is F152 of SEQ ID NO: 2, or corresponding to F152,
   Xₓ is any amino acid,
   n1 is an integer of 25 to 35,
   n2 is an integer of 40 to 45,
   n3 is an integer of 5 to 20,
   X₁ is N or F,
   X₂ is L or F,
   X₃ is G, T, or S, and
   X₄ is I or V;
b) contacting the sample with the electrode; and
c) detecting the oxidation of lactate by the LDH.

Specifically, the oxidation of lactate is performed in the presence of a redox mediator.

Specifically, the sample is selected from the group consisting of any one of food; beverage; fermented food; fermented beverage; chemicals; water; soil; and a sample provided by a human or animal, specifically from any one of body fluid, interstitial fluid, blood, plasma, dermal fluid, urine, tears, sweat, saliva, skin, flesh, tissue, eyeballs, cornea, and gastric fluid.

According to the invention there is also provided an electrode comprising an LDH, wherein said LDH comprises flavin mononucleotide (FMN) and an amino acid sequence of SEQ ID NO: 2, or an amino acid sequence having at least 50% sequence identity to SEQ ID NO: 2, comprising the following sequence
T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S,
X₄ is I or V.

Specifically, said electrode further comprises a redox mediator.

Specifically, according to said method or said electrode, the LDH is immobilized on the electrode, preferably by adsorption, complex formation, preferably via an additional complexing linker, covalent or ionic linkage, and/or wherein preferably the LDH is cross-linked, in particular by bifunctional agents.

Specifically, the LDH is immobilized on the electrode.

Specifically, the LDH is immobilized by adsorption or complex formation.

Specifically, complex formation is via a complexing linker, covalent or ionic linkage.

Specifically, the LDH is cross-linked.

Specifically, the LDH is cross-linked by bifunctional agents.

According to the invention there is also provided a biosensor comprising an electrode according to the invention.

According to the invention there is also provided a device comprising an electrode according to the invention, or a biosensor according to the invention.

According to the invention there is also provided an enzyme composition comprising an LDH and a redox mediator, wherein said lactate dehydrogenase comprises a flavin mononucleotide (FMN) and an amino acid sequence of SEQ ID NO: 2, or an amino acid sequence having at least 50% sequence identity to SEQ ID NO: 2, comprising the following sequence
T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S, and
X₄ is I or V.

Specifically, according to the method of the invention, the electrode of the invention, or the enzyme composition of the invention, the redox mediator is selected from the group consisting of any one of organic redox mediators, soluble redox mediators, insoluble redox mediators, redox polymers, transition metal complexes, polymeric transition metal complexes, wired redox mediators, sandwich compounds, and derivatives of these redox mediators.

According to the invention there is also provided a recombinant LDH comprising a flavin mononucleotide (FMN), wherein the amino acid sequence of said LDH comprises from N- to C-terminus
i. the dipeptide GP, and
ii. SEQ ID NO: 2, or an amino acid sequence having at least 50% sequence identity to amino acids 2 to 373 of SEQ ID NO: 2, comprising the sequence T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S,
X₄ is I or V.

Specifically, according to the recombinant LDH comprising a flavin mononucleotide (FMN) described herein, the dipeptide “GP” forms the N-terminus of the sequence. Thereby, the “G” forms the N-terminus of the recombinant LDH and the following sequence is linked to the C-terminus of the dipeptide “GP”:

SEQ ID NO: 2, or an amino acid sequence having at least 50% sequence identity to amino acids 2 to 373 of SEQ ID NO: 2, comprising the sequence T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S,
X₄ is I or V.

Specifically, the technical effect of the dipeptide “GP” in the a recombinant LDH comprising a flavin mononucleotide (FMN) described herein is an increased activity.

Specifically, according to the method of the invention, the electrode of the invention, the enzyme composition of the invention, or the recombinant LDH of the invention, the amino acid sequence of the LDH comprises a sequence having at least 70%, 80%, 90%, or 95% sequence identity to amino acids G35 to K360 of SEQ ID NO: 2, wherein the LDH comprises SEQ ID NO: 1.

Specifically, according to the method of the invention, the electrode of the invention, the enzyme composition of the invention, or the recombinant LDH of the invention, the LDH is selected from SEQ ID NO: 2, 13, 59, 73, 74, 155 or a sequence with at least 70%, 80%, 90%, or 95% sequence identity to at least one of SEQ ID NO: 2, 13, 59, 73, 74, 155, wherein the sequence comprises SEQ ID NO: 1.

Specifically, according to the method of the invention, the electrode of the invention, the enzyme composition of the invention, or the recombinant LDH of the invention, the LDH is selected from SEQ ID NOs: 2 to 249, or a sequence with at least 90%, or 95% sequence identity to at least one of SEQ ID NOs: 2 to 249, wherein the LDH comprises SEQ ID NO: 1.

According to the invention there is also provided a kit for detecting and/or quantifying lactic acid or a salt thereof comprising an electrode of the invention, the biosensor of the invention, or the device of the invention, further comprising an instruction manual.

### FIGURES

FIG. 1. Maximum likelihood phylogenetic tree of the LOx cluster. Positions of some enzyme sequences and their relative activities with L-lactate are shown. The tree shows a clear separation of oxidases and dehydrogenases into two different clades.

FIG. 2. UV-Vis absorption spectra showing the oxidized (full lines) and reduced form (dashed lines) of the purified flavoproteins. Maxima of the oxidized FMN cofactor are indicated in each spectrum. n.d.—not determinable.

FIG. 3. Effect of an overnight digest of the enzymes' purification tags, using the protease HRV 3C, on their specific activities. Activities were measured with L-lactate and DCIP.

FIG. 4. pH activity curves of LDHs and AvLOx measured with 10 mM L-lactate and oxygen (dashed lines) or DCIP (full lines) as electron acceptors. Activities are given relative to the highest respective activity in %. Measurements were done in quadruplicates in 40 mM Britton-Robinson universal buffer (BRB) pH 4.5-10.

FIG. 5. Current response of biosensor prototype at increasing lactate concentration (left) and calibration function (right) using EaLDH

FIG. 6. Current response of biosensor prototype at increasing lactate concentration (left) and calibration function (right) using LjLDH

FIG. 7. Current response of biosensor prototype at increasing lactate concentration (left) and calibration function (right) using SsLDH

FIG. 8. Current response of biosensor prototype at increasing lactate concentration (left) and calibration function (right) using EaLDH

FIG. 9. Current response of biosensor prototype at increasing lactate concentration (left) and calibration function (right) using AvLOX

FIG. 10. Comparison of catalytic currents in biosensor prototypes in presence and absence of oxygen.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017); Berg et al, “Stryer Biochemie” Springer Verlag, 2018; and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods, and uses of the invention, e.g., specifically referring to isolated nucleic acid sequences, amino acid sequences, expression constructs, transformed host cells and modified proteins and enzymes, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

As used herein, amino acids refer to twenty naturally occurring amino acids encoded by sixty-one triplet codons. These 20 amino acids can be split into those that have neutral charges, positive charges, and negative charges:

The “neutral” amino acids are shown below along with their respective three-letter and single-letter code and polarity: Alanine(Ala, A; nonpolar, neutral), Asparagine (Asn, N; polar, neutral), Cysteine (Cys, C; nonpolar, neutral), Glutamine (GIn, Q; polar, neutral), Glycine (Gly, G; nonpolar, neutral), Isoleucine (IIe, I; nonpolar, neutral), Leucine (Leu, L; nonpolar, neutral), Methionine (Met, M; nonpolar, neutral), Phenylalanine (Phe, F; nonpolar, neutral), Proline (Pro, P; nonpolar, neutral), Serine (Ser, S; polar, neutral), Threonine (Thr, T; polar, neutral), Tryptophan (Trp, W; nonpolar, neutral), Tyrosine (Tyr, Y; polar, neutral), Valine (Val, V; nonpolar, neutral), and Histidine (His, H; polar, positive (10%) neutral (90%)).

The “positively” charged amino acids are: Arginine (Arg, R; polar, positive), and Lysine (Lys, K; polar, positive).

The “negatively” charged amino acids are: Aspartic acid (Asp, D; polar, negative), and Glutamic acid (Glu, E; polar, negative).

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

The term “enzyme” as used herein refers to any substance composed wholly or largely of protein or polypeptides that catalyzes or promotes, more or less specifically, one or more chemical or biochemical reactions.

The term “lactate” as used herein refers to a lactic acid or a salt thereof. The preferred enantiomer of lactate is the L-lactate.

According to the invention, a method for detecting and/or quantifying lactate is provided. Thereby the term “detecting” lactate refers to the general determination if lactate is present. Detection does not require the exact quantification of lactate but rather provides the user of the method with the information if e.g., lactate is present with a concentration above a certain threshold. These thresholds are to be adapted to the respective application and sample. The term “quantification” refers to the determination of the concentration or amount of lactate.

According to the invention, detecting the oxidation of lactate by the LDH is carried out by a sensor, specifically a bio-electrochemical sensor, configured to detect and/or quantify lactate in a sample via (bio)electrochemical redox reactions. These reactions typically can be transduced to an electrical signal that can be correlated to an amount or concentration of the analyte lactate.

Electrochemical biosensors can be impedimetric, potentiometric or amperometric. In an amperometric biosensor, a biochemical signal is transduced into a quantifiable amperometric signal.

As described in Rocchitta G. et al. (2016) amperometric biosensors are commonly divided into three main generations depending on the electron transfer method used for the measurement of the biochemical reaction or the degree of separation of the biosensor components (transducer, enzyme, mediators and cofactors). First-generation biosensors measure the concentration of analytes and/or products of enzymatic reactions that diffuse to the transducer surface and generate an electrical response. They are also called mediatorless amperometric biosensors. Commonly, oxidases are used in first-generation biosensors. Oxidases need molecular oxygen as a second substrate so the oxidase-based biosensors are oxygen dependent. First-generation biosensors that use oxygen as an electron acceptor are thus subject to errors arising from changing or low concentration of dissolved oxygen impacting on sensor response and reducing linearity. This oxygen dependence limits the applicability of first-generation amperometric biosensors in biological systems—for example, they are not suitable for use under ischemic conditions. Ischemia is a condition in which the blood flow (and thus oxygen) is restricted or reduced in a part of the body. Cardiac ischemia is a condition with decreased blood flow and oxygen to the heart muscle. It has been described that lactate concentrations in incisions indicate ischemic-like conditions and may contribute to postoperative pain (Kim, T. J. et al., 2007).

According to one embodiment, the sample may be any material for which the lactate concentration is relevant or interesting. In particular, the sample is selected from the group consisting of any one of food; beverage; fermented food; fermented beverage; chemicals; water; soil; and a sample provided by a human or animal, specifically from any one of body fluid, interstitial fluid, blood, plasma, dermal fluid, urine, tears, sweat, saliva, skin, flesh, tissue, eyeballs, cornea, and gastric fluid.

For the detection and/or quantification according to the herein provided method, the electrode is contacted with the sample. This contact between electrode and sample can be performed by any approach which brings the electrode and the sample in contact in order that the enzyme is allowed to react with the lactate or with the sample suspected to contain lactate.

The term “lactate dehydrogenase” is abbreviated herein as LDH and refers to an enzyme catalyzing the oxidation of lactate to pyruvate whereby two electrons are transferred from lactate to the cofactor FMN of the LDH and the subsequent transport of the so gained electrons towards a suitable electron acceptor like DCIP whilst commonly being irresponsive or almost irresponsive to accepting dioxygen as an electron acceptor.

According to a specific embodiment, the LDH is characterized by its enantiomer selectivity and specificity towards the natural lactate substrate L-lactate.

According to a specific embodiment, the LDH of the invention may be active at acidic, neutral, or alkaline pH ranges. Specifically, the LDH of the invention may be used at a pH of 3.0, 3.5, 4.0, 4.5, 5.0, 5.5, 6.0, 6.5, 7.0, 7.5, 8.0, 8.5, 9.0, 9.5, or at any pH of a body fluid e.g., of sweat or blood. Usually, blood has a pH between pH 7.35 and pH 7.45.

The LDH according to the invention comprises an amino acid sequence of SEQ ID NO: 2, or an amino acid sequence having at least 50% sequence identity to SEQ ID NO:2, comprising the sequence T-(Xₓ)ₙ₁-F-(Xₓ)ₙ₂-X₁-X₂-(Xₓ)ₙ₃-X₃-X₄ (SEQ ID NO: 1), wherein T is T122 of SEQ ID NO: 2, or corresponding to T122; F is F152 of SEQ ID NO: 2, or corresponding to F152; Xₓ is any amino acid; n1 is an integer of 25 to 35; n2 is an integer of 40 to 45; n3 is an integer of 5 to 20; X₁ is N or F; X₂ is L or F; X₃ is G, T, or S; and X₄ is I or V.

According to a specific embodiment of the invention, the LDH of the invention comprises an amino acid sequence of SEQ ID NO: 2, or an amino acid sequence comprising an amino acid sequence with at least 50%, 60%, 70%, 75%, 80%, 85%, 90%, or even 95% sequence identity to SEQ ID NO: 2, or an amino acid sequence comprising an amino acid sequence with at least 50%, 60%, 70%, 75%, 80%, 85%, 90%, or even 95% sequence identity to amino acids Gly35 to Lys360 of SEQ ID NO: 2, wherein the amino acid sequence of the LDH further comprises the sequence T-(Xₓ)ₙ₁-F-(Xₓ)ₙ₂-X₁-X₂-(Xₓ)ₙ₃-X₃-X₄ (SEQ ID NO: 1), wherein T is T122 of SEQ ID NO: 2, or corresponding to T122; F is F152 of SEQ ID NO: 2, or corresponding to F152; Xₓ is any amino acid; n1 is an integer of 25 to 35; n2 is an integer of 40 to 45; n3 is an integer of 5 to 20; X₁ is N or F; X₂ is L or F; X₃ is G, T, or S; and X₄ is I or V.

The LDH sequences according to the invention comprise SEQ ID NO: 1 as defined herein, wherein said LDH sequences comprising SEQ ID NO: 1 are characterized in that these LDHs catalyze the lactate dehydrogenase reaction as defined herein.

SEQ ID NO: 2 is the amino acid sequence of LDH from Pediococcus acidilactici (PaLDH). In the present invention, it has been surprisingly found that the enzyme from Pediococcus acidilactici “PaLCTO” described in Ashok Y. et al. (2020) is actually capable of oxidizing lactate when using a redox mediator other than molecular oxygen as electron acceptor. According to Ashok Y. et al. (2020) the PaLCTO does not use lactate as a substrate. Even more surprisingly, in the present invention it has been found that the enzyme termed PaLCTO in Ashok Y. et al., (2020) is a lactate dehydrogenase enabling the detection and/or quantification of lactate by an electrode using mediated electron transfer.

According to one embodiment, in the sequence T-(Xₓ)ₙ₁-F-(Xₓ)ₙ₂-X₁-X₂-(Xₓ)ₙ₃-X₃-X₄ (SEQ ID NO: 1) as defined herein, the amino acids and the variables are the following amino acids and integers in SEQ ID NO: 2: T is T122, F is F152, n1 is 29, n2 is 43, n3 is 11, X₁ is N196, X₂ is L197, X₃ is G209, X₄ is 1210. In SEQ ID NO: 1 of the LDH of the invention, the amino acids and integers correspond to these amino acids and integers.

The term “corresponding to” as used herein refers to the respective corresponding amino acid or integer determined by a sequence alignment and/or by structure alignment of two or more sequences. For example, for the determination of the corresponding amino acids in a LDH, this LDH amino acid sequence is aligned in a sequence and/or structure alignment and/or by superposition with e.g., SEQ ID NO: 2.

According to a specific embodiment, n1 is an integer of 25, 26, 27, 28, 29, 30, 31, 32, 33, 34, or 35.

According to a specific embodiment, n2 is an integer of 40, 41, 42, 43, 44, or 45.

According to a specific embodiment, n3 is an integer of 5, 6, 7, 8, 9, 10, 11, 12, 13, 14, 15, 16, 17, 18, 19, or 20.

According to a specific embodiment of the invention, the electrode comprises a LDH comprising a flavin mononucleotide (FMN) and an amino acid sequence of SEQ ID NOs: 3 to 249, or an amino acid sequence having at least 70%, at least 80%, at least 90%, or 95% sequence identity to SEQ ID NOs: 3 to 249, comprising the sequence of T-(Xₓ)ₙ₁-F-(Xₓ)ₙ₂-X₁-X₂-(Xₓ)ₙ₃-X₃-X₄ (SEQ ID NO: 1), wherein T is T122 of SEQ ID NO: 2, or corresponding to T122; F is F152 of SEQ ID NO: 2, or corresponding to F152; Xₓ is any amino acid; n1 is an integer of 25 to 35; n2 is an integer of 40 to 45; n3 is an integer of 5 to 20; X₁ is N or F; X₂ is L or F; X₃ is G, T, or S; and X₄ is I or V.

In the following Table 1, amino acid sequences SEQ ID NO 2 to SEQ ID NO: 249 are given.

TABLE 1

| | |
|---|---|
| SEQ ID | MTMINGYEQSDREEKIDILNLESLEKQAEEIIPAGGFGYIAGGSEDEWTLKQN |
| NO: 2 | RMAFHHRQIAPKALSGIEKPELNTEIFGIPLNTPVMMAPAAAQGLAHSQGEK |
| E0NE46 | DTARGLAAVGGLMAQSTYSSVSIAETAAAGGDAPQFFQLYMSKDWNFNES |
| Pediococcus | LLDEAKKANVKAIILTVDATVDGYREADIKNKFTFPLPMANLIKFSEGNGQGK |
| acidilactici | GIEEIYASAAQNIRPEDVKRIADYTNLPVIVKGIQTPEDAIRAIDAGAAGIYVSN |
| DSM 20284 | HGGRQLNGGPASFDVLEDIATAVNKQVPIIFDSGVRRGSDVFKALASGADLV |
| | ALGRPVIYGLALGGAKGVQSVFEHLNHELEIVMQLAGTKTIEDVKNNSLLNIK |
| | Y |
| SEQ ID | MTHYIKGFPQSEADEDLKFVNVDELEEKAKEVMPEGAYYYVASGSEYEWT |
| NO: 3 | WRNNTTAFNHYQIVPRALTGMDNPSTETEFLGMKLKTPIMISPIACHGISHAD |
| A0A061CP | AEVATQKGAALAGAMFTSSTYGNKPVEEIAAAAPDAPRMFQLYLSKNWDFN |
| W4 | KMVFDAINAAGYKAILLTVDALVSGYREANLRTNFAFPVPLDFFTRFQGAKG |
| Lactobacillus | EGQTVAQMYASSAQNIGPDDIKRIKEMSGLPVIVKGVNCAEDVEVALTAGAD |
| delbrueckii | GVYVTNHGGREIDGAPATIDVLPEVVEAVNGRCPVIFDGGVRRGSHVFKALA |
| subsp. | LGADLVGIGRPYLYGLALGGPHGVASVINELNDELKIDMQLTGCKTIEDVKHA |
| lactis | RLTDFQYAGDTKPSSTDPRVRKPYPVTAVNQIKSEPDAATGASHH |
| SEQ ID | MAYVTSNDEQKVEIVNIASLERRVKDRMEAQGNKGAFGYIRGGAEDEWTM |
| NO: 4 | RENTASFNTKTISPRVLRGIDSADLRTSIFGIDLKTPIIQAPVAAQGLAHMEGE |
| A0A098CY | VDTAKAMEEMGSLFSISTYGSTSVEDAAAAVNGAPQFFQLYMSKDDQFNQF |
| 34 | LLEKAVKSGVKAIILTADSTLGGYREEDVINHFQFPLPMPNLAAFSESDGVGK |
| Lactococcus | GIFEIYAEAKQGLVLSDIQKIKNWTNLPVIVKGIQDPVDAMEAIAAGADGIWVS |
| garvieae | NHGGRQLDGGPASFTVLPRIAQVVNKRVPIIFDSGVRRGEHVFKALASGADL |
| | VAIGRPVLYGLNLGGKEGVKSVFEHLNKELSITMQLAGAKDIEAIKNTDLL |
| SEQ ID | MSYQASNEEKDLKIVNLASLEARVKPRMEAGAFGYIRGGSEDEWTMQQNT |
| NO: 5 | VAFQHKKIMPHVLKGIDSADLHTNIFGIDLKTPIIEAPSAAQGLAHTKGEIDTAK |
| A0A0A1GX | GVADAGSIFSISTYANTKIEDAAAAVPDAPQFFQLYMSKDDGFNKFILDKAVK |
| Y9 | AGAKAIILTADSTLGGYREADVINGFQFPLPMPNLAAYSDQTDSGDGQGKGI |
| Paucilacto- | SEIYAAAKQGLVLADIRKVKEWTHLPVIVKGIQAPEDAEAVILSGADAIWVSN |
| bacillus | HGGRQLDGGPASFDVLPAIAHTVAKRVPIIFDSGVRRGEHVFKALASGADLV |
| hokkaidonensis | AIGRPVIYGLNLGGAKGVKSVFDHLNMELSITMQLAGTKDVTAIKNTTLID |
| JCM | |
| 18461 | |
| SEQ ID | MGYHTSEAENPIDILNLASLEGRVKERMEAGAFGYIRGGAEDEWTMAANTS |
| NO: 6 | AFNTKKIMPRVLKGIDHADLHTKLWDIDLKTPIIQAPSAAQGLAHEKGEVDTA |
| A0A0B2XQ | KGIAAAGSIFSISTYANTLVEDAAAAAPDAPQFFQLYMSKDDQFNEFLLKKAV |
| D5 | KAGVKAIILTVDSTLGGYREADIETQFQFPLPMPNLAAYSNSDGAGKSISEIYA |
| Latilactobacillus | AAKQGLVPEDIQKIKQITNLPVFVKGIQSPVDAEIAIQAGADGIWVSNHGGRQ |
| curvatus | LDGGPASFEVLPLIAQQVAKRVPIVFDSGIRRGEHVFKALASGADLVAIGRPII |
| | YGLNLGGAQGVQSVFEHLNHELSITMQLAGTKTIDEVKETTLLD |
| SEQ ID | MKKTIFTLTAITLMTSAFADVVTNDSTTKTVFQDKNKKGKGYQASTADKKLKI |
| NO: 7 | VNLHELEGQVKAEMDQGAFGYIVGGAEDQNNLKINTENFDKKYIMPRVLKGI |
| A0A0B5S2 | KHEDIDLSTSLFGIPLKTPIIQAPMAAQGLSHVDGEIATAKGMIAAGSLFSLST |
| T4 | YGNKTIEEVAEGINGAPFFFQLYMSKNDDFNKFTLDRAKKYGAKAIILTVDSP |
| Myroides | VGGYREEDIKTGFTFPLGMGNLELFAAQQADGNKTGKGAGITEIYAQAKQD |
| profundi | FKPSDIKYVKDLTGLPVIIKGIQSPEDAEIAIQAGADAIWVSNHGGRQLDAGPS |
| | SFDVLPLVAKTVNKRVPIIFDSGVRRGSHIFKAIASGADIVAIGRPILYALHLGG |
| | SQGVTSVIDQLNKELTINMFLGGAKNIKEIQSTKLYTDKDFQL |
| SEQ ID | MTYKTSTENKALEIVNVKSLEGKVKQSMEAAGNKGAFGYIRGGSEDEWTLN |
| NO: 8 | ENTSAFNKKQIMPRVLRGIDSADLSTSLFGIKLKTPIIQAPVAAQGLAHEEGEV |
| A0A0B8R2 | ATAKAMAEVGSIFSISTYGSTSVEDVAKAAPGAPQFFQLYMSKDDKFNEFLL |
| E5 | KKAVSAGVKAIILTADSTLGGYREEDIVNHFQFPLPMPNLAAFSESDGTGKGI |
| Lactococcus | SEIYAAAKQGLVLEDIQKIKKITNLPVIVKGVQSPIDADDAINAGADGIWVSNH |
| lactis | GGRQLDGGPASIDVLPLIAKSVNHRVPIVFDSGVRRGEHVFKALAQGADVVA |
| subsp. | VGRPVLYGLNLGGAKVVQSVFEHLSKELSITMQLAGTKNIEEIKHTSLID |
| lactis | |
| SEQ ID | MTMINGYEQSDREESLDILNLPSLEARAKQIIPAGGFGYISGGSEDEWTLKEN |
| NO: 9 | TEAFNHVQIIPRALTNIEHPSTQTQIFGVDLQTPIMMAPAAAQGLAHSRGEMA |
| A0A0C1M6 | TAQGIAAAGALMAQSTYSSTSIADTAAAGQGAPQFFQLYMSKDWHFNESLL |
| B4 | DEAIKANVKAIILTVDATVDGYRESDIVNKFQFPIPMANLTKFSEGDGKGKGIQ |
| Levilactobacillus | EIYAAAAQKINADDVRRIAEYTHLPVIVKGIQSVEDAMTAIGAGAAGIYVSNHG |
| brevis | GRQLNGGPASFDVLHDIALAVNHRVPIIFDSGVRRGSHVFKALANGADLVAL |
| | ARPVIYGLALGGAQGVTSVINHLNDELLIDMQLAGTKTIEDVKQAKLLRH |
| SEQ ID | MTYQGSTKEEKLNIIDLPRLRDAVKRDTEAGAFGYVDGGSSDEQVLRDNEQ |
| NO: 10 | AFRHYQLIPRMLQDIAAPDLSTTLFDIPLTMPVIAAPIAAHGLMHQDGEQVTV |
| A0A0D1LH | KGVGAAGSIFSLSTYGNSRIADVASAAPDTPKFFQLYMSRDDDFNQYLLDEA |
| D3 | VNNGYKAIILTADATLGGYREADIINNFTFPLPMENLAAFSNAAGSGEGLGIA |
| Weissella | DIYARAKQDLSLRDIKKVKEMAHGLPVIVKGIQDPDDALAAIAAGADGIWVSN |
| cibaria | HGGRELNGAPASIDTLAAIAKAVNRRVPVIFDSGIRRGEDVAKALALGADVVA |
| | LGRPMLWGLNQGGAAGVQSVYEHLATELKIVMQLTGAHTVAELQRAKIINAK |
| | F |
| SEQ ID | MTSYYNGFPQSDRDETLKMINLDELEERAKKVMPEGAYYYIASGSENEWTW |
| NO: 11 | RNNTTAFNHFQIVPRALTNMDNPKLDTEFMGMKLKTPVMISPIACHGIAHKD |
| A0A0F4LA | AEIATQKGAAAAGALFASSTYANKSVEEIAAAAPNAPRFFQLYLSKDWGFNK |
| 18 | MVFDAVKKAGYKGIFLTVDALVSGFREANLRTKFAYPVPLDFFTRYQGAKGE |
| Lactobacillus | GQTVAQMYASSAQKIGPEDVRRIKEMSGLPVFVKGVVCAEDAYLAMGAGA |
| kullabergensis | DGIYVTNHGGREVDCGPATIDMLPEIAKAVNHRVPIVFDSGVRRGSHVFKAL |
| | ALGADMVGVGRPYLYGLALGGAKGVQSVIEQLNQELLIDMQLTGCKTIEDVK |
| | HAKITHINYTADNLKSNTDPTRVKPYPVTAENQLKEDDSDAVTGASQA |
| SEQ ID | MTYYYNGFPQSERNEALNMVNLDELEERAKEVMPEGAYYYIASGSENEWT |
| NO: 12 | WRNNTTAFNHFQIVPRALTNMDHPQLDTEFMGMKLKTPVMISPIACHGIAHK |
| A0A0F4LD | DAEIATQKGAAAAGALFSSSTYANKSVEDIAAAAPDAPRFFQLYLSKDWDFN |
| V1 | QMVFDAVKKAGYKGIFLTVDALISGFREANLRTNFAYPVPLDFFTRYQGAKG |
| Lactobacillus | EGQTVAQMYASSAQKIGPQDVRRIKEMSGLPVFIKGVVCAEDAYIAMGAGA |
| melliventris | DGIYVTNHGGREVDCGPATIDMLPEIAKAVNHRVPIVFDSGVRRGSHVFKAL |
| | ALGADMVGIGRPYLYGLALGGAKGVQSVIEQLNQELLIDMQLTGCKTIDDVK |
| | HAKITHINYTADNLKSNTDPSRVKPYPVTAENQLKNNGSDAVTGASQA |
| SEQ ID | MTSYYNGFPQSDRDEAIDMINLDELEERAKKVMPEGAYYYIASGSENEWTW |
| NO: 13 | RNNISAFNHFQIVPRALTNMDHPQLDTEFMGMKLKTPVMISPIACHGIAHKDA |
| A0A0F4LW | EIATQKGAAAAGALFSSSTYANKSVEDIAAAAPDAPRFFQLYLSKDWGFNKM |
| 09 | VFDAVKKAGYKGIFLTVDALVSGFREANLRTKFAYPVPLDFFTRYQGAKGEG |
| Lactobacillus | QTVAQMYASSAQKIGPEDIKRIKEMSGLPVFVKGVVCAEDAYLAMGAGADGI |
| helsingborgensis | YVTNHGGREVDCGPATIDMLPEIAKAVNHRVPIVFDSGVRRGSHVFKALALG |
| | ADMVGIGRPYLYGLALGGAKGVQSVIEQLNQELLIDMQLTGCKTIEDVKHAKI |
| | THINYTADNLKSNTDPSRVKPYPVTAENQLKENDSDAVTGASQA |
| SEQ ID | MTVINGYEQSDREQKLTILNLPSLEAAAKKIIPSGGFGYISGGSEDEWTLKQN |
| NO: 14 | TMAFNHVQIVPRALTDMEQPSTQTQAFGIDLKTPIMMAPAAAQGLAHARGE |
| A0A0G2ST | AATAEGMAQVGALMAQSTYSSTSIADTASAGKGAPQFFQLYMSKDWDFNQ |
| 72 | SLLDEAVKAGAKAIILTVDATVDGYREADIINNFQFPIPMANLTKFSEGDGKGK |
| Lactiplanti- | GIMEIYAAAAQKISPADVRRITEYTNLPVIVKGVQSPEDALLAIGAGAQGIYVS |
| bacillus | NHGGRQLNGGPASFDVLHEIAQAVNGRVPIIFDSGVRRGSHVFKALANGAD |
| plantarum | LVALARPIIYGLALGGAQGVASVVSHLNDELLIDMQLAGTKTIEDVKRAKLLR |
| SEQ ID | MKSKILKTTAIAMALSVGVAQAAEYKASIAEGPIKIVNLKAMEAQVQANMEKG |
| NO: 15 | AFGYIRGGAEDENNLRSNTTAFDKKYIMPRSLQGIEFSDLNLKTEFLGIKLDT |
| A0A0G4Q8 | PIIQAPMAAQGLAHQQGEVATAKGMAKAGSIFSLSTYGNKTIKEVADAQPGY |
| W7 | PFFFQLYMSKNDAFNEYILSQAKQYGAKGIIMTIDSSVGGYREDDVKNNFQF |
| Proteus | PLGFANLEAFAKISDDKSKTGKGAGISEIYAQAKQAFTPEDIQYVKKMSGLPV |
| penneri | IVKGIESPEDADTAIKAGADAIWVSNHGGRQLDSAPATIDVLPAIAKVVNKRV |
| | PIVFDSGVRRGSHVFKALASGADVVAVGRPILYGLNLGGAEGVNSVIQHLNK |
| | ELKINMMLGGAKTVKDIQATHLYTDASFNQ |
| SEQ ID | MTVVNGYEQSDAEKKLNILNLPSLADEAKKIIPTGGFGYIVGGSEDEWTLAEN |
| NO: 16 | TKAFNHAQIVPKALSNIDSPDLSTSFLGIDLKTPVMMAPTAAQGLAHSQGEK |
| A0A0H0YS | DTARGVAAVGGLMAQSTYSSTSIADTAAAGNGAPQLFQLYMSKDWDFNKS |
| 91 | LLDEAKKAGVKGIILTVDATVDGYREEDIINNFQFPIPMPNLEKYSEGDGKGK |
| Lacticasei- | GIGEIYASAAQKIGEDDVRRIAEYTDLPVIVKGIQSPEDALRAIGAGAAAVYVS |
| bacillus | NHGGRQLNGGPASFDVLPAIAKAVNKRVPIIFDSGIRRGSHAFKALAAGADL |
| casei | VAFGRPVIYGLALGGAQGVQSVFEQIDHELEIIMQLAGTKTIEDVKHAPLTHF |
| | NYAD |
| SEQ ID | MAKYEASTEEKHIDIVNIASLEQRVKDHMSNEKGAFGYIRGGSEDEWTMKQ |
| NO: 17 | NTEAFNKKRIMPRVLQGIDHADLSTKFWDIDLKTPIIEAPSAAHGLAHAKGEV |
| A0A0H4L7 | DTAKGVAAAGSIFSMSTYGSTSIEDGAAAAPDAPQFFQLYMSKDDKFNEFLI |
| 08 | KKAVKAGVKAIILTVDSTLGGYREEDVINKFQFPLPMPNLAAFSDDDGEGKGI |
| Companilac- | SEIYAAAKQGIVPSDIQKIKDMSGLPVFVKGIQSPDDAELAIEFGADGIWVSN |
| tobacillus | HGGRQLDGGPASFDVLPAIAQVVDKRVPVVFDSGVRRGEHVFKALASGADL |
| farciminis | VAVGRPIIYGLNLGGAQGVTDVIEHLNKELSITMQLAGTKTIHDVKNVDLL |
| SEQ ID | MTEINGYTQSDNEKELHILNLPSLEAEAKKIIPTGGFGYISGGSEDEWTLNEN |
| NO: 18 | TKAFNNVQIVPRVLSDIDDPQTDTSIFGIDLTMPIITSPSAAQGLAHSQAEKDT |
| A0A0H4QF | ARGIAKAGTIMSQSTYASTSIADTAEAGNGAPQFFQLYMSKDWTFNESLLNE |
| B6 | AKRVGAKAILLTADATVDGYRESDIINNFQFPLPMANLEKFSSGAGKGKGIGE |
| Companilac- | IYAAAAQKISPDDVQRIKDITGLPVVVKGVQSPEDALIAIGAGADGVYVSNHG |
| tobacillus | GRQLNGGPASFDVLADVAKAVNHKVPVIFDSGIRRGSHVFKALASGADIVAL |
| ginsenosidimutans | ARPIIYGLALGGADGVYSVIEHLNNEFKTVMQLAGTKTIDDVKHAKLIKK |
| SEQ ID | MKSSHLIRVVASLAMLATSGLAYAEEYKASTDEKTIKMTNVASLEARVQARM |
| NO: 19 | DKGAFGYIRGGAEDENNLRSNTESFDKKYIMPRVLQGIELKEIDLSTQLLGIP |
| A0A0K0HA | LKTPIIQAPMAAQGLAHASGELATAKGMAQVGSIFSLSTYGNKTIADVAKVSG |
| W2 | KSPFFFQLYMSKNNKFNEFILSQAVKHGAKAIILTVDSPVGGYREEDIKNDFQ |
| Salmonella | FPLGFANLEMFARKNDDGSKTGKGAGISEIYAQAKQAFTPEDIAYVHRISGLP |
| bongori | VIIKGIQSPEDAEIAIQAGAAGIWVSNHGGRQLDSGPSSFDMLPAIAKVVNKR |
| NCTC | VPVIFDSGVRRGSHVFKALASGADIVAIGRPVLYGLNLGGAQGVASVIEQLN |
| 12419 | KELTINMMLGGARNIEQVKNTRLLSEKDL |
| SEQ ID | MSNKKYEASTAENHIDIINLASLEARVKDHMSNEKGAFGYISEGAEDEWTKK |
| NO: 20 | QNTEAFNKKQIMPRVIQGIDHADLSTQIFGIDLKSPIIQAPSAAHGLAHAKGET |
| A0A0K2LB | DTAKGVAAAGSIYSISTYASTSVEDAAAAAPDAPMFFQLYMSKDDKFNEFLIK |
| B9 | KAVKAGAKAIIMTVDSTLGGYREADAINKFQFPLPMPNLAGYSAGDGEGKGI |
| Companilac- | GEIYASAKQGIVPTDIQKIKDMSGLPVFVKGIQSPDDAALAIEFGADGIWISNH |
| tobacillus | GGRQLDGGPASFDMLPYIAEVVNKRVPIVFDSGVRRGEHVFKALASGADLV |
| heilongjiangensis | ALGRPIIYGLNLGGAQGVTDVIEHLNMELSITMQLAGTKTINDVKNTKLIG |
| SEQ ID | MTYQTSTENKAIEIVNIKSLEGKVKESMESAGNKGAFGYIRGGAEDEWTMG |
| NO: 21 | ENTSAFNKKQIMPRVLRGIDSADLSTSLFGIDLSTPIIQAPVAAQGLAHEEGEV |
| A0A0M2ZS | ATAKAMAEVGSIFSISTYGSTSVEDAAKAAPGAPQFFQLYMSKDDKFNEFFL |
| S8 | KKAVAAGVKAIILTADSTLGGYREEDIVNHFQFPLPMPNLAAFSESDGTGKGI |
| Lactococcus | SEIYAAAKQGLVLEDIKKIKEITKLPVIVKGIQSPIDADDAIKAGADGIWVSNHG |
| cremoris | GRQLDGGPASIDVLPLVAKIVNHRVPLIFDSGVRRGEHVFKALAQGADIIAIG |
| | RPVLYGLNLGGARGVQSVFEHLNKELSITMQLAGTKNIDEVKRTALID |
| SEQ ID | MGVTKLAYQASQEEHSIEITNVQALEGLVKARMEKGAFGYIAGGAEDEWTL |
| NO: 22 | RENGVAFDHKHIAPRVLKNIEKPDLSTTFMGIDLKIPMIAAPIAAQGLAHEQGE |
| A0A0R1HH | IDTAKGVAAVGSIMSLSTYSNKTISETCDGGTGGPQFFQLYMSKDEKFNRFL |
| 70 | LDQAKKVGMKAIILTADSTLGGNREADSINHFTFPIPMANLAEYGEGEGQGIA |
| Dellaglioa | AIYANAKQTLSLEDIKTISDYTDLPVFVKGIQSPLDIDDIIEAGAKGVWVSNHG |
| algida DSM | GRQLDGGPASFDVLESIAKKVNKRVPIVFDSGIRRGSHIFKALASGADVVAIG |
| 15638 | RPMVYGLHLGGAQGVQSVFEHLSKELTIDMQLAGTKTVDDIKKTELI |
| SEQ ID | MTYYYKGFPQSDRDEKISMINVDELEERAKKVMPEGAYYYIASGAENEWTW |
| NO: 23 | RANTSAFNHYQIVPRALTDMDDPQTDTEFMGMKLKTPIMISPIACHGIAHKDA |
| A0A0R1HI | EVATQKGAAAAGALFSSSTYANKSVEDIATAAPEAPRFFQLYLSKDWDFNK |
| G1 | MIFDAIKKAGYKGIFLTVDALVSGYREANLRTHFTYPVPLDFFTRYLGGKGEG |
| Lactobacillus | QSVAQMYASSAQKIGPEDVARIKKESGLPVFVKGVMCAEDAYKAIGAGADGI |
| amylovorus | YVTNHGGREVDGAPATIDVLPEIAKAVNHRVPIVFDSGVRRGSHVFKALALG |
| DSM 20531 | ADIVGIGRPYLYGLALGGPKGVESVINQLNTELKIDMQLTGCKTIDDVKRAKID |
| | RIHYGLDTMPSNTDPSRIKPYPVTADNQIKSEEPDATSGASKH |
| SEQ ID | MAIINGYEQSENEKELDILNLPSLEAQAKEIIPKGGFGYIVGGSEDEWTLHAN |
| NO: 24 | RSAFTHKQIVPKALSNIENPLTDTSVFGLNLKTPIMMAPAAAQGLAHVKGEVD |
| A0A0R1I08 | TAKGVASVGGLMAQSTYSSTSIADTAAAGDGAPQFFQLYMSKDWTFNESLL |
| 9 | DEAKRAGVKAIILTVDATVDGYREEDIVNHFQFPIPMANLTKFSEGDGAGKGI |
| Schleiferila- | GEIYAAAAQKIGPDDVRRIIEYTDLPVIVKGIESAEDALYAVGAGAAGVYVSNH |
| ctobacillus | GGRQLNGGPASFDVLEDVAKAVHGRVPVIFDSGVRRGSDVFKALASGADLV |
| kimchicus | AIGRPAIYGLALGGAQGVASVFNHLNDELKIIMQLAGTQTIEDVKHAPLLNIRY |
| JCM 15530 | |
| SEQ ID | MGFLMKKKYEASTAENHVDILNIAGLEARVKDHMSNEKGAFGYISGGSEDE |
| NO: 25 | WTKKQNTESFNKRKIEPRVLQGIDHADLSTKLWDIDLKTPIIQAPSAAQGLAH |
| A0A0R1I35 | AKGETDTAKGVAAAGSIFSISTYASTSIEDAAAAAPDAPQFFQLYMSKDDNF |
| 1 | NEFLIKKAVAAGAKAIVLTVDSTLGGYREADVANKFQFPLPMPNLAGYSAGD |
| Companilac- | GEGKGISEIYASAKQGIVPTDIQKIKNMSGLPVIVKGIQSPDDAELAIEFGADGI |
| tobacillus | WVSNHGGRQLDGGPASFEVLPDIADAVDQRVPVIFDSGVRRGEHVFKALAS |
| kimchii | GADLVAIGRPIIYGLNLGGAQGVTDVIEHLNMELSITMQLAGTKTINDVKNTEL |
| DSM 13961 | LW |
| SEQ ID | MMNKEKYETSTAENHVDIINLASLEDRVKDHMSNEKGAFGYIRGGAEDEWT |
| NO: 26 | LNENTEAFNDKEIMPRVLQGIDHADLSTKLWDIDLKTPIIQAPSAAHGLAHVK |
| A0A0R1IY4 | GETDTAKGVAAAGSIYSISTYASTSVEDASAAAPDSPMFFQLYMSKDDKFNE |
| 7 | FLIKKAVKAGAKAIIMTVDSTLGGYRESDVINKFQFPLPMPNLAGYSAGDGEG |
| Companilac- | KGIGEIYASAKQGIVPTDIQKIKDMSGLPVFVKGIQSPDDAELAIQFGADGIWV |
| tobacillus | SNHGGRQLDGGPASFALLPYIAEVVDKRVPIVFDSGVRRGEHVFKALASGA |
| tucceti | DLVALGRPIIYGLNLGGAQGVTDVIEHLNMELAITMQLAGTKTIDDVKNAELLD |
| DSM 20183 | |
| SEQ ID | MTVINGYEQSDREQKLDILNLPSLEEQAKAIIPTGGFGYIVGGAEDNWTLRQ |
| NO: 27 | NTKAFAHAQIVPKALSNIENPDLSTSIFGIPLKTPVMMAPAAAQGLAHSQGEK |
| A0A0R1JX | DTAKGVAAVGGLMSQSTYSSTSIADTQAAAPDAPQFFQLYMSKDWTFNESL |
| M9 | LDEAKKAGAKAIILTVDATVDGYREADIINNFQFPIPMANLTKFSEGDGKGKGI |
| Lacticasei- | GEIYASAAQKISEADVRRIAEYTDLPVIVKGIESPEDALRAIGAGAAGIYVSNH |
| bacillus | GGRELNGGPAAFDVLPAIAKAVNHQVPIIFDSGVRRGSHVFKALAAGADLVA |
| nasuensis | FARPVIYGLALGGALGVQSVFEEIDHELAITMQLAGTQTIADVKHAPLTHFNY |
| JCM 17158 | ED |
| SEQ ID | MTETNGYFQNDNEKELNIVNLPSLEAEAEKIIPKGGFGYIAGGSEDNWTLKA |
| NO: 28 | NTEAFNHVQIVPHVLSDIEDPQTNTSIFGIDVKTPIMMTATAAQGLAHAKGEM |
| A0A0R1KH | DTAKGIAKAGALMEQSTYSSTSIADTMAAGNGAPQFFQLYMSKDWTFNESL |
| M3 | LKEAKQAGAKAIVLTADATVDGYRESDIINDFQFPTPMANLTKFSEGDGEGK |
| Companilac- | GIGEIYAAAAQKISPKDIQRIKDIAGLPVIVKGVQSPEDALLAIGAGADVIQVSN |
| tobacillus | HGGRQLNGGPASFDVLSDVAKAVNHRVPIIFDSGVRRGSHVFKALASGADM |
| nodensis | VAMGRPVIYGLALGGADGVYSVVEHLNDEFKTIMQLAGTKTIEDVKHAKLLK |
| DSM 19682 | K |
| SEQ ID | MTVVNGYKQNDTEQKLDILNLPELEEKAKQIIPTGGFGYIAGGSENNWTLKA |
| NO: 29 | NRTAFTHKQIVPRALSNIEKPELDTNVFGIPLKTPIMMAPTAAQGLAHSQGEK |
| A0A0R1L6 | DTAKGVAAVGGLMAQSTYSSTSIADTAAAGNGAPQFFQLYMSKDWDFNYS |
| X4 | LLDEAKKAGVKGIILTVDATVDGYREDDIKNNFQFPIPMANLTKFSEGDGKGK |
| Lentilacto- | GIGEIYASAAQKIGPDDVKKIADYTDLPVIVKGIESPEDALYAIGAGAAGVYVS |
| bacillus | NHGGRQLNGGPASFDVLEDVAKAVNGQVPIIFDSGVRRGSDVFKALASGAD |
| sunkii DSM | LVAMGRPVIYGLALGGAEGVQSVFEHLGDELKIIMQLAGTKTIADVKKANLLN |
| 19904 | IKY |
| SEQ ID | MTIFNGYEQSDREQKLNILNLPSLEAEAKKIIPTGGFGYISGGSEDEWTLHEN |
| NO: 30 | TSAFNHIQIIPRALTNVDQPSTNTKVFGLNLKTPIMMAPAAAQGLAHSRGEKA |
| A0A0R1MH | TAEGLAEVGGLMAQSTYSSTSIADTAAAGNGAPQFFQLYMSKDWEFNYSLL |
| 81 | DEAVKAGAKGIILTVDATVDGYRESDIINNFQFPIPMANLAKFSEGDGKGKGI |
| Liquorilacto- | MEIYAAAAQKISPDDVRRIVKYTKLPVIVKGIESAEDALLAIGAGAEGIYVSNH |
| bacillus | GGRQLNGGPASIDVLHDVAKAVNHQVPVIFDSGVRRGSHVFKALASGADLV |
| hordei DSM | ALARPIIYGLALGGAKGVSSVINHLNDELKIDMQLAGTKTIEDVKKAKILRK |
| 19519 | |
| SEQ ID | MTVTNGYEQSDREQKLDFINVNDLEKEAEKIIPKGGFGYIRGGSEDEWTLRE |
| NO: 31 | NTLAFNHAQIIPRALTDMENPSTDTEAFGLHFKTPLMMAPTAAQGLAHAKGE |
| A0A0R1N4 | TDTARGVAAAGALMAQSTYSSTSIADTAAAANGAPQFFQLYMSKDWDFNH |
| P4 | HLLDEAKKAGIKGIILTLDAPVDGFREDDMRNHFQFPIPMANLTEYSEGDGA |
| Schleiferilacto- | GKGIGEIYAAAAQKIGPKDIERIAEYTDLPVIAKGIMSPEDALKAIGAGAAGVY |
| bacillus | VSNHGGRQLNGGPASFDVLPSIAAAVNHQVPIIFDSGVRRGSHVFKALASGA |
| perolens | DLVAFGRPAIYGLALGGAEGVQGVFEHLNAELKIDMQLAGTKTIADVQHAKL |
| DSM 12744 | AHFPA |
| SEQ ID | MKKKYEASTAENHVDILNIAGLEARVKDHMSNEKGAFGYISGGSEDEWTKK |
| NO: 32 | QNTESFNKRKISPRVLQGIDYADLSTKLWDIDLKTPIIQAPSAAQGLAHVKGE |
| A0A0R1PG | TDTAKGVAAAGSIFSISTYASTSIEDAAAAAPDAPQFFQLYMSKDDKFNEFLI |
| 98 | KKAVAAGAKAIVLTVDSTLGGYREADVANKFQFPLPMPNLAGYSAGDGEGK |
| Companilacto- | GISEIYASAKQGIVPTDIEKIKNMSGLPVIVKGIQSPDDAELAIDFGADGIWVSN |
| bacillus | HGGRQLDGGPASFEVLPDIADAVDQRVPVIFDSGVRRGEHVFKALASGADL |
| paralimentarius | VAIGRPIIYGLNLGGAQGVTDVIEHLNMELSITMQLAGTKTINDVKNTELLW |
| DSM 13238 | |
| SEQ ID | MTTINGYEQSDREEKLDILNLPSLEAEAKKIIPKGGFGYISGGSEDEWTLHEN |
| NO: 33 | TTAFNHVQIIPRALTDMEQPTTATSVFGINLKTPIMMAPAAAQGLAHSRGEEA |
| A0A0R1QC | TAEGLAQAGGLMAQSTYSSTSIADTAAAGKGAPQFFQLYMSKDWDFNRSLL |
| G7 | DEAVKAGAKGIILTVDATVDGYRESDIINNFQFPIPMANLAKFSEGDGKGKGI |
| Liquorilac- | MEIYAAAAQKIAPADVRKIAEYTHLPVIVKGIESAEDALLAIGAGAQGIYVSNH |
| tobacillus | GGRQLNGGPAAFDVLHEVAQAVDHRVPVIFDSGVRRGSHVFKALASGADL |
| nagelii | VALARPVIYGLALGGAQGVASVINHLNDELKIDMQLAGTKTIEDVKQAKLIRH |
| DSM 13675 | |
| SEQ ID | MNKKKYEASTAENHVEIVNIASLEKRVQDHMSNEKGAFGYIRGGAEDEYTM |
| NO: 34 | KQNTEAFNKAKIMPRVIQGIDHADLSTKLWDIDLKTPIIQAPSAAQGLAHAKG |
| A0A0R1QQ | ETDTAKGVAAAGSIFSISTYASTSVEDAAAAAPDAPQFFQLYMSKDDKFNEF |
| G9 | LIKKAVAAGVKAIVLTVDSTLGGYREADVINKFQFPLPMPNLAGYSAGDGEG |
| Companilacto- | KGISEIYASAKQGIVPADIQKIKDMSGLPVFVKGIQSPDDAELAIEFGADGIWV |
| bacillus | SNHGGRQLDGGPASFDVLPDIADRVDKRVPIVFDSGVRRGEHVFKALASGA |
| mindensis | DLVAIGRPIIYGLNLGGAQGVTDVIDHLNMELSITMQLAGTKTINEVKNTDLYY |
| DSM 14500 | |
| SEQ ID | MTVINGYEQSDRNEKLDILNLPSLEARAKKIIPQGGFGYIVGGAEDDWTLQQ |
| NO: 35 | NTKAFTHAQIVPKALSNIENPDLSTNVFGIDLKTPVMMAPAAAQGLSHSLGEK |
| A0A0R1RA | ATAQGVAAVGALMSQSTYSSTSIADTQAAAPDAPQFFQLYMSKDWDFNNAL |
| 27 | LDEAMKAGVKGIILTVDATVDGYREQDIINNFQFPIPMANLERFAAGDGKGK |
| Lacticasei- | GIGEIYAAAAQKISEDDVRRIAEYTKLPVIVKGIESPEDAMRAIGAGAAGIYVS |
| bacillus | NHGGRELNGGPAAFDTLSAIAKAVNHQVPIIFDSGVRRGSHVFKALASGADL |
| manihotivorans | VAFARPVLYGLALGGALGVQSVFEQIDHELEIVMQLAGTKDIEAVKHAPLTHF |
| DSM 13343 | HYEN |
| SEQ ID | MTVFYKGFPQSDRNQAIKMVNVDELEERVKKVMPEAAYYYIASGSENEWT |
| NO: 36 | WRNNTAAFNHFQIIPRSLTDMDNPSTDTKFMGMDLKTPVMICPIACHGIAHK |
| A0A0R1S3 | DAEVATAEGAKAAGALFSSSTYANRSVEDIAGATGDSPKFFQLYLSKDWDF |
| 57 | NKMVLDAVKAAGYKGIMLTVDALVSGYREANLRTNFTYPVPLDFFTRYGKG |
| Lactobacillus | EGMSVAQMYANSAQKIGPKDVEKIKELSDLPVFVKGIMNAEDAYLAMGAGA |
| psittaci | DGIVVSNHGGREIDTAPATIDMLPEIAAAVNGRVPIILDSGVRRGSHVFKALAL |
| DSM 15354 | GADLVGIGRPFLYGLALGGAKGVESVINQINNEFKILMQLTGCKTVEDVKHA |
| | DIRQINYTADNLPSNTDPAVRKPYPVTKDNQLEGTQDAVSGASKH |
| SEQ ID | MAVVNGYKQSENEKKLNILNLPELEQQAKEIIPTGGFGYISGGSEDEWTLRA |
| NO: 37 | NRKAFTHKQIVPRALSNIEKPVLDTTVFGFPLKTPVMMAPTAAQGLAHAKGE |
| A0A0R1SK | VDTAKGVAAVGGLMAQSTYSSTSISDTAAAGDGAPQFFQLYMSKDWTFNE |
| 65 | SLLDEAKKAGVKGIILTVDATVDGYREADIINNFQFPIPMPNLTQYSEGNGQG |
| Lentilacto- | KGIAEIYASAAQKIGPKDVERIASYTDLPVIVKGIESPEDALYAIGAGAAGVYV |
| bacillus | SNHGGRQLNGGPASFDVLADVAKAVNGKVPVIFDSGVRRGSDVFKAIASGA |
| diolivorans | DLVAIGRPAIYGLALGGAQGVQSVFKHLDQELEIIMQLAGTKTIDDIKKTNLLGI |
| DSM 14421 | NY |
| SEQ ID | MVLTNGYEQNENENPLDILNLTELEGKAKAIIPTGGFGYISGGSEDEWTLRA |
| NO: 38 | NRTAFQHRQIVPKALSNIENPSTDTTVFGIDLKTPIMMAPTAAQGLAHAKGEV |
| A0A0R1TL | DTAAGVAAAGGLMAQSTYSSTSIADTAAGGHGAPQFFQLYMSKDWSFNES |
| V1 | LLDEAKKAGVKGIILTVDATVDGYREADIINNFQFPIPMANLTKFSEGDGKGK |
| Schleiferilacto- | GIADIYAAAAQKIGPDDVRRIAEYTDLPVIVKGVESPEDALYAIGAGAAGIYVS |
| bacillus | NHGGRQLNGGPASFDVLESVAKAVNHRVPVIFDSGVRRGSDVFKALASGA |
| paracollinoides | DLVAMGRPAIYGLALGGAQGVESVFTHLNDELKIIMQLAGTKTIADVKHAPLL |
| DSM 15502 | DIKY |
| SEQ ID | MTLTNGYEQSDREEKLNILNLPSLEGEAKKIIPTGGFGYIFGGAEDEWTLKEN |
| NO: 39 | TQAFNHAQIVPRALSNIDHPDLSTDVFGLHLKTPVMMAPTAAQGLAHAKGET |
| A0A0R1U6 | DTAKGVAAVGGLMAQSTYSSTSIADTAAAGNGAPQFFQLYMSKDWTFNESL |
| S2 | LDEAKKAGVKAIILTVDATVDGYREEDIINNFQFPIPMANLEKFASGDGKGQGI |
| Lacticasei- | GEIYAAAAQKIGPDDVRRIAEYTDLPVIVKGIQSPEDALRAIGAGAAGIYVSNH |
| bacillus | GGRQLNGGPASFDVLADIAKAVNHQVPIIFDSGVRRGSHVFKALASGADLVA |
| pantheris | FGRPVIYGLALGGAEGVQSVFEAIDHELEITMQLAGTKTIEDVKHAPLTHFQY |
| DSM 15945 | ND |
| SEQ ID | MTSYFKGFPQSDRNEKIDMINVDELEERAKKVMPEGAYYYIASGSENEWTW |
| NO: 40 | RANTSAFNHYQIVPRALTGMDNPSTETEFMGMKLKTPIMISPIACHGIAHKDA |
| A0A0R 1UD | EVATQEGAAAAGALFSSSTYANKSVEDIAAAAPDAPRFFQLYLSKDWDFNK |
| M0 | MVFEAINKAGYKGIFLTVDALVSGYREANLRTKFTYPVPLDFFTRYLGGKGE |
| Lactobacillus | GQSVAQMYASSAQNISPADVERIKKESGLPVFIKGVMCAEDAYKALGAGAD |
| kalixensis | GIYVTNHGGREVDGAPATIDVLPEIAKAVNHRVPIIFDSGVRRGSHVFKALAM |
| DSM 16043 | GADLVGIGRPYLYGLALGGAKGVESVIDQLDDELKIDMQLTGCKTIDDVKHA |
| | KIDSIHYGRDDLPSNTDPSRIKPYPKTADNQIKQEAETDATSGASHH |
| SEQ ID | MTTINGYEQSDREQKLNILNLPALEAEAKKIIPKGGFGYISGGSEDEWTLREN |
| NO: 41 | TKAFNHVQIVPRALTEMEHPTTETTVFGIKLKTPIMMAPAAAQGLAHSRGEE |
| A0A0R1VN | ATAEGLAQAGGLMAQSTYSSTSIAATAAAGKGAPQFFQLYMSKDWDFNRSL |
| Y0 | LDEAVKAGAKGIILTVDATVDGYREADIINQFQFPIPMANLAKFSEGDGKGKG |
| Liquorilac- | IMEIYAAAAQKIGPDDVRKIAEYTNLPVIVKGIESAEDALLAIGAGAQGIYVSNH |
| tobacillus | GGRQLNGGPAAFDVLHEVAQAVDHQVPVIFDSGVRRGSHVFKALANGADL |
| ghanensis | VALARPIIYGLALGGAQGVASVVNHLNDELKIDMQLAGTKTIADVKQAKLIRH |
| DSM 18630 | |
| SEQ ID | MTYYYKGFPQSDRDEKISMINVDELEERAKKVMPEGAYYYIASGAENEWTW |
| NO: 42 | RANTSAFNHYQIVPRALTDMDDPQTDTEFMGMKLKTPIMISPIACHGIAHKDA |
| A0A0R1VW | EVATQKGAAAAGALFSSSTYANKSVEDIAAAAPEAPRFFQLYLSKDWDFNK |
| F9 | MVFDAIKKAGYKGIFLTVDALVSGYREANLRTHFTYPVPLDFFTRYLGGKGE |
| Lactobacillus | GQSVAQMYASSAQKISPEDVARIKKESGLPVFVKGVMCAEDAYKAIGAGAD |
| kitasatonis | GIYVTNHGGREVDGAPATIDVLPEIAKAVNHRVPIVFDSGVRRGSHVFKALAL |
| DSM 16761 | GADIVGIGRPYLYGLALGGPKGVESVIDQLDNELKIDMQLTGCKTIADVKHAK |
| | IDRIHYGLDTMPSNTDPSRMEPYPVTADNQIKSQEPDATSGASKH |
| SEQ ID | MGFLMMNKKKYEASTAENHVDIVNLASLEARVKDHMSNEKGAFGYIRGGAE |
| NO: 43 | DEWTLNENTEAFNDKEIMPRVLQGIDHADLSTKLWDIDLKTPIIQAPSAAHGL |
| A0A0R1W | AHIKGETDTAKGVAAAGSIYSISTYASTSVEDAAAAAPDSPMFFQLYMSKDD |
| GZ3 | KFNEFLIKKAVKAGAKAIILTVDSTLGGYRESDVINKFQFPLPMPNLAGYSAG |
| Companilacto- | DGEGKGIGEIYASAKQGIVPTDIQKIKDMSGLPVFVKGIQSPDDAELAIQFGA |
| bacillus | DGIWVSNHGGRQLDGGPASFALLPYIAEVVDKRVPIVFDSGVRRGEHVFKA |
| nantensis | LASGADLVALGRPIIYGLNLGGAQGVTDVIEHLNMELAITMQLAGTKTIDDVK |
| DSM 16982 | NTELLD |
| SEQ ID | MTYYYKGFPQSTRDEVLHMVNVEELARKAKDVMPEAAYYYVASGAENEWT |
| NO: 44 | WRANTSAFNHYQIIPRALTNMSHPQLDTEFMGMKLKTPVMIAPIACHGIANK |
| A0A0R1XW | AAEVGTQKGAAAAGALFSSSTYANKSVEDIAAAAPNAPRYFQLYLSKDWKF |
| H7 | NRMVFDAIKKTGYKGIWLTVDALVSGYREANLRTHFTYPVPLDFFTRYLGGK |
| Lactobacillus | GEGQTVAQMYAASAQKIGPKDVQRIKDESGLPVFIKGITNAEDAYKAIGAGA |
| intestinalis | DGVYVTNHGGREVDGEPATVDDLPEVAKAVAHRVPVVFDSGVRRGSHVFK |
| DSM 6629 | ALALGADMVGIGRPFLYGLALGGAKGVKSVIDQYNKELLIDMELTGCKTIEDV |
| | KHAKIAHIRYTADNLPSDTDPTRMKPYPVTKYNQMKSDEADASSGASEA |
| SEQ ID | MAIVNGYKQNENEKKLDILNLPELEEKAKQIIPTGGFGYIVGGSEDEWTLRAN |
| NO: 45 | RQAFTHKQIVPRALSNIEKPELDTNVFGLPLKTPIMMAPTAAQGLAHVEGEK |
| A0A0R1Z0 | DTARGVAAAGGLMAQSTYSSTSISDTSAAGNGAPQFFQLYMSKDWTFNES |
| F7 | LLDEAKKAGVKGIILTVDATVDGYREADIINNFQFPIPMANLTKFSEGDGKGK |
| Lentilacto- | GIAEIYASAAQKIGPDDVKRIADYTDLPVIVKGIESPEDALYAIGAGAAGVYVS |
| bacillus | NHGGRQLNGGPASFDVLEDVAKAVNGRVPIIFDSGVRRGSDVFKALASGAD |
| parafarraginis | LVALGRPAIYGLALGGAQGVQSVFEHLGDELKIIMQLAGTKTIADVKKTNLLNI |
| DSM 18390 | KY |
| SEQ ID | MTITNGYKQNENEKELDILNLPALEQEAQKIIPTGGFGYIAGGSEDEWTLHAN |
| NO: 46 | RTAFQHKQIVPKALSDIEDPQTDTRVFNLDLKTPIMMAPAAAQGLAHVKGEID |
| A0A0R2A1 | TAAGVAAAGGLMAQSTYSSTSIADTAEAGNGAPQFFQLYMSKDWNFNESLL |
| Z3 | DEAKKAGVKGIILTVDATVDGYREADIINHFQFPIPMANLTKFSEGDGKGKGI |
| Paucilacto- | GEIYASAAQKIGPKDVARIIQYTDLPVIVKGIESAEDALYAIGAGAAGIYVSNHG |
| bacillus | GRQLNGGPASFDVLTDVASAVNHRVPIIFDSGVRRGSDVFKALAAGADLVAI |
| vaccinostercus | GRPAIYGLALGGAQGVTSVFNHLNDELKIIMQLAGTKTIEDVKHAPLLDIKY |
| DSM 20634 | |
| SEQ ID | MTVVNGYEQSDNEKKLDILNLPALEDEAKKIIPTGGYGYIFGGSEDDWTLKQ |
| NO: 47 | NTLAFEHAQIVPKALSDIEKPDLSTNVFGIDLKTPVMMAPTAAQGLAHAQGE |
| A0A0R2AX | DDTARGVAAVGGLMAQSTYSSTSIADTAAAGNGAPQFFQLYMSKDWSFNE |
| 80 | SLLDEAKKAGVKAIILTVDATVDGYREQDIINNFQFPIPMANLTKFSEGDGKG |
| Lacticasei- | KGIADIYASAAQKIGPDDVRRIAEYTDLPVIVKGVQSPEDAMRAIGAGAAGIYV |
| bacillus | SNHGGRQLNGGPASFDVLESIAKAVNHQVPIIFDSGVRRGSHVFKALAAGA |
| brantae | DLVAFGRPVIYGLALGGAQGVQSVFEHIDHELEIIMQLAGTKTIEDVKHAPLL |
| DSM 23927 | HIHYND |
| SEQ ID | MVLTNGYEQNENEKALDILNLTDLEKQAKAIIPTGGFGYISGGSEDEWTLRA |
| NO: 48 | NRTAFQHRQIVPKALSNIENPRTDTSVFGLDLKTPLMMAPAAAQGLAHAKGE |
| A0A0R2BF | VDTAAGVAAAGALMAQSTYSSTSIADTAAGGNGAPQFFQLYMSKDWAFNE |
| C9 | SLLDEAKKAGVKGIILTVDATVDGYREADIINNFQFPIPMANLTKFSEGDGKG |
| Schleiferilacto- | KGIGEIYAAAAQKIGPDDVRRIADYTDLPVIVKGVESAEDALYAIGAGAAGIYV |
| bacillus | SNHGGRQLNGGPASFDVLESVAKGVNHRVPVIFDSGVRRGSDAFKAIALGA |
| collinoides | DLVAIGRPAIYGLALGGAQGVESVFNHLNDELKIIMQLAGTQTIADIHGAPLLD |
| DSM 20515 | IKY |
| SEQ ID | MMTIVNGYEQSDREEHINVLNLESLEARAEKIIPKGGFGYISGGSEDNWTLA |
| NO: 49 | QNRKAFTHKQVYPRALANIDQPDLSANVFGLDLKTPVMMPPLAAQGLAHAK |
| A0A0R2BJ | GEMDTAKAFAAEGALMAQSTYSSASIADTAAAGNGAPQFFQLYMSKDWKF |
| 23 | NEAVIDEAKKAGVKGIILTVDATVDGYREADIINHFQFPIPMANLEKFSQGAGK |
| Lapidilacto- | GQGIAEIYAAAAQKISADDVRRIAEYSDLPVIVKGIQTPEDAELAIGAGASGIYV |
| bacillus | SNHGGRQLNGGPGSFDTLSAIAQAVNGRVPVIFDSGVRRGSDVFKALAAGA |
| dextrinicus | DLVAIGRPFVYALALGGALGVQDALQEINHEFATIMQLAGTKTIADVKKAKLA |
| DSM 20335 | DFNY |
| SEQ ID | MTLTNGYEQSDREEQLHILNLPSLEAAAKKIIPTGGFGYIYGGAEDEWTLKAN |
| NO: 50 | TQAFNHVQIVPRALANIDHPDLSTEIFGLHLDTPVMMAPAAAQGLAHAKGEV |
| A0A0R2CI | DTAKGVAAVGGLMAQSTYSSTSIADTAAAGDGAPQFFQLYMSKDWTFNASL |
| W2 | LDEAKRAGVKAIILTVDATVDGYREEDIVNNFQFPIPMANLEKFANGDGKGQ |
| Lacticasei- | GIGEIYAAAAQKISPDDVRRIADYTDLPVIVKGIQSPADALRAIGAGAAGIYVS |
| bacillus | NHGGRQLNGGPASFDVLADIAKAVNHQVPIIFDSGVRRGSHVFKALAAGADL |
| thailandensis | VALARPVIYGLALGGAQGVQSVFEALDHELAITMQLAGTKTIEDVKHAPLTHF |
| DSM 22698 | RYDD |
| SEQ ID | MTVVNGYEQSDREEHINVLNLESLEKRAKEIIPTGGFGYIVGGSEDDWTLAQ |
| NO: 51 | NRKAFTHKQIYPRTLANIDNPDMSTNVFGIDLKTPVMMAPLAAQGLAHSKGE |
| A0A0R2CP | VDTAKAFANEGALMAQSTYSSASIADTAAAGEGAPQFFQLYMSKDWKFNEA |
| H0 | ILDEAKKAGVKGIILTADATVDGYREADIINDFQFPIPMANLEKFSEGAGKGQ |
| Lentilacto- | GIAEIYAAAAQKISPDDVRRIAEYSDLPVIVKGIQTPEDAELAIGAGAAGIYVSN |
| bacillus | HGGRQLNGGPASFDALSAIADSVAGRVPIIFDSGVRRGSDVFKALAAGADLV |
| senioris | ALGRPFVYALALGGALGVQDAIQELNHEFATTMQLAGTKTIDEVKQSKLADF |
| DSM 24302 | KY |
| SEQ ID | MTIFNGYEQSDREQKLDILNLPSLEEEAKKIIPTGGFGYISGGSEDEWTLHEN |
| NO: 52 | TSAFNHIQIIPRALTDVDQPSTDTEVFGLKFKTPIMMAPAAAQGLAHSRGEEA |
| A0A0R2CX | TAEGLAEVGALMAQSTYSSTSIADTAAAGKGAPQFFQLYMSKDWEFNYSLL |
| A0 | DEAVKAGAKGIILTVDATVDGYRESDIINNFQFPIPMANLAKFSEGDGKGKGI |
| Liquorilacto- | MEIYAAAAQKISPADVRRIAEYTKLPVIVKGIESAEDALLAIGAGAKGIYVSNH |
| bacillus | GGRQLNGGPASIDVLHEVAVAVNHQVPVIFDSGVRRGSHVFKALASGADLV |
| cacaonum | ALARPIIYGLALGGAQGVASVINHLNDELKIDMQLAGTKTIEDVKKAKVIRK |
| DSM 21116 | |
| SEQ ID | MTTYYKGFPQSTREEKLHMVNLNELENEAKYVIPEAAYYYIASGAENEWTW |
| NO: 53 | RNNTQAFNHFQIVPRALTGMQDPELNTEFLGMKLKTPVMICPIACHGIANAE |
| A0A0R2D91 | AEIDTAKGAKAAGSLFGMSTYANKSVQEVQAAVGDSPRYMQLYLSKNWDF |
| 5 | NKMVIEESVKAGFTGFFLTVDALVSGYREANLRTNFTYPVPLAFFNEWNGG |
| Lactobacillus | KGEGQSVAQMYASSAQNIGPDDIRKIKEIADVPVIVKGIECAEDAMLAIGAGA |
| taiwanensis | DGIVVSNHGGREVDGAPATIDVLPEIAKAVKSYDHRVPIILDGGVRRGSHVFK |
| DSM 21401 | ALALGADLVGIGRPFLYGLALGGAQGVQSVIEQLNKELLIDMQLTGCKTIEDIK |
| | HAKIDHIDYTADWGISSTSKSVMKPYPVTKENQLTGEAADAVSGASRH |
| SEQ ID | MTYNASTAEEKLDIIDLPRLRALVESRTEAGAFGYVDGGSSDEQVLQDNETA |
| NO: 54 | FRHYQLIPRMLQNIAEPDMTTTLFDIPLGMPIIAAPIAAQGLMHEGGESVTVK |
| A0A0R2FC | GVGAAKSIFSASTYGNASVAAVAEASPETPKFFQLYMSRDDEFNQFLINQAV |
| 09 | ETGYKAIILTADSTLGGYREADIINNFEFPLPMDNLAAFSNAAGTGEGLSIAEI |
| Weissella | YARAKQDLALSDIKKIKDMASGLPVIVKGIQDPEDALAAIAGGADGIWVSNHG |
| confusa | GRELNGAPASIDVLADIAKAVNHRVPIIFDSGIRWGEDIAKAIALGADVVALGR |
| | PMLWALNLGGAAGVQSAFEHLAEELKIVMQLTGSHTVAELKHAKIIEAKF |
| SEQ ID | MTMVNGYEQSDREEKIDILNLESLEGRAKEIIPTGGFGYIVGGSEDEWTLKK |
| NO: 55 | NRDAFHHRQIAPKALSGMENPDLSTNVFGIPLKTPVMMAPTAAQGLAHSQG |
| A0A0R2KZ | EKDTAKGVAAVGGLMAQSTYSSTSIADTAAAADGAPQFFQLYMSKDWSFN |
| Y0 | ESLLDEAKEAGVKAIILTVDATVDGYREADIINDFAFPIPMANLTKFSEGDGQG |
| Pediococcus | KGIEEIYASAAQNIKPEDVRRIAEYTDLPVIVKGIQTPEDAIRAIDAGAAGIYVS |
| stilesii | NHGGRQLNGGPASFDVLESISKAVNGQVPIIFDSGVRRGSDVFKALASGADL |
| | VAFGRPVIYGLALGGAQGVQAVFDHIDHELEIVMQLAGTKTIEDVKKTHLLDI |
| | KY |
| SEQ ID | MKKKYEASTAENHVEIVNIAGLEARVKDHMSNEKGAFGYIRGGAEDEYTMK |
| NO: 56 | ENTAAFNKAKIMPRVLQGIDHADLSTNLWDIALKTPIIESPSAAQGLAHANGE |
| A0A0R2LM | KDTAKGVAAAGSIFSMSTYGSTSLEDGAAAAPDAPQFFQLYMSKDDKFNEF |
| 15 | LINKAVKAGVKAIVLTVDSTLGGYREEDVINKFQFPLPMPNLAAYSEGDGEG |
| Companilacto- | KGIGEIYAAAKQGIVPSDIQKIKDMSGLPVIVKGIQSPDDADLAIEFGADGIWV |
| bacillus | SNHGGRQLDGAPASFDILPDIADRVDKRVPVIFDSGVRRGEHVFKALASGAD |
| kimchiensis | LVAVGRPIIYGLNLGGAQGVTDVIEHLNMELSITMQLAGTKTINDVKNTDLYY |
| SEQ ID | MTVVNGYEQSDNEKKLDILNLPALEDEAKKIIPTGGYGYIFGGSEDDWTLRQ |
| NO: 57 | NTKAFEHAQIVPKALSDIESPDLSTNVFGLDLKTPVMMAPTAAQGLAHAKGE |
| A0A0R2MZ | ADTARGVAAVGGLMAQSTYSSTSIADTAAAGNGAPQFFQLYMSKDWSFNE |
| Z8 | SLLDEAKKAGVKAIILTVDATVDGYREQDIINNFQFPIPMANLTKFSEGDGKG |
| Lacticasei- | KGIGEIYASAAQKIGPDDVRRIAEYTDLPVIVKGIQSPEDALRAIGAGAAGIYV |
| bacillus | SNHGGRQLNGGPASFDVLESIAKAVNHQVPIIFDSGVRRGSHVFKALAAGA |
| saniviri | DLVAFGRPVIYGLALGGAEGVQSVFEHIDHELEIIMQLAGTKTIEDVKHAPLLH |
| JCM 17471 | INYAD |
| SEQ ID | MTHYYEGFPQSDRDEKIKMVNVDELEERVKKVMPEGAYYYIASGAENEWT |
| NO: 58 | WRNNTAAFNHFQIVPRALTEMADPQTDTDFMGMHLKTPIMIAPIACHGIAHK |
| A0A109DE | DAEVATQKGAAMAGALFSSSTYANKSVEEIAAAAPEAPRFFQLYLSKDWNF |
| J7 | NQMVFDAIKKAGYQGIFLTVDALVSGYREANLRTNFTYPVPLDFFKRYLGAK |
| Lactobacillus | GKGQSVAQMYASSAQKIGPEDVKRIKKESGLPVFVKGVMCAEDAYKAIGAG |
| crispatus | ADGIYVTNHGGREVDGAPATIDVLPEIAQAVNHRVPIIFDSGVRRGSHIFKAL |
| | ALGADIVGIGRPYLYGLALGGAHGVASVIEQLNDELKIDMQLTGCKTIDDVKH |
| | AKLTHIAYTADNLPSNTDPSRRKEYPVTDENQVKQTDAVSGASKLEGKPAP |
| | EPETDTTTGASVR |
| SEQ ID | MTTYYKGFPQSTREEKLHMVNLDELENEAKYVMPEAAYYYVASGAENEWT |
| NO: 59 | WRNNTQAFNHFQIVPRALTGMQNPELNTEFLGMKLKTPVMICPIACHGIANA |
| A0A137PM | EAEIDTAKGAKAAGALFAMSTYANKSVQEVQSAVGDSPRFMQLYLSKNWDF |
| L5 | NKMVIEESVKAGFTGFFLTVDALVSGYREANLRTNFTYPVPLAFFNEWNGG |
| Lactobacillus | KGEGQSVAQMYASSAQNIGPDDIRKIKEIADVPVIVKGVECAEDAMLAIGAGA |
| johnsonii | DGIVVSNHGGREVDGAPATIDVLPEIAKAVRSCDHRVPIILDGGVRRGSHVF |
| | KALALGADLVGIGRPFLYGLALGGAQGVQSVIEQLNKELLIDMQLTGCKTIEDI |
| | KHAKIDHINYSADWGISSTSRSVMKPYPVTKENQLTGEAADAVSGASRH |
| SEQ ID | MLHRAKKQPIAVMSSAILALTLSVGAVQAADYQASTKEGPIKIINLDELEDQVA |
| NO: 60 | KNMEKGAFGYIRGGAEDELNLDKNTRSFDRKYIMPRVMQGIEIKDIDLSTQFL |
| A0A140NL | GINLKTPIIQAPMAAQGLAHQDGEIATAKGMAKAGSIFSLSTYGNKTIEEVAEV |
| X7 | SGESPFFFQLYMSKNNAFNEFTLKRAKESGAKAIILTVDSPVGGYREDDIRN |
| Providencia | NFQFPLGFANLELFAKQNDDGSKTGKGAGISEIYAQAKQAFTPADIAYVKKL |
| stuartii | SGLPVIVKGIQSPEDADRVIKAGADAIWVSNHGGRQLDSGPASFDVLPSIAK |
| MRSN | VVNKRVPIVFDSGVRRGSHVFKALASGADVVAVGRPILYGLNLGGSEGVNS |
| 2154 | VIQQLNKELSINMMLGGAKNIESVKATKLYTDMDFQ |
| SEQ ID | MKKIIMTLTAITLTTSVFAHTTTETVFQNKDKKTKGYQASTADKKLKIVNLYEL |
| NO: 61 | EGQVKAEMDRGAFGYIVGGAEDQNNLKINTENFDKKYIMPRVLKGIKHEDID |
| A0A161SL | LSTSLFGIPLKTPIIKAPMAAQGLSHVDGEIATAKGMIAAGSLFSLSTYGNKTIE |
| A2 | EVAEGINGAPFFFQLYMSKNDEFNKFTLERAKKYGAKAIILTVDSPVGGYRE |
| Myroides | EDIKTGFTFPLGMGNLELFAAQQADGNKTGKGAGITEIYAQAKQDFKPSDIK |
| marinus | YVKDMTGLPVIIKGIQSPEDAEIAIQAGADAIWVSNHGGRQLDAGPSSFDVLP |
| | LVAKTVNKRVPIIFDSGVRRGSHIFKALASGADIVAIGRPILYALHLGGSQGVT |
| | SVIDQLNKELTINMFLGGAKNIKEIQNTKLYTDKDFQL |
| SEQ ID | MTYQTSTENKAIEIVNIKSLEGKVKESMESAGNKGAFGYIRGGAEDEWTMG |
| NO: 62 | ENTSAFNKKQIMPRVLRGIDSADLSTSLFGIDLSTPIIQAPVAAQGLAHEEGEV |
| A0A166JM | ATAKAMAEVGSIFSISTYGSTSVEDAAKAAPGAPQFFQLYMSKDDKFNEFLL |
| C9 | KKALAAGVKAIILTADSTLGGYREEDIVNHFQFPLPMPNLAAFSESDGTGKGI |
| Lactococcus | SEIYAAAKQGLVLEDIKKIKEITKLPVIVKGIQSPIDADDAIKAGADGIWVSNHG |
| cremoris | GRQLDGGPASIDVLPLVAKSVNHRVPIIFDSGVRRGGHVFKALAQGADIIAIG |
| | RPVLYGLNLGGAKGVQSVFEHLNKELSITMQLAGTKNIDEVKRTALID |
| SEQ ID | MKYQTSQENKKIEIINLQSLEARVKKSMEAAGNGGAFGYIRGGAEDEWTMR |
| NO: 63 | ENTLAFNKKKIMPRVLRGIDHADLSTSLFGIPLTTPIIQAPVAAQGLAHEEGEV |
| A0A166UNI | ATAKALAKIGSIFSISTYGSTAVEEAAQAVPGAPQFFQLYMSKEDHFNEFLLA |
| 9 | KAVAAGVKAIILTADSTLGGYREEDVVNHFQFPLPMPNLAAFSESDGKGRGI |
| Lactococcus | SEIYAAAKQELVMEDIQKIKAFTHLPVIVKGIQSPIDAAEAIEAGADGIWVSNH |
| cremoris | GGRQLDGGPASIEVLPLIAEQVNHRVPIIFDSGIRRGSHVFKALALGADVVAI |
| | GRPVLYGLNLGGAQGVEAVFEHLNKELSITMQLAGTKTIDEVKHTPLMD |
| SEQ ID | MTYHTSNEEHPIEIVNIASLEDRVKERMEAGAFGYIRGGSEDEWTMKENTSA |
| NO: 64 | FNTKKIMPRVLRGIDHADLHTSVFGIDLDTPIIQAPSAAQGLAHEKGEADTAK |
| A0A179ES | GVAAAGSIFSISTYANTTIKDAAAAAPNAPQFFQLYMSKDDGFNEFILTQAME |
| E3 | AGAKAIILTADSTLGGYREEDVINQFQFPLPMPNLAAYSEQSASGSGEGKGI |
| Enterococcus | AEIYAAAKQGLVPDDIKKIKEFTHLPVFVKGIQSPEDAEVAIAAGADGIWVSNH |
| thailandicus | GGRQLDGGPASFEVLPMIAATVNKRVPIVFDSGVRRGEHVFKALASGADLV |
| | AIGRPVIYGLNLGGAEGVTSVFEHFNKELSITMQLAGTKTIEEVKNTTLLD |
| SEQ ID | MTVVNGYEQSDAEKKLDILNLPSLEGESKKIIPTGGFGYIVGGSEDEWTLAE |
| NO: 65 | NTKAFNHAQIVPKALSNIDSPDLSTNFLGIDLKTPVMMAQTAAQGLAHSQGE |
| A0A180A8 | KDTARGLAAVGGLMAQSTYSSTSIADTAAAGNGAPQLFQLYMSKDWDFNK |
| K0 | SLLDEAKKAGVKGIILTVDATVDGYREEDIINNFQFPIPMPNLEKYSEGDGKG |
| Lacticasei- | KGIGEIYASAAQKINEDDVRRIAEYTDLPVIVKGIQSPEDALRAIGAGAAAIYVS |
| bacillus | NHGGRQLNGGPASFDVLPAIAKAVNKQVPIIFDSGIRRGSHVFKALASGADL |
| rhamnosus | VAFGRPVIYGLALGGAQGVQSVFEQIDHELEIIMQLAGTKTIEDVKHAPLTHF |
| | NYAD |
| SEQ ID | MTMINGYEQSDREEKIDILNLESLEKRAEEIIPAGGFGYIAGGSEDEWTLKQN |
| NO: 66 | RMAFHHRQIAPKALSGIEKPELNTEIFGIPLNTPVMMAPAAAQGLAHSQGEK |
| A0A1A5VQ | DTARGLAAVGGLMAQSTYSSVSIAETAAAGGDAPQFFQLYMSKDWNFNES |
| D4 | LLDEAKKANVKAIILTVDATVDGYREADIKNKFTFPLPMANLIKFSEGNGQGK |
| Pediococcus | GIEEIYASAAQNIRPEDVKRIADYTNLPVIVKGIQTPEDAIRAIDAGAAGIYVSN |
| acidilactici | HGGRQLNGGPASFDVLEDIATAVNKQVPIIFDSGVRRGSDVFKALASGADLV |
| | ALGRPVIYGLALGGAKGVQSVFEHLNHELEIVMQLAGTKTIEDVKNNSLLNIK |
| | Y |
| SEQ ID | MKKVKAGFFAATVLGLAMTMTTAYAAEYKASIKEGPLKIVNLNDLESQVKAN |
| NO: 67 | MDKGAFGYIRGGAEDEKNMRDNTASFDRKYIMPRVMQGIELKDINISTSLLGI |
| A0A1B8GY | PLDTPVIQAPMAAQGLAHRDGEIATARGMAQAGSIFTLSTYGNKTIEEVAAV |
| V1 | SDGHPFFFQLYMSKNDAFNEFTLKRAKESGAKAIILTVDSPVGGWREDDLR |
| Morganella | NNFQFPLGFANLELFAKQNNDGAKTGKGAGISEIYAQAKQAFTPSDIQYVKK |
| psychrotolerans | MSGLPVIVKGIQSPEDADRVIEAGADAIWVSNHGGRQLDSGPASFDVLPAIA |
| | KTVNKRVPVVFDSGVRRGSHVFKALASGADIVAVGRPVLYGLNLGGAQGVN |
| | SVIQQLNKELRINMMLGGAKDINAVKQTKLYTDSELQ |
| SEQ ID | MSYHTSNEEHAIEIVNIASLEALVKARMEPGAFGYIRGGAEDEWTMRENTLA |
| NO: 68 | FNRKKIIPRVLQGIDHANLSTKLWDISLKTPIIQAPSAAQGLAHEQGEKDTAKG |
| A0A1B9KI2 | VAAAGSIFCISTYANTSIEDAANAAPNVPYFFQLYMSKDDDFNRFIIDKAVKA |
| 4 | GAKAIILTVDSTLGGYREEDIVNKFQFPLPMKNLSAYSQSNGNGDGSGKGIS |
| Gilliamella | EIYAAAKQGIVPSDIQKIKAMANLPVIVKGIQSPEDASIAISAGADGIWISNHGG |
| apicola | RQLDGAPASFEVLPSISAAVAKRVPIIFDSGIRRGEHVFKALASGADLVAIGRP |
| | ILYGLNLGGAEGVKSVFDHLNKELSITMQLAGTKTIEDIKNTLLI |
| SEQ ID | MSYHTSNEEHAIEIVNIASLEALVKARMEAGAFGYIRGGAEDEWTMRENTLS |
| NO: 69 | FNRKKIVPRVLQGVDHADLTTKLWDIPLKTPIIQSPSAAQGLAHEKGEMDTAK |
| A0A1B9L52 | GVAAAGSIFSISTYANTSIEEAASAAPNVPYFFQLYMNKDDGFNRFIVDKAVK |
| 0 | AGAKAIILTVDSTLGGYREEDIINKFQFPLPMKNLSAYSQSSGNGDGSGKGIS |
| Gilliamella | EIYAAAKQGIVPSDIQKIKEMAKLPVIVKGIQSPEDAAIAISAGADGIWVSNHG |
| apicola | GRQLDGGPASFDVLPNIAAVVAKRVPIIFDSGVRRGEHVFKALASGADLVAIG |
| | RPILYGLNLGGAEGVKSVFDHLNKELSITMQLAGTKTIEDIKNTPLI |
| SEQ ID | MSYHTSNEEHAIEIVNLASLEALVKVRMEAGAFGYIRGGAEDEWTMRENTLA |
| NO: 70 | FNRKKIVPRVLQGIDHADLSTKLWDISLKTPIIQAPSAAQGLAHEQGEKDTAK |
| A0A1B9LG | GVAAAGSIFCISTYANTSIEDAANAAPNVPYFFQLYMSKDDDFNRFIIDKAVK |
| 94 | AGAKAIILTVDSTLGGYREEDIVNKFQFPLPMKNLSAYSQSNGNGDGSGKGI |
| Gilliamella | SEIYAAAKQGIVPSDIQKIKDMANLPIIVKGIQSPEDASIAISAGADGIWISNHG |
| apicola | GRQLDGAPASFEVLPSISAAVAKRVPIIFDSGIRRGEHVFKALASGADLVAIG |
| | RPILYGLNLGGAEGVKSVFDHLNKELSITMQLAGTKTIEDIKNTLLI |
| SEQ ID | MSYHTSNEEHAIEIVNIASLEALVKARMQAGAFGYIRGGAEDEWTMRENTLS |
| NO: 71 | FNRKKIIPRVLQGIDRADLSTKLWDISLKTPIIQAPSAAQGLAHEQGEKDTAKG |
| A0A1B9N2 | VAAAGSIFCISTYANTSIEDAANAAPNVPYFFQLYMSKNDDFNRFIIDKAVKA |
| 02 | GAKAIILTVDSTLGGYREEDIVNKFQFPLPMKNLSAYSQSNGNGDGSGKGIS |
| Gilliamella | EIYAAAKQGIVPSDIQKIKAIANLPVIVKGIQSPEDASIAISAGADAIWISNHGGR |
| apicola | QLDGAPASFEVLPNIAATVAKRVPIIFDSGVRRGEHVFKALASGADLVAIGRPI |
| | LYGLNLGGAEGVKSVFDHLNKELSITMQLAGTKTIEDIKNTLLI |
| SEQ ID | MTSYYNGFPQSDRDEAIDMINLDELEERAKEVMPEGAYYYIASGSENEWTW |
| NO: 72 | RNNIAAFNHFQIVPRALTNMDNPQLDTEFMGMKLKTPVMISPIACHGISHKDA |
| A0A1C4AC | EVATQKGAAAAGALFSSSTYANKSVEDIAAAAPDAPRFFQLYLSKDWDFNE |
| V3 | MVFEAIKKAGYKGIFLTVDALVSGYREANLRTNFAYPVPLDFFTRYQGGKGE |
| Lactobacillus | GQTVAQMYASSAQKIGPEDVKRIKEMSGLPVFVKGVVCAEDAYLAMGAGA |
| apis | DGIYVTNHGGREVDCGPATIDMLPEIAKAVNHRVPIVFDSGVRRGSHVFKAL |
| | ALGADLVGVGRPYLYGLALGGAKGVQSVIEQLNKELLIDMQLTGCKTIEDVK |
| | HAKITHINYAADNLKSNTDPSRIKPYPVTAENQMKESAADAVSGASHH |
| SEQ ID | MSYHTSNEEHAIEIVNIASLEALVKARMEAGAFGYIRGGAEDEWTMRENTLS |
| NO: 73 | FNRKKIIPRVLQGIDHADLSTKLWDIPLKTPIIQAPSAAQGLAHEQGEKDTAKG |
| A0A1C4CJ | VAAAGSIFCISTYANTSIEDAANAAPNVPYFFQLYMSKDDDFNRFIIDKAVKA |
| 33 | GTKAIILTVDSTLGGYREEDIVNKFQFPLPMKNLSAYSQSNGNGDGSGKGIS |
| Gilliamella | EIYAAAKQGIVPSDIQKIKDMANLPVIVKGIQSPEDASIAISAGADGIWISNHGG |
| bombicola | RQLDGAPASFEVLPSIAATVAKRVPIIFDSGVRRGEHVFKALASGADLVAIGR |
| | PILYGLNLGGAEGVKSVFDHLNKELSITMQLAGTKTIEDIKNTLLI |
| SEQ ID | MKRQILKTTALAMALSVGVAQAAEYKASTAEGPIKIVNLKAMEAQVQANMEK |
| NO: 74 | GAFGYIRGGAEDENNLRSNTSAFDKKYIMPRSLQGIEFSDLDLTTEFLGIKLD |
| A0A1D3DH | TPIIQAPMAAQGLAHQQGEVATAKGMAKAGSIFSLSTYGNKTIKEVADAQPG |
| R6 | YPFFFQLYMSKNDAFNEYILSQAKQYGAKGIIMTIDSSVGGYREDDVKNNFQ |
| Shigella sp. | FPLGFANLEAFAKISDDKSKTGKGAGISEIYAQAKQAFTPADIQYVKKMSGLP |
| FC1655 | VIVKGIESPEDADTAIKAGADAIWVSNHGGRQLDSAPATIDVLPAIAKVVNKR |
| | VPIVFDSGVRRGSHVFKALASGADVVAVGRPILYGLNLGGAEGVNSVIQHLN |
| | KELKINMMLGGAKTVKDIQATQLYTDASFNQ |
| SEQ ID | MAYYTSNAEHPIDILNIKGLEARVKARMEPGAFGYIREGAEDEWTLRENTRS |
| NO: 75 | FEDKYIVPRVLRGISHADLSTSIFGIPLSTPVIEAPSAAHGLAHVKGEVDTAIGA |
| A0A1G5V6 | AKAGTLFAMSTYGSTDLEEAAKAAPGAPQFFQIYMSKDDGFNEFLIRKAVRA |
| H0 | GVKAIIMTVDSTLGGYREEDIATHFQFPLPMPNLAAYGNADGKGKGISEIYAA |
| Allisonella | AKQDFVPSDIGKLKRLSGLPVIVKGIQSPEDALIALDAGADGIWVSNHGGRQL |
| histaminiformans | DGGPASFAVLPSIAKAVNKRAPIIFDSGVRRGNHVFKALASGADLVAIGRPVI |
| | YGLNLGGAEGVKSVFDHENKELSITMQLAGTKTIEDVKKTELI |
| SEQ ID | MTVTNGYEQSDREQAIKIINLDELEDQARQIIPQGGFGYISEGSEDEWTKAR |
| NO: 76 | NRAAFNTVQIAPRVLHSVEAPSTSTSVFGVSIKTPVIMAPTAAQGLAHTRGEA |
| A0A1H9PH | ATAEGVAAAGTIMSQSTYGTTSIAETAEASKGAPWFFQLYMSNDWEFNEAL |
| D3 | LDEAKSHGAAAVVLTVDSMQGGYREPDIRNEFQFPLPMANLAAFSETSGKG |
| Propionibacterium | KGIFEIYAAAKQKITGADVRRVAEYVGLPVIVKGIQDPCDAALALGSGASGIW |
| cyclohexanicum | VSNHGGRQLNGGPGSFDVLPSIARAVNGRVPVIFDSGVRRGSQVFKALASG |
| | ADLVAIGRPAIYALALGGAQGVRTVFEYLTHEFRIVMQLAGTQTVDDVKRAK |
| | LLHY |
| SEQ ID | MTVYYNDFPQSDRDEYIKMINLDELEERAKKVMPEGAYYYIASGSENEWTW |
| NO: 77 | RNNTVAFNHFQIVPRALTNMADPQLDTDFMGMKLKTPIMISPIACHGIAHQD |
| A0A111RXP | AEVATQKGAAAAGALFSSSTYANKSVEDISAAAPEAPRFFQLYLSKDWNFN |
| 2 | QMVFDAIKKAGYQGIFLTVDALVSGFREANLRTQFAFPVPLDFFTRYQGTKG |
| Lactobacillus | EGQTVAQMYASSAQKIGPEDIKRIKEMSGLPVFVKGVVCAEDAYLAIGAGAD |
| bombicola | GIVVTNHGGREIDGGPATIDMLPEIAKAVDHRVPIVFDSGVRRGSHVFKALAL |
| | GADLVGIGRPYLYGLALGGAKGVQSVIEQLNKELLIDMQLTGCKTIADIRHAKI |
| | TNINYTADNLKSNTDPERIKPYPVTKENQLKAGGTDTVSGASHL |
| SEQ ID | MLSFTRKNTFVTSTLFAVALTLGTAQAAEYKASDKEGPIKIVNLDELESQVAK |
| NO: 78 | SMDKGAFGYIRGGAEDELNLKKNTQHFDNKYIMPRVMQGIEISDIDLSTDFL |
| A0A1J0E26 | GIKLKTPIIQAPMAAQGLAHKDGEIATAKGMAKAGSIFSLSTYGNKTIEEVAEV |
| 1 | SGENPFFFQLYMSKNNAFNEFTLKRAKASGAKAIILTVDSPVGGYREDDIRN |
| Providencia | NFQFPLGFANLELFAKQNSDGSKTGKGAGISEIYAQAKQAFTPADIQYVKKL |
| rettgeri | SGLPVIVKGIQSPEDADVVIKAGADAIWVSNHGGRQLDSGPASFDVLPSIAKV |
| | VNKRVPIVFDSGVRRGSHVFKALASGADVVAVGRPILYGLNLGGAEGVNSVI |
| | QQLNKELSINMMLGGAKNIEGVKATKLYTDKDFQ |
| SEQ ID | MYHTSNEEHPIDIVNIASLEGRVKERMEAGAFGYIRGGSEDEWTMKENTTSF |
| NO: 79 | MNKKIMPRILQGIDHADLSTKLWDIELKTPIIQAPSAAQGLAHEKGEADTAKG |
| A0A1L8R8J | VAAAGSIFSISTYANTTIEDAAAAAPDAPQFFQLYMSKDDGFNEFILDKAVKA |
| 5 | GAKAIILTADSTLGGYREEDVINQFQFPLPMPNLAAYSAQSASGDGEGKGIA |
| Enterococcus | EIYAAAKQGLVPEDIKKIKDLTHLPVFVKGIQSPEDADLAIKAGADGIWVSNHG |
| canintestini | GRQLDGGPASFEVLPAIAEVVDKRVPIVFDSGVRRGEHVFKALASGADLVAI |
| | GRPVIYGLNLGGAEGVKSVFDHLNKELSITMQLAGTKTIADVKKTKLID |
| SEQ ID | MAYHTSNEEHPIEIVNIASLEGRVKERMEAGAFGYIRGGSEDEWTMRENTA |
| NO: 80 | AFNKKTIMPRVLRGIDHADLSTKLWDIELATPIIQAPSAAQGLAHEKGEADTA |
| A0A1L8RG | KGVAAAGSIFSISTYANTTIEDAAKAAPEAPQFFQLYMSKDDGFNKFILDKAV |
| N0 | QAGAKAIILTADSTLGGYREEDIINQFQFPLPMPNLAAYSAQSASGNGEGKGI |
| Enterococcus | AEIYAAAKQGLVPEDIKAIKDLTDLPVFVKGIQSPEDAEVAIAAGADGIWVSNH |
| canis | GGRQLDGGPASFDVLPKIAAVVNKRVPIVFDSGVRRGEHIFKALASGADLVAI |
| | GRPVIYALNLGGAEGVKSVFDHLNKELSITMQLAGTKTIEEVKNTVLFD |
| SEQ ID | MAYQTSNEEHPVEIVNIASLEGRVKERMDKGAFGYIRGGSEDEWTMKENTS |
| NO: 81 | SFNNKTIMPRVLRGVDHADLSTKLWDIELATPIIQAPSAAQGLAHEKGEADTA |
| A0A1L8SR | KGVAEAGSIFSISTYANTTVEDAAATAPEAPQFFQIYMSKDDGFNEFILDKAV |
| 64 | KAGAKAIILTADSTLGGYREEDIVNNFQFPLPMPNLAAYSEQSASGDGEGKG |
| Enterococcus | IAEIYAAAKQGLVPEDIKKIKDLTNLPVLVKGIQSPEDAEVAIAAGADGIWISNH |
| devriesei | GGRQLDGGPASFDVLPLIAGVVNKRVPIIFDSGVRRGEHVFKALASGADLVAI |
| | GRPVIYGLNLGGAAGVKSVFDHLNKELSITMQLAGTRTIEEVKNTKLNDAK |
| SEQ ID | MTETNGYFQNDNEKEINVLNLQSLEAEAEKIIPKGGFGYIAGGSEDNWTLKE |
| NO: 82 | NTEAFNHVQIVPHVLSNVEDPQTDTSIFGINVKTPIMMSPAAAQGLAHAKGEI |
| A0A1P8Q41 | DTAAGIAKAGALMSQSTYSSTSIADTMKAGNGAPQFFQLYMSKDWNFNENL |
| 3 | LKEAKEAGAKAIILTSDATVDGYRESDVVNDFQFPIPMANLTKFSEGDGKGK |
| Companilac- | GIGEIYAAAAQKIGPDDIKRIKDIAGLPVIVKGVQSPEDALLAIGAGADGIYVSN |
| tobacillus | HGGRQLNGGPASFDVLSDVAKAVNHRVPIIFDSGIRRGSHVFKALASGADLV |
| allii | ALARPIIYGLALGGADGVYSVVEHLNDEFKTTMQLAGTKTIEDVKNSKLLKK |
| SEQ ID | MSNTYKQSTNEQAIEIVNLDELQERAKAIIPAGGFGYISSGSEDEWTLRANRE |
| NO: 83 | AFNHKLIVPRSLTDMEKPLIDTSIFGIPLKTPVMMAPTAAQGLAHIEGEADTAR |
| A0A1Q8FC | GVAAVGGLMAQSTYSSRTITETTTAGNGAPQFFQLYMSKDWSFNNALLDQA |
| V9 | KAAGIKAIILTVDATVGGYREADVRNKFSFPIPMANLENFSKDNGEGKGISEIY |
| Loigolacto- | AAAAQKISPKDIARIANYTDLPVIVKGIQSPEDAELAISSGAAGVYVSNHGGR |
| bacillus | QLNGGPASFDVLASVAQVVNHRVPVIFDSGIRRGSHVFKALASGADLVAFG |
| backii | RPAIYGLALGGAQGVQSVFEHIDDELKIVMQLAGTQTIAAVKQTKLLDNHF |
| SEQ ID | MKHTLLKTTAIAMALSVGVVQAAEYKASTAEGPIKIVNLKAMEAQVEANMDK |
| NO: 84 | GAFGYIRGGAEDENNLRANTRAFDKKYIMPRSLQGIEFSDINLKTEFLGIKLD |
| A0A1S6Q8 | TPIIQAPMAAQGLAHQQGEVATAKGMAKAGSIFSLSTYGNKTIKEVAQAQPG |
| R2 | YPFFFQLYMSKNDAFNQYILSQAKQYGAKGIIMTIDSSVGGYREDDVKNNFQ |
| Proteus | FPLGFANLEAFAKISDDKSKTGKGSGISEIYAQAKQAFTPADIQYVKKMSGLP |
| mirabilis | VIVKGIESPEDADTAIKAGADAIWVSNHGGRQLDSAPATIDVLPAIAKVVNKR |
| | VPIVFDSGVRRGSHVFKALASGADVVAVGRPILYGLNLGGAEGVNSVIQQLN |
| | KELRINMMLGGARNVKEIQATHLYTDADFK |
| SEQ ID | MTVVNGYEQSDREEKLNILNLPSLEAEAKKIIPTGGFGYIVGGSEDEWTLNH |
| NO: 85 | NSEAFNHVQIVPRALTDMENPSTATKVFGLDLKTPIMMAPTAAQGLAHSRGE |
| A0A1S6QH | EATAEGVAAAGALMAQSTYSSTSIADTAAAGKGAPQFFQLYMSKDWDFNES |
| N1 | LLDEAKKAGVKAIILTVDATVDGYREADIINNFQFPIPMANLTKFSEGDGEGK |
| Lentilacto- | GIEEIYASAAQKINADDVKRIADYTDLPVIVKGIESPEDALLAIGAGAKGIYVSN |
| bacillus | HGGRQLNGGPASFDVLEDVAKAVNHQVPIIFDSGIRRGSHVFKALASGADLV |
| curieae | AMGRPVIYGLALGGAQGVQSVFEELNHELEIDMQLAGTKDIEAVKHAKLVKV |
| | HY |
| SEQ ID | MAYQGSTKEEALNIIDLPRLRAAVQRDTEAGAFGYVDGGSSDEQVLHDNET |
| NO: 86 | AFRHYQLIPRMLQNISAPDLSTTLIDIPLSMPVIAAPIAAHGLMHENGERVTVK |
| A0A1T4J6V | GVGAAGTIFSLSTYGNSRIADVAASSPDTPKFFQLYMSRDDEFNQYLLDEAV |
| 0 | QNGYKAIILTADATLGGYREADIINNFAFPLPMENLAAFSNAAGSGEGLGISEI |
| Weissella | YARAKQDLALSDITKVKQMANGLPVFVKGIQDPDDALAAIAAGADGIWVSNH |
| confusa | GGRELNGAPASIDTLAAVAKAVNHRVPVVFDSGIRRGEDVAKAIALGADVVA |
| | LGRPMLWGLNQGGAAGVQSVYEHLAEELKIVMQLTGSHTVAELQHAKIIDA |
| | KF |
| SEQ ID | MTTYYKGFPQSTREEKLHMVNLDELENEAKYVIPEAAYYYIASGAENEWTW |
| NO: 87 | RNNTQAFNHFQIVPRALTGMQDPELNTEFLGMKLKTPVMICPIACHGIANAE |
| A0A1V3Y2 | AEIDTAKGAKAAGALFGMSTYANKSVQDVQNAVGDSPRFMQLYLSKNWDF |
| U8 | NKMVIEESVKAGFTGFFLTVDALVSGYREANLRTNFTYPVPLAFFNEWTGGK |
| Lactobacillus | GEGQSVAEMYASSAQNIGPDDIRKIKDIADVPVIVKGVECAEDAMLAIGAGAD |
| gasseri | GIVVSNHGGREVDGAPATIDVLPEIAKAVKSCDRPVPIILDGGVRRGSHVFKA |
| | LALGADLVGIGRPFLYGLALGGAQGVQSVIEQLNKELLIDMQLTGCKTIDDIK |
| | HAKIDHLDYTADWGISSTSKSVMKPYPVTKENQLTGEAADAVSGASRH |
| SEQ ID | MTMINGYEQSDREEKIDILNLESLEERAEKIIPTGGFGYISGGSEDEWTLRQN |
| NO: 88 | RTAFQHRQIAPKALSGIEKPELNTEIFRIPLNTPVMMAPAAAQGLAHSQGEKD |
| A0A1YOVZ | TARGLAAVGGLMAQSTYSSVSIADTAAAGEGAPQFFQLYMSKDWNFNESLL |
| 69 | DEAKKAHVKAIILTVDATVDGYREADIKNKFAFPLPMANLTKFSEGDGQGKGI |
| Pediococcus | EEIYASAAQNIRPEDVKRIADYTQLPVIVKGIQTPEDAIRAIDAGAAGIYVSNH |
| pentosaceus | GGRQLNGGPGSFDVLEDIATSVNKQVPIIFDSGVRRGSDVFKALASGADIVA |
| | LGRPVIYGLALGGAKGVQSVFEHIDHELEIVMQLAGTKTIDDIKNNPLLNIKY |
| SEQ ID | MTVYYKGFPQSDREEYLHMINVDELEQRVKKIMPEGAYYYIASGSENEWTW |
| NO: 89 | RNNTAAFNHWQIIPRALTDMENPSTATEFMGMKLKTPVMISPIACHGIAHTM |
| A0A1Y4Q5 | AEVDTQKGAAAAGALFASSTYANKSVEDIAKAAPDAPRFFQLYLSKDWKFN |
| J8 | EMVFDAIKKTGYKGIYLTVDALVSGYREANLRTKFTYPVPLDFFTRYLDGKG |
| Lactobacillus | EGQSVAQMYASSAQKIGPEDVRRIKEETGLPVIVKGVESVEDAYKAMGAGA |
| gallinarum | DGIYVTNHGGRQLDGGPAAIDVLPAIAKAVNHQVPIIFDSGVRRGSHVFKALA |
| | LGADMVGIGRPYLYGLALGGAPGVQSVIDQLDKELLIDMQLTGCKTIEDVKH |
| | AKIAHINYTADNLDSITSPDRMKPYPKTADNQIKTNGASDATSGASEH |
| SEQ ID | MKHTLLKTTAIAMALSVGVVQATEYKASTAEHPIKIVNLDALENQVKENMDK |
| NO: 90 | GAFGYIRGGAEDENNLRSNTNAFNKKYIMPRALQGIEFSDLNLKTEFLGIKLD |
| A0A1Z1T10 | TPIIQAPMAAQGLAHQQGEVATAKGMAKAGSIFSLSTYGNKTIKEVAQAQPG |
| 8 | YPFFFQLYMSKNDAFNQYILSQAKQYGAKGIILTVDSPVGGYREDDIKNSFQ |
| Proteus | FPLGFANLEAFAKISDDKSKTGKGSGISEIYAQAKQAFTPADIQYVKKMSGLP |
| mirabilis | VIVKGIESPEDADTAIKAGADAIWVSNHGGRQLDSAPATIDVLPAIAKVVNKR |
| | VPIVFDSGVRRGSHVFKALASGADVVAVGRPILYGLNLGGAEGVNSVIEQLN |
| | KELRINMMLGGARNVKEIQATHLYTDADFK |
| SEQ ID | MVITNGYKQNENERLIDIVNLTQLEAEAKQIIPAGGFGYITSGSEDEFTLQANR |
| NO: 91 | KAFQHRQIVPRSLSNIEKPQTDTNVFGIDLKTPIIMAPAAAQGLAHVKGEADT |
| A0A1Z5H3 | AKGVAKAGALMTESTYSSASIADVAAGGGGAPQFFQLYMSKNWDFNRSILD |
| C7 | EAKKAGVKAIILTVDATVGGYREADIVNHFQFPIPMANLVKFSQGDGEGKGIS |
| Schleiferilac- | EIYAAAAQKIGPKDVQRIIDYTDLPVIVKGIESPEDALYAIGAGAAGVYVSNHG |
| tobacillus | GRQLNGGPASFDVLESVAKAVNHQVPVLFDSGVRRGSDVFKALALGADLV |
| silagei JCM | GIGRPAFFGLALGGADGVFSVFEHLNNELKIVMQLAGTKTVADIKHAKLLNIQ |
| 19001 | Y |
| SEQ ID | MVMTNGYEQNENEKTLDILNLTQLEEDAKKIIPTGGFGYIVSGSEDEWTLNA |
| NO: 92 | NRKAFQHRQIVPKALSNIENPQTDTTVFGLDLKTPIMMAPAAAQGLAHAKGE |
| A0A1Z5H5 | VDTAKGVAKAGGLMAQSTYSSTSIADTAAGGDGAPQFFQLYMSKDWDFNK |
| J1 | SLLDEAKQAGVKAIILTVDATVGGYREADIINHFQFPIPMANLIKFSEGDGEGK |
| Secundilac- | GISEIYASAAQKIGPDDVKRIIDYTDLPVIVKGIESPEDALYAIGAGAAGIYVSN |
| tobacillus | HGGRQLNGGPASFDVLESVTKAVNHQVPVIFDSGVRRGSDVFKAIALGADL |
| silagincola | VAIGRPAIYGLALGGADGVYSVFEHLNNELKIVMQLAGTKTIEDIKHAELLHIQ |
| | Y |
| SEQ ID | MVMTNGYEQNENEKTLDILNLTQLEADAKKIIPTGGFGYIVSGSEDEWTLQA |
| NO: 93 | NRKAFQHRQIVPKALSNIEDPQTDTNVFGLGLKTPIMMAPAAAQGLAHAKGE |
| A0A1Z5IBR | VDTAKGVAKAGGLMAQSTYSSTSIADTAAGGEGAPQFFQLYMSKDWDFNR |
| 7 | SLLDEAKQAGVKAIILTVDATVGGYREADIINHFQFPIPMANLIKFSEGDGEGK |
| Secundilac- | GISEIYASAAQKIGPDDVKRIIDYTDLPVIVKGIESPEDALYAIGAGAAGVYVSN |
| tobacillus | HGGRQLNGGPASFDVLESVAKAVNHQVPVIFDSGVRRGSDVFKAIALGADL |
| mixtipabuli | VGIGRPAIYGLALGGADGVYSVFEHLNNELKIVMQLAGTKTIEDIKHAELLNV |
| | QY |
| SEQ ID | MTYQASNEEHPIDIVNIASLEQRVKERMEKGAFGYIRGGSEDEWTMKENTT |
| NO: 94 | SFNNKTIMPRVLRGIDRADLRTKLWDMDLATPIIQAPSAAQGLAHENGEADT |
| A0A200HV | AKGVADAGSIFSISTYANTTIEDAAAAAPNAPQFFQLYMSKDDGFNEFILDKA |
| S6 | VKAGAKAIILTADSTLGGYREDDVINHFQFPLPMPNLAAYSAKSDSGDGKGK |
| Enterococcus | GIAEIYAAAKQGLVPEDIKKIKDLTNLPVFVKGIQSPEDAEVAIAAGADGIWISN |
| sp. | HGGRQLDGGPASFNVLPLIAGVVNKRVPIVFDSGVRRGEHVFKALASGADL |
| 12F9_DIV0 | VAIGRPVIYGLNLGGAEGVKSVFDHLNKELSITMQLAGTKTIEEVKNTTLHDA |
| 723 | K |
| SEQ ID | MTYPTSDAEHPVDVLNLVSLEGRVKERMEAGAFGYIRGGSEDEWTMAENT |
| NO: 95 | SAFNQKKIMPRVLQGVEHADLHTKLWDIDLKTPIIQAPSAAQGLAHEKGEVD |
| A0A221MX | TAKGMAAAGSIFSISTYANTLVEDAAAAAPDAPQFFQLYMSKDDQFNEFLLQ |
| 79 | KAVNAGVKGIVLTVDSTLGGYRETDIETKFQFPLPMPNLAAYSNSDGAGKGI |
| Latilacto- | SETYATAKQGIVPEDIQKIKEITHLPVIIKGIQSPVDAELTVQAGADGIWVSNH |
| bacillus | GGRQLDGGSASFEVLPLVAQQVAKRVPIIFDSGVRRGEHVFKALASGADLV |
| sakei | AIGRPIIYGLNLGGAQGVTSVIDHMNHELSITMQLAGTKTIDAVKETTLLD |
| SEQ ID | MSYITSTQERHIDILDIPSLEPKVGANMAKGAFGYLSGAAEDELVLKANPLAF |
| NO: 96 | NHKLIAPRVLQDIENPDLTTEFLGLKLSAPIIAVPIAAHGLVHEKAELDTAQGV |
| A0A224WY | ADAGTIFSLSTYGNATVDDVAKVVPEAPKFFQLYMSKDDNFNRWILDKAVKG |
| I3 | GYKAIILTADSTLGGYRESDIVNNFTFPNVMRNLEEWSKLSSDGETGSGEGI |
| Lactococcus | AAIYAKAKQALSLKDIAFIKDYTHLPVFVKGVQSPKDVDPLIAAGVDGIWVSN |
| reticulitermitis | HGGRQLDGGPASFDVLADIAKVVNKRVPIVFDSGVRRGQHIFKALASGADV |
| | VGIGRPMLWGLNLGGRQGVTDVFDHFKKELMITMQLAGTHNVAEIKATELID |
| | AK |
| SEQ ID | MKSKHHKIVLSISTLAIAIATSFSNAAETSYKASSVNQPIKIVNIASLEAQAKAQ |
| NO: 97 | MQPGAFGYIRGGAEDENNLRSNTSAFDKKYIMPRIMQAIELKDINLNTKFLGI |
| A0A241V1 | DLKTPIIQAPMAAQGLANVDGELSTAKGMAKAGSIFSLSTYGNKTIEEVAQVS |
| K1 | GKNPFFFQLYMSKNDKFNEFTLKRAKEHGAKAIILTVDSPVGGYREDDIKNN |
| Acinetobacter | FQFPLGFANLELFAQQNKDGSKTGKGAGISEIYAQAKQAFTPSDIQYVKKLT |
| sp. ANC | GLPVIVKGIQSPEDADMAIRAGADAIWVSNHGGRQLDSGPSSFDVLPGIAKV |
| 4204 | VNKRVPIVFDSGVRRGSHVFKALASGADVVAIGRPILYGLQLGGAEGVNSVM |
| | EQFNKELRISMMLGGAKDIEAVKKTKLYTDKDFE |
| SEQ ID | MTYQASNEEHPIEIVNIASLEGRVKERMDKGAFGYIRGGSEDEWTMKENTS |
| NO: 98 | SFNNKTIMPRVLRGIDHADLSTKLWDIDLATPIIQAPSAAQGLAHEKGEADTA |
| A0A242D9 | KGVAAAGSIFSISTYANTTIEDAAAAAPDAPQFFQLYMSKDDGFNEFILEKAV |
| 69 | KAGAKAIILTADSTLGGYREEDIINKFQFPLPMPNLAAYSAKSASGDGEGKGI |
| Enterococcus | AEIYAAAKQGLVPEDIKKIKDLTNLPVFVKGIQSPEDAEVAIAAGADGIWISNH |
| sp. | GGRQLDGGPASFDVLPLIAGVVNKRVPIVFDSGVRRGEHVFKALASGADLV |
| 3H8_DIV06 | AIGRPVIYGLNLGGTEGVKSVFDHLNKELSITMQLAGTKNIDEVKNTRLND |
| 48 | |
| SEQ ID | MSYITSTQERHIDILDIPALETKVAANMEKGAFGYLSGAAEDELVLKANPLAF |
| NO: 99 | NHKLIAPRVLRDIENPDLTTEFLGLKLSAPIIAVPIAAHGLVHEHAELDTAQGV |
| A0A290PZ | ADAGTIFSLSTYGNATVDDVAKIAPESPKFFQLYMSKDDNFNRWILDKAVKG |
| U2 | GYKAIILTADSTLGGYREADIVNNFTFPNVMRNLEEWSKLSSEGETGSGEGI |
| Lactococcus | AAIYAKAKQALSLKDIAFIKDYTKLPVFVKGVQSSKDVEPLIEAGVDGLWVSN |
| raffinolactis | HGGRQLDGGPASFDVLADIAKVVNKRVPIVFDSGVRRGQHIFKALASGADV |
| | VGIGRPMLWGLNLGGSQGVTDVFDHFKKELMITMQLAGTHNVAEIKATELLD |
| | AK |
| SEQ ID | MSYITSTQERHIDILDIPALETKVAANMEKGAFGYLSGAAEDELVLKANPLAF |
| NO: 100 | NHKLIAPRVLQDIENPDLTTEFLGLKLSAPIIAVPIAAHGLVHEHAELDTAQGV |
| A0A2A5SG | ADAGTIFSLSTYGNATVDDVAKIAPESPKFFQLYMSKDDNFNRWILDKAVKG |
| Z5 | GYKAIILTADSTLCGYREADIVNNFTFPNVMRNLEEWSKLSSDGETGSGEGI |
| Lactococcus | AAIYAKAKQALSLKDIAFIKDYTKLPVFVKGVQSSKDGEPLIEAGVDGIWVSN |
| raffinolactis | HGGRQLDGGPASFDVLADIAKVVNKRVPIVFDSGVRRGQHIFKALAIGADVV |
| | GIGRPMLWGLNLGGSQGVTDVFNHFKKELMITMQLAGTHNVAEIKATELLDA |
| | KLTIG |
| SEQ ID | MKSKILKTTAIAMALSVGVAQAAEYKASTAEGPIKIVNLKAMEAQVQANMDK |
| NO: 101 | GAFGYIRGGAEDENNLRSNTTAFDKKYIMPRSLQGIEFSDLDLKTEFLGIKLD |
| A0A2J9L50 | TPIIQAPMAAQGLAHQQGEVATAKGMAKAGSIFSLSTYGNKTIKEVADAQPG |
| 5 | YPFFFQLYMSKNDAFNEYILSQAKQYGAKGIILTVDSPVGGYREDDIKNSFQF |
| Proteus | PLGFANLEAFAKISDDKSKTGKGAGISEIYAQAKQAFTPADIQYVKKMSGLPV |
| mirabilis | IVKGIESPEDADMAIKAGADAIWVSNHGGRQLDSAPATIDVLPAIAKVVNKRV |
| | PIVFDSGVRRGSHVFKALASGADIVAVGRPILYGLNLGGAEGVNSVIQHLNK |
| | ELKINMMLGGAKTVKDIQATQLYTDASFNQ |
| SEQ ID | MTYHTSDAEHPIDVLNLASLEGRVKERMEADAFGYIRGGSEDEWTMAENTS |
| NO: 102 | AFNQKKIMPRVLQGVEHADLHTKLWDIDLKTPIIQAPSAAQGLAHEKGEVDT |
| A0A2K4QB | AKGMAAAGSIFSISTYANTLVEDAAAAAPDAPQFFQLYMSKDDQFNEFLLQK |
| F8 | AVKSGVKGIILTVDSTLGGYRETDIETKFQFPLPMPNLAAYSNSDGAGKGISE |
| Latilacto- | IYAAAKQGIVPEDIQKIKEITHLPVIVKGIQSPVDAELAVQAGADGIWVSNHGG |
| bacillus | RQLDGGPASFEVLPLVAQQVAKRVPIIFDSGVRRGEHVFKALASGADLVAIG |
| sakei | RPIIYGLNLGGAQGVTSVIDHLNHELSITMQLAGTKTIDAVKETTLLD |
| SEQ ID | MKKKYEASTEEKHLEIVNIASLEKRVKDHMSNEKGAFGYIRGGSEDEWTMK |
| NO: 103 | QNTEAFSKKKIMPRVLQGIDHADLSTKLWDIDLKTPIIEAPSAAHGLAHAKGE |
| A0A2K9HG | VDTAKGVADAGSIFSMSTYGSTSVQDAAAAAPDAPQFFQLYMSKDDKFNEF |
| K2 | LIKEAVKAGVKAIVLTVDSTLGGYREEDIINKFQFPLPMPNLAAFSEGDGEGK |
| Companilac- | GISEIYAAAKQGIVPSDIQKIKDMSGLPVIVKGIQSPDDAELAIDFGADGIWISN |
| tobacillus | HGGRQLDGGPASFDVLPTIASAVDKRVPVIFDSGVRRGEHVFKALASGADL |
| alimentarius | VAVGRPIIYGLNLGGAQGVTDVINHLNMELSITMQLAGTKTINDVKNNQLLD |
| DSM 20249 | |
| SEQ ID | MTVYYKGFPQSDRDEYLHMINLQELEEKVKQVMPEAAYYYIASGAENEWT |
| NO: 104 | WRNNTAAFNHYQIVPRALTNMQDPQTDTEFMGMKLKTPVMISPIACHGIAH |
| A0A2N5KY | KDAEVATQKGAAAAGALFASSTYANKSVEDIAAAAPNAPRFFQLYLSKDWK |
| D1 | FNRMVFDAIKKTGYKGIYLTVDALVSGYREANLRTHFTYPVPLDFFTRYIGAK |
| Lactobacillus | GEGQSVAQMYASSAQKIGPKDVQRIKDETGLPVIVKGIECPEDAFKAIGAGA |
| crispatus | DGVYVTNHGGREVDGGPATIDVLPSIAKAVAHRVPIIFDSGVRRGSHVFKAL |
| | ALGADMVGIGRPYLYGLALGGAKGVESVIDQLDKELKIDMQLTGCKTIEDVK |
| | HAKINHISYTADNLPSNTSPSRMKPYPVTADNQIKKDSAADASSGASEH |
| SEQ ID | MNKKYEASTAENHVDIVNIASLEARVKDHMSNEKGAFGYIAGGSEDEWTKK |
| NO: 105 | QNTESFNHKSIMPRVIQGIDHADLSTKLWDIDLKTPIIQAPSAAQGLAHAKGE |
| A0A2N7AR | TDTAKGVAAAGSIFSISTYASTSVEDAAAAAPDAPQFFQLYMSKDDKFNEFLI |
| B3 | KKAVAAGAKAIILTVDSTLGGYREADVVNKFQFPLPMPNLAGYSAGDGEGK |
| Companilac- | GISEIYASAKQGIVPSDIQKIKDMSGLPVFVKGIQSPDDAELAIDFGADGIWVS |
| tobacillus | NHGGRQLDGGPASFDVLPYIAEVVDKRVPIVFDSGVRRGEHVFKALASGAD |
| nuruki | LVAIGRPIIYGLNLGGAQGVTDVIDHLNMELSITMQLAGTKTINDVKNTELLD |
| SEQ ID | MTYQASNEEHPIEIVNIASLEGRVKERMEKGAFGYIRGGSEDEWTMKENTS |
| NO: 106 | SFNKKTIMPRVLRGIDHADLSTKLWNIDLLTPIIQAPSAAQGLAHEKGEADTA |
| A0A2N8PX | KGVAAAGSIFSISTYANTTIEDAAAAAPNAPQFFQLYMSKDDGFNEFILEKAV |
| 97 | KAGAKAIILTADSTLGGYREEDIVNKFQFPLPMPNLAAYSAKSASGDGEGKGI |
| Enterococcus | AEIYAAAKQGLVPEDIKKIKDLTSLPVFVKGIQSPEDAEVAIAAGADGIWVSNH |
| avium | GGRQLDGGPASFDVLPLIAGVVNKRVPIVFDSGVRRGEHVFKALASGADLV |
| | AIGRPVIYGLNLGGAEGVKSVFDHLNKELSITMQLAGTKTINEVKNTRLHDAK |
| SEQ ID | MKKKYEASTAENHVDIVNIAGLEARVKDHMSNEKGAFGYIRGGSEDEWTMD |
| NO: 107 | QNTKAFNSRKIVPRVLQGIDHADLSTSLWDIALKTPIIQAPSAAQGLAHANGE |
| A0A2P4R6 | KDTAKGVADAGSIFSISTYASTSVEDAAAAAPDAPQFFQLYMSKDDKFNEFLI |
| 59 | KKAVAAGVKAIVLTVDSTLGDYREADVINKFQFPLPMPNLVGYSAGDGEGK |
| Companilac- | GISEIYASAKQGIVPTDIQKIKDMSGLPVIVKGIQSADDAELAVDFGADGIWVS |
| tobacillus | NHGSRQLDGGPASFDVLPAIAGTVDKRVPIIFDSGVRRGEHVFKALASGADL |
| formosensis | VAIGRPIIYGLNLGGAQGVTDVINHLNMELSITMQLAGTKTIADVKNTQLIY |
| SEQ ID | MQRSKAGVFAVAALGLAMTLSTAYAAEYKASTKEGPVKIVNLNDLESQVKA |
| NO: 108 | NMDKGAFGYIRGGAEDEKNMRDNTAGFDRKYIMPRVMQGIELKDIDLSTSF |
| A0A2T4HZ | LGIPLATPVIQAPMAAQGLAHRDGEIATAKGMAKAGSVFSLSTYGNKTIEEVA |
| V2 | AVSDGHPFFFQLYMSKNNAFNEFTLKRAKESGAKAIILTVDSPVGGWREDDL |
| Morganella | RNNFQFPLGFANLELFAAQNNDGSKTGKGAGISEIYAQAKQAFTPDDIQYVK |
| morganii | NLSGLPVIVKGIQSPEDADRVIEAGADAIWVSNHGGRQLDSGPASFDVLPSIA |
| | KVVNKRVPIVFDSGVRRGSHVFKALASGADVVAVGRPVLYGLNLGGAEGVN |
| | SVIQQLNKELRINMMLGGTKDINAVKQTRLYSDRDFQ |
| SEQ ID | MTEVNGYEQSDREQHIDVLNLESLEPEAEKIIPTGGFGYISGGSEDDWTLAA |
| NO: 109 | NREAFTHKQIYPRVLSDMDNPDLSTTVFGVNVKTPIMMSPLAAQGLANSQG |
| A0A2V1MX | EKDTARAFAAEGAFMGQSTYSSTSIADTAAAGNGGPQFFQLYMSKDWDFN |
| E9 | EALLDEAKKAGVKGIILTADATVDGYREDDIVNDFQFPIPMANLIQFSEASGQ |
| Levilacto- | GKGIKEIYAAAAQRISPDDVKRIADYSGLPVIVKGIQDPNDADLAIGAGAAGIY |
| bacillus | VSNHGGRQLNGGPASFDVLPAIAEAVNGRVPVIFDSGVRRGSDVFKALASG |
| bambusae | ADLVAMGRPFVYALALGGAMGVQDALQEINEEFKTVMQLAGTKTIEDVKHA |
| | QLADFRY |
| SEQ ID | MTVYYKGFPQSTREKKLNLVNLNQLEDEVKYVMPEAAYYYIASGSENEWTW |
| NO: 110 | RNNTQAFNHFQIVPRALTDRSNPSTDTTFMGMDLKTPVMISPIACNGISHAE |
| A0A2Z6T6 | AEVDVAKGAKAAGALYSMSTYANKSVEDVQKAVGESPRFFQLYLSKDWDF |
| P6 | NRMVITDALKAGFSGFFLTVDATVSGYREANLRTHFTYPVPLAFFNKWSGG |
| Lactobacillus | KGEGQSVAQMYASSAQKIGPEDIKKIKEIAGKDVPVFVKGVECAEDAFLAIGA |
| rodentium | GADGIVVSNHGGRQLDGAPATIDVLPEIAKEVHHSDHRIPIILDSGVRRGSHV |
| | FKALALGADLVGIGRPYLYGLALGGALGVESVIEQLNKELLIDMQLTGCKTIE |
| | DVKHAKIDHISYSADWGVSSTSPSVRKPYPVTKENQLEKDADSTDAESGAS |
| | EHE |
| SEQ ID | MLNFKKNTVVTSTLLAVALTLGTAQAAEYKASDKEGPIKIVNLDELESQVAKN |
| NO: 111 | MEKGAFGYIRGGAEDELNLNKNTQHFDKKYIMPRVMQGIEISDIDLSTDFLGI |
| A0A345LZ1 | KLKTPIIQAPMAAQGLAHKEGEIATAKGMAKAGSIFSLSTYGNKTIEEVAEVS |
| 5 | GNNPFFFQLYMSKNEAFNEFTLKRAKESGAKAIILTVDSPVGGYREDDIRNN |
| Providencia | FQFPLGFANLELFAQQNSDGSKTGKGAGISEIYAQAKQAFTPADIQYVKKLS |
| huaxiensis | GLPVIVKGIQSPEDADVVIKAGADAIWVSNHGGRQLDSGPASFDVLPSIAKVV |
| | NKRVPIVFDSGVRRGSHVFKALASGADVVAVGRPILYGLNLGGAEGVNSVIQ |
| | QLNKELSINMMLGGAKNIEGVKATQLYTDKDFQ |
| SEQ ID | MTKFMSKKSVLSLSAIALALSLGVAHAADYKASTAEGPVKITNLNELEGQVKA |
| NO: 112 | NMDKGAFGYIRGGAENEQNLRSNTSVFDKKYIMPRVMQGIELSEIDLKTNYL |
| A0A366E1 | GIDLKTPIIQAPMAAQGLAHQDGEIATAKGMAKAGSIFSLSTYGNKTIEEVAAV |
| E2 | SDGNPFFFQLYMSKNEAFNEFTLKRAKDSGAKAIILTVDSPVGGYREDDIKN |
| Pseudochro- | DFQFPLGFANLELFAKQNDDGSKTGKGAGISEIYAQAKQAFTPSDIKYVKDM |
| bactrum | TGLPVIVKGIQSPEDADVVIKAGADSIWVSNHGGRQLDSGPASFDVLPSIAKV |
| asaccharolyticum | VNKRVPIVFDSGVRRGSHVFKALASGADVVAVGRPILYGLNLGGAEGVNSVI |
| | QQLNKELTINMMLGGAKNIEAVKKTKLYSDKDFE |
| SEQ ID | MAYQASTEEHSIDIVNIASLEGKVKERMEAGAFGYIRGGSEDEWTMKENTTS |
| NO: 113 | FNTKKIMPRVLRGIDSADLSTSIFGIDLKTPIIQAPSAAQGLAHEKGEANTAKG |
| A0A367CB | VAAAGSIFSISTYANTTIQDAAAAAPGAPQFFQLYMSKDDGFNEFILNKAVEA |
| W9 | GAKAIILTADSTLGGYREEDIINHFQFPLPMPNLAAYSEQSASGNGEGKGISEI |
| Enterococcus | YAAAKQGLTPEDIKKIKDLTNLPVIVKGIQSPEDAEVAISAGADGIWVSNHGG |
| durans | RQLDGGPASFEVLPKVAEVVNKRVPVIFDSGVRRGEHVFKALASGADLVAIG |
| | RPVIYGLNLGGAQGVTSVFDHLNKELSITMQLAGTRTVNEIKNTKLLD |
| SEQ ID | MMKTNDLIRVVVSLAMLATSGLTYAEEYKASADEKAIKMTNVASLEARVQAR |
| NO: 114 | MEKGAFGYIRGGAEDENNLRSNTESFDKKYIMPRVLQGIELKEIDLSTQLLGI |
| A0A379XQ | PLKTPIIQAPMAAQGLAHVSGELATAKGMAQVGSIFSLSTYGNKTIEEVANVS |
| X7 | GESPFFFQLYMSKNNQFNEFILAQAVKHGAKAIILTVDSPVGGYREEDIKNNF |
| Salmonella | QFPLGFANLEMFARENDDGSKTGKGVGISEIYAQAKQAFTPEDIAYVHRVSG |
| enterica | LPVIVKGIQSPEDAETAIQAGAAGIWVSNHGGRQLDSGPSSFDMLPAIAKVV |
| subsp. | NKRVPVIFDSGVRRGSHVFKALASGADIVAVGRPILYGLNLGGAQGVASVIE |
| indica | QLNKELTINMMLGGARNIEQVKTTRLLTENELPQ |
| SEQ ID | MNKKKYEASTAENHVDIVNLASLEGRVKDHMSNEKGAFGYIRGGAEDEWTL |
| NO: 115 | NENTEAFNDKEIMPRVLQGIDHADLSTKLWDIDLKTPIIQAPSAAQGLAHIKGE |
| A0A386PW | TDTAKGVAAAGSIYSISTYASTSVEDAAAAAPDSPMFFQLYMSKDDKFNEFLI |
| Q7 | KKAVKAGAKAIIMTVDSTLGGYREADVINKFQFPLPMPNLAGYSAGDGEGKG |
| Companilac- | ISEIYASAKQGIVPTDIQKIKDMSGLPVFVKGIQSPDDAELAIQFGADGIWVSN |
| tobacillus | HGGRQLDGGPASFALLPYIAEVVDKRVPIVFDSGVRRGEHVFKALASGADLV |
| zhachilii | ALGRPIIYGLNLGGAQGVTDVIEHLNKELAITMQLAGTKTIDDVKNTELLD |
| SEQ ID | MAYITSSDEEKVEIVNIKSLETRVKERMEAQGNKGAFGYIRGGSEDEWTMAE |
| NO: 116 | NTTAFNHKKIVPRVLRGVDSADLSTSVLGINLKTPIIQAPVAAQGLAHMKGEV |
| A0A387BD | DTAKAMAEVGSLFSISTYGSTSVEEAAAVAPGAPQFFQLYMSKDDHFNEFLL |
| L4 | KKAVEAGVKAIILTADSTLGGYREEDVINHFQFPLPMPNLAAFSESDGTGKGI |
| Lactococcus | GEIYAAAKQGLVLEDIQKIKAITGLPVLVKGIQSPDDALLAIEAGADGIWVSNH |
| allomyrinae | GGRQLDGGPASFDVLPAIAKAVNKRVPVIFDSGVRRGEHVFKALASGADLV |
| | AVGRPVLYGLNLGGALGVQSVFEHLNKELSITMQLAGTKTIDEIKNTALID |
| SEQ ID | MTYYTSMEEHPIDILNLPSLEERVKANMERGAFGYIRGGSEDEWTLRENTRA |
| NO: 117 | FDDLQIIPRVLQGLSGADLSTSIFGISLKTPVIEAPSAAHGLAHVKGEIDTAIGT |
| A0A3A5RK | AAAGSLFSLSTYGSTDLRDVAAAAPGAPQFFQLYMSKDDGFNAYLVKKAVK |
| 32 | AGVKAIILTVDSTLGGYREEDVRNHFQFPLPMPNLAAYSSQDGVGKGIAEIYA |
| Acidaminococcus | AAKADFVPSDIDKIKTLSGLPVLVKGIQSPEDAEAAIKAGADGIWVSNHGGRQ |
| sp. | LNGGPASITVLPSIASVVRRRVPIVFDSGVRRGSHVFKALASGADLVALGRPL |
| AM33- | IYGLNLGGAEGVKSVFDQINHELSIVMQLAGTKDIEAIKRTPLLHKAAPLV |
| 14BH | |
| SEQ ID | MKKVKTGLFTATVLGLAMTLTTAYAAEYKASIKEGPLKIVNLNDLESQVKANM |
| NO: 118 | DKGAFGYIRGGAEDEKNMRDNTASFDRKYIMPRVMQGIELKDINISTSFLGIP |
| A0A3D3HP | LDTPVIQAPMAAQGLAHRDGEIATAKGMAKAGSIFTLSTYGNKTIEEVAAVSD |
| G0 | GHPFFFQLYMSKNDAFNEFTLKRAKESGAKAIILTVDSPVGGWREDDLRNN |
| Morganella | FQFPLGFANLELFAKQNNDGAKTGKGAGISEIYAQAKQAFTPSDIQYVKKMS |
| sp. in | GLPVIVKGIQSPEDADRVIEAGADAIWVSNHGGRQLDSGPASFDVLPAIAKTV |
| Bacteria | NKRVPVVFDSGVRRGSHVFKALASGADIVAVGRPVLYGLNLGGAQGVDSVI |
| | QQLNKELRINMMLGGAKDINAVKQTKLYTDNDFQ |
| SEQ ID | MAYQTSNEEHPIEIVNIASLEGRVKERMDKGAFGYIRGGSEDEWTMKENTT |
| NO: 119 | SFNNKTIMPRVLRGVDQADLSTKLWDIELATPIIQAPSAAQGLAHEKGEADTA |
| A0A3D9A2 | KGVAAAGSIFSISTYANTTIEDAAAAAPEAPQFFQLYMSKDDGFNEFILEKAV |
| L2 | KAGAKAIILTADSTLGGYREEDIINNFQFPLPMPNLAAYSEKSASGDGEGKGI |
| Enterococcus | AEIYAAAKQGLVPEDIKKIKDLTNLPVFVKGIQSPEDAEVAIAAGADGIWVSNH |
| pseudoavium | GGRQLDGGPASFDVLPLIAAVVNKRVPIVFDSGVRRGEHVFKALASGADLVA |
| | IGRPVIYGLNLGGAEGVKSVFDHLNKELSITMQLAGTKTLDEIKNTKLM |
| SEQ ID | MAYYTSNAEHPIDIVNIASLENRVKARMDRGAFGYIREGAEDEWTLRENTRA |
| NO: 120 | FNDLRIAPRVLQGIDHVDLSTSIFGIDLKTPIIEAPSAAHGLAHVKGEVDTAIGA |
| A0A3G9J8 | AKAGTLFAMSTYGSTPVEEAAAAAPEAPQFFQLYMSKDDGFNEFLIKKAVKA |
| G5 | GVKAIIMTVDSTLGGYREEDIVSHFQFPLPMPNLAAYSASDGVGKGISEIYAA |
| Intestinibaculum | AKQDFVPSDIAKVKDMSGLPVFVKGIQSPEDAIVAIQAGADGIWVSNHGGRQ |
| porci | LDGGPASISVLPAIAKAVNHQVPVIFDSGVRRGQHVFKALASGADLVAIGRPV |
| | IYGLNLGGAEGVKSVFDHLNMELSITMQLAGTKTIEEIKKTTLL |
| SEQ ID | MTSYYNGFPQSDRDEAINMINLDELEERAKNVMPEGAYYYIASGSENEWTW |
| NO: 121 | RNNTTAFNHFQIIPRALTNMTDPQLDTEFMGMKLKTPVMISPIACHGIAHKDA |
| A0A3M0LW | EVATQIGAAAAGALFSSSTYANKSVEDIAAAAPEAPRFFQLYLSKDWDFNKM |
| F6 | VFDAVKKAGYKGIFLTVDALVSGFREANLRTKFAYPVPLDFFTRYQGAKGEG |
| Lactobacillus | QTVAQMYASSAQKIGPEDVKRIKEMSGLPVFVKGVMCAEDAYLAMGAGAD |
| sp. | GIVVTNHGGREVDCGPATIDMLPEIAKAVNHRVPIIFDSGVRRGSHVFKALAL |
| ESL0246 | GADLVGIGRPYLYGLALGGAKGVQSVIEQLNKELLIDMQLTGCKTIEDVKRAR |
| | ITHINYTADNLRSNTDPTRIKPYPVTKENQMKVDDSDTVSGASHS |
| SEQ ID | MTAYYNDFPQSDRDEYIKMINLDELEERAKKVMPEGAYYYIASGSENEWTW |
| NO: 122 | RNNTVAFNHFQIVPRALTNMADPQLDTDFMGMKLKTPIMISPIACHGIAHQD |
| A0A3M0NI | AEVATQKGAAAAGALFSSSTYANKSVEDIAAAAPEAPRFFQLYLSKDWNFN |
| S1 | QMVFDAIKKAGYQGIFLTVDALVSGFREANLRTQFAFPVPLDFFTRYQGTKG |
| Lactobacillus | EGQTVAQMYASSAQKIGPEDVKRIKEMSGLPVFIKGVVCAEDAYLAMGAGA |
| sp. | DGIVVTNHGGREIDGGPATIDMLPEIAKAVDHRVPIVFDSGVRRGSHVFKALA |
| ESL0225 | LGADLVGIGRPYLYGLALGGAKGVQSVIEQLNKELLIDMQLTGCKTVADIQHA |
| | KITNINYTADNLSSNTDPERIKPYPVTKENQLKAGGMDTVSGASHS |
| SEQ ID | MVVVNGYKQNENEKKLNVLNLDQLEKQAKEIIPTGGFGYISGGSEDEWTLR |
| NO: 123 | ENRRAFTHKQIVPRALTNIEKPELETNVFGIPLKTPLFMVPAAAQGLAHAKGE |
| A0A3R817H | VDTAKGVAAVGGLMAQSTYSSTSIADTAASGNGAPQFFQLYMSKDWDFNE |
| 0 | ALLDEAKRAGVKGIILTVDATVDGYREADIINNFQFPIPMANLTKYSEGDGQG |
| Lactobacillus | KGIAEIYASAAQKIGPDDVARIANYTDLPVIVKGIESPEDALYAIGAGASGIYVS |
| sp. | NHGGRQLNGGPASFDVLEDVAKAVNGKVPVIFDSGVHRGSDVFKALASGA |
| | DLVGIGRPVIYGLALGGAQGVQSVFEHLDHELEIIMQLAGTKTISDVKNAKLL |
| | NIRY |
| SEQ ID | MTVINGYEQSDREEKLNVLNLPSLEAEAKKIIPTGGFGYISGGSEDEWTLNH |
| NO: 124 | NSEAFNHVQIVPRALTNMENPSTATSIFGLDLKTPIMMAPAAAQGLAHSRGE |
| A0A401FJ3 | VATAEGVSAAGALMGQSTYSSTSIADTAIAGHGAPQFFQLYMSKDWSFNES |
| 2 | LLDEAKRSGVKAIILTVDATVDGYREADIINKFQFPIPMANLTKFSEGDGQGK |
| Lentilacto- | GIEEIYASAAQKISAEDVKRIADYTDLPVIVKGIQSPEDALLAIGAGAQGIYVSN |
| abcillus | HGGRQLNGGPASFDVLADIAKAVNHRVPIIFDSGVRRGSHVFKALASGADIV |
| curieae | ALARPIIYGLALGGAQGVQSVIEELNHELEIDMQLAGTKDIEAVKHAKLIKINY |
| SEQ ID | MKNKILKTTAIAMALSIGVAQAAEYKASTAEGPIKIVNLNAMENQVKGNMEKG |
| NO: 125 | AFGYIRGGAEDENNLRANTKAFDKKYIMPRALQGIEFSDLDLKTEFLGIKLDT |
| A0A410XA | PIIQAPMAAQGLAHQQGEVATAKGMAKAGSIFSLSTYGNKTIKEVADAKPGY |
| C2 | PFFFQLYMSKNDAFNEYILSQAKQYGAKGIIMTIDSSVGGYREDDVKNNFQF |
| Proteus | PLGFANLEAFAKISDDKSKTGKGAGISEIYAQAKQAFTPADIQYVKKMSGLPV |
| hauseri | IVKGIESPEDADTAIKAGADAIWVSNHGGRQLDSAPATIDVLPAIAKVVNKRV |
| | PIVFDSGVRRGSHVFKALASGADVVAIGRPILYGLNLGGAEGVNSVIQQLNK |
| | ELKINMMLGGTKTVKDIQATQLYTEADFK |
| SEQ ID | MSYQTSNEEKDIEIVNVAGLEEEVRPRMDAGAFGYIRGGAETEWTMKENTA |
| NO: 126 | AFNKRQITPRVLQGISSADLSTKLWDIDLKTPIIEAPSAAQGLAHEKGELDTAK |
| A0A413AP | GVAAAGSIFSISTYANTTIEAAAQAAPGAPQFFQLYMSKDDGFNEFIIKKAVA |
| M7 | AGAKAIVLTVDSTLGGYREADVINHFQFPLPMPNLAAYSAQSASGDGEGKGI |
| Enterococcus | AEIYAAAKQDFVPEDIQKIKDLSGLPVIVKGIQSPLDAEVAIQAGADGIWVSNH |
| asini | GGRQLDGGPASFDVLPGIAKVVAKRVPVIFDSGVRRGEHVFKALASGADLV |
| | AIGRPVIYGLNLGGAQGVKAVFDHLNKELAITMQLAGTPTIDAVKKTPLVQF |
| SEQ ID | MTYYYNGFPQSERDQKLNMVNVDDLEEKVKQIMPEGAYYYIASGSENEWT |
| NO: 127 | WRNNTAAFNHYQIVPRALTDMSDPQTDTEFFGMKLKTPVMISPIACHGIAHK |
| A0A437SV | DAEVATQKGAAAAGALFSSSTYANKSVEDIAAAAPDAPRFFQLYLSKDWNF |
| F6 | NKMVFDAIKKAGYKGIFLTVDALVSGYREANLRTNFTYPVPLDFFTRYMGAK |
| Lactobacillus | GEGQSVAQMYAASAQKIGPEDVRRIKEETGLPVIVKGVMCAEDAFKAIGAGA |
| xujianguonis | DGVYVTNHGGREVDGAPATIDVLPEIAKAVDHRVPIIFDSGVRRGSHVFKAL |
| | ALGADMVGIGRPYLYGLALGGAKGVESVIEQYNKELKIDMQLTGCKTIEDVK |
| | HAKIDHIPYTADNLKSNTDPSRMKPYPKTEENQIKGSADATSGASHH |
| SEQ ID | MTYQASNEEHPIEIVNIASLEGRVKERMEKGAFGYIRGGSEDEWTMKENTS |
| NO: 128 | SFNKKTIMPRVLRGIDHADLSTKLWDMDLATPIIQAPSAAQGLAHKKGEADT |
| A0A437UR | AKGVAAAGSIFSISTYANTTIEDVAAAAPSAPQFFQLYMSKDDGFNAFILEKA |
| 51 | VKAGAKAIILTADSTLGGYREEDIVNKFQFPLPMPNLAAYSAKSASGDGEGK |
| Enterococcus | GIAEIYAAAKQGLVPEDIKKIKDLTSLPVFVKGIQSPEDAEVAIAAGADGIWVS |
| avium | NHGGRQLDGGPASFDVLPLIAGVVNKRVPIVFDSGVRRGEHVFKALASGAD |
| | LVAIGRPVIYGLNLGGAEGVKSVFDHLNKELSITMQLAGTKTINEVKNTRLHD |
| | AK |
| SEQ ID | MTTYYKGFPQSTREEKLHMVNLDELENEAKYVMPEAAYYYVASGAENEWT |
| NO: 129 | WRNNTQAFNHFQIVPRALTGMQDPELNTEFLGMKLKTPVMICPIACHGIANA |
| A0A451F3I | EAEIDTAKGAKVAGALFAMSTYANKSVQEVQNAVGDSPRFMQLYLSKNWD |
| 8 | FNKMVIEESVKAGFSGFFLTVDALVSGYREANLRTNFTYPVPLAFFNEWNG |
| Lactobacillus | GKGEGQSVAQMYASSAQNIGPDDIRRIKEIADVPVIVKGVECAEDAMLAIGA |
| johnsonii | GADGIVVSNHGGREVDGAPATIDVLPEIAKAVKSCDHRVPIILDGGVRRGSH |
| | VFKALALGADLVGIGRPFLYGLALGGAQGVQSVIEQLNKELLIDMQLTGCKTI |
| | EDIKKAKIDHINYGADWGISSTSRSVMKPYPVTKENQLTGEAADAVSGASRH |
| SEQ ID | MTYHTSNEEHPIDIVNIASLEELVKQRMDKGAFGYFRGGSEDEWTMKENTF |
| NO: 130 | AFTKKSIMPRVLRGIDQPDLRTTLWDIDLATPIIQAPSAAQGLAHEKGEVDTA |
| A0A4P5P8 | KGVAAAGSIFSISTYANTTIEHAAAAAPEAPQFFQLYMSKDDGFNRFILEKAV |
| Y5 | DAGAKAIILTADSTLGGYREEDVINHFQFPLPMPNLAAYSEQSNQSDGEGKG |
| Enterococcus | IAEIYAAAKQALVPEDIKKIKELTNLPVFVKGIQSPEDAEVAISAGADGIWISNH |
| florum | GGRQLDGGPASFDVLPLVAGVVNKRVPIVFDSGVRRGEHVFKALASGADLV |
| | AIGRPVIYGLNLGGAEGVKSVFDHLNKELAITMQLAGTKTVEEIKNTNLL |
| SEQ ID | MAYHTSTEERPIDIVNIPALEPIVKAKMNKGAFGYLAGGAESEITLRENVTSFE |
| NO: 131 | HKKILPRVLRNVEKPDMSTELFGIHIDAPIIAAPIAAHGLVHEQAEKDTVQGVG |
| A0A4P6YR | AAGSIFSLSTYGNATVDEVAQASPDTAKFFQLYMSKDDDFNHWILDMAVND |
| 47 | GYKAIILTADSTLGGYRESDIMNNFAFPLPMKNLAAWAAKTAGPDSGEGEGI |
| Weissella | AAIYAKAKQKLSLADIKAIKDYTHLPVIVKGVQSPLDIEDLLNAGADGIWVSNH |
| cryptocerci | GGRQIDGAPGSFDTLAEIAAVVAKRVPVIFDSGVRRGQHIFKALASGADIVAI |
| | GRPMLWGLSLGGVQGVTDVYNHFKKELTIDMQLAGTQTIADVKNTVLADAK |
| SEQ ID | MTVINGYEQSDREQKLAILNLPSLEAAAKKIIPTGGFGYISGGSEDEWTLKQN |
| NO: 132 | TAAFNHVQIVPRALTDMEKPSTQTQVFGIDLKTPIMMAPAAAQGLAHARGEA |
| A0A4Q9XX | ATAEGMAQVGALMAQSTYSSTSIADTAAAGKGAPQFFQLYMSKDWDFNQS |
| Y3 | LLDEAVKAGAKAIILTVDATVDGYREADIINNFQFPIPMANLTKFSEGDGKGK |
| Lactiplanti- | GIMEIYAAAAQKISPADVRRITEYTNLPVIVKGIQSPEDALLAIGAGAQGIYVSN |
| bacillus | HGGRQLNGGPASFDVLHAIAQAVSGRVPIIFDSSVRRGSHVFKSLANGADLV |
| paraplantarum | ALARPVIYGLALGGAQGVASVISHLNEELLVDMQLAGTKTIEDVKHAKLLRK |
| SEQ ID | MKTPPLSKMALASGLLTAVLSLNVAQAQEYRASDAEKAIKIVNIGALEAEVKA |
| NO: 133 | SMDKGAFGYIRGGAEDENNMRSNTEHFDKKYIMPRIMQGIERTDIDLSTELL |
| A0A4R5K2 | GIKLKTPIIQAPMAAQGLAHKEGEIATARGMAKAGSIFSLSTYGNKTIEEVATA |
| 06 | SKNNPFFFQLYMSKNEKFNEFTLKKARQFGAKAIILTVDSPVGGHREDDIRN |
| Pseudomonas | NFQFPLGFANLELFAKQNSDGSKTGKGSGISEIYAQAKQAFTPADIAYVQKL |
| sp. H9 | SGLPVIVKGVQSPEDADMAIKAGAAAIWVSNHGGRQLDSGPSSFDVLPGIAK |
| | VVNKRVPIIFDSGVRRGSHVFKALASGADIVAVGRPILYGLHLGGSEGVNSVI |
| | EHLNKELSINMMLGGAKNIEAVKSTKLYTDKDFEG |
| SEQ ID | MTVVNGYKQNDNEEKINILNLEESEERAKQIIPTGGFGYIVGGSENNWTLKA |
| NO: 134 | NRKAFTHKQIVPRALSNIENPSLDTNVFGIPLKTPIMMAPTAAQGLAHSQGEK |
| A0A4R5NI | DTAKGVAAVGGLMAQSTYSSVSISDTAAAGNGAPQFFQLYMSKDWDFNYS |
| Q5 | LLDEAKKAGVKGIILTVDATVDGYREDDIKNNFQFPIPMANLTKFSEGDGKGK |
| Lentilacto- | GIAEIYAAAAQKIGPDDVKKIADYTDLPVIVKGIESPEDALYAIGAGAAGVYVS |
| bacillus | NHGGRQLNGGPASFDVLEDVAKAVNGRVPIIFDSGVRRGSDAFKALASGAD |
| buchneri | LVAMGRPVIYGLALGGAEGVQAVFEHLGDELKTTMQLAGTKTIADVKKTHLL |
| DSM 20057 | SINY |
| SEQ ID | MTYYYNGFPQSEHDEKLNLINVDQLEEDAKRVIPEGAYYYIASGAENEWTW |
| NO: 135 | RNNTAAFNHYQIVPRALTDMEDPQLDTEFMGIKLKTPVMIAPIACHGIAHKDA |
| A0A4S2BH | EVATQKGAAAAGALFSSSTYANKSVEDIAAAAPDAPRFFQLYLSKDWNFNK |
| N9 | MVFDAVKKAGYKAIMLTVDALVSGYREANLRTKFTYPVPLDFFTRYLGAKGE |
| Lactobacillus | GQSVAQMYAASAQKIGPKDVARIKEESGLPVFIKGITCVEDAYKALGAGADGI |
| intestinalis | YVTNHGGREIDCSPATIDVLPDIAKAVNHRVPIIFDSGVRRGSHVFKALALGA |
| | DLVAIGRPYLYGLALGGAKGVESVIEQYNKELKIDMQLTGCKTIDDVKKAKIM |
| | HINYTADNLPSNTDPSRMKPYPVTSENQPQEDLDATSGASHH |
| SEQ ID | MKVNKKILAIASVAMMISSGLAQAEAYKASTEEHPIKMVNIDSLEDQVKARME |
| NO: 136 | KGAFGYIRGGAEDENNLRSNTESFDKKFIVPRVLQGIELKNINLSTSLLGIPLK |
| A0A4U6LS | TPIIQAPMAAQGLAHEAGELATARGMAQVGSIFSLSTYGNKTIEDVSRASEG |
| 33 | NPFFFQLYMSKNNKFNEFILNEAVKHGAKAIILTVDSPVGGYREDDIKNNFQF |
| Citrobacter | PLGFANLEMFAKQSDDGSKTGKGSGISEIYAQAKQAFTPEDIKYVHQISGLP |
| sp. wls619 | VIVKGIQSPEDAEVAIKAGAAAIWVSNHGGRQLDSGPSSFDVLPSIAKVVNKR |
| | VPIIFDSGVRRGSHVFKAIASGADIVAIGRPVLYGLNLGGAQGVASVIEQLNK |
| | ELVINMMLGGTKDIEQVKKTKLLTLRDLEE |
| SEQ ID | MKKYEASTEEKHIEIVNIAGLEKRVRDHMSNEKGAFGYIRGGSEDEWTMKQ |
| NO: 137 | NTEAFNKKKIMPRVLQGINHADLSTKLWDIDLKTPIIEAPSAAHGLAHAKGEV |
| A0A4Z0JR | DTAKGVADAGTIFSMSTYGSTSLEDGAAAAPGAPQFFQLYMSKDDKFNEFLI |
| Y6 | KKAVKAGVKAIVLTVDSTLGGYREEDVISKFQFPLPMPNLAAYSNGDGEGKG |
| Companilac- | ISEIYAAAKQGIVPSDIQKIKDLSGLPVFVKGIQSPDDAELAIDFGADGIWISNH |
| tobacillus | GGRQLDGGPASFDVLPAIASVVDKRVPIVFDSGVRRGEHVFKALASGADLV |
| suantsaicola | AIGRPIIYGLNLGGAQGVTDVIEHLNMELSITMQLAGTKTINDVKNTQLLD |
| SEQ ID | MAYQGSTKEEALNIIDLPRLRAAVQRDTEAGAFGYVDGGSSDEQVLHDNET |
| NO: 138 | AFRHYQLIPRMLQNISAPDLSTTLLDIPLSMPVIAAPIAAHGLMHENGERVTVK |
| A0A4Z0RW | GVGAAGTIFSLSTYGNSRIADVAASSPETPKFFQLYMSRDDEFNQYLLDEAV |
| W1 | KNGYKAIILTADATLGGYREADIINNFAFPLPMENLAAFSNAAGSGEGLGIAEI |
| Weissella | YARAKQDLALSDITKVKQMANGLPVFVKGIQDPDDALAAIAAGADGIWVSNH |
| confusa | GGRELNGAPASIDTLAAVAKAVNHRVPLVFDSGIRRGEDVAKAIALGADVVA |
| | LGRPMLWGLNQGGAAGVQSVYEHLAEELKIVMQLTGSHTVAELQRAKIIDA |
| | KF |
| SEQ ID | MTVTNGYEQSDREQKLDFINLNDLEKEAEAIIPKGGFGYIRGGSEDEWTLRE |
| NO: 139 | NTAAFNHAQIVPRALTDMENPATETDAFGLHFKTPLMMAPTAAQGLAHSQG |
| A0A510TU | EKDTARGVAAAGALMAQSTYSSTSIADTAAAGNGAPQFFQLYMSKDWDFN |
| B4 | HHLLDEAKKAGIKGIILTLDAPVDGYREDDLRNHFQFPIPMANLTEYSEGDGS |
| Schleiferilac- | GKGIAEIYAAAAQKIGPKDIERIAAYTDLPVIAKGIMSPEDALKAIGAGAAGVYV |
| tobacillus | SNHGGRQLNGGPASFDVLPSIAAAVNHQVPIIFDSGVRRGSHVFKALAAGA |
| harbinensis | DLVAFGRPAIYGLALGGAQGVQSVFEHLNDELKIDMQLAGTKTIADVKHAKL |
| | THFPA |
| SEQ ID | MAYITSSDEEKVEIVNIKSLETRVKERMEAQGNKGAFGYIRGGSEDEWTMVE |
| NO: 140 | NTKAFNHKKIVPRVLRGVDSADLSTSIFGINLKTPIIQAPVAAQGLAHMKGEV |
| A0A514Z8 | DTAKAMAEVGSLFSISTYGSTSVEEAAAVAPGAPQFFQLYMSKDDHFNEFLL |
| C9 | KKAVEAGVKAIILTADSTLGGYREEDVINHFQFPLPMPNLAAFSESDGTGKGI |
| Lactococcus | GEIYAAAKQGLVLEDIQKIKAITGLPVLVKGIQSPDDALLAIEAGADGIWVSNH |
| sp. KACC | GGRQLDGGPASFDVLPAIAKVVNKRVPVVFDSGVRRGEHVFKALASGADLV |
| 19320 | AVGRPVLYGLNLGGALGVQSVFEHLNKELSITMQLAGTKTIDEVKNTALID |
| SEQ ID | MTHYYEGFPQSDRNQYLHMVNLDQLEQDVKKVMPEGAYYYIASGAENEWT |
| NO: 141 | WRNNTAAFNHYQIVPRALTDMQDPVTDTEFMGMKLKTPVMISPIACHGIAH |
| A0A556UD | MDAEVATQKGAAAAGALFSSSTYANKSVADIAKAAPNAPRFFQLYLSKDWK |
| X1 | FNEMVFDEIKKAGYKGIFLTVDALVSGYREANIRTHFTYPVPLAFFQEYMGAK |
| Lactobacillus | GKGQSVAQMYASSAQKIGPEDVRRIKEATGLPVIVKGVECAEDAFKAIGAGA |
| sp. LL6 | DGIYVTNHGGREVDGAPATIDVLPSIAKAVNHRVPIVFDGGVRRGSHVFKAL |
| | ALGADIVGFGRPYLYALALGGAKGVESAINQLNKELKIDMQLTGCKTIDDVKH |
| | AKIDQFRYSADNLDSNTSFDRMKPYPKTSENQIKTGSQADATSGASEH |
| SEQ ID | MTVYYKGFPQSDRNEAIKMVNVDELEDRVRKVMPEAAYYYIASGSENEWT |
| NO: 142 | WRNNTTAFNHFQIVPRSLTNMDSPSTATQFMGMDLKTPIMICPIACHGIAHK |
| A0A558LX | DAEVATAQGAKAAGALFSSSTYANKSVEDIAAATGDSPKFFQLYLSKDWDF |
| H1 | NKMVFDAVKSAGYKGIMLTVDALVSGYREANLRTNFTFPVPLDFFTRYVGAE |
| Lactobacillus | GEGMSVAQMYANSAQKIGPADVAKIKEMSGLPVFVKGIMNAEDAYMAIGAG |
| jensenii | ADGIVVSNHGGREIDTAPATIDMLPEITAAVNGRVPIILDSGVRRGSHVFKALA |
| | LGADLVGIGRPFLYGLALGGAKGVESVINQINNEFKILMQLTGCKTVEDVKHA |
| | DIRQINYTADNLPSNTDPSVRRAYPVTKENQMEGTQDAATGASKH |
| SEQ ID | MTYYYKGFPQSDRDEKISMINVDELEERAKKVMPEGAYYYIASGAENEWTW |
| NO: 143 | RANTSAFNHYQIVPRALTDMQDPQTDTQFMGMKLKTPIMISPIACHGIAHKD |
| C2EPE3 | AEVATQKGVAAAGALFSSSTYANKSVEDIAAVAPEAPRFFQLYLSKDWDFN |
| Lactobacillus | KMVFDAIKKADYKGIFLTVDALVSGYREANLRTKFTYPVPLDFFTRYLGAKGE |
| ultunensis | GQSVAQMYAASAQKIGPEDVARIKKESGLSVFVKGVMCAEDAYKAIGAGAD |
| DSM 16047 | GIYVTNHGGREIDGSPATIDVLPEIAKAVNHRVPIVFDSGVRRGSHVFKALAL |
| | GADLVGIGRPYLYGLALGGPKGVESVIDQLNTELKIDMQLTGCKTIEDIKHAKI |
| | SRIHYGLDAMPSNTDPSRMKPYPTTAENQIKSTNTDAVTGASQH |
| SEQ ID | MDYTTSKEEHPIKIVNLSSLEKAVKEDMDKGAFGYIRGGSEDEWTLRENTQA |
| NO: 144 | FSKKKIIPRVLQGIDHADLSTELFGIPLKTPIIQAPSAAQGLAHVKGEVDTAIGV |
| E6LE88 | AKAGSIFAISTYANTKIEDAAAAAPDAPQFFQLYMSKDDAFNQFLLDKAVQS |
| Enterococcus | GAKAIILTVDSTLGGYREEDIVNDFTFPLPMPNLVAFSEASGAGEGKGKGISE |
| italicus | IYVAAKQAIVPEDIQKIKKMSGLPVIVKGIQSAEDGEVAIQFGADGIWVSNHG |
| DSM 15952 | GRQLDGAPASFDVLPQIAQVVRKRVPIIFDSGVRRGEHVFKALASGADVVAL |
| | GRPIIYGLFLGGAEGVTSVFDHLNKELAITMQLAGTKTIEDVKQTTLVD |
| SEQ ID | MTYYYNGFPQSDRNEKLDMVNVEELEERVKDVMPEGAYYYIASGSENEWT |
| NO: 145 | WRNNTAAFNHYQIVPRALTDMDNPSTETEFMGMKLKTPIMISPIACHGIAHK |
| I7IY70 | DAEIATQQGAAAAGALFSSSTYANKSVEEIAAAAPEAPRFFQLYLSKDWEFN |
| Lactobacillus | RMVFDAIKKAGYKGIFLTVDALISGYREANLRTNFTYPVPLDFFTRYLGGKGE |
| pasteurii | GQSVAQMYASSAQKIGPADVRRIKEESGLPVFVKGVMCAEDAYLAMGAGA |
| DSM 23907 | DGIYVTNHGGREVDTAPATIDVLPEIAAAVDHRVPIIFDSGVRRGSHIFKALAM |
| | GADLVGIGRPYLYGLALGGAKGVESVIDELNEELIINMQLTGCKTIEDVKHAKI |
| | SNIRYTADNLPSNTDPTRREAYPKTAENQIKVEADADATSGASKH |
| SEQ ID | MTVYYKGFPQSTREEKLKMTNLLELPEKVKKIMPEGAYYYIASGAENEWTW |
| NO: 146 | RNNTQAFNHFQIVPRALTNMQDPQLDTTFMGMNLKTPVMICPIACHGIANAE |
| I7KHU9 | AEIDTAKGAKAAGALFGMSTYCNKSVQDVQKAVGKSHRFMQLYLSKDWDF |
| Lactobacillus | NKMVIEESEKAGFEGFYLTVDALVSGYREANIRTNFTYPVPLAFFQEWTGGK |
| hominis | GEGQSVAQMYANSAQKIGPDDVRKIKELTDLPLIVKGVKCAEDAYKALGAGA |
| DSM 23910 | DGIEVSNHGGREVDGGPATIDVLPGIAREVRHAGRKVPIIFDGGVRRGSHVF |
| | KALALGADLVGIGRPFLYGLALGGAQGVQSVIEQLNKELLIDMQLTGCKTIED |
| | VKHAKIDHFTYSADWGISSTSKSVMPPYPVTEDNQLKGEAADAVSGASEH |
| SEQ ID | MTHYYNGFPQSDRDEKLDMVNVDELEERVKQVMPEGAYYYIASGSENEWT |
| NO: 147 | WRNNTAAFNHYQIIPRALTNMDNPSTETEFMGMKLKTPIMISPIACHGIAHMD |
| I7LDV0 | AEVATQKGAAAAGALFASSTYANKSVEDIAAAAPDAPRFFQLYLSKDWEFN |
| Lactobacillus | RMVFDAIKKAGYKGIFLTVDALISGYREANLRTNFTYPVPLDFFTRYLGGKGE |
| gigeriorum | GQSVAQMYASSAQKIGPDDVRRIKEESGLPVIVKGVMCAEDAYLAIGAGAD |
| DSM 23908 | GVYVTNHGGREVDTAPATIDVLPEVVKAVNHRVPVIFDSGVRRGSHIFKALA |
| | MGADLVGIGRPYLYGLALGGAKGVQSVIEELNEELIINMQLTGCKTIEDVKHA |
| | RIDHIRYTADNLPSNTDPTRRAAYPKTAENQIKVEADTDATSGASHH |
| SEQ ID | MTTINGYEQSDREQKLDILNLSSLEEKAKKIIPAGGFGYISGGSEDEWTLHEN |
| NO: 148 | TSAFNHIQIIPRALTNVEQPTTATEVFGLKLKTPIMMAPAAAQGLAHSRGEKA |
| J1F3A0 | TAEGLTKVGGLMAQSTYSSTSIADTAAAGNGTPQFFQLYMSKDWDFNYSLL |
| Liquorilacto- | DEAVKAGAKGIILTVDATVDGYRESDIINNFQFPIPMANLAKFSEGDGKGKGI |
| bacillus | MEIYAAAAQKIGPDDVRRIAEYTNLPVIVKGIESAEDALLAIGAGAKGIYVSNH |
| mali KCTC | GGRQLNGGPASIDVLHEVAQAVNHRVPVIFDSGVRRGSHVFKALASGADLV |
| 3596 | ALARPIIYGLALGGAQGVASVISHLNDELKIDMQLAGTKTIEDVKKAKVIRK |
| SEQ ID | MTRYIKGFPQSEADEDLKFVNVDELEERAKEVMPEGAYYYVASGSEYEWT |
| NO: 149 | WRNNTNAYNHYQIVPRSLTGMDNPSTETEFMGMKLKTPIMISPIACHGISHA |
| KONIV8 | DAEVATQKGAALAGAMFSSSTYGNKPVEEIAAAAPDAPRMFQLYLSKNWDF |
| Lactobacillus | NQMVFDAIKAAGYKAILLTVDALVSGYREANLRTDFAFPVPLDFFTRFQGAK |
| equicursoris | GEGQTVAQMYASSAQNIGPDDIKRIKEMSGLPVIVKGINCAEDVEVALTAGA |
| DSM 19284 | DGVYVTNHGGREIDGAPATIDVLPEVVKAVNGRVPVIFDGGVRRGSHIFKAL |
| | ALGADLVGIGRPYLYGLALGGPHGVASVINELNDELKIDMQLTGCKTIEDVKH |
| | ARLTHFEYAGDTRPSSTDPRVRKPYPVTAVNQIKKDGTDAVSGASHH |
| SEQ ID | MTYVTSKDEQKVEIVNIASLEARVKNRMEEQGNKGAFGYMRGGSEDEWTM |
| NO: 150 | RENTASFNTKTIVPRILRGIDSADLSTSIFGINLKTPFIQAPVAAQGLAHVAGEV |
| K2PXL4 | DTAKAMEEVGSLFSISTYGSTSVEDAAKAVNGAPQFFQLYMSKDDQFNQFL |
| Lactococcus | LEKAVKAGVKAIILTADSTLGGYREEDVINHFQFPLPMPNLAAFSETDGVGKG |
| garvieae | IFEIYAAAKQGLVLSDIQKIKKWTNLPVIVKGIQDPTDAMEAIAAGADGIWVSN |
| DCC43 | HGGRQLDGGPASFTVLPNIAKVVNKRVPIIFDSGVRRGAHVFKALASGADLV |
| | AVGRPVLYGLNLGGKEGVKSVFEHLNKELSITMQLAGAKDIEAVKKTELL |
| SEQ ID | MTYYYNGFPQSEHDEKLNLINVDQLEEDAKRVIPEGAYYYIASGAENEWTW |
| NO: 151 | RNNTAAFNHYQIVPRALTDMEDPQLDTEFMGMKLKTPVMIAPIACHGITHKD |
| N2A7U5 | AEVATQKGAAAAGALFSSSTYANKSVEDIAAAAPDAPRFFQLYLSKDWNFN |
| Lactobacillus | KMVFDAVKKAGYKAIMLTVDALISGYREANLRTEFTYPVPLDFFNRYLGAKG |
| sp. | EGQSVAQMYAASAQKIGPKDVARIKEESGLPVLIKGITCVEDAYKALGAGAD |
| ASF360 | GIYVTNHGGREIDGSPATIDVLPDIAKAVNHRVPIIFDSGVRRGSHVFKALALG |
| | ADLVAIGRPYLYGLALGGAKGVESVIEQYNKELKIDMQLTGCKTIDDVKKAKI |
| | MHINYTADNLPSNTDPSRMKPYPVTSENQPQEDLDATSGASHH |
| SEQ ID | MTYQTSNEEHPIEIVNIASLEGRVKERMDKGAFGYIRGGSEDEWTMKENTS |
| NO: 152 | SFNNKTITPRVLRGIDHADLNTKLWDIDLATPIIQAPSAAQGLAHEKGEADTA |
| R2RJX2 | KGVADAGSIFSISTYANTTIEDAAAVAPNAPQFFQLYMSKDDGFNEFILEKAV |
| Enterococcus | KAGAKAIILTADSTLGGYREEDIINNFQFPLPMPNLAAYSAKSASGNGEGKGI |
| malodoratus | AEIYAAAKQGLVPEDIKKIKDLTNLPVLVKGIQSPEDAEVAIAAGADGIWISNH |
| ATCC | GGRQLDGGPASFDVLPLIAGVVNKRVPIIFDSGIRRGEHVFKALASGADLVAI |
| 43197 | GRPVIYGLNLGGAEGVKSVFDHLNKELAITMQLAGTKTIDEVKNTRLNDVK |
| SEQ ID | MTYQASNEEHPIEIVNIASLEARVKERMEKGAFGYIRGGSEDEWTMKENTAS |
| NO: 153 | FNKKSIMPRVLRGIDHADLRTKLWDMELKTPIIQAPSAAQGLAHEKGESDTA |
| R2SI35 | KGVAAVGSIFSISTYANTTIEDAAEAAPGAPQFFQLYMSKDDGFNEFILAKAV |
| Enterococcus | QAGAKAVILTADSTLGGYREEDIINHFEFPLPMPNLAAYSEKDAAGNGEGKGI |
| pallens | AEIYAAAKQGLVPEDIKKIKDITNLPVFVKGIQSPEDAEVAISAGADGIWISNH |
| ATCC BAA- | GGRQLDGGPASFDVLPLIAEVVNKRVPIVFDSGVRRGEHVFKALASGADLVA |
| 351 | IGRPVIYGLNLGGAEGVTSVFEHLNKELSITMQLAGTKTIEEVKNTKLI |
| SEQ ID | MAYQASTEEHPIEIVNIASLENRVKENMERGAFGYIRGGSEDEWTMAENTH |
| NO: 154 | AFKKKKIIPRVLRGIDHVSLKTTLWDIELDTPIIQAPSAAQGLAHEKGETNTAK |
| R3TNG2 | GVADAGSIFSISTYANTTIEDAAKAAPNAPQFFQIYMSKDDGFNQFILTKAME |
| Enterococcus | AGAKAIILTVDSTLGGYREEDVVNKFQFPLPMPNLAAYSEQSASGDGEGKGI |
| phoeniculic | SEIYAAAKQGLVPEDIRKIKAFTKLPVIVKGIQAPEDAEVAISAGADGIWVSNH |
| ola ATCC | GGRQLDGGPASFDMLPLIASIVNKRVPIIFDSGIRRGEHVFKALASGADLVAI |
| BAA-412 | GRPIIYGLNLGGAQGVKSVFDHLNKELSITMQLAGTKTIEDVKNTTLMD |
| SEQ ID | MTYQASNEEHPIEIVNIASLEGRVKERMEKGAFGYIRGGSEDEWTMKENTS |
| NO: 155 | SFNKKTIMPRVLRGIDHADLSTKLWDMDLATPIIQAPSAAQGLAHEKGEADT |
| S0K8K6 | AKGVADAGSIFSISTYANTTIEDAAAAAPNAPQFFQLYMSKDDGFNAFILEKA |
| Enterococcus | VKAGAKAIILTADSTLGGYREEDIVNKFQFPLPMPNLAAYSAKSASGDGEGK |
| avium | GIAEIYAAAKQGLVPEDIKKIKDLTSLPVFVKGIQSPEDAEVAIAAGADGIWVS |
| ATCC | NHGGRQLDGGPASFDVLPLITGVVNKRVPIVFDSGVRRGEHVFKALASGAD |
| 14025 | LVAIGRPVIYGLNLGGAEGVKSVFDHLNKELSITMQLAGTKTINEVKNTRLHD |
| | TK |
| SEQ ID | MYHTSNEEHPIDIVNIASLEGRVKERMEAGAFGYIRGGSEDEWTMKENTASF |
| NO: 156 | MNKKIMPRILQGIDHADLSTKLWDIELQTPIIEAPSAAQGLAHEKGEADTAKG |
| S0KGS3 | VAAAGSIFSISTYANTTVEDAAAAAPDAPQFFQLYMSKDDGFNEFILDKAVKA |
| Enterococcus | GAKAIILTADSTLGGYREEDVINQFQFPLPMPNLAAYSAQSASGNGEGKGIA |
| dispar | EIYAAAKQGLVPEDIKKIKDLTHLPVLVKGIQSPEDADLAIKAGADGIWVSNHG |
| ATCC | GRQLDGGPASFDVLPAIAEVVDKRVPIVFDSGVRRGEHVFKALASGADLVAI |
| 51266 | GRPVIYGLNLGGAEGVKSVFDHENKELSITMQLAGTKTIADVKKTKLID |
| SEQ ID | MTVVNGYKQNDNEEKINILNLEELEERAKQIIPTGGFGYIAGGSENNWTLKAN |
| NO: 157 | RQAFTHKQIVPRALSNIEDPQLDTNVFGIPLKTPIMMAPTAAQGLAHSQGEK |
| S4NIEO | DTAKGVAAVGGLMAQSTYSSVSIADTAAAGNGAPQFFQLYMSKDWDFNYS |
| Lentilacto- | LLDEAKKAGVKGIILTVDATVDGYREDDIKNNFQFPIPMANLTKFSEGDGKGK |
| bacillus | GIGEIYAAAAQKIGPDDVKKIADYTDLPVIVKGIESPEDALYAIGAGAAGVYVS |
| otakiensis | NHGGRQLNGGPASFDVLEDVAKAVNGRVPIIFDSGVRRGSDAFKALASGAD |
| DSM 19908 | LVAMGRPVIYGLALGGAEGVQAVFEHLGDELKTIMQLAGTKTIDDVKKTDLL |
| | NIKY |
| SEQ ID | MKSSHLIRVVASLAMLATSGLAYAEEYKASTDEKTIKMTNVASLEARVQARM |
| NO: 158 | DKGAFGYIRGGAEDENNLRSNTESFDKKYIMPRVLQGIELKEIDLSTQLLGIP |
| S5MQ29 | LKTPIIQAPMAAQGLAHASGELATAKGMAQVGSIFSLSTYGNKTIEEVANVSG |
| Salmonella | ENPFFFQLYMSKNNKFNEFILAQAVKHGAKAIILTVDSPVGGYREEDIKNNFQ |
| bongori | FPLGFANLEMFARKNDDGSKTGKGAGISEIYAQAKQAFTPEDIAYVHRISGLP |
| N268-08 | VIVKGIQSPEDAEIAIQAGAAGIWVSNHGGRQLDSGPSSFDMLPAIAKVVNKR |
| | VPVIFDSGVRRGSHVFKALASGADIVAIGRPVLYGLNLGGAQGVASVIEQLN |
| | KELTINMMLGGARNIEQVKNTRLLSEKDL |
| SEQ ID | MTMTNGYEQSDREQKLDFINVNDLAKEAQAIIPKGGYGYIRGGSEDEWTLR |
| NO: 159 | ENTLAFNHAQIIPRALTDMENPATDTEVFGLKLKTPLMMAPLAAQGLAHAKG |
| U4TVI1 | ETDTARGVAAVGGLMAQSTYSSTSIADTAAAGNGAPQFFQLYMSKDWDFN |
| Schleiferilac- | HHLLDEAKKAGVKGIILTLDAPVDGFREDDLRNHFQFPIPMANLTEYSEGDG |
| tobacillus | SGKGIAEIYAAAAQKIGPKDIERIAEYTDLPVIAKGIMSPDDAMKAIGAGAAGIY |
| shenzhenensis | VSNHGGRQLNGGPASFDVLPSIAQAVNHKVPIIFDSGVRRGSHIFKALAAGA |
| LY-73 | DLVALGRPVVYGLALGGAQGVQSVFEHLADELKIDMQLAGTKTIADVKNTRL |
| | AHFPA |
| SEQ ID | MKRQILKATAIAMALSVGVAQAAEYKASTAEGPIKIVNLKAMEAQVQANMEK |
| NO: 160 | GAFGYIRGGAEDENNLRANTRAFDKKYIMPRSLQGIEFSDINLKTEFLGIKLD |
| V6MF26 | TPIIQAPMAAQGLAHQQGEVATAKGMAKAGSIFSLSTYGNKTIKEVADAQPG |
| Proteus | YPFFFQLYMSKNDAFNEYILSQAKQYGAKGIIMTIDSSVGGYREDDVKNNFQ |
| hauseri | FPLGFANLEAFAKISDDKSKTGKGAGISEIYAQAKQAFTPADIQYVKKMSGLP |
| ZMd44 | VIVKGIESPEDADTAIKAGADAIWVSNHGGRQLDSAPATIDVLPAIAKVVNKR |
| | VPIVFDSGVRRGSHVFKALASGADVVAVGRPILYGLNLGGSEGVNSVIQHLN |
| | KELRINMMLGGAKTVKDIQATPLYTDASFNQ |
| SEQ ID | MAIVNGYKQNENEKALDILNLDQLEAQAKEIIPTGGFGYIAGGSEDEWTLQA |
| NO: 161 | NRRAFTHKQIVPRALTNIEKPELDTNVFGLPLKTPIFMVPAAAQGLAHAKGEV |
| X0PJP5 | DTAKGLAAVGGLMAQSTYSSTSIADTASAGAGAPQFFQLYMSKDWEFNKSL |
| Lentilacto- | LDEAKKAGVKGIILTVDATVDGYREADIVNNFQFPIPMANLTKYSEGDGQGK |
| bacillus | GIAEIYASAAQKIGPDDVARIADYTDLPVIVKGIESPEDALYAIGAGAAGIYVSN |
| farraginis | HGGRQLNGGPASFDVLADVARAVNGKVPIIFDSGVRRGSDVFKALASGADL |
| DSM 18382 | VGIGRPVIYGLALGGAQGVQSVFEHLDHELEIIMQLAGTKTIADVKKAKLLNIH |
| | Y |
| SEQ ID | MTYKTSTENKALEIVNVKSLEGKVKQSMEAAGNKGAFGYIRGGSEDEWTLN |
| NO: 162 | ENTSAFNKKQIMPRVLRGVDSADLSTSLFGIKLKTPIIQAPVAAQGLAHEEGE |
| WP_03860 | VATAKAMAEVGSIFSISTYGSTSVEDAAKAAPDAPQFFQLYMSKDDRFNEFL |
| 3448.1 | LKKAVSAGVKAIILTADSTLGGYREEDIVNHFQFPLPMPNLAAFSESDGTGKG |
| Lactococcus | ISEIYAAAKQGLVLEDIQKIKKITNLPVIVKGVQSPIDADDAINAGADGIWVSNH |
| lactis | GGRQLDGGPASIDVLPLIAKSVNHRVPIVFDSGVRRGEHVFKALAQGADVVA |
| | VGRPVLYGLNLGGAKGVQSVFEHLNKELSITMQLAGTKNIEEIKHTSLID |
| SEQ ID | MKTHHLIRVVASLAMLATSGLAYAEEYQSQHGRKAIKMTNVASLEARVQAR |
| NO: 163 | MEKGAFGYIRGGAEDENNLRSNTESFDKKYIMPRVLQGIELKEIDLSTQLLGI |
| WP_00084 | PLKTPIIQAPMAAQGLAHASGELATAKGMAQVGSIFSLSTYGNKTIEEVANVS |
| 8087.1 | GKNPFFFQLYMSKNNQFNEFILAQAVKHGAKAIILTVDSPVGGYREEDIKNNF |
| Salmonella | QFPLSFANLEMFARKNDDGSKTGKGAGISEIYAQAKQAFTPEDIAYVHRISGL |
| enterica | PVIVKGIQSPEDAEIAIQAGAAGIWVSNHGGRQLDSGPSSFDMLPAIAKVVNK |
| | RVPVIFDSGVRRGSHVFKALASGADIVAVGRPVLYGLNLGGAQGVASVIEQL |
| | NKELTINMMLGGARNIEQVKTTRLLTEKDLPQ |
| SEQ ID | MTVYYKGFPQSDRNEAIKMVNVDELEDRVRKVMPEAAYYYIASGSENEWT |
| NO: 164 | WRNNTAAFNHFQIVPRSLTNMDNPSTETQFMGMDLKTPIMICPIACHGIAHK |
| WP_00658 | DAEVATAQGAKAAGALFSSSTYANRSVEDIATATGDSPKFFQLYLSKDWDF |
| 8101.1 | NKMVFDAVKSAGYKGIMLTVDALVSGYREANLRTNFTFPVPLDFFTRYVGAE |
| Lactobacillus | GEGMSVAQMYANSAQKIGPADVAKIKEMSGLPVFVKGVMNAEDAYMAIGA |
| jensenii | GADGIVVSNHGGREIDTAPATIDMLPEIAAAVNGRVPIILDSGVRRGSHVFKA |
| | LALGADLVGIGRPFLYGLALGGAKGVESVINQINNEFKILMQLTGCKTVEDVK |
| | HADIRQINYTADNLPSNTDPSVRRAYPVTKENQMEGTQDAATGASKH |
| SEQ ID | MSYVTSNDEQKVEIVNIASLEGRVKERMEAQGNKGAFGYIRGGAEDEWTM |
| NO: 165 | RENTASFNTKTISPRVLRGIDSANLSTNIFGIELKTPIIQAPVAAQGLAHMEGE |
| WP_01736 | VDTAKAMEEVGSLFSISTYGSTSVEDAAAAVNSAPQFFQLYMSKDDQFNQF |
| 9343.1 | LLEKAVKSGVKAIILTADSTLGGYREEDVINHFQFPLPMPNLAAFSESDGVGK |
| Lactococcus | GIFEIYAEAKQGLVLSDIQKIKNWTNLPVIVKGIQDPVDAMEAIAAGADGIWVS |
| garvieae | NHGGRQLDGGPASFTVLPRIAQVVNKRVPIIFDSGVRRGEHVFKALASGADL |
| | VAIGRPVLYGLNLGGKEGVKSVFEHLNKELSITMQLAGAKDIEAIKNTDLL |
| SEQ ID | MTAYYNGFPQSDKDEKITMVNVDELEEKVKEVMPEPAYYYIASGSENEWTW |
| NO: 166 | RNNTAAFNHFQIVPRSLTDMDNPSTATEFMGMKLKTPIMIAPIACHGIAHKDA |
| WP_02502 | EIATAQGAKAAGALFSSSTYANKSVEEIAAATGDAPRFFQLYLSKDWEFNQM |
| 1424.1 | VFDAVKAAGYKGIFLTVDALVSGYREANLRTNFTFPVPLDFFTRYLGGKGEG |
| Ligilacto- | QSVAQMYASSAQKIGPADVARIKEMSGLPVFIKGVMNADDAYLALGAGADGI |
| bacillus | VVSNHGGREIDGAPATIDMLPEIARAVNKRVPIVFDSGVRRGSHVFKALALG |
| equi | ADLVGIGRPYLYGLALGGASGVQSVIEQINSELEIDMQLTGCKTIEDVKHADV |
| | RNLSYGLDNLPSSTSPKVRKPYPVTAENQMKVEAETDATSGASSH |
| SEQ ID | MKTFLSKKYAVSLSAIALALSLGVAHAADYKASTAEGPVKITNLGELEAQVKA |
| NO: 167 | NMDKGAFGYIRGGAENEQNLRSNTSVFDKKYIMPRVMQGIELSDIDLKTNYL |
| WP_02823 | GIDLKTPVIQAPMAAQGLAHQDGEIATAKGMAKAGSIFSLSTYGNKTIEEVAA |
| 3538.1 | VSDGNPFFFQLYMSKNEAFNEFTLKRAKDSGAKAIILTVDSPVGGYREDDIK |
| Brucellaceae | NNFQFPLGFANLELFAKQNDDGSKTGKGAGISEIYAQAKQAFTPADIKYVKD |
| | MTGLPVIVKGIQSPEDADVVIKAGADSIWVSNHGGRQLDSGPASFDVLPSIA |
| | KVVNKRVPIVFDSGVRRGSHVFKALASGADVVAVGRPILYGLNLGGSEGVN |
| | SVIQQLNKELTINMMLGGVKNIEEVKKTKLYSDKDFE |
| SEQ ID | MAYQASNEEHPINIVNIASLEGRVKERMEAGAFGYIRGGSEDEWTMRENTS |
| NO: 168 | SFNTKKIMPRVLRGIDSADLRTSVFGIDLDTPIIQAPSAAQGLAHEKGEAATAK |
| WP_03486 | GVADAGSIFSISTYANTTIKDAAAAAPDAPQFFQLYMSKDDGFNEFILNKAVE |
| 2474.1 | AGAKAVILTADSTLGGYREEDIINNFQFPLPMPNLAAYSEQSASGNGAGKGI |
| Enterococcus | AEIYAAAKQELTPADIRKIKELTNLPVLVKGIQSPEDAEVAISAGADGIWVSNH |
| faecalis | GGRQLDGGPASFEVLPKIAEVVSKRVPVIFDSGVRRGEHVFKALASGADLVA |
| | IGRPVIYGLNLGGAAGVTSVFEHLNKELAITMQLAGTKTIDEVKNTKLLD |
| SEQ ID | MTITNGYEQSDREEKLDIINLNELEAQAKAIIPQGGFGYISGGSEDEWTLREN |
| NO: 169 | TNAFEHAQIVPRALTDMDNPETDTTVFGLNLKTPIMMAPTAAQGLAHAKGEV |
| WP_03545 | DTAKGVAAAGALMAQSTYSSTSIADTAAAGNGAPQFFQLYMSKDWDFNNH |
| 1825.1 | LLDQAKAAGVKAIILTVDATIDGYREADLRNKFQFPIPMANLTEYSEGDGAGK |
| Agrilacto- | GIAEIYAAAAQKISAADVKRIADYTDLPVIVKGIESPEDALFAIGAGAKGIYVSN |
| bacillus | HGGRQLNGGPASFDVLADVAKAVDHRVPIIFDSGVRRGSHVFKALASGADL |
| composti | VAMGRPVIYGLALGGAQGVQSVFEELNGELKIDMQLAGTKTIEDVKKAKLLHI |
| | HY |
| SEQ ID | MTYKTSTENKALEIVNVKSLEGKVKQSMEAAGNKGAFGYIRGGSEDEWTLN |
| NO: 170 | ENTSAFNKKQIMPRVLRGVDSADLSTSLFGIKLKTPIIQAPVAAQGLAHAEGE |
| WP_04223 | VATAKAMAEVGSIFSISTYGSTSVEDAAKTAPGAPQFFQLYMSKDDKFNEFL |
| 0388.1 | LKKAVDAGVKAIILTADSTLGGYREEDIVNHFQFPLPMPNLAAFSESDGTGK |
| Lactococcus | GISEIYAAAKQGLVLEDIQKIKKITNLPVIVKGVQSPIDADDAINAGADGIWVSN |
| | HGGRQLDGGPASIDVLPLIAKSVNHRVPVIFDSGVRRGEHVFKALAQGADVV |
| | AVGRPVLYGLNLGGAKGVQSVFEHLNKELSITMQLAGTKNIEEIKHTSLID |
| SEQ ID | MTYQASKEEHPIEIVNIASLEKRAQETMEAGAFGYIRGGSEDEWTMAENTSS |
| NO: 171 | FNKKTIMPRVLRGIDSADLHTKLWDIQLETPIIQAPSAAQGLAHTNGEADTAK |
| WP_04860 | GVAAAGSIFSISTYANTTIEDAAAAAPNAPQFFQLYMSKDDGFNKFILDKAVK |
| 3977.1 | AGAKAIVLTVDSTLGGYREEDVINHFQFPLPMPNLAAYSEQSESGDGAGKGI |
| Enterococcus | AEIYAAAKQAIEPEDIQKIKTLTNLPVVVKGIQSPEDAEVAIAAGADGIWVSNH |
| massiliensis | GGRQLDGGPASFDVLPEIAAVVNKRVPIVFDSGVRRGEHVFKALASGADLV |
| | AIGRPVIYGLNLGGAEGVKSVFDHLNKELSITMQLAGTKTIEEVKNTKLFSY |
| SEQ ID | MTTYYNGFPQSDRNETIHMVNVDELEERAKKVMPEGAYYYIASGAENEWT |
| NO: 172 | WRNNIASFNHFQIVPRALTNMDNPQTDTEFMGMKLKTPIMISPIACHGIAHKD |
| WP_05468 | AEVATQEGAAAAGALFSSSTYANKSVEDIASAAPNAPRFFQLYLSKNWDFN |
| 2195.1 | KMVFDAVKKAGYKGIFLTVDALVSGYREANLRTKFAYPVPLDFFTRYQGAKG |
| Lactobacillus | EGQTVAQMYAASAQKIGPKDVQRIKKMSGLPVFVKGVMCAEDAFKAIGAGA |
| acetotolerans | DGIYVTNHGGREVDSGPATIDMLPSIAKAVNHRVPIVFDSGVRRGSHVFKAL |
| | ALGADMVGIGRPYLYGLALGGAKGVKSVIDQLDMELKIDMQLTGCKTIEDVK |
| | HAKIEHINYAADNLKSNTDPSRFKGYPVTKDNQIKEDKSSDAVSGASQA |
| SEQ ID | MGIFMAKYEASTEEKHIDIVNIASLEQRVKDHMSNEKGAFGYIRGGSEDEWT |
| NO: 173 | MKQNTEAFNKKRIMPRVLQGIDHADLSTKLWDIDLKTPIIEAPSAAHGLAHAK |
| KRK61221. | GEVDTAKGVAAAGSIFSMSTYGSTAIEDGAAAAPDAPQFFQLYMSKDDKFN |
| 1 | EFLIKKAVKAGVKAIILTVDSTLGGYREADVINKFQFPLPMPNLAAFSDGDGE |
| Companilac- | GKGISEIYAAAKQGIVPSDIQKIKDMSGLPVFVKGIQSPDDAELAIEFGADGIW |
| tobacillus | VSNHGGRQLDGGPASFDVLPSIAQVVDKRVPIVFDSGVRRGEHVFKALASG |
| farciminis | ADLVAVGRPIIYGLNLGGAQGVTDVIEHLNKELSITMQLAGTKTIHDVKNVDLL |
| KCTC 3681 | |
| SEQ ID | MGIFMAKYEASTEEKHIDIVNIASLEQRVKDHMSNEKGAFGYIRGGSEDEWT |
| NO: 174 | MKQNTEAFNKKRIMPRVLQGIDHADLSTKLWDIDLKTPIIEAPSAAHGLAHAK |
| KRK95764. | GEVDTAKGVAKAGSIFSMSTYGSTSIEDGAAAAPDAPQFFQLYMSKDDKFN |
| 1 | EFLIKKAVKAGVKAIILTVDSTLGGYREADVINKFQFPLPMPNLAAFSDGDGE |
| Companilac- | GKGISEIYAAAKQGIVPSDIQKIKDMSGLPVFVKGIQSPDDAQLAIEFGADGIW |
| tobacillus | VSNHGGRQLDGGPASFDVLPAIAEAVDKRVPIVFDSGVRRGEHVFKALASG |
| futsaii JCM | ADLVAIGRPIIYGLNLGGAQGVTDVIEHLNKELSITMQLAGTKTIHDVKNVDLL |
| 17355 | |
| SEQ ID | MVMTNGYEQNENEQALDILNLPQLEADAKKIIPTGGFGYISGGSEDEWTLRA |
| NO: 175 | NRTAFQHRQIVPKALSNIENPQTDTTVFGLNLKTPIMMAPAAAQGLAHAKGE |
| WP_05694 | VDTAKGVAKVGGLMAQSTYSSTSIADTAAAGEGAPQFFQLYMSKDWEFNK |
| 8709.1 | SLLDEAKKAGVKAIILTVDATVDGYREADIINNFQFPIPMANLTKFSEGDGKGK |
| Secundilac- | GIGEIYAAAAQKIGPADLQQIIDYTDLPVIVKGVESAEDALYAIGAGAAGIYVSN |
| tobacillus | HGGRQLNGGPASFDVLESVAKAVNHQVPVIFDSGVRRGSDVFKAIALGADL |
| odoratitofui | VAIGRPAIYGLALGGAEGVASVFTHLNDELKIIMQLAGTKTIDDIKQAELLNLN |
| | Y |
| SEQ ID | MTVVNGYKQNDNEQKLDILNLEDLEEKAKQIIPTGGFGYIAGGSENNWTLKA |
| NO: 176 | NRKAFTHKQIVPRALSNIEKPELDTNVFGIPLKTPIMMAPTAAQGLAHSQGEK |
| WP_05698 | DTAKGLAAVGGLMAQSTYSSTSIADTAAAGDGAPQFFQLYMSKDWDFNYSL |
| 1377.1 | LDEAKKAGVKGIILTVDATVDGYREDDIKNNFQFPIPMANLTKFSEGDGKGK |
| Lentilacto- | GIGEIYASAAQKIGPDDVKKIADYTDLPVIVKGIESPEDALYAIGAGAAGVYVS |
| bacillus | NHGGRQLNGGPASFDVLEDVAKAVNGQVPVIFDSGVRRGSDAFKALASGA |
| kefiri | DLVAMGRPAIYGLALGGAQGVQSVFEHLGDELKIIMQLAGTKTIADVKKTNLI |
| | NIKY |
| SEQ ID | MTLTNGYEQSDREQKLNIVNLPSLEAEAKKIIPTGGFGYIAGGSEDDWTLRQ |
| NO: 177 | NTAAFGHLQIVPKALSNIEQPSLATHVFGLDLKTPIMMAPTAAQGLAHSQGE |
| WP_05698 | KDTARGVAAVGGLMAQSTYSSTSIADTAAAGNGAPQFFQLYMSKDWTFNE |
| 8761.1 | SLLDEAVKAGVKAILLTVDATVDGYREADIVNNFQFPIPMANLTKFSEGDGKG |
| Lacticasei- | KGIGEIYASAAQKISEDDVRRIADYTHLPVIVKGIQSPEDALRAIGAGAAGIYVS |
| bacillus | NHGGRQLNGGPASIDVLPAIAKAVNHQVPIIFDSGVRRGSHVFKALAAGADL |
| camelliae | VAMGRPVIYGLALGGAQGVQSVFEELNHELEITMQLAGTKTIEDVKHAPLTH |
| | FNYAE |
| SEQ ID | MAYHTSDAEAPVDILNLDSLEGRVKERMEAGAFGYIRGGSEDEWTMAANTS |
| NO: 178 | AFNSKKIMPRVLQGIDHANLHTKLWDIDLKTPIIQAPSAAQGLAHEKGEVDTA |
| WP 05790 | KGVAAAGSIFSISTYANTLVEDAAAAAPDAPQFFQLYMSKDDQFNEFLLKKA |
| 7736.1 | VKAGVKAIILTVDSTLGGYREADIENHFQFPLPMPNLAAYSASDGEGKGISEI |
| Latilacto- | YAAAKQGLVPADIQKIKEITNLPVLVKGIQSPVDAEIAIQAGADGIWVSNHGG |
| bacillus | RQLDGGPASFEVLPLIAQQVAKRVPIVFDSGIRRGEHVFKALASGADLVAIGR |
| graminis | PIIYGLNLGGAQGVKSVFDHLNEELSITMQLAGTKTIDEIKETTLLD |
| SEQ ID | MTVVNGYKQNDNEQKIDILNLEELEERAKQIIPTGGFGYIVGGSENNWTLKA |
| NO: 179 | NRKAFTHKQIVPRALSNIEDPQLDTNVFGIPLKTPIMMAPTAAQGLAHSQGEK |
| WP_05790 | DTAKGVAAVGGLMAQSTYSSASISDTAAAGDGAPQFFQLYMSKDWDFNYS |
| 9428.1 | LLDEAKKAGVKGIILTVDATVDGYREDDIKNNFQFPIPMANLTKFSEGDGKGK |
| Lentilacto- | GIAEIYAAAAQKIGPDDVKKIADYTDLPVIVKGIESPEDALYAIGAGASGVYVS |
| bacillus | NHGGRQLNGGPASFDVLEDVAKAVNGRVPIIFDSGVRRGSDAFKALASGAD |
| parabuchneri | LVAMGRPVIYGLALGGAQGVQSVFEHLGDELKITMQLAGTKTIADVKKTHLL |
| | NIQY |
| SEQ ID | MKHTLLKTTAIAMALTVGVAQAAEYKASTAEHPIKIVNLDALENQVKENMDK |
| NO: 180 | GAFGYIRGGAEDENNLRSNTSAFNKKYIMPRALQGIEFSDLNLKTEFLGIKLD |
| WP_06055 | TPIIQAPMAAQGLAHQQGEVATAKGMAKAGSIFSLSTYGNKTIKEVAQAQPG |
| 7719.1 | YPFFFQLYMSKNDAFNQYILSQAKQYGAKGIILTVDSPVGGYREDDIKNSFQ |
| Proteus | FPLGFANLEAFAKISDDKSKTGKGSGISEIYAQAKQAFTPADIQYVKKMSGLP |
| mirabilis | VIVKGIGSPEDADIAIKAGADAIWVSNHGGRQLDSAPATIDMLPAIAKVVNKR |
| | VPIVFDSGVRRGSHVFKALASGADVVAVGRPILYGLNLGGAEGVNSVIQQLN |
| | KELRINMMLGGARNVKEIQATQLYTDADFK |
| SEQ ID | MSYHTSNEEHAIEIVNIASLEALVKARMELGAFGYIRGGAEDEWTMRENTLA |
| NO: 181 | FNRKKIIPRVLQGIDHADLSTKLWDISLKTPIIQAPSAAQGLAHEEGEKDTAKG |
| WP_06558 | VAAAGSIFCISTYANTSIEDAANAAPNVPYFFQLYMSKDDDFNQFIIDKAVKA |
| 5809.1 | GAKAIILTVDSTLGGYREEDIVNKFQFPLPMKNLSAYSQSNGNGDGSGKGIS |
| Gilliamella | EIYAAAKQGIVPSDIQKIKDMANLPIIVKGIQSPEDASIAISAGTDGIWISNHGG |
| apicola | RQLDGAPASFEVLPSISAAVAKRVPIIFDSGIRRGEHVFKALASGADLVAIGRP |
| | ILYGLNLGGAEGVKSVFDHLNKELSITMQLAGTKTIEDIKNTLLI |
| SEQ ID | MAYQGSTKEEALNIIELPRLRAAVQRDREAGAFGYVDGGSSDEQVLHANET |
| NO: 182 | AFRHYQLIPRMLQNISAPDLSTTLLDIPLSMPVIAAPIAAHGLMHENGERVTVK |
| WP_07170 | GVGAAGTIFSLATYGNSRIADVAASSPNTPKYFQLYMSRDDEFNQYLLDEAV |
| 7325.1 | QNGYKAIILTADATLGGYREADIINNFAFPLPMENLAAFSNAAGSGEGLGISEI |
| Weissella | YARPKQDLALSDITKVKQMANGLPVFVKGIQYPDDALAAIAAGADGIWVSNH |
| confusa | GGRELNGAPASIDTLAAVAKAVNHRVPVVFDSGIRRGEDVAKAIALGADVVA |
| | LGRPMLWGLNQGGAAGVQSVYEHLAEELKTVMQLTGSHTVAELQHAKIIDA |
| | KF |
| SEQ ID | MVYQTSNEEHPIEIVNIASLEKRVKERMDKGAFGYIRGGSEDEWTMKENTSS |
| NO: 183 | FNNKTIMPRVLRGVDHADLTTKLWNIDLSTPIIQAPSAAQGLAHEKGEADTAK |
| WP_07185 | GVAAAGSIFSISTYANTTIEAAAASAPEAPQFFQLYMSKDDRFNEFILNKAVH |
| 7665.1 | AGAKAIILTADSTLGGYREEDIINDFQFPLPMPNLAAYSEQSASGDGAGKGIA |
| Enterococcus | EIYAAAKQGLIPEDIKKIKELTDLPVFVKGIQAPEDAEVAIAAGADGIWVSNHG |
| hermanniensis | GRQLDGGPASFDVLPLIAAVVNKRVPIVFDSGVRRGEHVFKALASGADLVAI |
| | GRPVIYGLNLGGAEGVTSVFEHLNKELAITMQLAGTKTIEEIKNVTLK |
| SEQ ID | MKNKLLKTTAIAMALSVGVAQAAEYKASTAEGPIKIVNLKAMEAQVQANMDK |
| NO: 184 | GAFGYIRGGAEDENNLRSNTTAFDKKYIMPRSLQGIEFSDLDLKTEFLGIKLD |
| WP_07567 | TPIIQAPMAAQGLAHQQGEVATAKGMAKAGSIFSLSTYGNKTIKEVADAKPG |
| 2938.1 | YPFFFQLYMSKNDAFNEYILSQAKQYGAKGIIMTIDSSVGGYREDDVKNNFQ |
| Proteus | FPLGFANLEAFAKISDDKSKTGKGAGISEIYAQAKQAFTPADIQYVKKMSGLP |
| | VIVKGIQSPEDADVAIKAGADAIWVSNHGGRQLDSGPASIDVLPSIAKVVNKR |
| | VPIVFDSGVRRGSHVFKALASGADVVAVGRPILYGLNLGGAEGVNSVIQHLN |
| | KELKINMMLGGAKTVKDIQATQLYTDASFNQ |
| SEQ ID | MISLHGRKYAASLSAVVLAFSLGVAHASDYKASTAEGPIKIVNLNDLEAQVQN |
| NO: 185 | NMEKGAFGYIRGGAENEQNLRSNTSAFDKKYIMPRVMQGIELSNIDLSTSFL |
| WP_09454 | GLKLKTPVIQAPMAAQGLAHQDGEIATAKGMAKAGSIFSLSTYGNKTIEEVAE |
| 2538.1 | VSGDNPFFFQLYMSKNNAFNEFTLKRAKDSGAKAIILTVDSPVGGYREDDIK |
| Brucella | NSFQFPLGFANLELFAKQKDDGSKTGKGAGISEIYAQAKQAFTPTDIKYVKDL |
| grignonensis | TNLPVIVKGIQSPDDADTVIKAGADAIWVSNHGGRQLDSGPASFEVLPSIAKV |
| | VNKRVPIVFDSGVRRGSHVFKALASGADIVAVGRPILYGLNLGGAEGVNSVV |
| | QQLNKELTINMMLGGAQNIEAVKKTRLYTDKDFE |
| SEQ ID | MTTYYKGFPQSTREEKLHMVNLDELEKEAKYVIPEAAYYYIASGAENEWTW |
| NO: 186 | RNNTQAFNHFQIVPRALTGMQDPELNTEFLGMKLKTPVMICPIACHGIANAE |
| WP_09518 | AEVDTAKGAKVAGALFGMSTYANKSVQDVQSAVGDSPRFMQLYLSKNWDF |
| 3719.1 | NKMVIEESVKAGFTGFFLTVDALVSGYREANLRTNFTYPVPLAFFNEWTGGK |
| Lactobacillus | GEGQSVAQMYASSAQNIGPDDIRKIKEIADVPVIVKGVECAEDAVLAIGAGAD |
| johnsonii | GIVVSNHGGREVDGAPATIDVLPEIAKAVKSCDHPVPIILDGGVRRGSHVFKA |
| | LALGADLVGIGRPFLYGLALGGAQGVQSVIDQLNKELLIDMQLTGCKTIEDIK |
| | HAKIDHINYSADWGISSTSRSVMKPYPVTKENQLTGEAADAVSGASRH |
| SEQ ID | MKRQILKATAIAMALSVGVAQAAEYKASTAEGPIKIVNLKAMEAQVQANMEK |
| NO: 187 | GAFGYIRGGAEDENNLRSNTTAFDKKYIMPRSLQGIEFSDLDLKTEFLGIKLD |
| WP_09907 | TPIIQAPMAAQGLAHQQGEVATAKGMAKAGSIFSLSTYGNKTIKEVADAQPG |
| 4037.1 | YPFFFQLYMSKNDAFNEYILSQAKQYGAKGIIMTIDSSVGGYREDDVKNNFQ |
| Proteus | FPLGFANLEAFAKISDDKSKTGKGAGISEIYAQAKQAFTPADIQYVKKMSGLP |
| alimentorum | VIVKGIESPEDADTAIKAGADAIWVSNHGGRQLDSAPATIDVLPAIAKVVNKR |
| | VPIVFDSGVRRGSHVFKALASGADIVAVGRPILYGLNLGGAEGVNSVIQHLN |
| | KELKINMMLGGAKTVKDIQATQLYTDASFNK |
| SEQ ID | MSYKASNAEEHIKIVNLKSLEKEVKDRMEPGAFGYIRGGSEDEWTMDQNTE |
| NO: 188 | SFNSKKIVPRVFQGIDHADLSTKLWDVDLKTPIIQAPSAAQGLSHEKGEINTAI |
| WP_09997 | GMAKAGSIFSISTYANTLIKDAAAAAPDAPQFFQLYMSKDDGFNEFIIKKAIEA |
| 5166.1 | KVKAIVLTVDSTLGGYREEDVINDFEFPLPMPNLAEYSQTDGTGKGIGEIYAS |
| Lactobacillus | AKQNFVPEDIQKIKDLSGLPVIVKGIQSPDDAEAAIQAGADGIWVSNHGGRQL |
| terrae | DGGPASFEVLPLIAEKVAKRVPVIFDSGIRRGEHVFKALASGADLVAIGRPIIY |
| | GLNLGGSQGVYDVMEHINMELSITMQLAGTKTVEDVKNNKLMD |
| SEQ ID | MTSYYNGFPQSDRDEAINMINLDELEERAKEVMPEGAYYYIASGSENEWTW |
| NO: 189 | RNNTAAFNHFQIVPRALTNMDSPQLDTEFMGMKLKTPVMISPIACHGIAHKD |
| WP_10375 | AEVATQKGAAAAGALFTSSTYANKSVEDIAAAAPNAPRFFQLYLSKDWDFN |
| 2960.1 | KMVFDAVKKAGYSGILLTVDALVSGFREANLRTKFAYPVPLDFFTRYQGAKG |
| Lactobacillus | EGQTVAQMYASSAQKIGPEDIKRIKEMSGLPVFVKGVVCAEDAFLAMGAGA |
| panisapium | DGIYVTNHGGREIDCGPATIDMLPEIAKAVDHRVPIIFDSGVRRGSHVFKALA |
| | LGADLVGIGRPYLYGLALGGAKGVQSVIEQLNQELLIDMQLTGCKTIADIKHA |
| | RLTHFNYTADNLKSNTDPSRITPYPVTNDNQLKEEDSATDAVSGASQE |
| SEQ ID | MAYYTSNAEHPIEIVNIAGLEERVKARMDRGAFGYIREGAEDEWTLRENTRA |
| NO: 190 | FNDIRIVPRVLQGIDHADLSTSIFGIQLKTPIIEAPSAAHGLAHVKGEVDTAIGA |
| WP_10877 | AKAGTLFSMSTYGSTSVEEAAAAAPGAPQFFQLYMSKDDQFNEFLIRKAVK |
| 5660.1 | AGVKAIIMTVDSTLGGYREEDVVSHFQFPLPMPNLAAYSSSDGVGKGISEIY |
| Lactimicrobium | AAAKQDFVPEDVAKVKEMSGLPVLVKGIQSPQDALTAIQAGADGIWVSNHG |
| massiliense | GRQLDGGPASIAVLPSIAEAVSHRVPIIFDSGVRRGQHVFKALASGADLVAV |
| | GRPVIYGLNLGGAEGVKSVFDHLNMELSITMQLAGTKTIEDVKKAKLLR |
| SEQ ID | MKNKLLKTTAIAMALSVGVAQAAEYKASTAEGPIKIVNLKAMEAQVQANMEK |
| NO: 191 | GAFGYIRGGAEDENNLRSNTTAFDKKYIMPRSLQGIEFSDINLKTEFLGIKLDT |
| WP_11535 | PIIQAPMAAQGLAHQQGEVATAKGMAKAGSIFSLSTYGNKTIKEVADAQPGY |
| 0578.1 | PFFFQLYMSKNDAFNEYILSQAKQYGAKGIIMTIDSSVGGYREDDVKNNFQF |
| Proteus | PLGFANLEAFAKISNDKSKTGKGVGISEIYAQAKQAFTPADIQYVKKMSGLPV |
| vulgaris | IVKGIESPEDADTAIKAGADAIWVSNHGGRQLDSAPATIDMLPAIAKVVNKRV |
| | PIVFDSGVRRGSHVFKALASGADVVAVGRPILYGLNLGGAEGVNSVIQHLNK |
| | ELRINMMLGGAKTVKDIQATQLYTDASFNK |
| SEQ ID | MSTKYFQSENEKEINIINFTILEEEAKKIIPAGGYGYINGGAEDEWTLRMNTEA |
| NO: 192 | FNHKQIVPRSLTDVEKPDLTTTIYGEKISMPIFMTTVASHGLAHKDGEIATAKG |
| WP_11800 | TAAAGTIMGISTYSTKSLDEIMTASTGPKWFQLYMSKDDKFNEYMIGKAVAN |
| 6560.1 | GAKAVILTVDATLGGYREADLKNNFIFPLPMGNLESLGEGLGQSISEIFANAK |
| Fusobacterium | QKIGIKDIEKIVAMTDLPVIVKGIESPEDALLAIGAGAKGIYVSNHGGRQLDGG |
| ulcerans | PASFDVLESIAKAVNKKVPIIFDSGVRRGQHVFKALASGADLVGIGRPAIYGL |
| | AVGGSKGVTSVFKHFAKELKIVMQLAGCQTVEDIKKAKLLSIKY |
| SEQ ID | MKKKYEASTAENHVDIVNIAGLEARVKDHMSNEKGAFGYIRGGSEDEWTMK |
| NO: 193 | QNTEAFNKKKIMPRVIQGIDHADLSTKLWDIDLKTPIIQAPSAAQGLAHAKGE |
| WP_11932 | KDTAKGVADAGSIFSISTYGSTSVEDAAEAAPDAPQFFQLYMSKDDKFNEFLI |
| 6432.1 | KKAVKAGVKAIILTVDSTLGGYREADIVNKFQFPLPMPNLAGYAAGDGEGKGI |
| Companilac- | SEIYASAKQGIIPSDIQKIKDMSGLPVFVKGIQSPDDADLAIEFGADGIWVSNH |
| tobacillus | GGRQLDGGPASFDVLPYIAEVVDKRVPIVFDSGVRRGEHVFKALASGADLV |
| musae | AIGRPIIYGLNLGGAQGVTDVIDHLNMELSITMQLAGTKTINDVKNTELLD |
| SEQ ID | MKKTIVTLIAVTLMTSTYAHVPTTETTTQGKEKKGKGYQASTAEKKLKIVNLH |
| NO: 194 | DLEGQVKLEMDKGAFGYIVGGAEDQNNLKINTENFDKKYIMPRVLKGIKHEDI |
| WP_12196 | DLSTSLFGISLKTPIIQAPMAAQGLSHVDGEVATAKGMIAAGSLFSLSTYGNK |
| 4295.1 | TIEEVAEGINGAPFFFQLYMSKNDDFNKFTLERAKKHGAKAIILTVDSPVGGY |
| Myroides | REEDIKTGFTFPLVMGNLELFAAQQTDGNKTGKGAGITEIYAQAKQDFKPTDI |
| sp. N17-2 | KYVKDMTGLPVIVKGIQSPEDAEIAIQAGADAIWVSNHGGRQLDAGPSSFDV |
| | LPLVAKTVNKRVPIIFDSGVRRGSHIFKALASGADIVAIGRPILYALHLGGSQG |
| | VTSVIDQLNKELIINMFLGGAKNIQEIQNTKLYTDKDLSI |
| SEQ ID | MAIVNGYKQNENENSLDILNLDQLEKQAKEIIPTGGFGYISGGSEDEWTLRA |
| NO: 195 | NRQAFTHKQIVPRALTNIEKPELDTNVFGLPLKTPVFMVPAAAQGLAHVKGE |
| WP_12197 | VDTAKGVAAVGGLMAQSTYSSTSIADTAAAGNGAPQFFQLYMSDDWDFNE |
| 8072.1 | ALLDEAKAAGVKGIILTVDATVDGYREADIINNFQFPIPMANLTKYSEGDGQG |
| Lentilacto- | KGIAEIYASAAQKIGPDDIARIADYTDLPVIVKGIESPEDALYAIGAGAAGIYVS |
| bacillus | NHGGRQLNGGPASFDVLEDVAKAVNGKVPIIFDSGVRRGSDVFKALASGAD |
| raoultii | LVGIGRPVIYGLALGGAEGVQSVFEHLDHELEIIMQLAGTKTIEDVKKAKLLNL |
| | HY |
| SEQ ID | MTKFISKKYAVSLSAVALALSLGVANAADYKASTAEGPVEITNLNELEGQVKA |
| NO: 196 | NMDKGAFGYIRGGAENEQNLRSNTSVFDKKYIMPRVMQGIELSDIDLKTNYL |
| WP_12199 | GIDLKTPIIQAPMAAQGLAHQDGEIATAKGMAKAGSIFSLSTYGNKTIEEVAAV |
| 2380.1 | SDGNPFFFQLYMSKNEAFNEFTLKRAKDSGAKAIILTVDSPVGGYREDDIKN |
| Hyphomicro- | DFQFPLGFANLELFAKQNDDGSKTGKGAGISEIYAQAKQAFTPADIKYVKDM |
| biales | TGLPVIVKGIQSPEDADVVIKAGADSIWVSNHGGRQLDSGPASFDVLPSIAKV |
| bacterium | VNKRVPIVFDSGVRRGSHVFKALASGADVVAVGRPILYGLNLGGAEGVNSVI |
| | QQLNKELTINMMLGGAKNIEAVKKTKLYSDKDFE |
| SEQ ID | MSYQASKEEHPIEIVNIASLEKAVKERMEAGAFGYIRGGAEDEWTMTENTHS |
| NO: 197 | FNKKQIVPRVLRGIDSADLSTKLWDIELKTPIIQAPSAAQGLAHENGEVDTAK |
| WP_12264 | GVAEVGSIFSISTYANTTIEDAAAAAPDAPQFFQLYMSKDDKFNEFILEKAVK |
| 5598.1 | AGAKAIILTVDSTLGGYREEDIINKFQFPLPMPNLAAYSAQTESGDGEGKGIA |
| Enterococcus | EIYAAAKQAIVPEDIQKIKELTSLPVFVKGIQSSEDAEVAIAAGADGIWVSNHG |
| mediterraneensis | GRQLDGAPASFDLLPSIAEVVNKRVPIVFDSGIRRGEHVFKALASGADLVAIG |
| | RPVIYGLNLGGAEGVKSVFDHLNKELSITMQLAGTKTIEEVKNTKLVSF |
| SEQ ID | MTVINGYEQSDREQKLNIVNLPSLEAEAKKIIPTGGFGYIAGGSEDDWTLSAN |
| NO: 198 | TKAFNHAQIVPKALSNIEQPDLSTSIFGIDLKTPVMMAPTAAQGLAHSRGEMA |
| WP_12557 | TAEGVAAAGALMAQSTYSSTSIADTAAAGKGAPQFFQLYMSKDWDFNRSLL |
| 0549.1 | DEAKKAGVKAILLTVDATVDGYREADIVNNFQFPIPMANLTKFSEGDGQGKG |
| Lacticasei- | IGEIYASAAQKINEDDVRRIAEYTDLPVIVKGIQSPEDALRAIGAGAAGIYVSNH |
| bacillus | GGRQLNGGPASFDVLPTIAKAVNKQVPIIFDSGVRRGSHVFKALAAGADLVA |
| songhuajiangensis | FGRPVIYGLALGGAQGVQSVFEEIDHELEITMQLAGTKTIEDVKRAPLTHFNY |
| SEQ ID | MTMTNGYEQSDNEKKLEIVNLPSLEAEAKKIIPTGGFGYIAGGSEDDWTLKQ |
| NO: 199 | NTAAFGHVQIVPKALSNIEQPSTATKVFGLDLKTPIMMAPTAAQGLAHAQGE |
| WP_12558 | KDTAKGVAAVGGLMAQSTYSSTSIADTAVAGDGAPQFFQLYMSKDWSFNE |
| 2444.1 | SLLDEAVKAGVKGIILTVDATVDGYREADIINNFQFPIPMANLEKFSEGDGKG |
| Lacticasei- | KGIGEIYAAAAQKINEDDVRRIAEYTSLPVIVKGIESPEDAMRAIGAGAAGIYV |
| bacillus | SNHGGRQLNGGPASIDVLPAIAKAVNHQVPIIFDSGVRRGSHVFKALASGAD |
| suibinensis | LVAMGRPVIYGLALGGAQGVQSVFEELNHELEITMQLAGTKTIEDVKHAPLT |
| | HFNYAD |
| SEQ ID | MTLTNGYEQSDREQKLNIVNLPSLEAEAKKIIPTGGFGYIAGGSEDNWTLRQ |
| NO: 200 | NTAAFGHLQIVPKALSNIEQPSTATRVFGIDLKTPIMMAPTAAQGLAHSQGEK |
| WP_12558 | DTARGVAAVGGLMAQSTYSSTSIADTAAAGNGAPQFFQLYMSKDWTFNES |
| 5313.1 | LLDEAVKAGAKAIILTVDATVDGYREADIINNFQFPIPMANLTKFSEGDGKGK |
| Lacticasei- | GIGEIYASAAQKISEADVRRIADYTHLPVIVKGIESPEDALRAIGAGAAGVYVS |
| bacillus | NHGGRQLNGGPAAIDVLPAIAKAVNHQVPIIFDSGVRRGSHVFKALAAGADL |
| jixianensis | VAMGRPVIYGLALGGAQGVQSVFEELNHELEITMQLAGTKTIEDVKHAPLTH |
| | FNYAD |
| SEQ ID | MTETNGYFQNDNEKEIKIVNLPSLEAEAEKIIPKGGFGYIAGGSEDNWTLKAN |
| NO: 201 | TEAFNHVQIVPHVLSDIENPQTNTSIFGIDVKTPIMMTATAAQGLAHAKGEMD |
| WP_12558 | TAKGIAKAGALMEQSTYSSTSIADTMAAGNGAPQFFQLYMSKDWTFNESLL |
| 8711.1 | KEAKQAGAKAIVLTADATVDGYRESDIINDFQFPIPMANLTKFSEGDGEGKGI |
| Companilac- | GEIYAAAAQKISSKDIQRIKDIAGLPVIVKGVQSPEDALLAIGAGADGIQVSNH |
| tobacillus | GGRQLNGGPASFDVLSDVAKSVNHKVPIIFDSGVRRGSHVFKALASGADMV |
| jidongensis | ALGRPVIYGLALGGADGVYSVVEHLNDEFETIMQLAGTKTIEDVKHAQLLKK |
| SEQ ID | MTEVNGYIQSDREEKIDVLNLASLEKRAEKIIPAGGFGYISGGSEDNWTLKEN |
| NO: 202 | TEAFNHAQIVPRVLSDIDDPQTKTSIFGIDLDTPIMMSPAAAQGLAHSQGEKD |
| WP_12559 | TAKGMAKAGALMAQSTYSSTSISDAAKAGDGAPQFFQLYMSKDWEFNKSLL |
| 1830.1 | EEAKKAGVKAIVLTSDATVDGYRESDIVNDFQFPIPMANLTKFSEGDGKGKGI |
| Companilac- | GEIYAAAAQKISPKDIKRIKDIVDLPVIVKGVQSPEDALLAIGAGADGIYVSNHG |
| tobacillus | GRQLNGGPASFDVLADIAKAVNHKVPIIFDSGVRRGSHVFKALASGADMVAL |
| baiquanensis | ARPIIYGLALGGADGVYSVVEHLNDEFKTIMQLAGTKTIDDVKHAKLLHK |
| SEQ ID | MTEINGYIQSDREEKIDVLNLASLEKRAEEIIPTGGFGYISGGSEDNWTLKEN |
| NO: 203 | TEAFNHVQIVPRVLSDIDDPQTKTSILGINLETPIMMSPAAAQGLAHSQGEKD |
| WP_12567 | TAKGMAKAGALMGQSTYSSTSIADTAAAGNGSPQFFQLYMSKDWDFNKEL |
| 7189.1 | LNEAKKAGAKAILLTADATVDGYRESDVINDFQFPIPMANLTKFSEGDGKGK |
| Companilac- | GIGEIYAAAAQKIRPDDIQRIKDISGLPVIVKGIQSPEDALLAIGGGTDGIYVSN |
| tobacillus | HGGRQLNGGPASFDVLADVAKAVNHKVPIIFDSGVRRGSHVFKALASGADIV |
| keshanensis | ALARPMIYGLALGGADGVYSVVEHLNDELKTIMQLAGTKTIDDVKHAKLLHK |
| SEQ ID | MTIVNGYEQSDNENKLDILNLPSLEAEAKKIIPAGGFGYIVGGSEDEWTLAEN |
| NO: 204 | TKAFSHAQIVPKVLSNIEQPDLSTSIFGISLKTPVMMAPTAAQGLAHSQGEKD |
| WP_12569 | TARGVAAVGGLMAQSTYSSTSIADTAAAGEGAPQFFQLYMSKDWTFNESLL |
| 6134.1 | DEAKKAGAKAILLTSDATVDGYREEDIVNNFQFPIPMANLTKFSEGDGKGKGI |
| Lacticasei- | GEIYASAAQKISEDDVRRIAAYTDLPVIVKGVQSPEDALRAIGAGAAGIYVSN |
| bacillus | HGGRQLNGGPASFDVLPAIAKAVNHQVPIIFDSGVRRGSHVFKALAAGADLV |
| yichunensis | AIGRPAIYGLALGGALGVQSVFEQLDHELTITMQLAGTKTIEDVKRAPLTHEN |
| | YAD |
| SEQ ID | MTIVNGYEQSDNEKQLAIVNLPSLEKQAQAIIPTGGFGYIVGGAEDDWTLAQ |
| NO: 205 | NTKAFTHAQIVPKALSEIEAPSTATTVFGLDLKTPIMMAPVAAQGLAHAKGET |
| WP_12570 | DTAKGVAAVGGLMSQSTYASTSIADTAAAGAGAPQFFQLYMSKDWDFNYS |
| 6480.1 | LLDEAKKAGVKAILLTVDATVDGYREQDIINNFQFPIPMANLQKFSDGDGKGK |
| Lacticasei- | GIGEIYAAAAQKIGPDDVRRIAEYTDLPVIVKGIESPEDALRAIGAGAAGIYVSN |
| bacillus | HGGRQLNGGPASFDVLPAIAKAVNHQVPIIFDSGVRRGSHVFKALASGADLV |
| daqingensis | AIGRPVVYGLALGGAQGVQSVFEALNHELEITMQLAGTKTIEDVKHAPLTHF |
| | SYTD |
| SEQ ID | MTEINGYTQSDNEKELDILNLPSLEAEAKKIIPTGGFGYISGGSEDEWTLSEN |
| NO: 206 | TKAFNHVQIVPRVLSNIEDPQTDTEIFGIKVKTPIMMSPAAAQGLAHSQGEKD |
| WP_12570 | TAKGMAKAGALMSQSTYSSTSIADTAKAGAGSPQFFQLYMSKDWEFNESLL |
| 8235.1 | TEAMNAGAKAIILTADATVDGYRESDIINNFQFPIPMANLEKFSTGDGKGKGI |
| Companilac- | GEIYAAAAQKISPADVKRIKDFTNLPVIVKGVQSPEDALLAIGAGADGIYVSNH |
| tobacillus | GGRQLNGGPASFDVLKSVADAVNHQVPIIFDSGVRRGSHVFKALANGADMV |
| zhongbaensis | ALARPIIYGLALGGADGVYSVVEHLNDEFKTIMQLAGTKTIEDVKKAKLLR |
| SEQ ID | MTEINGYIQSDREGKIDVLNLTSLEKRAEEIIPAGGFGYISGGSEDNWTLKEN |
| NO: 207 | TEAFNHVQIVPRVLSDIDDPQTKTSIFGINVETPIMMSPAAAQGLAHSQGEKD |
| WP_12571 | TAKGMAKAGALMGQSTYSSTAIADTAAAGNGSPQFFQLYMSKDWDFNKEL |
| 1831.1 | LNEAKKAGAKAIILTADATVDGYRESDVINDFQFPIPMANLTKFSEGDGKGKG |
| Companilac- | IGEIYAAAAQKISPDDIQRIKDISGLPVIVKGIQSPEDALLAIGGGADGIYVSNH |
| tobacillus | GGRQLNGGPASFDVLADVAKAVNHKVPIIFDSGVRRGSHVFKALASGADIVA |
| kedongensis | LARPMIYGLALGGADGVYSVVEHLNDELKTIMQLAGTKTIDDVKHAKLLHK |
| SEQ ID | MTEVNGYQQSDNEKKLTILNLPALEAEAKKIIPTGGFGYIVGGAEDDWTLRQ |
| NO: 208 | NTLAFQHAQIVPKALSNIESPDLSTQIFGIDLKTPVMMAPAAAQGLAHSQGEK |
| WP_12574 | ATAKGVAAVGGLMSQSTYSSTSIADTAAAAPGAPQFFQLYMSKDWDFNYSL |
| 8340.1 | LDEAVKAGVKGIILTVDATVDGYREQDIINNFQFPIPMANLEKFSAGDGKGKG |
| Lacticasei- | IGEIYASAAQKIGEDDVRRIAEYTKLPVIVKGIESPEDALRAIGAGASGIYVSNH |
| bacillus | GGRELNGGPAAFDVLPSIAKAVNHQVPIIFDSGVRRGSHVFKALAAGADLVA |
| baoqingensis | FARPVLYGLALGGAQGVQSVFEQIDHEFEIVMQLAGTKTIADVKQAPLTHEN |
| | YQD |
| SEQ ID | MTETNGYFQNDNEKMIDVLNLPSLEARAEKIIPKGGFGYIAGGSEDNWTLKA |
| NO: 209 | NTEAFNHVQIIPHVLSDIEDPQTNTSFFGINVKTPIMMTATAAQGLAHAKGEID |
| WP_12576 | TAKGIAKAGTLMEQSTYSSTSIADTMAAGNGAPQFFQLYMSKDWTFNESLL |
| 3154.1 | KEAKKAGAKAIVLTADATVDGYRESDIINDFQFPIPMANLTKFSEGDGEGKGI |
| Companilac- | GEIYAAAAQKISPADVQRIKDIAGLPVIVKGVQSPEDALLAIGAGADGIQVSNH |
| tobacillus | GGRQLNGGPASFDVLTDVAKAVNHRVPIIFDSGVRRGSHVFKALASGADMV |
| hulinensis | AMGRPVIYGLALGGADGVYSVVEHLNDELKTIMQLAGTKTIEDVKHAKLLKT |
| | N |
| SEQ ID | MSNTYKQSTNEQAIEIVNLDELQERAKAIIPAGGFGYISSGSEDEWTLRANRE |
| NO: 210 | AFNHKLIVPRALTNMEKPLIDTSVFGISLKTPVMMAPTAAQGLAHVEGEADTA |
| WP_12598 | RGVAAVGGLMAQSTYSSRTITETMAAGNGAPQFFQLYMSKDWSFNNALLD |
| 0578.1 | QTKAAGIKAIILTVDATVGGYREADVRNKFSFPIPMANLEDFSKDSGEGKGIS |
| Loigolacto- | EIYAAAAQKISPKDVARITDYTDLPVIVKGVQSPEDAELAINSGAAGIYVSNHG |
| bacillus | GRQLNGGPASFDVLASVAQVVNHRVPVIFDSGIRRGSHVFKALASGADLVA |
| iwatensis | FGRPAIYGLALGGAKGVQSVFEHINDELKIVMQLAGTQTIAAVKQTKLLDNHF |
| SEQ ID | MTIVNGYEQSDREQKLDILNLPSLENAAKKIIPAGGFGYISGGSEDNWTLGAN |
| NO: 211 | TSAFNHAQIVPKALSNIENPDTSTSIFGIDLKTPVMMAPAAAQGLAHSRGEVS |
| WP_12785 | TAEGVAAAGALMAQSTYSSTSIAATAAAAGGAPQFFQLYMSKDWDFNYALL |
| 0085.1 | DEAKKAGVKAIILTVDATVDGYRESDIINDFQFPIPMANLQKFAEGDGKGKGI |
| Lacticasei- | GEIYASAAQKINEDDVRRIAEYTKLPVIVKGIQSPEDALRAIGAGAAGIYVSNH |
| bacillus | GGRQLNGGPASFDVLPAIAKAVNKRVPIIFDSGVRRGSHVFKALAAGADLVA |
| hulanensis | FARPAIYGLALGGSQGVQSVFEHITHELTITMQLAGTKTIADVKQAPLTHFNY |
| SEQ ID | MTYQGSTKEEKLTIIDLPRLREAVKRDTEAGAFGYVDGGSSDEQVLRDNEQ |
| NO: 212 | AFRHYQLIPRMLQDISAPNLSTALFDIPLAMPVIAAPIAAHGLMHQDGEQVTV |
| WP_13464 | KGVGAAGSIFSLSTYGNSRIADVASAAPDTPKFFQLYMSRDDDFNQYLLDEA |
| 4060.1 | VNNGYKAIILTADATLGGYREADIINNFTFPLPMENLAAFSNAAGSGEGLGIA |
| Weissella | DIYARAKQNLSLRDINKVKDMAHGLPVIVKGIQDPDDALAANGAGADGIWVS |
| cibaria | NHGGRELNGAPASIDTLAAITKAVNRRVPVIFDSGIRRGEDVAKALALGADVV |
| | ALGRPMLWGLNQGGAAGVQSVYEHLATELKIVMQLTGAHTVAELQHAKIIDA |
| | KF |
| SEQ ID | MSYQASNDERKLKVVNLASLESQVKPRMEAGAFGYIRGGAEDEWTMQQNT |
| NO: 213 | EAFKHKKILPHILKGIDNANLHTSIFGIDLDTPVIEAPSAAQGLAHEKGEVDTAK |
| WP_13759 | GVAAAGSIFSISTYANTKIEDAAAAAPNAPQFFQLYMSKDDGFNKFILDKAVK |
| 7765.1 | AGAKAIILTADSTLGGYREADVLNDFQFPLPMPNLAAYSDKTGSGDGKGKGI |
| Paucilacto- | AEIYAAAKQGLVLEDIQRVKQWTNLPVIVKGIQAAEDAEASILAGADAIWVSN |
| bacillus | HGGRQLDGGPASFDVLPAVAKTVAKRVPIIFDSGVRRGEHVFKALASGADL |
| kaifaensis | VAIGRPVIYGLNLGGAEGVKSVFDHINMELSITMQLAGTKTIDAVKNTTLLD |
| SEQ ID | MAYQTSNEEHPIDIVNIASLEGRVKERMDKGAFGYIRGGSEDEWTMKENTT |
| NO: 214 | SFNHKTIMPRVLRGVDHADLSTKLWGIDLDTPIIQAPSAAQGLAHEKGEADT |
| WP_13761 | AKGVAAAGSIFSISTYANTTVEDAAAAAPNAPQFFQLYMSKDDGFNEFILEKA |
| 0019.1 | VKAGAKAIILTADSTLGGYREEDVINNFQFPLPMPNLAAYSEKSASGNGEGK |
| Enterococcus | GIGEIYAAAKQGLVPEDIKKIKDLTNLPVFVKGIQSPEDAEVAIAAGADGIWVS |
| pingfangensis | NHGGRQLDGGPASFDVLPMIAGVVNKRVPIVFDSGVRRGEHIFKALASGAD |
| | LVAIGRPVIYGLNLGGAEGVKSVFDHLNKELSITMQLAGTKTIEEVKNTKLG |
| SEQ ID | MSNKKYEASTAENHVEIVNLASLEARVKDHMSNEKGAFGYIRGGAEDEWTL |
| NO: 215 | KQNTEAFNNKEIMPRVIQGIDHADLSTKLWDIDLKTPIIQAPSAAHGLAHVKG |
| WP_13761 | ETDTAKGVAAAGSIFSISTYASTSVEDAAAAAPDSPLFFQLYMSKDDKFNEFL |
| 0913.1 | IKKAVKAGAKAIVLTVDSTLGGYRESDIINKFQFPLPMPNLAGYSAGDGEGKG |
| Companilac- | ISEIYASAKQGIVPTDIQKIKDMSGLPVLVKGIQSPDDAELAIEFGADGIWVSN |
| tobacillus | HGGRQLDGGPASFEMLPYIAEVVDKRVPIVFDSGVRRGEHVFKALASGADL |
| huachuanensis | VALGRPIIYGLNLGGAQGVTDVIEHLNMELSITMQLAGTKTINDVKNTKLLG |
| SEQ ID | MSYITSTQERHIDILDIPALEAKVAANMEKGAFGYLSGAAEDELVLKANPLAF |
| NO: 216 | NHKLIAPRVLQDIENPDLTTEFLGLKLSAPIIAVPIAAHGLVHEHAELDTAQGV |
| WP_13849 | ADAGTIFSLSTYGNATVDDVAKIAPESPKFFQLYMSKDDNFNRWILDKAVKG |
| 1883.1 | GYKAIILTADSTLGGYREADIVNNFTFPNVMHNLEEWSKLSSDGETGSGEGI |
| Lactococcus | AAIYAKAKQALSLKDITFIKDYTKLPVFVKGVQSPKDVEPLIEAGVDGIWVSNH |
| raffinolactis | GGRQLDGGPASFDVLADIAKVVNKRVPIVFDSGVRRGQHIFKALASGADVV |
| | GIGRPMLWGLNLGGSQGVTDVFDHFKKELMITMQLAGTHNVAEIKATELLDA |
| | K |
| SEQ ID | MKGDYLANDLVRELRIIDLTELEREAARVIGKGAMGYIRGGAGDEWTMRRN |
| NO: 217 | TACFEERPILPRVLASLAKPDTKCRILGIDLPFPIIMAPVAAQGLAHVSAEAGT |
| WP_14763 | ARGTAEAGTIMCVSTYAGMTLEEIALAGNGASQWFQFYPSKDAGFNRHLLD |
| 8117.1 | QALAGGYRAVVLTADATVGGNREADLRNRFVFPLKMANLEQYGSGQGRSI |
| Rikenellaceae | EQIYADAMQQIGPAEVERIAAYTRLPVIVKGIQAPEDALRAIDAGAAGVQVSN |
| bacterium | HGGRQLDGGPGSFEALPAVARAVNGRVPVIFDSGIRRGQHIFKALASGADV |
| | VAIGRPAIYGLALGGWMGVRSVFEFFRHELEMVMQLAGTPDIEAVKKTRLFD |
| | PRADAQAGDPA |
| SEQ ID | MTYLTSTDEHAVEIVNLKSLEALVKERMVAQGNRGAFGYLRGAAEDEWTLR |
| NO: 218 | ENTESFNRKTIVPRVLRGIDSADLSTSIFGIPLKTPIIQAPVAAQGLAHMEGEV |
| WP_14777 | DTAKGMEKVGSLFSISTYGSTTVEEAAEAVNGAPQFFQLYMSKDDDFNQYL |
| 8843.1 | LNKAVKAGVKAIILTADSTLGGYREEDVVSKFQFPIPMKNLESYSVTDGVGK |
| Lactococcus | GIFEIFGAAKQGLVLDDIQKIKNWTNLPVLVKGIQSPIDALEAITAGADGIWVS |
| | NHGGRQLDGGPASFDVLPAIAAVVKKRVPIVFDSGVRRGEHVFKALASGAD |
| | LVAIGRPIIWGLNLGGADGVASVVEHLNHELSITMQLAGAKDIESVKTTELL |
| SEQ ID | MKKVKAGLFTATVLGLAMTLTTAYAAEYKASIKEGPLKIVNLNDLESQVKANM |
| NO: 219 | DKGAFGYIRGGAEDEKNMRDNTASFDRKYIMPRVMQGIELKDINITTSFLGIP |
| WP_15038 | LDTPVIQAPMAAQGLAHRDGEIATAKGMAKAGSIFTLSTYGNKTIEEVAAVSD |
| 4809.1 | GHPFFFQLYMSKNDAFNEFTLKRAKESGAKAIILTVDSPVGGWREDDLRNN |
| Morganella | FQFPLGFANLELFAKQNNDGAKTGKGAGISEIYAQAKQAFTPSDIQYVKKMS |
| psychrotolerans | GLPVIVKGIQSPEDADRVIEAGADAIWVSNHGGRQLDSGPASFDVLPAIAKTV |
| | NKRVPVVFDSGVRRGSHVFKALASGADIVAVGRPVLYGLNLGGAQGVNSVI |
| | QQLNKELRINMMLGGAKDINAVKQTRLYTDSDLQ |
| SEQ ID | MKKKYEASTAENHIDIVNIASLEGRVKDHMSNEKGAFGYIRGGSEDEWTMR |
| NO: 220 | QNTEAFNDKEIMPRVLQGIDHADLSTKLWDIDLKTPIIQAPSAAQGLANAKGE |
| WP_15338 | VDTAKGVADAGSIFSISTYGSTAVEDAAKAAPDAPQFFQLYMSKDDKFNEFL |
| 5753.1 | LKKAVAAGVKAIILTVDSTLGGYREADIINKFQFPLPMPNLAAYSDSDGEGKGI |
| Companilac- | SEIYAAAKQGIVPSDIQKIKDITHLPVIVKGIQSPYDAQLAIEFGADGIWVSNHG |
| tobacillus | GRQLDGGPASFEVLPYIAEIVDKRVPVIFDSGVRRGEHVFKALASGADLVAIG |
| halodurans | RPILYGLNLGGAQGVTDVIEHLNKELSITMQLAGTKSINEVKNTDLID |
| SEQ ID | MLQRVKKHPIAMMSSAIVALALSVGAAQAVDYQASIKEGPIKIINLDELEDQVA |
| NO: 221 | KNMEKGAFGYIRGGAEDELNLDKNTRSFDRKYIMPRVMQGIEIKDIDLSTQFL |
| WP_15462 | GIDLKTPIIQAPMAAQGLAHQDGEIATARGMAQAGSIFSLSTYGNKTIEEVAE |
| 1855.1 | VSGESPFFFQLYMSKNNAFNEFTLKRAKESGAKAIILTVDSPVGGYREDDIR |
| Providencia | NNFQFPLGFANLELFAKQNDDGSKTGKGAGISEIYAQAKQAFTPADIAYVKK |
| | LSGLPVIVKGIQSPEDADRVIKAGADAIWVSNHGGRQLDSGPASFDVLPSIAK |
| | VVNKRVPIVFDSGVRRGSHVFKALASGADVVAIGRPILYGLNLGGAEGVNSV |
| | IQQLNKELSINMMLGGTKNIESVKATTLYTDKDFQ |
| SEQ ID | MTTVNGYEQSDREQKLDILNLPALEADAKKIIPVGGFGYIAGGSEDNWTLAE |
| NO: 222 | NTKAFEHAQIVPKALSNIENPDLSTSIFGIDLKTPVMMAPTAAQGLAHAKGEV |
| WP_15528 | DTAKGVAAAGALMAQSTYSSTSIADTAAAAPGAPQFFQLYMSKDWDFNYSL |
| 7576.1 | LDEAKKAGVKAILLTVDATVDGYRESDIINNFSFPIPMANLTKFSEGDGKGKGI |
| Lactobacillceae | GEIYASAAQKISEDDVRRIAEYTDLPVIVKGIESPEDALRAIGAGAAGIYVSNH |
| | GGRQLNGGPASFDVLPAIAKAVNKQVPIIFDSGVRRGSHVFKALASGADLVA |
| | FGRPAIYGLALGGAAGVQSVFEHIGHELAITMQLAGTKTIEDVKNAPLTHFHY |
| SEQ ID | MVMTNGYEQQENEKILNILNLTELEAEAKKIIPTGGFGYISSGSEDEWTLKAN |
| NO: 223 | RTAFTHRQIVPKSLSDMEDPQTNTNVFGLDLKTPIFMAPTAAQGLAHAKGEV |
| WP_15543 | DTAKGVAKAGGLMAQSTYSSTSIADTAAGGEGAPQFFQLYMSKDWDFNKS |
| 1292.1 | LLDEAKQAGMKAIILTVDATVGGYREADIINQFQFPIPMANLIKFSEGDGEGK |
| Secundilac- | GISEIYAAAAQKIGPADVKRIIDYSDLPVIVKGIESPEDAAYAIGAGAAGVYVSN |
| tobacillus | HGGRQLNGGPASFDVLESVAKAVNGQVPVLFDSGVRRGSDVFKAIALGADL |
| folii | VGIGRPAIYGLALGGADGVFSVFEHLNNELKIIMQLAGTKTVDDIKKTELLNIH |
| | Y |
| SEQ ID | MTMINGYEQSDREEKIDILNLESLEERAEEIIPAGGFGYIAGGSEDEWTLKQN |
| NO: 224 | RMAFHHRQIAPKALSGIEKPELNTEIFGIPLSTPIMMAPAAAQGLAHSQGEKD |
| WP_15922 | TARGLAAVGGLMAQSTYSSVSIAETATAGGDAPQFFQLYMSKDWNFNESLL |
| 1178.1 | DEAKKANVKAIILTVDATVDGYREADIKNKFTFPLPMANLIKFSEGNGQGKGI |
| Pediococcus | EEIYASAAQNIRPEDVKRIADYTNLPVIVKGIQTPEDAIRAIDAGAAGIYVSNHG |
| acidilactici | GRQLNGGPASFDVLEDIATAVNKQVPIIFDSGVRRGSDVFKALASGADLVAL |
| | GRPAIYGLALGGAKGVQSVFEHLNHELEIVMQLAGTKTIEDVKNTSLLNIKY |
| SEQ ID | MTYQASNEEHELEIVNIASLEAQVKDRMEKGAFGYIRGGSEDEWTMKENTV |
| NO: 225 | SFNKKAIMPRVLRGIDAADLSTKLWDIDLKTPIIQAPSAAQGLAHEKGETDTA |
| WP_15972 | KGMAEAGSIFSISTYANTTIEDAAAAAPNAPQFFQLYMSKDDGFNEFILDKAV |
| 2124.1 | KAGAKAIILTVDSTLGGYREEDVVNHFQFPLPMPNLAAYSERSASGNGEGK |
| Enterococcus | GIAEIYAAAKQGLLPADIKKIKDLTNLPVFVKGIQSPEDAEVAIAAGADGIWVS |
| sp. | NHGGRQLDGGPASFDVLPMIANVVNKRVPVVFDSGVRRGEHVFKALASGA |
| CSURQ083 | DLVAVGRPIIYGLNLGGAAGVKSVMDHLNKELSITMQLAGTKTIEEVKNTKLL |
| 5 | |
| SEQ ID | MSYHTSNEEHAIEIVNIASLEALVKARMEAGAFGYIRGGAEDEWTMRENTLS |
| NO: 226 | FNRKKIVPRVLQGIDHADLSTKLWDISLKTPIIQAPSAAQGLAHEQGEKDTAK |
| WP_16040 | GVAAAGSIFCISTYANTSIEDAANAAPNVPYFFQLYMSKDDDFNRFIIDKAVK |
| 3735.1 | AGAKAIILTVDSTLGGYREEDIVNKFQFPLPMKNLSAYSQSNGNGDGSGKGI |
| Gilliamella | SEIYAAAKQGIVPSDIQKIKAMANLPVIVKGIQSPEDASIAISAGADAIWISNHG |
| sp. Pas-s95 | GRQLDGAPASFEVLPSIAATIAKRVPIIFDSGVRRGEHVFKALASGADLVAIG |
| | RPILYGLNLGGAEGVKSVFDHLNKELSITMQLAGTKTIEDIKNTLLI |
| SEQ ID | MYQASNEEHPIEIVNIASLEGKVKADMEVGAFGYIRGGSEDEWTMRENTAS |
| NO: 227 | FNQKKIMPRVLRGIDHADLSTKLWDIDLKTPIIQAPSAAQGLAHEKGEADTAI |
| WP_16189 | GVAATGSIFSISTYANTTIEAAAAAAPGAPQFFQLYMSKDDGFNEFILEKAVK |
| 9215.1 | AGAKAIILTADSTLGGYREEDIINHFQFPLPMPNLAAYSEQSASGDGEGKGIA |
| Enterococcus | EIYAAAKQGLVPEDIKKIKDLTQLPVIVKGIQSPEDAEVAIAAGADGIWVSNHG |
| sp. | GRQLDGGPASFEVLPAIASVVNKRVPVIFDSGVRRGEHVFKALASGADLVAI |
| CU9D | GRPIIYGLNLGGAQGVASVLEHLNKELSITMQLAGTKTIEEVKNTQLLD |
| SEQ ID | MAYYTSNEEHPIDIVNIASLEERVKARMDRGAFGYIREGAEDEWTLKENTRA |
| NO: 228 | FNDISIAPRVLQGMSHADLSTSIFGIDLKTPIIEAPSAAHGLAHVRGEVDTAIGA |
| WP_16413 | AKAGTLFAMSTYGSTPVEEAAAAVDGAPQFFQLYMSKDDKFNEFLINKAVK |
| 0152.1 | AGVKAIIMTVDSTLGGYREEDIVSHFQFPLPMPNLAAYSNSDGVGKGISEVY |
| Sharpea | AAAKQDFVPSDIAKVKEMSGLPVIVKGIQSPEDAAVAIQAGADGIWVSNHGG |
| azabuensis | RQLDGGPASIKVLPSIANVVNHQVPVIFDSGVRRGQHVFKALASGADLVAIG |
| | RPVIYGLNLGGAEGVKSVFDHLNMELSITMQLAGTETIEDIKKTILLK |
| SEQ ID | MSYQASTEEHPIEIVNIASLEGRVKERMEAGAFGYIRGGSEDEWTMKENTSS |
| NO: 229 | FNTKKIMPRVLRGIDSADLHTSVFGIDLKTPIIQAPSAAQGLAHEKGEADTAK |
| WP_16500 | GVAAAGSIFSISTYANTTIKDAADAAPGAPQFFQLYMSKDDGFNEFILKKAVE |
| 5381.1 | AGAKAIILTADSTLGGYREEDVINNFQFPLPMPNLAAYSEQSASGNGEGKGI |
| Enterococcus | AEIYAAAKQGLTPEDIKKIKDITNLPVIVKGIQSPEDAEVAIAAGADGIWVSNH |
| | GGRQLDGGPASFEVLPKIAEVVNKRVPVIFDSGVRRGEHVFKALASGADLV |
| | AIGRPVIYGLNLGGAEGVTSVFEHLNKELSITMQLAGTKTIDEVKNTKLMD |
| SEQ ID | MKRKILKNTAIAMALSVSVAQAAEYKASTAEGPIKIVNLKTMEVQVQANMDK |
| NO: 230 | GAFSYIRGGAEDENNLRSNTTAFDKKYIMPRSLQGIEFSDLDLKTEFLGIKLD |
| WP_16512 | TPIIQAPMAAQGLAHQQGEVATAKGMAKAGSIFSLSTYGNKTIKEVADAQPG |
| 6355.1 | YPFFFQLYMSKNDAFNEYILSQAKQYGAKGIILTVDSPVGGYREDDIKNSFQF |
| Proteus sp. | PLGFANLEAFAKISDDKSKTGKGAGISEIYAQAKQAFIPADIQYVKKMSGLPVI |
| ZN5 | VKGIESPEDADTAIKAGADAIWVSNHGGRQLDSAPSTIDVLPAIAKVVNKRVP |
| | IVFDSGVRRGSHVFKALASGADVVAVGRPILYGLNLGGAEGVNSVIQHLNKE |
| | LKINMMLGGAKTVKDIQATQLYTDTSFNQ |
| SEQ ID | MVMTNGYEQNENEKVLDILNLNQLEADAKKIIPTGGFGYIVSGSEDEWTLQA |
| NO: 231 | NRKAFQHRQIVPKALSNIEDPQTDTTVFGLDLKTPIMMAPAAAQGLAHAKGE |
| WP_16892 | VDTAKGVAKAGGLMAQSTYSSTSIADTAAGGEGAPQFFQLYMSKDWDFNR |
| 4737.1 | SLLDEAKKAGVKAIILTVDATVGGYREADIINHFQFPIPMANLIKFSEGDGEGK |
| Lactobacillus | GISEIYASAAQKIGPDDVKRIIDYTDLPVIVKGIESPEDALYAIGAGAAGIYVSN |
| sp. | HGGRQLNGGPASFDVLESVTKAVNHQVPVIFDSGVRRGSDVFKAIALGADL |
| HBUAS513 | VGIGRPAIYGLALGGADGVFSVFEHLNNELKIVMQLAGTKTIDDIKHAELLHIQ |
| 83 | Y |
| SEQ ID | MKSSHLIQALASLAMLATSGFTYAEEYKASTDEKAIKMTNVASLEERVQARM |
| NO: 232 | EKGAFGYIRGGAEDENNLRSNTESFDKKYIMPRVLQGIELKEIDLSTQLLGIPL |
| WP_17192 | KTPIIQAPMAAQGLAHASGELATAKGMAQVGSIFSLSTYGNKTIEEVANVSG |
| 3452.1 | KNPFFFQLYMSKNNQFNEFILAQAVKHGAKAIILTVDSPVGGYREEDIKNNFQ |
| Salmonella | FPLGFANLEMFARKNDDGSKTGKGAGISEIYAQAKQAFTPEDIAYVHRISGLP |
| bongori | VIVKGIQSPEDAEIAIQAGAAGIWVSNHGGRQLDSGPSSFDMLPAIAKVVNKR |
| | VPVIFDSGVRRGSHVFKALASGADIVAIGRPVLYGLNLGGAQGVASVIEQLN |
| | KELTINMMLGGAKNIEQVKNTRLLSEKDL |
| SEQ ID | MTYHTSNEEHAIDIVNVASLETKVRDRMEKGAFGYIRGGAEDEWTMRENTR |
| NO: 233 | SFQSKKIYPRVLKGIDHADLRTSLWDLALDTPIIQAPSAAQGLAHEKGEVDTAI |
| WP_17196 | GVARSGSIFSISTYANTSITDAAAAAPGAPQFFQLYMSKDDGFNSFIVGEAIQ |
| 9741.1 | AGAKAIILTVDSTLGGYREEDIVNKFQFPLPMPNLAAYSTANGAGTGKGIAEI |
| Megasphaera | YANAKQGIVLSDIVKIKNMSQLPVFVKGIQAPADAEAAIAAGADGIWVSNHGG |
| cerevisiae | RQLDGGPASFECLPKVAEAVHKRVPIVFDSGVRRGEHVFKALASGADLVAIG |
| | RPVIYGLNLGGAAGVQSVFEHLNKELSITMQLAGTKTIDDVKNTTLVD |
| SEQ ID | MTVINGYEQSDREEHINVLNLASLEKRAQAIIPTGGFGYIVGGSEDDWTLAQ |
| NO: 234 | NRKAFTHKQIYPRTLANIDNPDLSTNVFGIDLQTPVMMAPLAAQGLAHSKGE |
| WP_17218 | VATAKAFADEGALMAQSTYSSASIADTAAAGAGAPQFFQLYMSKDWKFNEA |
| 8100.1 | ILDEAKKAGVKGIILTADATVDGYREADIINNFQFPIPMANLEKFSQGAGKGQ |
| Lentilacto- | GIAEIYAAAAQKISPDDVRRIAEYSGLPVIVKGIQTPEDAELAIGAGAAGVYVS |
| bacillus | NHGGRQLNGGPASFDALSAIATAVAGRVPIIFDSGVRRGSDVFKSLAAGADL |
| kribbianus | VALGRPFVYALALGGSLGVQDAIQELNHEFATTMQLAGTKTIAEVKQAKLAD |
| | FNY |
| SEQ ID | MTMVNGYEQSDREKHIKVVDLESLEAEAQKIIPTGAFGYISGGSEDEWTLRQ |
| NO: 235 | NRKSFTHKQIYPRVLTDVEKPEISTNFMGIDLKTPIMMPPLAAQGLANSQSEK |
| WP_17218 | DTARAFAAAGGLMALSTYGSATIADFAKAGNGAPQFFQPYMSKNDDFNKSL |
| 8596.1 | LDEAKRNGMKAIILTADSTLGGYREADVINNFQFPIPMANLEKLSQGAGKGQ |
| Lentilacto- | GIAAIYAAAAQKIGPDEIKKIADYTDLPVIVKGIQNPVDAELAIDAGAAAIYVSN |
| bacillus | HGGRQLNGGPGSFDVLPEIAVAVAKRVPIIFDSGIRHGSDVFKALATGADLV |
| kribbianus | AIGRPFVYALALGGQLGVEDALAEINREFQIVMQLAGTQTIEDVKNTKLADLS |
| SEQ ID | MTYITSTQERHIDIVDIPSLEKLVQANMAKGAFGYLAGAAEDELVLKANPLAF |
| NO: 236 | NHKHIVPRVLQDIQDPDLTTEFLGLKLSSPIIGVPIAAHGLVHEKAELATAQGV |
| WP_17220 | AKAGTIFSLSTYGNATVDEVAAVAPDSPKFFQLYMSKDDDFNRWILDKAIKG |
| 8953.1 | GYKAIILTADSTLGGYRESDIINNFTFPNVMRNLEEWSKLSTDSENGAGEGIA |
| Lactococcus | AIYAKAKQALSLRDITFIKDYTHLPVFVKGIQSAHDVEPLINAGVDGIWVSNHG |
| hodotermopsidis | GRQLDAGPASFDVLADIAKAVNKRVPIIFDSGVRRGQHVFKALANGADIVGIG |
| | RPMLWGLNLGGSQGVTDVFEHFKKELRINMQLAGAHTIAEVKETKLIDA |
| SEQ ID | MAYTTSTDEQKVAIVNIKSLEGKVKARMEAQGNKGAFGYIRGGAEDEWTMS |
| NO: 237 | ENTRAFNDKKIVPRVLRGVDSADLSTSIFGIDLKTPIIQAPVAAQGLAHMAGE |
| WP_17235 | VDTAKAMAEVGSLFSISTYGSTSVEDAAAAAPGAPQFFQLYMSKDDQFNEF |
| 6310.1 | LLKKAVAAGVKAIILTADSTLGGYREEDVINGFEFPLPMPNLAAFSESDGVGK |
| Lactococcus | GIGEIYAAAKQGLVLEDIKKIKDITGLPVLVKGVQSAEDASAAIDAGADGIWVS |
| insecticola | NHGGRQLDGGPASIETLPAIASAVAKRVPIVFDSGVRRGEHVFKAIAQGADL |
| | VAVGRPVLYGLNLGGAQGVQSVIEHLNKELSITMQLAGTKNIAEIKTTDLID |
| SEQ ID | MYHTSNEEHPIDIVNIASLEGRVKERMEAGAFGYIRGGSEDEWTMKENTTSF |
| NO: 238 | MNKKIMPRILQGIDHADLSTKLWDIDLKTPIIQAPSAAQGLAHENGEADTAKG |
| WP_17310 | VAAAGSIFSISTYANTTIEDAAAAAPDAPQFFQLYMSKDDGFNEFILDKAVKA |
| 3965.1 | GAKAIILTADSTLGGYREEDIINQFQFPLPMPNLAAYSEQSASGNGEGKGIAEI |
| Enterococcus | YAAAKQGLVPEDIKKIKDLTHLPVFVKGIQSPEDADLAIKAGADGIWVSNHGG |
| saigonensis | RQLDGGPASFEVLPAIAEVVDKRVPIVFDSGVRRGEHVFKALASGADLVAIG |
| | RPVIYGLNLGGAEGVKSVFDHLNKELSITMQLAGTKTISDVKKAKLID |
| SEQ ID | MSTKYFQSENEKEINIINFRMLEEEAKKIIPAGGYGYISGGAEDEWTLKMNTE |
| NO: 239 | AFNHKQIVPRSLTDVEKPDLTTTIYGEKISMPIFMTTVASHGLAHRDGEIATAK |
| WP_17689 | GAAAAETIMGISTYSTKSLDEIMTASTGPKWFQLYMSKDDNFNRYMIEKAVA |
| 2287.1 | NGAKAVILTVDATLGGYREADLKNNFIFPLPMGNLESLGAGLGQSISEIFANA |
| Fusobacteriaceae | KQKIGIKDIEEIVSLTNLPVIVKGIESPEDALLAIGAGAKGIYVSNHGGRQLDGG |
| bacterium | PASFDVLESIAKAVNRKVPIIFDSGVRRGQHVFKALASGADLVGIGRPAIYGL |
| | AVGGSKGVTSVFKHFAKELKIVMQLAGCKTIEDIKKAKLLSIKY |
| SEQ ID | MTTVNGYEQSDNEQRLAILNLPSLEAKAKAIIPTGGFGYIVGGAEDDWTLRQ |
| NO: 240 | NTKAFTHAQIVPKALSDIEAPSTATTVFGLDLKTPIMMAPVAAQGLAHAKGET |
| WP_17939 | DTAKGVAAVGALMSQSTYSSTSIADTAAAGAGAPQFFQLYMSKDWAFNYSL |
| 6170.1 | LDEAKKAGVKAILLTVDATVDGYREQDIINDFQFPIPMANLQKFSEGDGAGK |
| Lacticaseibacillus | GIGEIYAAAAQKIGPDDVRRIAEYTDLPVIVKGIQSPEDAMRAIGAGAAGLYVS |
| absianus | NHGGRQLNGGPASFDVLPAIAKAVNHRVPIIFDSGIRRGSHVFKALASGADL |
| | VAIGRPVLYGLALGGAEGVQSVFEALNHELEITMQLAGTKTIEDVKHAPLTHF |
| | QYAD |
| SEQ ID | MKSSHLIQALASLAMLATSGFTYAEEYKASTDEKAIKMTNVASLEERVQARM |
| NO: 241 | EKGAFGYIRGGAEDENNLRSNTESFDKKYIMPRVLQGIELKEIDLSTQLLGIPL |
| EAN92264 | KTPIIQAPMAAQGLAHASGELATAKGMAQVGSIFSLSTYGNKTIEDVAKVSG |
| 08.1 | KNPFFFQLYMSKNNEFNEFILSQAVKHGAKAIILTVDSPVGGYREEDIKNDFQ |
| Salmonella | FPLGFANLEMFASKNDDGSKTGKGSGISEIYAQAKQAFTPVDIAYVHRISGLP |
| enterica | VIVKGIQSPEDAEIAIQAGAAGIWVSNHGGRQLDSGPSSFDMLPAIAKVVNKR |
| | VPVIFDSGVRRGSHVFKALASGADIVAIGRPVLYGLNLGGAQGVASVIEQLN |
| | KELTINMMLGGARNIEQVKNTRLLSEKDL |
| SEQ ID | MKSSHLIQALASLAMLATSGLAYAEEYKASTDEKTIKMTNVASLEARVQARM |
| NO: 242 | EKGAFGYIRGGAEDENNLRSNTESFDKKYIMPRVLQGIELKEIDLSTQLLGIPL |
| EBP376117 | KTPIIQAPMAAQGLAHASGELATAKGMAQVGSIFSLSTYGNKTIEDVAKVSG |
| 4.1 | KSPFFFQLYMSKNNKFNEFILSQAVKHGAKAIILTVDSPVGGYREEDIKNDFQ |
| Salmonella | FPLGFANLEMFASKNDDGSKTGKGSGISEIYAQAKQAFTPEDIAYVHRISGLP |
| enterica | VIIKGIQSPEDAEIAIQAGAAGIWVSNHGGRQLDSGPSSFDMLPAIAKVVNKR |
| subsp. | VPVIFDSGVCRGSHVFKALASGADIVAIGRPVLYGLNLGGAQGVASVIEQLN |
| arizonae | KELTINMMLGGARNIEQVKNTRLLSEKDL |
| SEQ ID | MKKNHLIRVVASLAMLVTSGFTYAEEYKASTDEKAIKMTNVASLEARVQARM |
| NO: 243 | EKGAFGYIRGGAEDENNLRSNTESFDKKYIMPRVLQGIELKEIDLSTQLLGIPL |
| EBT045654 | KTPVIQAPMAAQGLAHASGELATAKGMAQVGSIFSLSTYGNKTIEEVANVSG |
| 0.1 | KNLFFFQLYMSKNNQFNEFILAQAVKHGAKAIILTVDSPVGGYREEDIKNNFQ |
| Salmonella | FPLGFANLEMFARKNDDGSKTGKGAGISEIYAQAKQAFTPEDIAYVHSVSGL |
| enterica | PVIVKGIQTPEDADIAIQAGAAGIWVSNHGGRQLDSGPSSFDMLPAIAKVVNK |
| | RVPIIFDSGVRRGSHVFKALASGADIVAVGRPILYGLNLGGSQGVASVIEQLN |
| | KELTINMMLGGARNIEQVKMTRLLTEKDLQQ |
| SEQ ID | MKTNHLIRAVASLAMLATSSFTYAEEYKASTDEKAVKMTNVASLEARVQAR |
| NO: 244 | MEKGAFGYIRGGAEDENNLRSNTESFDKKYIVPRVMQGIELKEIDLSTQLLGI |
| ECG85915 | PLKTPVIQAPMAAQGLAHASGELATAKGMAQVGSIFSLSTYGNKTIEEVAKA |
| 23.1 | SGKNPFFFQLYMSKNNQFNEFILAQAVKHGAKAIILTVDSPVGGHREEDIKN |
| Salmonella | DFQFPLGFANLEMFARQNDDGSKTGKGSGISEIYAQAKQAFTPEDIAYVHRV |
| enterica | SGLPVIVKGIQSPEDAEIAIQAGAAGIWVSNHGGRQLDSGPSSFDMLPAIAKV |
| subsp. | VNKRVPVIFDSGVRRGSHIFKALASGADIVAVGRPILYGLNLGGAQGVASVIE |
| salamae | QLNKELTINMMLGGARNIEQVKTTRLLTEKELPQ |
| SEQ ID | MKTHHLIRVVVASLAMLAMLATSGLAYAEEYKASTDEKAIKMTNVASLEARV |
| NO: 245 | QAKMEKGAFGYIRGGAEDENNLRSNTESFDKKYIMPRVLQGIELKEIDLSTQ |
| EEG26412 | LLGIPLKTPIIQAPMAAQGLAHASGELATAKGMAQVGSIFSLSTYGNKTIEEVA |
| 60.1 | NVSGKNPFFFQLYMSKNNQFNEFILAQAVKHGAKAIILTVDSPVGGYREEDIK |
| Salmonella | NNFQFPLGFANLEMFARKNDDGSKTGKGAGISEIYAQAKQAFTPEDIAYVHR |
| enterica | ISGLPVIVKGIQSPEDAEIAIQAGAAGIWVSNHGGRQLDSGPSSFDMLPAIAK |
| | VVNKRVPVIFDSGVRRGSHVFKALASGADIVAVGRPVLYGLNLGGAQGVAS |
| | VIEQLNKELTINMMLGGARNIEQVKTTRLLTEKDLPQ |
| SEQ ID | MTVTNGYEQSDREESIDILNLDELEERARQIIPKGGFGYISEGSEDEWTKAR |
| NO: 246 | NREAFNAVQIVPRVLTEVDAPSTATSVFGIDVKTPVIMAPAAAQGLAHARGE |
| HHW50910 | AATAEGIAAAGSLMVQSTYGTTTIAQTATAAPGAPWFFQLYMSTDWDFNHA |
| .1 | LIDEAKKYGAAAIVLTVDSMQGGYREPDIANKFQFPLPMANLEQFSSRGSKG |
| Pseudoclavibacter | KGIAEIYAAAAQKITPTDVEKIIDYSGLPVIVKGIQDPDDANLALGAGAAGIWVS |
| sp. | NHGGRQLNGGPGSFDVLPSIARAVNGRVPVVFDSGVRRGSHVFKALASGA |
| | DLVATARPFIYALALGGAQGVQAAVEHLTHEFRIVMQLAGTNTVDDVKRAKL |
| | LRVS |
| SEQ ID | MAYQTSNEEHPIDIVNIASLEGRVKERMDKGAFGYIRGGSEDEWTMKENTT |
| NO: 247 | SFNHKTIMPRVLRGVDHADLSTKLWDIDLDTPIIQAPSAAQGLAHEKGEADTA |
| NBK09489. | KGVAAAGSIFSISTYANTTVEDAAAAAPNAPQFFQLYMSKDDGFNEFILEKAV |
| 1 | KAGAKAIILTADSTLGGYREEDVINNFQFPLPMPNLAAYSEKSASGDGEGKGI |
| Enterococcus | GEIYAAAKQGLVPEDIKKIKYLTNLPVFVKGIQSPEDAEVAIAAGADGIWISNH |
| asini | GGRQLDGGPASFDVLPMIAGVVNKRVPIVFDSGVRRGEHVFKALASGADLV |
| | AIGRPVIYGLNLGGAEGVKSVFDHLNKELSITMQLAGTKTIDEVKNTKLS |
| SEQ ID | MTPRVVFAQTSSDSASSTSAATGGYQASTDEGAIKIVNLEELEALVKPRMEE |
| NO: 248 | GAFGYIRSGAENEINLRANTTSFNRKYIMPRILQGLESTDINLSTQLLGIDLKT |
| NLB30434. | PIIQAPMAAQGLAHVEGEIATARGVAEAGSIFSLSTYGNKTIEDVAEAGGNNP |
| 1 | FFFQIYMSKNDAFNEFTLKRAKDSGAKAIILTVDSPVGGYREEDIRTKFTFPL |
| Alcaligenaceae | GMPNLELFAAQNDDGTKTGQGSGITEIYAQAKQAFTPEDIAYIKDLTGLPVLV |
| bacterium | KGVQAPEDATVAIKAGADGIWVSNHGGRQFDSGPASFDVLPHIAKVVNKRV |
| | PIIFDSGVRRGSHVFKALASGADIVAVGRPILYGLNLGGGAGVHSIIEHLNKEL |
| | RINMMLAGARDIEAIKKTPLYTDEDF |
| SEQ ID | MYYASNEEHPIEVINVASLEDKVKNHMDKGAFGYIRGGAEDEWTMRQNTRS |
| NO: 249 | FMNKKIIPRVLQGVDHADLSTNLWNIDLKTPIIQAPTAAQGLAHKKGEVNTAK |
| NSM32159. | AMAEVGSLFSISTYANTSIEDAASAAPGSPQFFQLYMSKDDNFNEFILEKAVK |
| 1 | VGAKAIILTADSTLGGYREEDIINNFRFPLPMPNLASYSKQSDSGDGKGKGIA |
| Enterococcus | EIYAAAKQGLVPEDIKKIKDISNLPVFVKGIQSSDDAEVAINAGADGIWVSNHG |
| faecalis | GRQLDGGPASFEVLPDIARVVNKRVPIIFDSGVRRGEHIFKALASGADLVAIG |
| | RPVLYGLNLGGASGVVSVLEHLNKELSITMQLAGTKTIDEIKTTILSE |

In the 3-dimensional structure of the LDH, SEQ ID NO: 1 is located in or is interacting with the active site lid (see Ashok Y. et al. 2020). The residues T, F, X₁, X₂, X₃, and X₄ of SEQ ID NO: 1 form a structural motif in the 3-dimensional structure of the LDH.

According to one embodiment of the invention, the residues T, F, X₁, X₂, X₃, and X₄ of SEQ ID NO: 1 form a structural motif in the 3-dimensional structure of the LDH. The ranges of distances of these residues in the structural motif are given in the following α-carbon distance matrix of Table 2. The distances are given in the unit Ångström (Å). The distances given in Table 2 are to be understood as including the decimal digits of the respective number. For example, if a distance of 10 Å is given, this specification of the distance includes 10.0, 10.1, 10.2, 10.3, 10.4, 10.5, 10.6, 10.7, 10.8, and 10.9. Furthermore, when considering distances between residues in a 3-dimensional structure of an enzyme, variabilities among different methods for determination of the protein structure and uncertainties of a single determined structure have to be considered, especially if computer simulated models of the structure are used for the determination of distances among residues. The distance between residues X₃ and X₄ and the residues T, F, X₁, and X₂ of SEQ ID NO: 1 may differ among distinct active site loop confirmations and thus, the distances for X₃ and X₄ may differ in these distinct confirmations of the 3-dimensional structure.

TABLE 2

| Residue | T | F | X₁ | X₂ | X₃ | X₄ |
|---|---|---|---|---|---|---|
| T | X | | | | | |
| F | 10, 11, 12 | X | | | | |
| X₁ | 5, 6, 7 | 9, 10, 11 | X | | | |
| X₂ | 7, 8, 9 | 8, 9, 10 | 3, 4, 5 | X | | |
| X₃ | 14, 15, 16, | 12, 13, 14, | 11, 12, 13, | 8, 9, 10, | X | |
| | 17, 18 | 15, 16, 17, | 14, 15, 16, | 11, 12, 13, | | |
| | | 18, 19, 20, | 17, 19 | 14, 15, 16 | | |
| | | 21, 22 | | | | |
| X₄ | 11, 12, 13, | 12, 13, 14, | 9, 10, 11, | 5, 6, 7, 8, | 3, 4, 5 | X |
| | 14 | 15, 16, 17, | 12, 13, 14, | 9, 10, 11, | | |
| | | 18, 19, 20 | 15, 16 | 12, 13, 14, | | |
| | | | | 15 | | |

In the method of the invention, an electrode equipped with a LDH is used. The term “electrode” refers to any suitable surface for accepting electrons from the LDH via mediated electron transfer. Thereby the electrode is of a material capable of accepting electrons from the redox mediator used in the invention. Furthermore, the electrode may be of any material suitable or modified with any material to adsorb or immobilize the LDH. Non-limiting examples of such a material are platinum, gold, boron doped diamond and carbons such as graphite, pyrolytic graphite and glassy carbon where all of them can additionally be modified with carbon nanotubes (single or multi-walled), carbon fibres, nanoparticles e.g. gold nanoparticles or promoters as e.g., thiols. The electrode may be also of any material to increase the specific surface are of the electrode.

According to one embodiment of the invention, the electrode may be used as single electrode or as a stack of electrodes of e.g., 2, 3, 4, 5, or more electrodes.

According to one embodiment of the invention, the electrode provided herein is a working electrode.

The electrode comprising the LDH of the invention enables the detection and/or quantification of lactate based on mediated electron transfer.

Mediated electron transfer in biosensors typically employs a two-step procedure in which the enzyme takes part in a first redox reaction with the substrate and is in turn re-oxidized by a redox mediator. Finally, the redox mediator is oxidized by the electrode.

Redox mediators are artificial electron transferring agents that can readily participate in the redox reaction with the biological component and thus help in rapid electron transfer to the electrode. A “redox mediator” is an electron-transfer agent for carrying electrons between an analyte, an analyte-reduced or analyte-oxidized enzyme, and an electrode, either directly, or via one or more additional electron-transfer agents. A redox mediator that includes a polymeric backbone may also be referred to as a redox polymer.

In general, oxygen is not a redox mediator.

According to one embodiment of the invention, the oxidation of lactate is performed in the presence of a redox mediator. The method of the invention can also be performed in the presence of more than one redox mediator e.g., in the presence of two or more different redox mediators. Thereby, the redox mediator may be present on the electrode, in an enzyme composition comprising the enzyme and the redox mediator, or may be present in the sample.

According to another embodiment of the invention, an enzyme composition comprises the LDH of the invention and a redox mediator as defined herein. In a specific embodiment, the enzyme composition comprises LDH and a redox polymer. Specifically, said redox polymer may comprise a transition metal complex, preferably an osmium-containing complex.

According to one embodiment, the redox mediator may be any molecule or material able to carry electrons between LDH and electrode. Specifically said redox mediator is selected from the group consisting of any one of organic redox mediators, soluble redox mediators, insoluble redox mediators, redox polymers, transition metal complexes, polymeric transition metal complexes, wired redox mediators, sandwich compounds, and derivatives of these redox mediators.

Polymeric transition metal complexes comprise a polymeric backbone, spacers, and transition metal complexes.

Specifically, redox polymers are polymers comprising redox species. Non-limiting examples of such redox species used in redox polymers are osmium (Os), ruthenium (Ru), iron (Fe), cobalt (Co), or any transition metal. Non-limiting examples of polymers used for redox polymers are poly(vinylpyridine), poly(thiophene), poly(aniline), poly(pyrrole), or poly(acetylene). An example of a redox polymer is Os-containing poly(vinylpyridine).

According to a specific embodiment, in the case the redox mediator comprises osmium the redox mediator may be an osmium transition metal complex with one or more ligands, each ligand having a nitrogen-containing heterocycle such as 2,2′-bipyridine, 1,10-phenanthroline, 1-methyl, 2-pyridyl biimidazole, or derivatives thereof. The redox mediator may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1, 10-phenanthroline for complexation with the osmium cation include but are not limited to 4,7-dimethyl-1, 10-phenanthroline and mono, di-, and polyalkoxy-1, 10-phenanthrolines, such as 4,7-dimethoxy-1,10-phenanthroline. Polymers for complexation with the osmium cation include but are not limited to polymers and copolymers of poly(1-vinyl imidazole) and poly(4-vinyl pyridine). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole). An example of a redox polymer is also derived from poly(1-vinylimidazole) or a copolymer of (1-vinyl imidazole) bound to a metal ion selected from the group consisting of Os.sup.3+/2+, Ru.sup.3+/2+, and Fe.sup.3+/2+. The term “transition metal” refers to an element whose atom has a partially filled d sub-shell, or which can give rise to cations with an incomplete d sub-shell. Thereby, transition metals are elements in the d-block of the periodic table and also lanthanides and actinides.

Non-limiting examples of transition metal complexes include complexes comprising titanium, vanadium, chromium, manganese, iron, cobalt, nickel, copper, zirconium, niobium, molybdenium, technetium, ruthenium, rhodium, palladium, silver, hafnium, tantalum, tungsten, rhenium, osmium, iridium, and platinum. Specific examples of transition metal complexes are ferrycyanide, ruthenium hexamine, metalloporphyrins such as heme b or heme c. In these complexes, the transition metal is coordinatively bound to one or more ligands, which are typically mono-, di-, tri-, or tetradentate.

Non-limiting examples of transition metal complexes include complexes comprising lanthanum, cerium, praseodymium, neodymium, promethium, samarium, europium, gadolinium, terbium, dysprosium, holmium, erbium, thulium, ytterbium, and lutetium.

Non-limiting examples of transition metal complexes include complexes comprising actinium, thorium, protactinium, uranium, neptunium, plutonium, americium, curium, berkelium, californium, einsteinium, fermium, mendelevium, nobelium, and lawrencium.

A sandwich compound is a chemical compound featuring a metal bound by haptic covalent bonds to two arene ligands. The arenes have the formula CₙHₙ, substituted derivatives (for example Cₙ(CH₃)ₙ) and heterocyclic derivatives (for example BCₙHₙ₊₁). A special class of sandwich complexes are metallocenes. A metallocene contains a transition metal and two cyclopentadienyl ligands coordinated in a sandwich structure, i.e., the two cyclopentadienyl anions are on parallel planes with equal bond lengths and strengths. Non-limiting examples of sandwich compounds and metallocenes are ferrocene, 1,1′-dimethyl ferrocene [DMF], and ferrocene monocarboxylic acid.

Organic redox compounds, herein also referred to as organic redox mediators, are organic molecules capable to act as a redox mediator. Non-limiting examples of organic redox mediators are organic molecules such as quinones, compounds having a quinoid structure such as benzoquinones or phenanthroline quinones, phenazine such as 1-methoxyphenazine methosulfate, tetracyanoquinodimethane (TCNQ), N,N,N′, N′-tetramethyl-p-phenylenediamine (TMPD), DCIP, tetrathiafulvalene (TTF), and derivative of these molecules.

According to one embodiment of the invention, a soluble redox mediator is a freely diffusing compound dissolved in the solution phase which is capable of transferring electrons between the enzyme and electrode by changing its redox state.

According to one embodiment of the invention, an insoluble redox mediator is a compound not dissolved in the solution phase immobilized to the electrode surface, which is capable of transferring electrons between the enzyme and electrode by changing its redox state.

According to one embodiment of the invention, a wired redox mediator is a polymer with several covalently attached, redox mediator units which limits their diffusion.

According to one embodiment of the invention, the LDH is immobilized on the electrode by adsorption, physical entrapment in a polymer, complex formation, preferably via an additional complexing linker, covalent binding, in particular cross-linking, or ionic binding and/or the immobilized LDH can be cross-linked, in particular by bifunctional agents, to increase stability or activity. Cross-linking agents are e.g., dialdehydes such as glutaraldehyde.

According to another embodiment of the invention, the oxygen interference of the electrode is below 10%. More specifically, the oxygen interference of the electrode is below 9, 8, 7, 6, 5, 4, 3, 2, 1%, or even not detectable.

The term “oxygen interference” as used herein refers to the phenomenon occurring in second-generation biosensor which rely on mediated electron transfer from enzyme to electrode. If an enzyme in this setup is capable to transfer electrons to oxygen and to the redox mediator used for mediated electron transfer, some of the electrons are transferred to oxygen and not to the redox mediator. Since the accurate determination of the concentration of an analyte depends on the transfer of electrons gained from the substrate to the redox mediator, variation in the oxygen levels leads to signal variation and thus false results. In other words, oxygen competes with the redox mediator at the active site of the enzyme and thereby “steals” electrons away from the detectable electron flow leading to a reduced sensor signal, that is dependent on the available oxygen concentration, which may vary in samples.

Oxygen interference can be measured by measuring the sensor in presence of a redox mediators at high and low oxygen concentrations in the measurement solution, which can be tuned by bubbling the solution with nitrogen or argon gas. The difference in the recorded current response of the sensor can be used to quantify the interference.

Alternatively, the oxygen interference can be measured at the enzyme level. Thereby, the electron transfer of LDH to oxygen (oxidase activity) and to other electron acceptors (dehydrogenase activity) can be determined by different enzyme assays. The ratio of oxidase activity and dehydrogenase activity can be determined from the specific activities determined by these assays.

According to one embodiment of the invention, the oxygen interference at the enzyme level is determined by determination of the oxidase activity using the Amplex Red (AR)-assay and the dehydrogenase activity using the DCIP-assay. Specifically, the ratio of the oxidase activity and the dehydrogenase activity (AR/DCIP) is below 0.1 or below 0.09, 0.08, 0.07, 0.06, 0.05, 0.04, 0.03, 0.02, or even below 0.01. Specifically, the ratio of the oxidase activity and the dehydrogenase activity (AR/DCIP) may also not be possible to be determined because of the undetectable oxidase activity. The Amplex Red- and DCIP-assays are described in the following sections.

According to another embodiment the invention, the oxygen interference at the enzyme level is determined by determination of the oxidase activity using the Amplex Red (AR)-assay and the dehydrogenase activity using the FcPF₆-assay. Specifically, the ratio of the oxidase activity and the dehydrogenase activity (AR/FcPF₆) is below 0.1 or below 0.09, 0.08, 0.07, 0.06, 0.05, 0.04, 0.03, 0.02, or even below 0.01. Specifically, the ratio of the oxidase activity and the dehydrogenase activity (AR/FcPF₆) may also not be possible to be determined because of the undetectable oxidase activity. The Amplex Red- and FcPF₆-assays are described in the following sections.

According to another embodiment the invention, the oxygen interference at the enzyme level is determined by determination of the oxidase activity using the Amplex Red (AR)-assay and the dehydrogenase activity using the 1-4-BQ-assay. Specifically, the ratio of the oxidase activity and the dehydrogenase activity (AR/1-4-BQ) is below 0.1 or below 0.09, 0.08, 0.07, 0.06, 0.05, 0.04, 0.03, 0.02, or even below 0.01. Specifically, the ratio of the oxidase activity and the dehydrogenase activity (AR/1-4-BQ) may also not be possible to be determined because of the undetectable oxidase activity. The Amplex Red- and 1-4-BQ-assays are described in the following sections.

The enzymatic activity of a LDH or a variant thereof can be determined by a DCIP assay assessing the enzymatic activity from the colorimetric reduction of 2,6-dichlorophenol-indophenol sodium salt hydrate (DCIP) at 30° C. and 520 nm or alternatively at 600 nm (molar extinction coefficient ε₅₂₀ₙₘ=6.8 mM−1 cm−1; molar extinction coefficient ε₆₀₀ₙₘ=8.98 mM-1 cm-1), e.g. as previously described (W. J. Bao, S. N. et al. (1993), Krondorfer I., et al. (2014), Harreither, W. et al. (2011)). The assay mixture is buffered at pH 7.4 with 11 mM potassium phosphate, 137 mM NaCl, 3 mM KCl and contains 10 mM lactate and 120 μM DCIP, which acts as an electron acceptor. The DCIP assay thereby provides a measure of the efficiency of the electron transfer between the LDH to external electron acceptors and provides an indication of the enzyme's response on electrodes. One unit of enzymatic activity is defined as the amount of enzyme that oxidizes 1 μmol of lactate per min under the assay conditions (U/mg). The reaction stoichiometry of lactate: DCIP is 1:1, since two electrons are gained per lactate molecule and transferred to a single DCIP molecule. For the detection of activity with other substrates, lactate can be exchanged for other compounds.

The enzymatic activity of LDH variants can also be determined by assessing the colorimetric reduction of 500 μM 1,4-benzoquinone (1-4-BQ) (molar extinction coefficient ε₂₉₀ₙₘ=2.24 mM⁻¹ cm⁻¹) or 160 μM ferrocenium hexafluorophosphate (FcPF₆) (molar extinction coefficient ε₃₀₀ₙₘ=4.3 mM⁻¹ cm⁻¹). The assay mixture is formulated as is described for the DCIP assay but contains 500 μM 1,4-benzoquinone or 160 μM ferrocenium hexafluorophosphate instead of DCIP. The reaction stoichiometry of lactate: 1,4-benzoquinone is 1:1, since two electrons are gained per lactate molecule and transferred to a single molecule of 1,4-benzoquinone. The reaction stoichiometry of lactate: ferrocenium hexafluorophosphate is 1:2, since two electrons are gained per lactate molecule and transferred individually to two molecules ferrocenium hexafluorophosphate. For the detection of activity with other substrates, lactate can be exchanged for other compounds (Brugger D, et al. (2014), Sygmund, C. et al. (2011)).

The determination of the enzymatic activity of a LDH or a variant thereof can be also determined for oxygen as electron acceptor. Thereby, specifically the oxidase activity of a LDH is measured. The oxidase activity might not be detectable if the capability of the LDH to transfer electrons to oxygen is very low. As an example of a suitable method, the Amplex Red assay can be used. Thereby, oxidase activity is measured using a peroxidase-coupled reaction containing 7.1 U/mL horseradish peroxidase (181 U/mg; Sigma) and 0.05 mM AmplexRed (resorufin: ε₅₆₀ₙₘ=54.0 mM⁻¹ cm⁻¹). Oxygen is present at ambient concentrations of ˜250 μM (Kadowaki, M. A. S. et al. (2020)).

Specifically, the recombinant LDH described herein comprises an enzymatic activity of at least 1 U/mg as determined by the DCIP assay described herein. One unit of enzymatic activity is defined as the amount of enzyme that oxidizes 1 μmol of lactate per min under the respective conditions. The specific activity is given in “U/mg” or “U per mg”.

The term “activity” as used herein e.g., in the context of an enzyme activity, shall refer to a functionally active molecule. A functional enzyme is specifically characterized by a catalytic centre recognizing the enzyme substrate and catalysing the conversion of the substrate to a conversion product. For LDH the main substrate is L-lactate and the conversion product is pyruvate. Enzyme variants are considered functional upon determining their enzymatic activity in a standard test system, e.g. wherein the enzymatic activity is at least 50% of the activity of the parent (not modified or wild-type enzyme), or at least any of 60%, 70%, 80%, 90%, 100%, or even more than 100%.

According to one embodiment of the invention, the electrode of the invention is part of a biosensor. Thereby, a specific use of the electrodes of the invention is in the provision of a biosensor, more specifically a second-generation lactate biosensor using mediated electron transfer properties (MET) to detect lactate and/or to measure the lactate concentration. The biosensor may be suitable for use at acidic, neutral, or alkaline pH. The biosensor may be suitable for use at room temperature or at body temperature. Specifically, the biosensor may be suitable for the detection and/or quantification at 4, 10, 15° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or higher.

According to another embodiment, the biosensor may have one or more electrodes comprising the LDH. In further embodiments, the lactate biosensor includes: a working electrode comprising a conductive material, wherein the LDH is in proximity to the conductive material. One or more other electrodes may be included such as one or more counter electrodes, one or more reference electrodes and/or one or more counter/reference electrodes.

As described in Rocchitta G. et al, 2016, a biosensor may be used as an off-line, in vivo, or on-line device. In an off-line device, a target analyte is measured in a collected biological sample. For example, an off-line biosensor may be used for the measurement of lactate in a food or blood sample. In an in vivo sensor, a biosensor is implanted and continuously detects extracellular changes in the concentrations of an analyte. In an on-line device, a biosensor is integrated with a sampling device implanted in the body or biological material.

The particular configuration of the biosensor may depend on the use for which the biosensor is intended and the conditions under which it will operate.

In a specific embodiment of the present invention, the biosensor may be a single use biosensor for the detection of lactate. Thereby, the biosensor may be a biosensor strip.

In a specific embodiment of the present invention, biosensors are in vivo wholly positioned biosensors or transcutaneous positioned configured for in vivo positioning in a subject. In one example, at least a portion of the sensor may be positioned in the subcutaneous tissue for testing lactate concentrations in interstitial fluid. In another example, at least a portion of the sensor may be positioned in the dermal tissue for testing analyte concentration in dermal fluid.

In a specific embodiment of the present invention, the biosensor may be used for the continuous measurement of lactate in vivo.

In a specific embodiment of the present invention, the biosensor may be an implantable biosensor. The biosensor may be placed subcutaneously in a subject.

In a specific embodiment of the present invention, biosensor may be a microneedle based sensor. This biosensor is recognized by an array of multiple needles placed subcutaneously in a subject. The subject may be a human subject.

According to one embodiment of the invention, the electrode of the invention is part of a device. A device may be a wearable device, such as a smart watch, fitness tracker or heart rate monitor belt or a device connecting the electrode to a stationary or portable analyzer.

According to another embodiment of the invention, the kit for detecting and/or quantifying lactate comprises the electrode of the invention, or the biosensor of the invention, or the device of the invention, and further comprises an instruction manual. The kit may also comprise auxiliary substances, like buffers, and containers such as a sample holding means and/or lactate standards. Lactate standards may be used to calibrate the assay. The kit may also comprise a reader for a signal, especially an electrochemical signal such as a potentiostat, a computer readable memory device with software for calibration and/or measurement calculations.

According to one embodiment of the present invention, the LDH may be recombinantly expressed by methods commonly known in the art. For example, the LDH of the invention may be expressed using standard methods for cloning, transformation, and recombinant production in Escherichia coli or in Pichia pastoris.

According to a specific invention, the recombinant LDH of the invention comprises the amino acids GP at the N-terminus. This specific N-terminus results from the N-terminal addition of a tag encoding for the amino acid sequence GSSHHHHHHGLEVLFQGP (SEQ ID NO: 250) or as an alternative a tag with a higher or lower number of His residues. Specifically, this tag is added between the start codon encoding the N-terminal M and the second codon encoding the second amino acid in the respective amino acid sequence. This tag comprises a HRV 3C protease cleavage site which leaves an N-terminal GP after digestion with the HRV 3C protease. Because of the addition of such a tag and the specific cleavage site of the protease, the LDH has a defined N-terminus comprising GP. Surprisingly, this defined N-terminus leads to an increase of the specific activity of the lactate dehydrogenase compared to the uncleaved N-terminus not having the defined N-terminal GP. Specifically, the LDH with GP at the N-terminus has an at least 4-fold increased specific activity compared to the undigested N-terminus. Specifically, said increase in specific activity is 4-, 5-, 6-, 7-, 8-, 9-, 10-, 11-, 12-, 13-, 14-, 15-, 16-, 17-, 18-, 19-, 20-fold or even higher increased compared to the undigested N-terminus not having the defined N-terminal GP.

The terms “increase in activity”, “increased activity”, or the like used herein may refer to a detectable increase in activity of an enzyme. The terms “increase in activity”, or “increased activity” used herein may mean that a modified enzyme, such as e.g. LDH variants comprising a defined N-terminus comprising the leader sequence GP at the N-terminus as described herein showing higher activity than a comparable enzyme of the same type, like an enzyme that does not have the particular modification. As another example, the modified LDH may comprise sequence alterations in the polypeptide or the nucleotide sequence encoding the LDH. For example, activity of a modified or engineered enzyme may be higher than activity of a non-engineered enzyme of the same type, for example, a wild-type enzyme by about 5% or more, about 10% or more, about 15% or more, about 20% or more, about 30% or more, about 50% or more, about 60% or more, about 70% or more, or about 100% or more. The activity of a particular protein or enzyme in a recombinant or engineered cell may be higher than the activity of a protein or enzyme of the same type in a parent cell, for example, a non-engineered cell by about 5% or more, about 10% or more, about 15% or more, about 20% or more, about 30% or more, about 50% or more, about 60% or more, about 70% or more, or about 100% or more. Increased activity of an enzyme or protein in a cell may be verified by any methods known in the art.

The term “functional variant” or “functionally active variant” also includes naturally occurring allelic variants, as well as mutants or any other non-naturally occurring variants. As is known in the art, an allelic variant, or also referred to as homologue, is an alternate form of a nucleic acid or peptide that is characterized as having a substitution, deletion, or addition of one or more nucleotides or amino acids that does essentially not alter the biological function of the nucleic acid or polypeptide. Specifically, a functional variant may comprise a substitution, deletion and/or addition of 1, 2, 3, 4, 5, 6, 7, 8, 9, 10, 11, 12, 13, 14, 15, 16, 17, 18, 19 or 20 amino acid residues, or a combination thereof. Specifically, substitutions, deletions and/or additions may be conservative modifications. Specifically, substitutions, deletions and/or additions do not decrease the enzyme's specific activity. Specifically, a functionally active variant of the LDH as described herein comprises specific enzymatic activity towards lactate of at least 1 U/mg, as determined by the DCIP assay as described herein.

Specifically, a functional variant as described herein comprises no more than or up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid substitutions, deletions and/or additions. Specifically, these modifications may be conservative modifications. Specifically, these modifications do not decrease the enzyme's specific activity. Specifically, a functionally active variant as described herein comprises up to 15, preferably up to 10 or 5, amino acid substitutions, deletions and/or additions. Specifically, these modifications may be conservative modifications. Specifically, these modifications do not decrease the enzyme's specific activity.

Specifically, a functionally active variant described herein comprises at least 40, 50, 60, 70, 80 or 90% or even more of the enzymatic activity of the respective wild type enzyme. According to a specific example, if the LDH described herein is derived from the Pediococcus acidilactici LDH (PaLDH), said recombinant LDH is a functionally active variant if it comprises at least 40, 50, 60, 70, 80 or 90% or even more of the enzymatic activity of the LDH comprising SEQ ID NO: 2.

Functional variants may be obtained by sequence alterations in the polypeptide or the nucleotide sequence e.g., by one or more point mutations, wherein the sequence alterations retain or improve a feature of the enzyme, such as its stability or activity for example. Such sequence alterations can include, but are not limited to, (conservative) substitutions, additions, deletions, mutations, and insertions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion, or insertion of one or more single (non-consecutive) or doublets of amino acids for different amino acids.

According to a specific embodiment, the LDH described herein comprises one or more tag sequences, specifically N-terminal tag sequences. Specifically, such tag sequence is C-terminal of the N-terminal methionine of the recombinant LDH described herein. Such tag sequence may comprise any number of amino acids of more than 2, 4, 5, 6 or 10 amino acids and up to 20 or 50 or more amino acids. Specifically, tag sequences used herein may be any tag sequence known to the person skilled in the art. Specifically, tag sequences used herein are selected from affinity tags, solubility enhancement tags or monitoring tags.

Affinity tags are amino acid sequences that can be used for example for the purification of proteins where they are attached to. These affinity tags have high affinity to appropriate ligands of a solid support, like chromatography resins or directly to the resins. By selectively binding of the protein having the affinity tag to the particular resin the protein can be purified highly effective by only one chromatography step. According to a specific embodiment, affinity tag sequences used herein are selected from histidine (His) tag, specifically a poly-histidine tag, poly-arginine tag, FLAG tag, Strep tag, streptavidin-binding peptide (SBP) tag, calmodulin-binding peptide (CBP) tag, S-tag, HA tag, c-Myc tag, and SUMO tag, or any other tag known to be useful for the efficient purification of a protein it is fused to. Preferably, the tag is a His tag comprising one or more H, specifically a hexahistidine tag. Specifically, proteins comprising a poly-, or hexa-histidine tag (His-tag) can be captured and purified using chromatography, e.g. by Immobilized Metal Affinity Chromatography (IMAC).

Solubility enhancement tags can be fused N-terminal to the LDH described herein. Solubility enhancement tags can increase the titer of the soluble protein when expressed in a host cell, e.g. in the cytosol of P. pastoris, compared to expression of the proteins without the tag. According to a further specific embodiment, solubility enhancement tag sequences used herein are selected from calmodulin-binding peptide (CBP), poly Arg, poly Lys, protein D tag (dTAG), Z domain of Staphylococcal protein A, and thioredoxin or any other tag known to improve the solubility of the protein it is fused to e.g. during expression in a host cell. Specifically, the solubility enhancement tag is a T7 tag, preferably selected from the group consisting of T7A, T7A1, T7A2, T7A3, T7A4, T7A5, T7B, T7B1, T7B2, T7B3, T7B3, T7B4, T7B5, T7B6, T7B6, T7B7, T7B8, T7B9, T7B10, T7B11, T7B12, T7B13, and T7C.

According to a further specific embodiment, the monitoring tag sequence used herein is m-Cherry, GFP or f-Actin or any other tag useful for detection or quantification of the recombinant enzyme during production steps including fermentation, isolation and purification by simple in-situ, inline, online or at line detectors, like UV, IR, Raman, fluorescence and the like.

The term “sequence identity” as used herein is understood as the relatedness between two amino acid sequences or between two nucleotide sequences and described by the degree of sequence identity or sequence complementarity. The sequence identity of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or polynucleotides by detecting excess similarity, and statistically significant similarity that reflects common ancestry. Homologues may encompass orthologues, which are herein understood as the same protein in different organisms, e.g., variants of such protein in different organisms or species.

To determine the % complementarity of two complementary sequences, one of the two sequences needs to be converted to its complementary sequence before the % complementarity can then be calculated as the % identity between the first sequence and the second converted sequences using the above-mentioned algorithm.

“Percent (%) identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, and not considering any conservative substitutions as part of the sequence identity. Those skilled in the art can determine appropriate parameters for the alignment, including any algorithms needed to achieve the highest scoring alignment over the full length of the sequences being compared. In case of percentages determined for sequence identities, it is possible that arithmetical decimal places may result which are not possible with regard to full nucleotides or amino acids. In this case, the percentages shall be rounded up to whole nucleotides or amino acids.

For purposes described herein, the sequence identity between two amino acid sequences is determined using standard methods, e.g. using the NCBI BLAST program version 2.2.29 (Jan.-06-2014) or online using the multiple sequence alignment tool EMBL-EBI Clustal Omega (Sievers, F. et al. Fast, scalable generation of high-quality protein multiple sequence alignments using Clustal Omega. Mol. Syst. Biol. 7, 539 (2011)).

“Percent (%) identity” with respect to a nucleotide sequence e.g., of a nucleic acid molecule or a part thereof, in particular a coding DNA sequence, is defined as the percentage of nucleotides in a candidate DNA sequence that is identical with the nucleotides in the DNA sequence, after aligning the sequence and introducing gaps, if necessary, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for the alignment, including any algorithms needed to achieve the highest scoring alignment over the full length of the sequences being compared.

Optimal alignment may be determined with the use of any suitable algorithm for aligning sequences, non-limiting examples of which include the Smith-Waterman algorithm, the Needleman-Wunsch algorithm, MAFFT based algorithms: multiple alignment using fast fourier transform, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomies.org.cn), and Maq (available at maq.sourceforge.net).

In a structure alignment the maximal set of corresponding pairs of amino acid residues that gives a good structural match when the structures are overlaid, i.e., superposed, is identified. Thereby, the positions of the protein's backbone C-alpha atoms and/or location of secondary structural elements are considered in this alignment. Tools for performing a structure alignment are available, e.g., the protein data bank provides a tool for pairwise structure alignment. Specifically, structure superposition is also a tool for determining corresponding amino acid positions in different enzymes. Structure superposition can be performed using the Molecular Graphics System PyMOL, (Schrödinger) using the command “align”.

The examples described herein are illustrative of the present invention and are not intended to be limitations thereon. Many modifications and variations may be made to the techniques described and illustrated herein without departing from scope of the invention. Accordingly, it should be understood that the examples are illustrative only and are not limiting upon the scope of the invention.

### EXAMPLES

### Materials

Phosphate-buffered saline (PBS) 11 mM, pH 7.4 with 137 mM NaCl and 3 mM KCl was used as standard buffer for all experiments unless stated otherwise. Britton-Robinson universal buffer (BRB) contains 40 mM phosphoric, boric and acetic acid. Cultivation of bacteria was routinely done in Luria-Bertani (LB) medium (10 g/L peptone from casein, 5 g/L yeast extract and 10 g/L NaCl) with 100 mg/L ampicillin. In case of cultivating bacteria carrying the pNIC-CH plasmid for PaLDH, ampicillin was replaced with 50 mg/L kanamycin. General media components were purchased from Carl Roth; sodium L-lactate, ferrocenium hexafluorophosphate (FcPF₆), isopropyl b-D-1-thiogalactopytanoside (IPTG), 2,6-dichlorophenol-indophenol sodium salt hydrate (DCIP), horseradish peroxidase (HRP), sodium glycolate, 1,4-benzoquinone (1,4-BQ), R-2-hydroxybutyric acid, S-2-hydroxybutyric acid from Sigma-Aldrich (Germany); S-2-hydroxyvaleric acid from BLD Pharmatech Ltd. (Shanghai); 2-hydroxypalmitic acid and 2-hydroxy-n-octanoic acid from TCI (Japan); (S)-2-hydroxybutyric acid and S-(+)-mandelic acid from Fluorochem Ltd. (United Kingdom); and 10-acetyl-3,7-dihydroxyphenoxazine (AmplexRed®) from Chemodex (Switzerland).

### Example 1: Phylogenetic Analysis

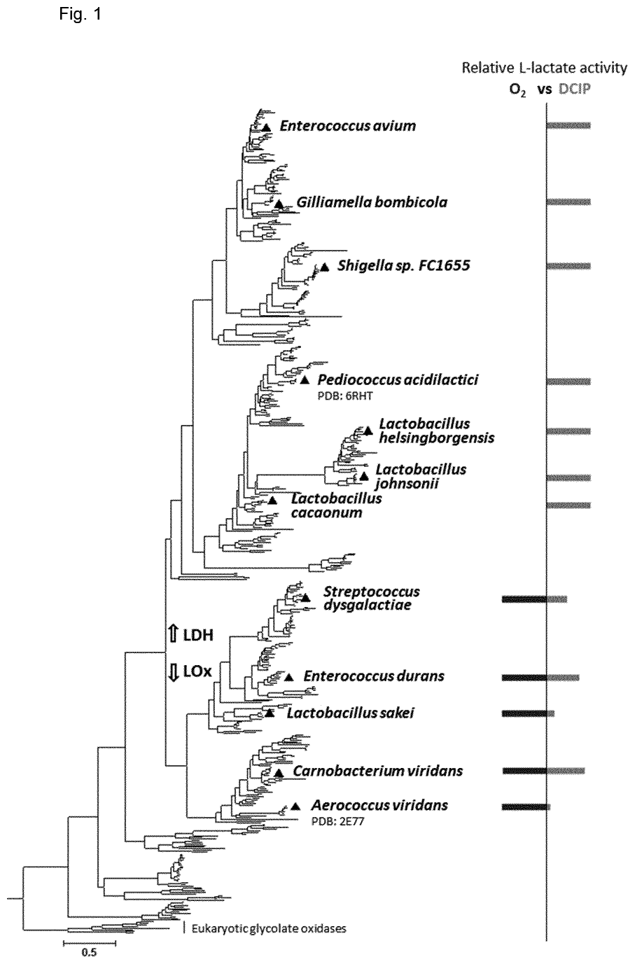

It was surprisingly found that there is a group of enzymes closely related but separate from characterized LOx sequences. Furthermore, by an activity screening approach of novel members of the LOx cluster, comprising of both enzyme groups, it was found that there is even a functional split of highly lactate specific oxidase vs. dehydrogenase sequences. To get a more detailed view of the LOx cluster and its sequence/function distribution, a phylogenetic tree of all sequences in the cluster was inferred using RAxML and sequences with their relative L-lactate activities from the screening were annotated in the tree (FIG. 1). Clearly a functional split of the tree into two main clades, with one clade showing oxygen reactivity (LOxs) and the other showing only dehydrogenase activity (LDHs) can be observed. The differences between the two groups found in activity, separating LOx and LDH sequences, are therefore also supported by phylogenetic information. Furthermore, the two previously characterized enzymes AvLOx and PaLCTO, fit well into the picture. While AvLOx appears in the LOx clade, PaLCTO appears in the LDH clade, which was surprising because PaLDH was reported not to utilize lactate in the past e.g., by Ashok Y. et al., 2020. Therefore, PaLCTO can be considered as a PaLDH. Apart from PaLDH, no other previously characterized enzymes were found in the LDH clade, including membrane bound and NAD+ dependent LDHs, as well as flavocytochrome b2, often referred to as LDH in literature. This shows that indeed a new type of LDH, which is closely related but separate from LOx, was invented.

Based on these new insights into the phylogenetic and functional nature of the HAOx enzyme family, a detailed sequence analysis revealed that LDHs can be separated from the rest of the HAOx family members and thus also from the LOx members by specific characteristics of the amino acid sequence of LDH members. Apparently, LDHs are separated from LOx by the sequence T-(Xₓ)ₙ₁-F-(Xₓ)ₙ₂-X₁-X₂-(Xₓ)ₙ₃-X₃-X₄ (SEQ ID NO: 1) as defined herein. The amino acid sequences of LOx enzymes do not comprise SEQ ID NO: 1 as defined herein. Surprisingly, also other HAOx family members do not comprise SEQ ID NO: 1 as defined herein while sharing at least 50% sequence identity to SEQ ID NO: 2.

Therefore, FMN-dependent LDHs with at least 50% sequence identity to SEQ ID NO: 2 and comprising SEQ ID NO: 1 as defined herein seem to form a distinct class of enzymes with the shared functional and technical property of oxidizing lactate and transferring the so gained electrons to electron acceptors other than oxygen.

Selected representatives of sub-clades of this distinct class of LDHs have been functionally expressed and characterized as described herein.

### Example 2: Analysis of Structural Motif

The sequence of T-(Xₓ)ₙ₁-F-(Xₓ)ₙ₂-X₁-X₂-(Xₓ)ₙ₃-X₃-X₄ (SEQ ID NO: 1) as defined herein forms a structural motif in the 3-dimensional structure of the folded enzyme. Said sequence is located in or interacting with the active site lid of the enzyme. The active site lid of a LDH of the invention is described in the crystal structure of the PaLDH (Ashok Y. et al., 2020). The crystal structure of PaLDH can be found under the protein data bank (pdb)—code 6RHT in two conformations which are referred to herein as the “open and closed” conformation of the active site lid. In the crystal structure of PaLDH, the α-carbons of amino acid residues T, F, X₁ being N, and X₂ being L of sequence T-(Xₓ)ₙ₁-F-(Xₓ)ₙ₂-X₁-X₂-(Xₓ)ₙ₃-X₃-X₄ (SEQ ID NO: 1) as defined herein are located within a distance of 12 Å among each other and these amino acids not substantially differ in the open and closed conformation. The position in the crystal structure of residues X₃ being G, and X₄ being I of sequence T-(Xₓ)ₙ₁-F-(Xₓ)ₙ₂-X₁-X₂-(Xₓ)ₙ₃-X₃-X₄ (SEQ ID NO: 1) as defined herein differ in the open and closed conformation. The specific distances in the crystal structure are given in the following Table 3 showing the α-carbon distance matrix for the residues of 6RHT in PaLDH, i.e., T122, F152, N196, L197, G209 and 1210 of T-(Xₓ)ₙ₁-F-(Xₓ)ₙ₂-X₁-X₂-(Xₓ)ₙ₃-X₃-X₄ (SEQ ID NO: 1) as defined herein. The distances are given in Å. The distances for G and I are given for their positions in the open and closed confirmation respectively.

TABLE 3

| Residue | T | F | N | L | G | I |
|---|---|---|---|---|---|---|
| T | X | | | | | |
| F | 11.1 | X | | | | |
| N | 6.5 | 9.9 | X | | | |
| L | 8.1 | 8.7 | 3.9 | X | | |
| G | 16.6/15.7 | 12.7/21.0 | 12.9/17.1 | 9.2/15.2 | X | |
| I | 13.3/12.8 | 11.7/18.9 | 9.9/15.4 | 6.2/13.9 | 3.9 | X |

Structural models have been calculated using the RoseTTAFold method on the Rosetta web-server (https://robetta.bakerlab.org/submit.php) for 5 LDH sequences according to the invention (Baek, Minkyung, et al. 2021). The distance of all motif-residues to all other motif-residues within a structure was measured in PyMOL as the distance between the residues' α-carbons in angstrom (Å).

The α-carbon distance matrix for motif-residues in LjLDH [Å] is given in the following Table 4.

TABLE 4

| residue | T | F | F | F | | |
|---|---|---|---|---|---|---|
| T | X | | | | | |
| F | 10.4 | X | | | | |
| F | 6.2 | 9.0 | X | | | |
| F | 8.1 | 8.9 | 3.7 | X | | |
| S | 14.6 | 16.9 | 13.2 | 10.0 | X | |
| V | 11.6 | 15.5 | 11.5 | 9.0 | 3.8 | X |

The α-carbon distance matrix for motif-residues in LhLDH [Å] is given in the following Table 5.

TABLE 5

| residue | T | F | F | F | T | V |
|---|---|---|---|---|---|---|
| T | X | | | | | |
| F | 11.1 | X | | | | |
| F | 6.1 | 10.0 | X | | | |
| F | 8.0 | 9.5 | 3.7 | X | | |
| T | 14.0 | 15.2 | 11.6 | 8.2 | X | |
| V | 11.3 | 14.5 | 10.4 | 7.5 | 3.8 | X |

The α-carbon distance matrix for motif-residues in SsLDH [Å] is given in the following Table 6.

TABLE 6

| residue | T | F | N | L | G | I |
|---|---|---|---|---|---|---|
| T | X | | | | | |
| F | 10.7 | X | | | | |
| N | 6.2 | 10.7 | X | | | |
| L | 7.0 | 9.6 | 3.8 | X | | |
| G | 14.2 | 18.2 | 14.1 | 11.2 | X | |
| I | 11.2 | 16.4 | 12.6 | 10.0 | 3.8 | X |

The α-carbon distance matrix for motif-residues in GbLDH [Å] is given in the following Table 7.

TABLE 7

| residue | T | F | N | L | G | I |
|---|---|---|---|---|---|---|
| T | X | | | | | |
| F | 10.4 | X | | | | |
| N | 5.7 | 10.2 | X | | | |
| L | 6.3 | 8.9 | 3.8 | X | | |
| G | 14.6 | 19.4 | 15.3 | 12.6 | X | |
| I | 12.3 | 17.9 | 14.3 | 11.8 | 3.8 | X |

The α-carbon distance matrix for motif-residues in EaLDH [Å] is given in the following Table 8.

TABLE 8

| residue | T | F | N | L | G | I |
|---|---|---|---|---|---|---|
| T | X | | | | | |
| F | 10.5 | X | | | | |
| N | 6.5 | 9.8 | X | | | |
| L | 7.1 | 8.7 | 3.8 | X | | |
| G | 14.5 | 18.5 | 14.9 | 12.1 | X | |
| I | 13.2 | 18.1 | 15.3 | 12.6 | 3.8 | X |

### Example 3: Recombinant Expression of LDH and Characterization Plasmids and Genes

Genes coding for the selected LDH enzymes and for AvLOx for comparison were ordered codon-optimized for E. coli expression in a pET-21(+) vector from Twist Bioscience (South San Francisco, USA). The selected LDH enzymes were Lactobacillus johnsonii LjLDH (SEQ ID NO: 59), Lactobacillus helsingborgensis LhLDH (SEQ ID NO: 13), Shigella sp. FC1655 SsLDH (SEQ ID NO: 74), Gilliamella bombicola GbLDH (SEQ ID NO: 73), Enterococcus avium EaLDH (SEQ ID NO: 155), Pediococcus acidilactici PaLDH (SEQ ID NO: 2). The N-terminal purification tag (-GSS-HHHHHH-G-LEVLFQGP-(SEQ ID NO: 250)) was added between the start-Met and the second amino acid according to the protocol of Gibson Assembly (New England Biolabs) using 146 and 110 bp overlaps at the 5′ and 3′ end respectively. The native form of PaLDH was cloned in a pNIC-CH vector as described previously (Ashok Y. et al. 2020). A predicted bacterial signal peptide of SsLDH was removed by amplifying the plasmids without the signal-peptide-coding region by PCR and re-ligating the linearized plasmid using the KLD Enzyme Mix (New England Biolabs). Plasmids were transformed into chemically competent E. coli BL21 (DE3) cells by heat-shock transformation. All plasmid modifications and transformations were verified by Sanger sequencing (Microsynth, Austria).

### Enzyme Expression

Expression of recombinant genes in E. coli BL21 (DE3) was done in baffled shake flasks at a scale of 40 or 250 mL expression medium. LB-amp (LB-kan for PaLDH) medium was inoculated with bacterial culture to an optical density at 600 nm (OD₆₀₀) of 0.05 and was incubated at 37° C. and 180 rpm until cells reached an OD₆₀₀ of 0.45-0.50, where expression was induced by 100 μM IPTG (250 μM in case of 250 mL expressions). Induced cultures were incubated overnight at 20° C. and 180 rpm for 19 h. Cells were harvested by centrifugation at 4000 rpm for 20 min at 4° C. and washed once with 50 mM potassium phosphate buffer (PPB) pH 6.5. Washed cell pellets were stored at −20° C. prior to cell disruption.

### Purification and Protein Concentration Measurements

Frozen cell pellets were thawed, resuspended in 50 mM PPB, 500 mM NaCl, 50 mM Imidazole pH 6.5 and disrupted by 4-5 passages in a French press. Cell debris was removed by centrifugation (3000 rcf at 4° C. for 30 min) and the resulting supernatant was filtered with a 0.22 μm membrane filter and loaded onto 2×5 mL IMAC HisTrap™ FF columns (Cytiva, USA) using an Äkta FPLC system (GE Healthcare, USA). His-tagged proteins were eluted by a linear imidazole gradient (50-750 mM) in 50 mM PPB, 500 mM NaCl, pH 6.5, and fractions were pooled according to activity and elution peaks measured at 280 and 450 nm. Pooled fractions were concentrated in Amicon centrifugal filters (MWCO 10 kDa), rebuffered to 11 mM PBS, pH 7.4 and stored at 4° C. Homogeneity of the enzymes was assessed by SDS-PAGE, and protein concentrations were calculated from their absorbance at 280 nm assuming theoretical extinction coefficients as determined by the ExPASy tool ProtParam (Gasteiger et al. 2005) from the amino acid sequence (48360, 41830, 24870, 29910, 28420, 25900 and 51340 M⁻¹ cm⁻¹ for LjLDH, LhLDH, SsLDH, GbLDH, EaLDH, PaLDH and AvLOx, respectively). Purified PaLDH was produced as described previously (Ashok Y. et al. 2020) using E. coli BL21 (DE3) expression, His-tag purification followed by cleavage of the tag by TEV protease digestion and size exclusion chromatography.

### Enzymatic Activity Measurements

Spectrophotometric enzyme activity assays were recorded at least in triplicates in 96-well microtiter plates at 30° C. using an EnSpire multimode plate reader (PerkinElmer) or Infinite M Quant plate reader (Tecan). Volumetric activities were calculated from linear correlations of changes in absorption over time (ΔAbs/Δt) at the monitored wavelength.

Activity assays contained the electron donor substrate L-lactate at 10 mM concentration dissolved in buffer unless stated otherwise. Oxidase activity was monitored using a peroxidase-coupled reaction containing 7.1 U/mL horseradish peroxidase (181 U/mg; Sigma) and 0.05 mM AmplexRed (10-Acetyl-3,7-dihydroxyphenoxazine, resorufin: ε₅₆₀ₙₘ=54.0 mM⁻¹ cm⁻¹). Oxygen was present at ambient concentrations of ˜250 μM. Dehydrogenase activities were measured by direct dye-mediated assays containing 120 μM (300 μM for screening of substrates) DCIP (2,6-dichlorophenol-indophenol sodium salt hydrate, ε₅₂₀ₙₘ=6.8 mM⁻¹ cm⁻¹ or ε₆₀₀ₙₘ=8.98 mM⁻¹ cm⁻¹), 500 μM 1,4-BQ (1,4-benzoquinone, ε_{290 nm}=2.24 mM⁻¹ cm⁻¹) or 160 μM FcPF₆ (ferrocenium hexafluorophosphate, ε₃₀₀ₙₘ=4.3 mM⁻¹ cm⁻¹). Measurements of relative substrate specificities and specific activities for FcPF₆ and 1,4-BQ were conducted in 50 mM potassium phosphate buffer (PPB) pH 6.5. One unit of enzyme activity was defined as the amount of enzyme that catalyzes the oxidation of 1 μmol α-hydroxy acid per minute at 30° C.

Apparent steady-state kinetic constants were determined by using 11 different concentrations of L-lactate from 0.125 to 64 mM (4-500 mM for PaLDH) with DCIP at a constant concentration of 120 μM and fitting the Michaelis-Menten model (v=(vₘₐₓ*[S]/(Kₘ+[S])) to the observed data using iterative least-square regression fitting with the Microsoft Excel Solver plugin. Turnover rates are calculated based on the monomeric masses of the respective enzymes.

Assessing the influence of pH on the L-lactate oxidizing activity with O₂ and DCIP was done in 40 mM Britton-Robinson universal buffer (BRB) by varying the pH from 4.5 to 10 in steps of 0.5.

### Thermostability Measurements

Enzyme samples were diluted to a concentration of 1 mg/mL and incubated in duplicates for 30 min in a temperature range from 30 to 60° C. (43 to 73° C. in case of SsLDH and EaLDH). Heat treated samples were cooled on ice for 15 min and centrifuged before residual activities with L-lactate and DCIP were measured in duplicates. Estimation of the thermal inactivation temperature (T₅₀) was done by an iterative least-square regression sigmoidal curve fit (min+(max−min)/(1+10\hat{ }(n*(log ₁₀(° C.)−log ₁₀(EC₅₀))))) using the Microsoft Excel Solver plugin.

### HRV 3C Protease Digest

Purification tag cleavage was conducted at small scale using 30 μg enzyme and 1 μg HRV 3C protease (in-house production; SwissProt ID: sp|P03303|1538-1719) at 20° C. for 22 h. Alternatively, the HRV 3C protease may be purchased (HRV-3C-Protease N-Terminal His tagged recombinant protein, aqueous solution, 0.8-1.2 mg/mL|Sigma-Aldrich). Determination of activity was conducted in quadruplicates using L-Lactate and DCIP. Blinds were incubated with buffer instead of HRV 3C.

### Results

### Expression, Purification and UV-Vis Spectra of LDHs

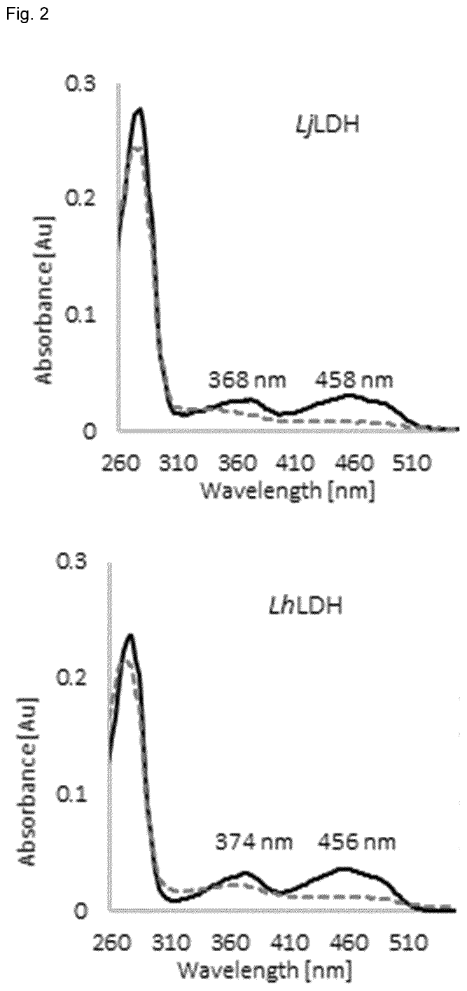

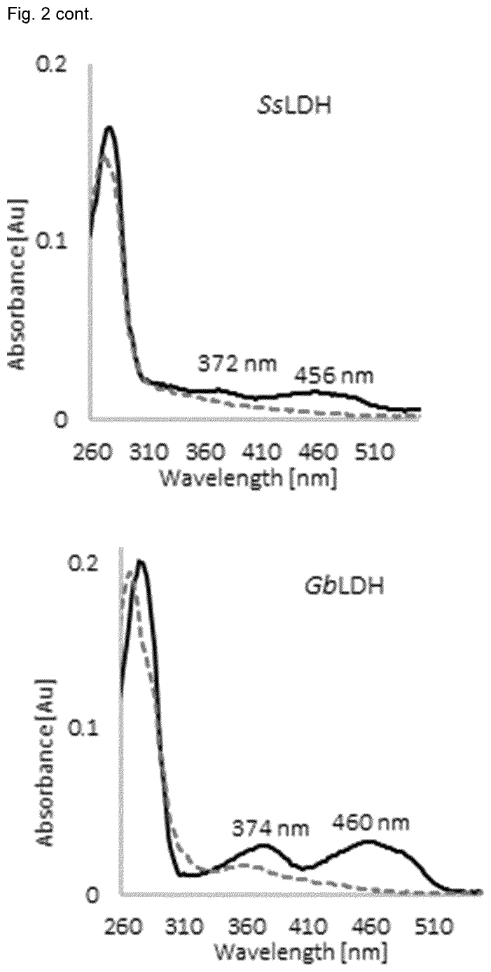

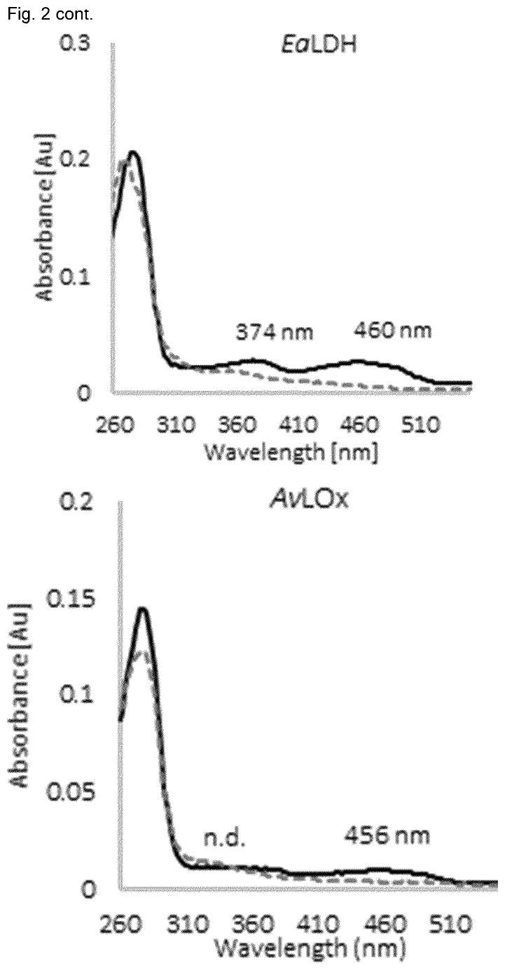

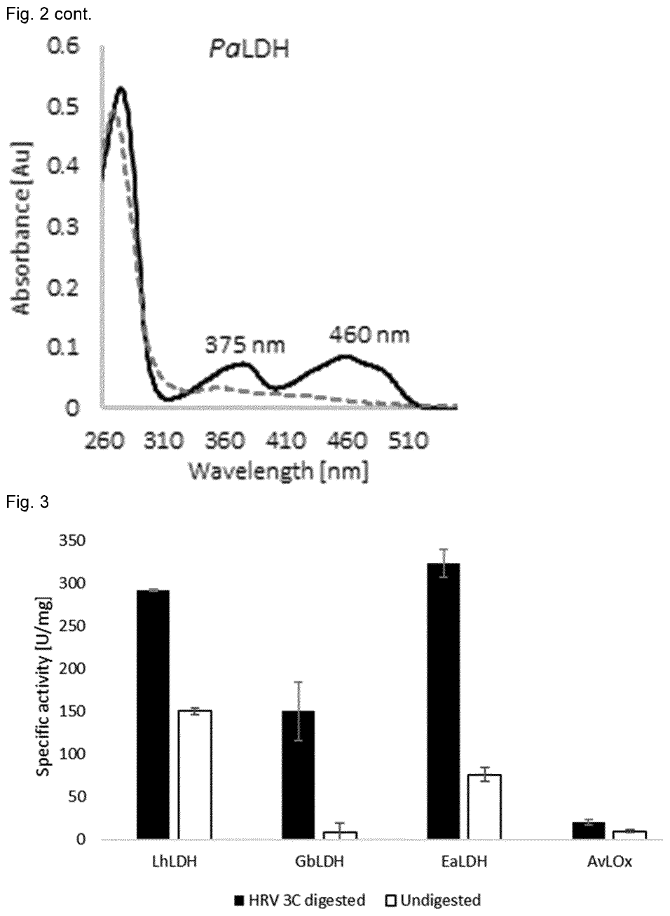

Five LDH sequences were expressed, purified, and characterized. Genes of the enzymes of L. johnsonii (LjLDH), L. helsingborgensis (LhLDH), Shigella sp. FC1655 (SsLDH), Gilliamella bombicola (GbLDH) and Enterococcus avium (EaLDH) together with AvLOx were modified with an N-terminal purification tag consisting of 6xHis and a recognition site for the 3C protease of the human rhinovirus (HRV 3C). These six genes were then expressed in E. coli BL21 (DE3) shaken flask cultures and purified using immobilized-metal affinity chromatography (IMAC). PaLDH was expressed and purified in the same way with an additional step of size exclusion chromatography, as described previously (Ashok Y. et al. 2020). Expression of LjLDH, LhLDH, SsLDH, GbLDH and EaLDH yielded significantly more recombinant protein than that of AvLOx (9, 20, 18, 119 and 7 times higher, respectively), when comparing expression yields normalized with the amount of harvested cell pellet (mg purified enzyme/g wet cell pellet) (Table 9). UV-Vis absorption spectra showed the typical peaks of flavoproteins with one maximum around 278 nm and two FMN-dependent maxima around 374 and 458 nm, which flattened upon the addition of 10 mM L-lactate due to the reduction of the cofactor (FIG. 2).

TABLE 9
Purification table of LDHs and AvLOx comparing various yields
and obtained purities. Activities were measured in 11 mM
PBS pH 7.4 using 120 μM DCIP and 10 mM L-lactate.

| | Cell | Purity by | Purified | Purified | Specific | U/g | |
|---|---|---|---|---|---|---|---|
| | pellet | SDS | enzyme | units | activity | cell | mg enzyme / |
| Enzyme | [g] | PAGE [%] | [mg] | [U] | [U/mg] | pellet | g cell pellet |
| LjLDH | 11.5 | 91.5 | 9.32 | 670 | 72 | 58.3 | 0.810 |
| LhLDH | 15.4 | 100.0 | 28.64 | 4346 | 152 | 282 | 1.86 |
| SsLDH | 15.9 | 50.4 | 26.7 | 4440 | 166 | 279 | 1.68 |
| GbLDH | 7.25 | 99.2 | 81.71 | 7569 | 93 | 1044 | 11.27 |
| EaLDH | 14.9 | 73.8 | 9.42 | 1376 | 146 | 92.3 | 0.632 |
| AvLOx | 13.0 | 41.9 | 1.23 | 16.9 | 13.7 | 1.30 | 0.095 |

### Substrate Specificities of LDHs

Specific activities were determined for all LDHs for the electron acceptors O₂ (air), dichlorophenol-indophenol (DCIP), 1,4-benzoquinone (1,4-BQ) and ferrocenium hexafluorophosphate (FcPF₆) with 10 mM L-lactate using spectrophotometric assays (Table 10). Highest specific dehydrogenase activities were measured with DCIP for LjLDH, LhLDH, SsLDH and EaLDH (72, 152, 166 and 146 U/mg, respectively), with FcPF₆ for GbLDH (207 U/mg), and with 1,4-BQ for PaLDH (25 U/mg). Oxygen reactivity was highest for AvLOx with 18 U/mg. This value is in good agreement with a previously studied N-terminally tagged AvLOx showing a vₘₐₓ of 21.81 U/mg (Taurino et al. 2013). The selected LDHs showed oxygen reactivities ranging from 0.07 to 0.71 U/mg, accounting for 0.37, 0.47, 0.07, 0.03, 0.05 and 0.37% of their maximal dehydrogenase activity for LjLDH, LhLDH, SsLDH, GbLDH, EaLDH and PaLDH, respectively.

TABLE 10
Specific activities of LDHs and AvLOx for four different electron
acceptors and 10 mM L-lactate.

| | Specific activity (U/mg) | | | |
|---|---|---|---|---|
| | FcPF₆ | 1,4-BQ | DCIP | O₂ |
| LjLDH | 24 ± 2 | 19.7 ± 0.2 | 72 ± 3 | 0.26 ± 0.01 |
| LhLDH | 31 ± 9 | 27 ± 2 | 152 ± 5 | 0.71 ± 0.04 |
| SsLDH | 41 ± 1 | 125 ± 30 | 166 ± 2 | 0.12 ± 0.02 |
| GbLDH | 207 ± 11 | 109 ± 11 | 93 ± 23 | 0.07 ± <0.01 |
| EaLDH | 65 ± 1 | 45 ± 6 | 146 ± 14 | 0.07 ± <0.01 |
| PaLDH | 11 ± 2 | 25 ± 4 | 6.9 ± 0.3 | 0.09 ± <0.01 |
| AvLOx | n.d. | n.d. | 14 ± 3 | 18.4 ± 1.8 |

n.d.-not determined

### Michaelis-Menten Kinetics

Steady-state reaction kinetics for L-lactate were measured for all LDHs using DCIP as electron acceptor at pH 7.4 (Table 11). LhLDH showed the highest activity for L-lactate as judged from the catalytic efficiency, mainly because of its low Kₘ value. The highest Kₘ value on the other hand was found for PaLDH, showing quite a large margin to all other LDHs. Turnover numbers of the studied LDHs for L-lactate varied less drastically, with the highest k_{cat} value measured for GbLDH.

TABLE 11
Apparent steady-state kinetic constants for the oxidation
of _{L}-lactate by various LDHs, measured in 11 mM PBS
at 30° C. and DCIP as electron acceptor used at
a constant concentration of 120 μM. L-lactate
concentrations were varied from 0.125 to
64 mM, or from 4 to 500 mM for PaLDH.

| | Kₘ [mM] | k_{cat} [s⁻¹] | k_{cat}/Kₘ |
|---|---|---|---|
| LjLDH | 1.35 ± 0.18 | 47.2 ± 2.0 | 35.0 |
| LhLDH | 0.52 ± 0.09 | 102 ± 8 | 196 |
| SsLDH | 5.67 ± 1.24 | 107 ± 10 | 18.9 |
| GbLDH | 16.9 ± 2.5 | 207 ± 29 | 12.3 |
| EaLDH | 21.6 ± 2.2 | 94.0 ± 7.8 | 4.35 |
| PaLDH | 235 ± 45 | 61.8 ± 8.5 | 0.263 |

### Effect of pH on LDH and AvLOx Activity

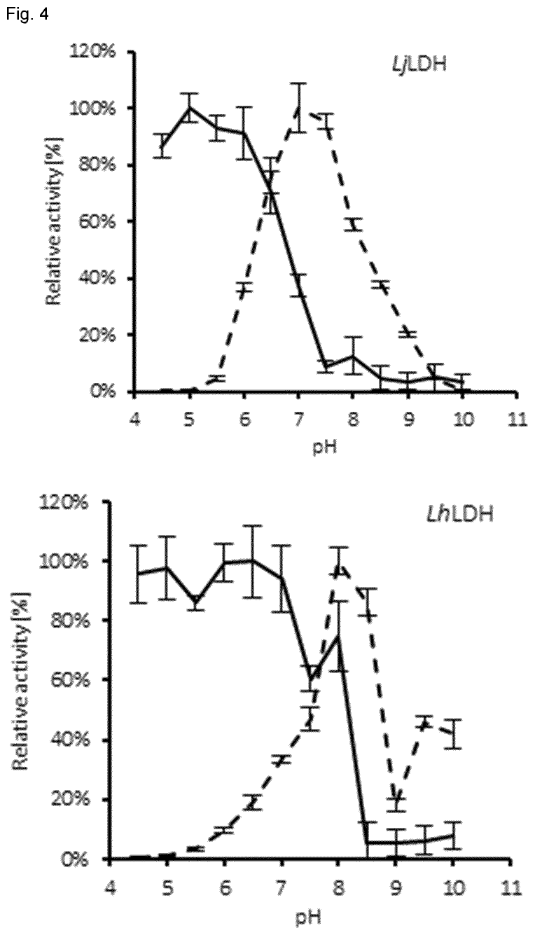

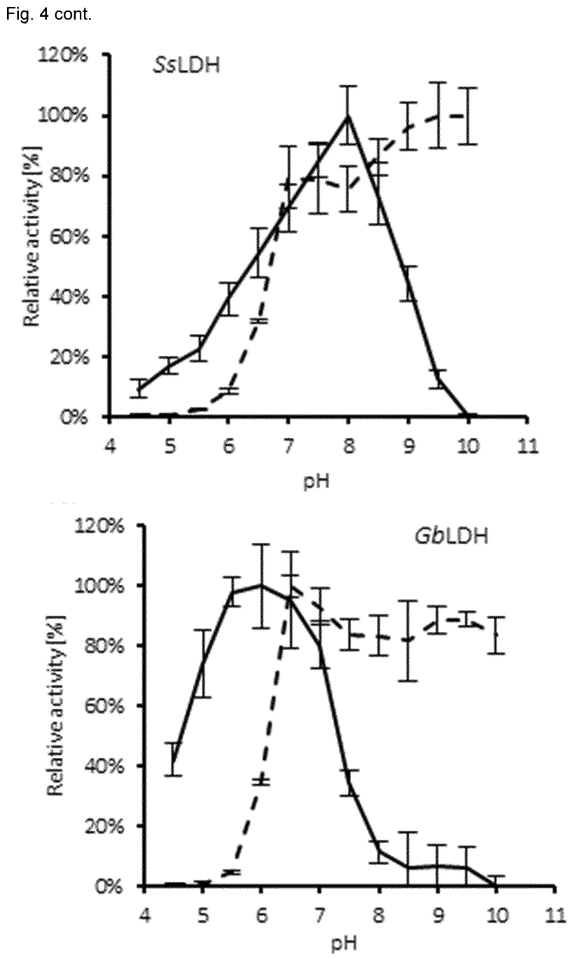

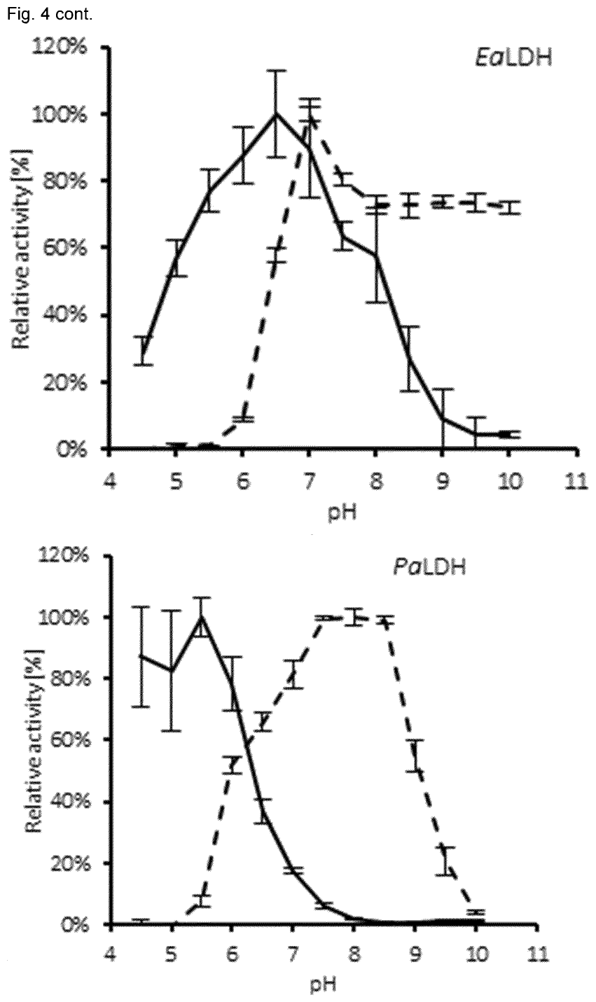

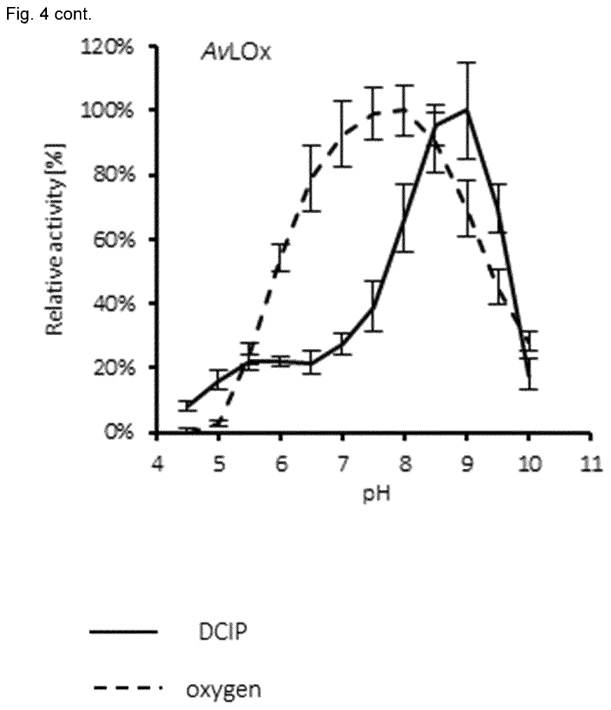

Determination of pH optima was done for all LDHs and AvLOx in Britton-Robinson buffer (BRB) from pH 4.5 to 10.0 using a dehydrogenase (DCIP as electron acceptor) as well as an oxidase (O₂/air coupled with Amplex Red) assay (FIG. 4). The resulting pH profiles differed substantially between the two activity assays for some of the enzymes studied. In general, all LDHs tended to have their optimal pH with DCIP at a lower value than the optimum for O₂. The pH range where enzymes show 80-100% activity with DCIP and O₂, respectively, is 4.5-6.0 and 7.0-7.5 for LjLDH, 4.5-7.0 and 8.0-8.5 for LhLDH, 7.5-8.0 and 8.5-10.0 for SsLDH, 5.5-7.0 and 6.5-10.0 for GbLDH, 6.0-7.0 and 7.0-7.5 for EaLDH, and 4.5-5.5 and 7.0-8.5 for PaLDH. Interestingly, AvLOx showed a pH profile for DCIP shifted to higher values when compared to O₂, with 80-100% activity in a pH range of 8.5-9.0 for DCIP and 7.0-8.5 for O₂. The pH profiles also revealed that for LjLDH, GbLDH, PaLDH and AvLOx, activities at pH 7.4 only display approximately 9, 35, 6 and 39% of the enzymes' maximum DCIP activity. This has to be taken into consideration when interpreting the enzymes' specific activities which were measured at the physiological relevant pH of 7.4. Additionally, the influence of different buffer species on the enzymes' activity was tested. To this end, activities in PBS at pH 7.4 and activities in BRB at pH 7.5 were compared and showed that for LjLDH, LhLDH and GbLDH activities in PBS were 7.8-, 2.0- and 2.1-fold higher than in BRB pH 7.5. Other tested enzymes were hardly affected in their activity when comparing the two buffers.

### Effect of Temperature on LDH Activity

Thermostability of LDHs was determined by incubating the enzymes at different temperatures for 30 minutes and determining their residual activities. The temperature at half-maximum activity (T₅₀) was obtained from an iterative sigmoidal fit of the observed data (Table 12). The two highest T₅₀ values were observed for SsLDH and EaLDH, while LhLDH showed the lowest T₅₀ value.

TABLE 12
T₅₀ values of LDHs after incubation at various temperatures for 30
minutes. Residual activities were measured with L-lactate and DCIP.

| | LjLDH | LhLDH | SsLDH | GbLDH | EaLDH |
|---|---|---|---|---|---|
| T₅₀ [° C.] | 47.2 | 36.6 | 61.0 | 39.7 | 59.4 |

### Effect of the N-Terminal Purification Taq on LDH and AvLOx Activity

The purification tag added contained a 6xHis-tag as well as a cleavage site for the HRV 3C protease, which leaves an N-terminal Gly-Pro after digestion, cleaving off the 6xHis-tag completely. Purified enzymes were incubated at 20° C. overnight (22 h) with and without HRV 3C, and their specific activities with L-lactate and DCIP were determined (FIG. 3). The thus obtained results varied considerably. AvLOx and LhLDH showed an approximately 2-fold increase in activity after the digest, compared to the undigested sample, and GbLDH and EaLDH showed 18- and 4-fold increases, respectively. We could therefore confirm that the nature of the N-terminus also affects the activity of an LDH.

### Example 4: Electrode Measurements

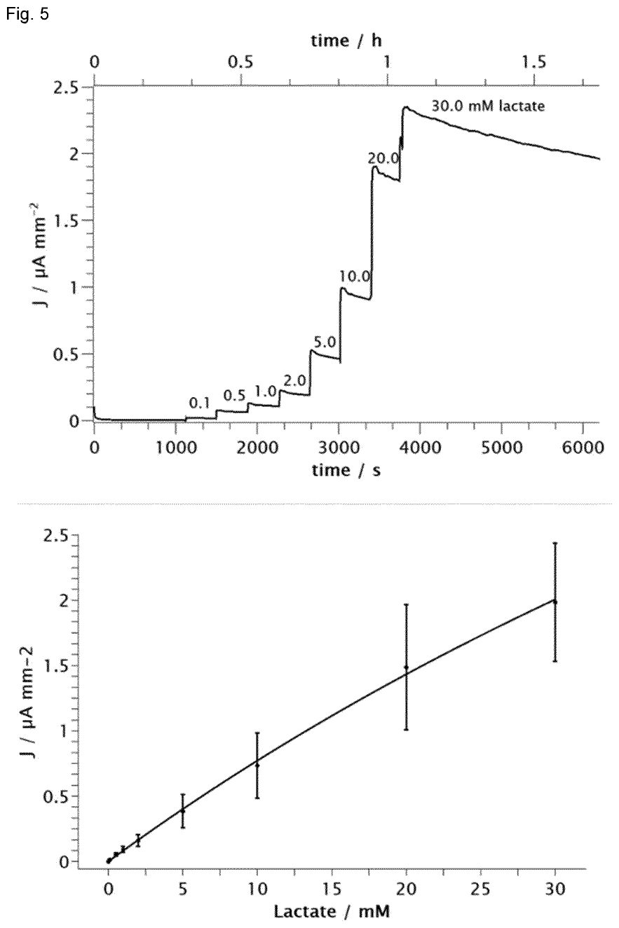

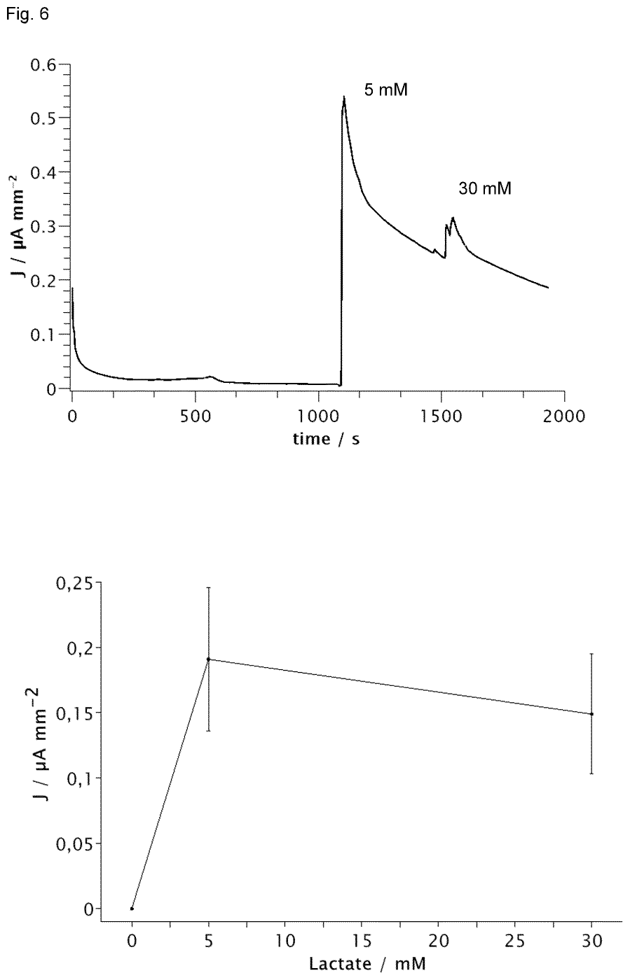

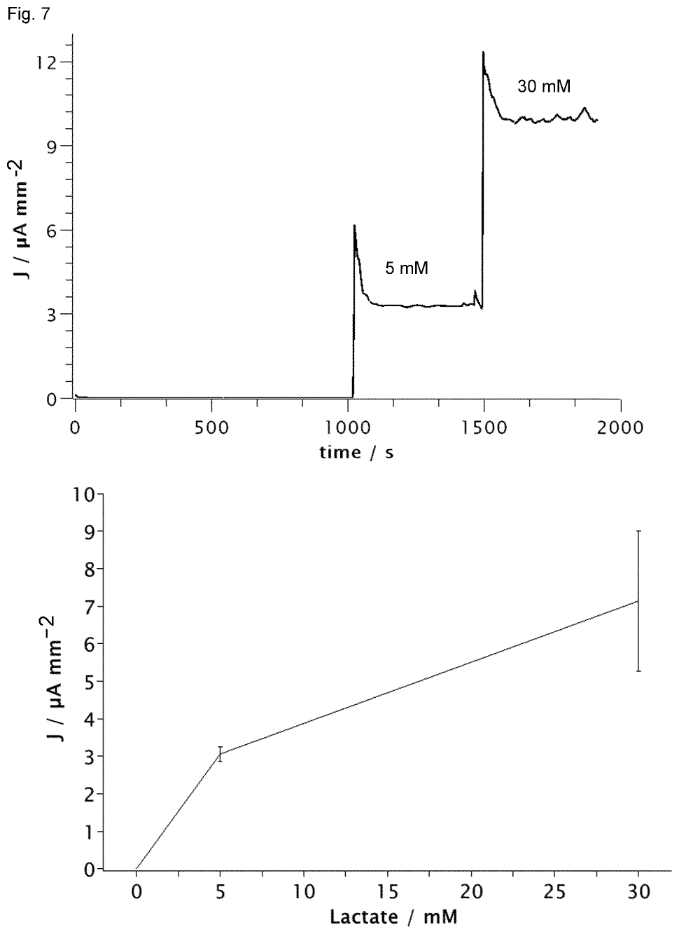

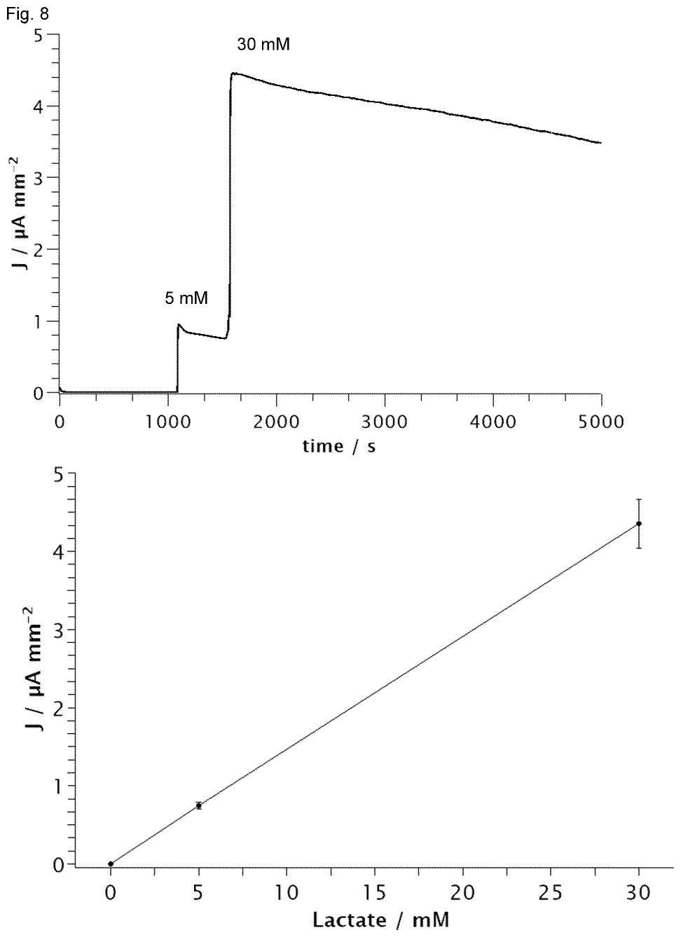

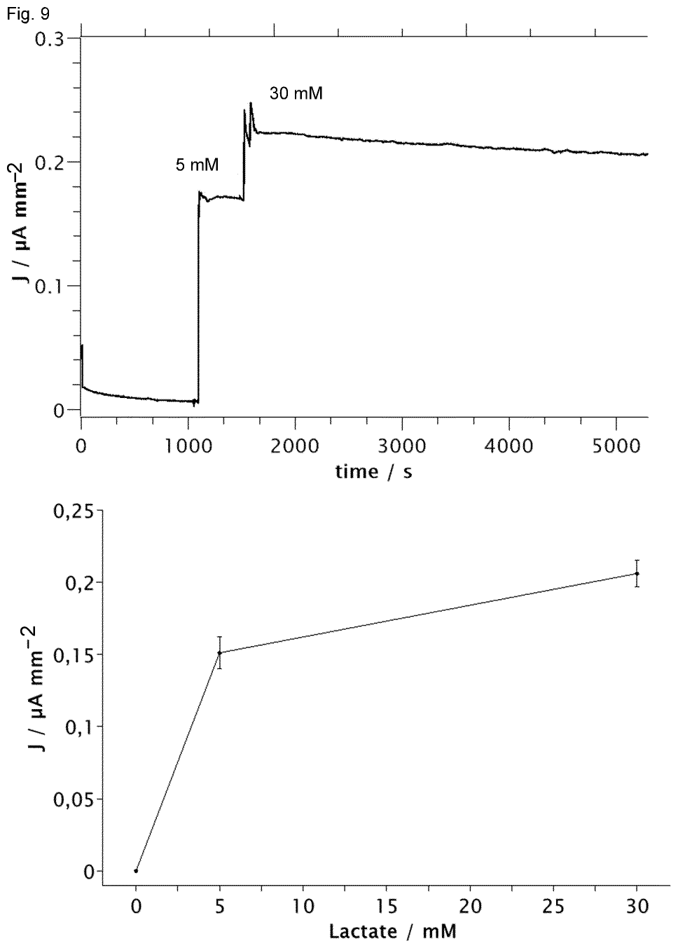

Electrochemical measurements were performed on three-electrode screen printed electrodes (DRP-C110, Metrohm). The sensors were modified with 1 μL enzyme (EaLDH) solution by drop coating and drying facilitating adsorption (5 mg/mL enzyme in 10 mM, pH 7.0 PB containing 0.2% Triton). The sensors were then immersed in 50 mM phosphate buffer containing 50 μM 1,4-benzoquinone as mediator and increasing lactate concentrations were added at 37° C. (FIG. 5). A clear correlation between increasing lactate concentrations and current response can be observed and was used to derive a calibration function for the biosensor. The same procedure was used to prepare and measure electrodes with LjLDH (FIG. 6), SsLDH (FIG. 7), EaLDH (FIG. 8), and AvLOX (FIG. 9).

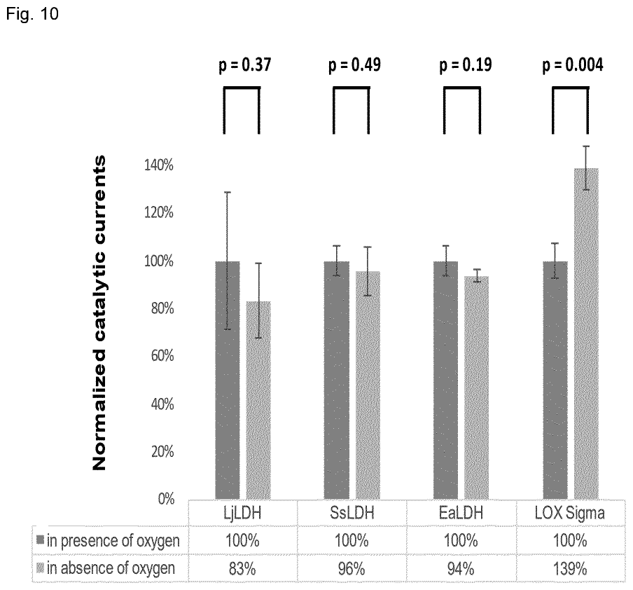

To test for oxygen interference, the buffer solution was purged with nitrogen for 15 minutes prior to measurement and a calibration with 5 mM lactate was done and current densities in absence and presence of oxygen were compared (FIG. 10). In a set of at least 3 independent sensor prototypes all LDH based sensors were not interfered by oxygen, while the prototype built with the LOx enzymes (AvLOx) did show highly significant (p=0.004) signal deviations in dependency of the oxygen availability.

### REFERENCES

Ashok, Y. et al. (2020) ‘FMN-dependent oligomerization of putative lactate oxidase from Pediococcus acidilactici’, PLoS ONE, 15(2), pp. 1-16. doi: 10.1371/journal.pone.0223870.
Baek, Minkyung, et al. 2021. “Accurate Prediction of Protein Structures and Interactions Using a Three-Track Neural Network.” Science 373(6557):871-76
Bao W. J., et al (1993), Purification and Characterization of Cellobiose Dehydrogenase, a Novel Extracellular Hemoflavoenzyme from the White-Rot Fungus Phanerochaete chrysosporium, Archives of Biochemistry and Biophysics, 300(2) 705-713. https://doi.org/10.1006/abbi.1993.1098.
Cardosi, M. and Liu, Z. (2012) ‘Amperometric Glucose Sensors for Whole Blood Measurement Based on Dehydrogenase Enzymes’, Dehydrogenases. doi: 10.5772/48491.
Gasteiger, E., et al. 2005. “Protein Identification and Analysis Tools on the ExPASy Server.” The Proteomics Protocols Handbook 571-608.
Goodwin, M. L. et al. (2007) ‘Blood lactate measurements and analysis during exercise: A guide for clinicians’, Journal of Diabetes Science and Technology, 1(4), pp. 558-569. doi: 10.1177/193229680700100414.
Harreither, W. et al. (2011) Catalytic Properties and Classification of Cellobiose Dehydrogenases from Ascomycetes, Applied and Environmental Microbiology, 77(5), 1804-1815. https://doi.org/10.1128/AEM.02052-10
Kadowaki, M. A. S. et al. (2020) ‘Enzymatic versatility and thermostability of a new aryl-alcohol oxidase from Thermothelomyces thermophilus M77’, Biochimica et Biophysica Acta, 1864(10), 0304-4165. https://doi.org/10.1016/j.bbagen.2020.129681.
Kim, T. J., et al. (2007). Lactate concentrations in incisions indicate ischemic-like conditions may contribute to postoperative pain. The journal of pain, 8(1), 59-66. https://doi.org/10.1016/j.jpain.2006.06.003
Kucherenko, I. S., et al. (2019) ‘Advances in the biosensors for lactate and pyruvate detection for medical applications: A review’, TrAC—Trends in Analytical Chemistry. Elsevier B. V., pp. 160-172. doi: 10.1016/j.trac.2018.11.004.
Brugger D, et al. (2014) Engineering Pyranose 2-Oxidase for Modified Oxygen Reactivity. PLOS ONE 9(10): e109242. https://doi.org/10.1371/journal.pone.0109242.
Krondorfer I, et al. (2014) Engineering of Pyranose Dehydrogenase for Increased Oxygen Reactivity. PLOS ONE 9(3): e91145. https://doi.org/10.1371/journal. pone.0091145.
Maeda-Yorita, K. et al. (1995) ‘L-lactate oxidase and L-lactate monooxygenase: Mechanistic variations on a common structural theme’, Biochimie, 77(7-8), pp. 631-642. doi: 10.1016/0300-9084(96)88178-8.
Mitsubayashi, K. et al. (1994) ‘Analysis of metabolites in sweat as a measure of physical condition. The increasing desire to naturally enhance the performance of sportsmen and athletes through’, Analytica Chiica Acta, 289, pp. 27-34.
Kavita Rathee, et al., Biosensors based on electrochemical lactate detection: A comprehensive review, Biochemistry and Biophysics Reports, Volume 5, 2016, pp. 35-54, ISSN 2405-5808, https://doi.org/10.1016/j.bbrep.2015.11.010.
Rocchitta, G., et al. (2016). Enzyme Biosensors for Biomedical Applications: Strategies for Safeguarding Analytical Performances in Biological Fluids. Sensors (Basel, Switzerland), 16(6), 780. https://doi.org/10.3390/si6060780
Sievers, F. et al. (2011) Fast, scalable generation of high-quality protein multiple sequence alignments using Clustal Omega. Mol. Syst. Biol. 7, 539
Sygmund, C. et al. (2011) ‘Reduction of Quinones and Phenoxy Radicals by Extracellular Glucose Dehydrogenase from Glomerella Cingulata Suggests a Role in Plant Pathogenicity’ Microbiology, 157(11):3203-12. https://doi.org/10.1099/mic. 0.051904-0.
Taurino, I. et al. (2013) ‘Comparative study of three lactate oxidases from Aerococcus viridans for biosensing applications’, Electrochimica Acta, 93, pp. 72-79. doi: 10.1016/j.electacta.2013.01.080.

1. A method for detecting and/or quantifying lactate in a sample comprising the steps of:
a) providing an electrode comprising a lactate dehydrogenase (LDH), said LDH comprising a flavin mononucleotide (FMN) and an amino acid sequence of SEQ ID NO: 2, or an amino acid sequence having at least 50% sequence identity to SEQ ID NO: 2, comprising the following sequence
T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S, and
X₄ is I or V;
b) contacting the sample with the electrode; and
c) detecting the oxidation of lactate by the LDH,
wherein the oxidation of lactate is performed in the presence of a redox mediator.

2. The method of claim 1, wherein the sample is selected from the group consisting of any one of food; beverage; fermented food; fermented beverage; chemicals; water; soil; and a sample provided by a human or animal, specifically from any one of body fluid, interstitial fluid, blood, plasma, dermal fluid, urine, tears, sweat, saliva, skin, flesh, tissue, eyeballs, cornea, and gastric fluid.

3. The method of claim 1, wherein the LDH is immobilized on the electrode.

4. The method of claim 3, wherein the LDH is immobilized by adsorption or complex formation.

5. The method of claim 4, wherein complex formation is via a complexing linker, covalent or ionic linkage.

6. The method of claim 3, wherein the LDH is cross-linked.

7. The method of claim 6, wherein the LDH is cross-linked by bifunctional agents.

8. The method of claim 1, wherein the redox mediator is selected from the group consisting of organic redox mediators, soluble redox mediators, insoluble redox mediators, redox polymers, transition metal complexes, polymeric transition metal complexes, wired redox mediators, sandwich compounds, and derivatives thereof.

9. The method of claim 1, wherein the amino acid sequence of the LDH comprises a sequence having at least 70%, 80%, 90%, or 95% sequence identity to amino acids G35 to K360 of SEQ ID NO: 2.

10. The method of claim 1, wherein the LDH is selected from SEQ ID NO: 2, 13, 59, 73, 74, 155, or a sequence with at least 70%, 80%, 90%, or 95% sequence identity to SEQ ID NO: 2, 13, 59, 73, 74, 155.

11. The method of claim 1, wherein the LDH is selected from SEQ ID NOs: 2 to 249, or a sequence with at least 90%, or 95% sequence identity to ID NOs: 2 to 249.

12. An electrode comprising an LDH, wherein said LDH comprises flavin mononucleotide (FMN) and an amino acid sequence of SEQ ID NO: 2, or an amino acid sequence having at least 50% sequence identity to SEQ ID NO: 2, comprising the following sequence
T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S,
X₄ is I or V.

13. The electrode of claim 12, wherein said electrode further comprises a redox mediator.

14. The electrode of claim 12, wherein the LDH is immobilized on the electrode.

15. The electrode of claim 14, wherein the LDH is immobilized by adsorption or complex formation.

16. The electrode of claim 15, wherein complex formation is via a complexing linker, covalent or ionic linkage.

17. The electrode of claim 14, wherein the LDH is cross-linked.

18. The electrode of claim 17, wherein the LDH is cross-linked by bifunctional agents.

19. The electrode of claim 12, wherein the redox mediator is selected from the group consisting of any one of organic redox mediators, soluble redox mediators, insoluble redox mediators, redox polymers, transition metal complexes, polymeric transition metal complexes, wired redox mediators, sandwich compounds, and derivatives thereof.

20. The electrode of claim 12, wherein the amino acid sequence of the LDH comprises a sequence having at least 70%, 80%, 90%, or 95% sequence identity to amino acids G35 to K360 of SEQ ID NO: 2.

21. The electrode of claim 12, wherein the LDH is selected from SEQ ID NO: 2, 13, 59, 73, 74, 155, or a sequence with at least 70%, 80%, 90%, or 95% sequence identity to SEQ ID NO: 2, 13, 59, 73, 74, 155.

22. The electrode of claim 12, wherein the LDH is selected from SEQ ID NOs: 2 to 249, or a sequence with at least 90%, or 95% sequence identity to SEQ ID NOs: 2 to 249.

23. A biosensor comprising an electrode of claim 12.

24. A device comprising an electrode of claim 12.

25. An enzyme composition comprising an LDH and a redox mediator, wherein said LDH comprises a flavin mononucleotide (FMN) and an amino acid sequence of SEQ ID NO: 2, or an amino acid sequence having at least 50% sequence identity to SEQ ID NO: 2, comprising the following sequence T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20, X₁ is N or F,
X₂ is L or F,
X₃ is G, T, or S, and
X₄ is I or V.

26. The enzyme composition of claim 25, wherein the redox mediator is selected from the group consisting of any one of organic redox mediators, soluble redox mediators, insoluble redox mediators, redox polymers, transition metal complexes, polymeric transition metal complexes, wired redox mediators, sandwich compounds, and derivatives of these redox mediators.

27. The enzyme composition of claim 25, wherein the amino acid sequence of the LDH comprises a sequence having at least 70%, 80%, 90%, or 95% sequence identity to amino acids G35 to K360 of SEQ ID NO: 2.

28. The enzyme composition of claim 25, wherein the LDH is selected from SEQ ID NO: 2, 13, 59, 73, 74, 155, or a sequence with at least 70%, 80%, 90%, or 95% sequence identity to SEQ ID NO: 2, 13, 59, 73, 74, 155.

29. The enzyme composition of claim 25, wherein the LDH is selected from SEQ ID NOs: 2 to 249, or a sequence with at least 90%, or 95% sequence identity to SEQ ID NOs: 2 to 2491.

30. A recombinant LDH comprising a flavin mononucleotide (FMN), wherein the amino acid sequence of said LDH comprises from N- to C-terminus
i. the dipeptide GP, and
ii. SEQ ID NO: 2, or an amino acid sequence having at least 50% sequence identity to amino acids 2 to 373 of SEQ ID NO: 2, comprising the sequence T-(Xₓ)ₙ₁-F-(Xₓ)ₙ₂-X₁-X₂-(Xₓ)ₙ₃-X₃-X₄ (SEQ ID NO: 1), wherein
T is T122 of SEQ ID NO: 2, or corresponding to T122,
F is F152 of SEQ ID NO: 2, or corresponding to F152,
Xₓ is any amino acid,
n1 is an integer of 25 to 35,
n2 is an integer of 40 to 45,
n3 is an integer of 5 to 20,
X₁ is N or F,
X₂ is L or F,
X₃ is G, T, or S,
X₄ is I or V.

31. The recombinant LDH of claim 30, wherein the amino acid sequence of the LDH comprises a sequence having at least 70%, 80%, 90%, or 95% sequence identity to amino acids G35 to K360 of SEQ ID NO: 2.

32. The recombinant LDH of claim 30, wherein the LDH is selected from SEQ ID NO: 2, 13, 59, 73, 74, 155, or a sequence with at least 70%, 80%, 90%, or 95% sequence identity to SEQ ID NO: 2, 13, 59, 73, 74, 155.

33. The recombinant LDH of claim 30, wherein the LDH is selected from SEQ ID NOs: 2 to 249, or a sequence with at least 90%, or 95% sequence identity to SEQ ID NOs: 2 to 249.

34. A kit for detecting and/or quantifying lactic acid or a salt thereof comprising an electrode of claim 12, further comprising an instruction manual.


SEQUENCE LISTING (250 sequences)

SEQ ID NO: 1 - skipped

SEQ ID NO: 2 - protein, 369 aa, Pediococcus acidilactici
MTMINGYEQSDREEKIDILNLESLEKQAEEIIPAGGFGYIAGGSEDEWTLKQNRMAFHHRQIAPKALSGIEKPELNTEIFGIPLNTPVMMAPAAAQGLAHSQGEKDTARGLAAVGGLMAQSTYSSVSIAETAAAGGDAPQFFQLYMSKDWNFNESLLDEAKKANVKAIILTVDATVDGYREADIKNKFTFPLPMANLIKFSEGNGQGKGIEEIYASAAQNIRPEDVKRIADYTNLPVIVKGIQTPEDAIRAIDAGAAGIYVSNHGGRQLNGGPASFDVLEDIATAVNKQVPIIFDSGVRRGSDVFKALASGADLVALGRPVIYGLALGGAKGVQSVFEHLNHELEIVMQLAGTKTIEDVKNNSLLNIKY

SEQ ID NO: 3 - protein, 408 aa, Lactobacillus delbrueckii
MTHYIKGFPQSEADEDLKFVNVDELEEKAKEVMPEGAYYYVASGSEYEWTWRNNTTAFNHYQIVPRALTGMDNPSTETEFLGMKLKTPIMISPIACHGISHADAEVATQKGAALAGAMFTSSTYGNKPVEEIAAAAPDAPRMFQLYLSKNWDFNKMVFDAINAAGYKAILLTVDALVSGYREANLRTNFAFPVPLDFFTRFQGAKGEGQTVAQMYASSAQNIGPDDIKRIKEMSGLPVIVKGVNCAEDVEVALTAGADGVYVTNHGGREIDGAPATIDVLPEVVEAVNGRCPVIFDGGVRRGSHVFKALALGADLVGIGRPYLYGLALGGPHGVASVINELNDELKIDMQLTGCKTIEDVKHARLTDFQYAGDTKPSSTDPRVRKPYPVTAVNQIKSEPDAATGASHH

SEQ ID NO: 4 - protein, 365 aa, Lactococcus garvieae
MAYVTSNDEQKVEIVNIASLERRVKDRMEAQGNKGAFGYIRGGAEDEWTMRENTASFNTKTISPRVLRGIDSADLRTSIFGIDLKTPIIQAPVAAQGLAHMEGEVDTAKAMEEMGSLFSISTYGSTSVEDAAAAVNGAPQFFQLYMSKDDQFNQFLLEKAVKSGVKAIILTADSTLGGYREEDVINHFQFPLPMPNLAAFSESDGVGKGIFEIYAEAKQGLVLSDIQKIKNWTNLPVIVKGIQDPVDAMEAIAAGADGIWVSNHGGRQLDGGPASFTVLPRIAQVVNKRVPIIFDSGVRRGEHVFKALASGADLVAIGRPVLYGLNLGGKEGVKSVFEHLNKELSITMQLAGAKDIEAIKNTDLL

SEQ ID NO: 5 - protein, 366 aa, Paucilactobacillus hokkaidonensis
MSYQASNEEKDLKIVNLASLEARVKPRMEAGAFGYIRGGSEDEWTMQQNTVAFQHKKIMPHVLKGIDSADLHTNIFGIDLKTPIIEAPSAAQGLAHTKGEIDTAKGVADAGSIFSISTYANTKIEDAAAAVPDAPQFFQLYMSKDDGFNKFILDKAVKAGAKAIILTADSTLGGYREADVINGFQFPLPMPNLAAYSDQTDSGDGQGKGISEIYAAAKQGLVLADIRKVKEWTHLPVIVKGIQAPEDAEAVILSGADAIWVSNHGGRQLDGGPASFDVLPAIAHTVAKRVPIIFDSGVRRGEHVFKALASGADLVAIGRPVIYGLNLGGAKGVKSVFDHLNMELSITMQLAGTKDVTAIKNTTLID

SEQ ID NO: 6 - protein, 362 aa, Latilactobacillus curvatus
MGYHTSEAENPIDILNLASLEGRVKERMEAGAFGYIRGGAEDEWTMAANTSAFNTKKIMPRVLKGIDHADLHTKLWDIDLKTPIIQAPSAAQGLAHEKGEVDTAKGIAAAGSIFSISTYANTLVEDAAAAAPDAPQFFQLYMSKDDQFNEFLLKKAVKAGVKAIILTVDSTLGGYREADIETQFQFPLPMPNLAAYSNSDGAGKSISEIYAAAKQGLVPEDIQKIKQITNLPVFVKGIQSPVDAEIAIQAGADGIWVSNHGGRQLDGGPASFEVLPLIAQQVAKRVPIVFDSGIRRGEHVFKALASGADLVAIGRPIIYGLNLGGAQGVQSVFEHLNHELSITMQLAGTKTIDEVKETTLLD

SEQ ID NO: 7 - protein, 415 aa, Myroides profundi
MKKTIFTLTAITLMTSAFADVVTNDSTTKTVFQDKNKKGKGYQASTADKKLKIVNLHELEGQVKAEMDQGAFGYIVGGAEDQNNLKINTENFDKKYIMPRVLKGIKHEDIDLSTSLFGIPLKTPIIQAPMAAQGLSHVDGEIATAKGMIAAGSLFSLSTYGNKTIEEVAEGINGAPFFFQLYMSKNDDFNKFTLDRAKKYGAKAIILTVDSPVGGYREEDIKTGFTFPLGMGNLELFAAQQADGNKTGKGAGITEIYAQAKQDFKPSDIKYVKDLTGLPVIIKGIQSPEDAEIAIQAGADAIWVSNHGGRQLDAGPSSFDVLPLVAKTVNKRVPIIFDSGVRRGSHIFKAIASGADIVAIGRPILYALHLGGSQGVTSVIDQLNKELTINMFLGGAKNIKEIQSTKLYTDKDFQL

SEQ ID NO: 8 - protein, 366 aa, Lactococcus lactis
MTYKTSTENKALEIVNVKSLEGKVKQSMEAAGNKGAFGYIRGGSEDEWTLNENTSAFNKKQIMPRVLRGIDSADLSTSLFGIKLKTPIIQAPVAAQGLAHEEGEVATAKAMAEVGSIFSISTYGSTSVEDVAKAAPGAPQFFQLYMSKDDKFNEFLLKKAVSAGVKAIILTADSTLGGYREEDIVNHFQFPLPMPNLAAFSESDGTGKGISEIYAAAKQGLVLEDIQKIKKITNLPVIVKGVQSPIDADDAINAGADGIWVSNHGGRQLDGGPASIDVLPLIAKSVNHRVPIVFDSGVRRGEHVFKALAQGADVVAVGRPVLYGLNLGGAKVVQSVFEHLSKELSITMQLAGTKNIEEIKHTSLID

SEQ ID NO: 9 - protein, 367 aa, Levilactobacillus brevis
MTMINGYEQSDREESLDILNLPSLEARAKQIIPAGGFGYISGGSEDEWTLKENTEAFNHVQIIPRALTNIEHPSTQTQIFGVDLQTPIMMAPAAAQGLAHSRGEMATAQGIAAAGALMAQSTYSSTSIADTAAAGQGAPQFFQLYMSKDWHFNESLLDEAIKANVKAIILTVDATVDGYRESDIVNKFQFPIPMANLTKFSEGDGKGKGIQEIYAAAAQKINADDVRRIAEYTHLPVIVKGIQSVEDAMTAIGAGAAGIYVSNHGGRQLNGGPASFDVLHDIALAVNHRVPIIFDSGVRRGSHVFKALANGADLVALARPVIYGLALGGAQGVTSVINHLNDELLIDMQLAGTKTIEDVKQAKLLRH

SEQ ID NO: 10 - protein, 368 aa, Weissella cibaria
MTYQGSTKEEKLNIIDLPRLRDAVKRDTEAGAFGYVDGGSSDEQVLRDNEQAFRHYQLIPRMLQDIAAPDLSTTLFDIPLTMPVIAAPIAAHGLMHQDGEQVTVKGVGAAGSIFSLSTYGNSRIADVASAAPDTPKFFQLYMSRDDDFNQYLLDEAVNNGYKAIILTADATLGGYREADIINNFTFPLPMENLAAFSNAAGSGEGLGIADIYARAKQDLSLRDIKKVKEMAHGLPVIVKGIQDPDDALAAIAAGADGIWVSNHGGRELNGAPASIDTLAAIAKAVNRRVPVIFDSGIRRGEDVAKALALGADVVALGRPMLWGLNQGGAAGVQSVYEHLATELKIVMQLTGAHTVAELQRAKIINAKF

SEQ ID NO: 11 - protein, 409 aa, Lactobacillus kullabergensis
MTSYYNGFPQSDRDETLKMINLDELEERAKKVMPEGAYYYIASGSENEWTWRNNTTAFNHFQIVPRALTNMDNPKLDTEFMGMKLKTPVMISPIACHGIAHKDAEIATQKGAAAAGALFASSTYANKSVEEIAAAAPNAPRFFQLYLSKDWGFNKMVFDAVKKAGYKGIFLTVDALVSGFREANLRTKFAYPVPLDFFTRYQGAKGEGQTVAQMYASSAQKIGPEDVRRIKEMSGLPVFVKGVVCAEDAYLAMGAGADGIYVTNHGGREVDCGPATIDMLPEIAKAVNHRVPIVFDSGVRRGSHVFKALALGADMVGVGRPYLYGLALGGAKGVQSVIEQLNQELLIDMQLTGCKTIEDVKHAKITHINYTADNLKSNTDPTRVKPYPVTAENQLKEDDSDAVTGASQA

SEQ ID NO: 12 - protein, 409 aa, Lactobacillus melliventris
MTYYYNGFPQSERNEALNMVNLDELEERAKEVMPEGAYYYIASGSENEWTWRNNTTAFNHFQIVPRALTNMDHPQLDTEFMGMKLKTPVMISPIACHGIAHKDAEIATQKGAAAAGALFSSSTYANKSVEDIAAAAPDAPRFFQLYLSKDWDFNQMVFDAVKKAGYKGIFLTVDALISGFREANLRTNFAYPVPLDFFTRYQGAKGEGQTVAQMYASSAQKIGPQDVRRIKEMSGLPVFIKGVVCAEDAYIAMGAGADGIYVTNHGGREVDCGPATIDMLPEIAKAVNHRVPIVFDSGVRRGSHVFKALALGADMVGIGRPYLYGLALGGAKGVQSVIEQLNQELLIDMQLTGCKTIDDVKHAKITHINYTADNLKSNTDPSRVKPYPVTAENQLKNNGSDAVTGASQA

SEQ ID NO: 13 - protein, 409 aa, Lactobacillus helsingborgensis
MTSYYNGFPQSDRDEAIDMINLDELEERAKKVMPEGAYYYIASGSENEWTWRNNISAFNHFQIVPRALTNMDHPQLDTEFMGMKLKTPVMISPIACHGIAHKDAEIATQKGAAAAGALFSSSTYANKSVEDIAAAAPDAPRFFQLYLSKDWGFNKMVFDAVKKAGYKGIFLTVDALVSGFREANLRTKFAYPVPLDFFTRYQGAKGEGQTVAQMYASSAQKIGPEDIKRIKEMSGLPVFVKGVVCAEDAYLAMGAGADGIYVTNHGGREVDCGPATIDMLPEIAKAVNHRVPIVFDSGVRRGSHVFKALALGADMVGIGRPYLYGLALGGAKGVQSVIEQLNQELLIDMQLTGCKTIEDVKHAKITHINYTADNLKSNTDPSRVKPYPVTAENQLKENDSDAVTGASQA

SEQ ID NO: 14 - protein, 366 aa, Lactiplantibacillus plantarum
MTVINGYEQSDREQKLTILNLPSLEAAAKKIIPSGGFGYISGGSEDEWTLKQNTMAFNHVQIVPRALTDMEQPSTQTQAFGIDLKTPIMMAPAAAQGLAHARGEAATAEGMAQVGALMAQSTYSSTSIADTASAGKGAPQFFQLYMSKDWDFNQSLLDEAVKAGAKAIILTVDATVDGYREADIINNFQFPIPMANLTKFSEGDGKGKGIMEIYAAAAQKISPADVRRITEYTNLPVIVKGVQSPEDALLAIGAGAQGIYVSNHGGRQLNGGPASFDVLHEIAQAVNGRVPIIFDSGVRRGSHVFKALANGADLVALARPIIYGLALGGAQGVASVVSHLNDELLIDMQLAGTKTIEDVKRAKLLR

SEQ ID NO: 15 - protein, 398 aa, Proteus penneri
MKSKILKTTAIAMALSVGVAQAAEYKASIAEGPIKIVNLKAMEAQVQANMEKGAFGYIRGGAEDENNLRSNTTAFDKKYIMPRSLQGIEFSDLNLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIIMTIDSSVGGYREDDVKNNFQFPLGFANLEAFAKISDDKSKTGKGAGISEIYAQAKQAFTPEDIQYVKKMSGLPVIVKGIESPEDADTAIKAGADAIWVSNHGGRQLDSAPATIDVLPAIAKVVNKRVPIVFDSGVRRGSHVFKALASGADVVAVGRPILYGLNLGGAEGVNSVIQHLNKELKINMMLGGAKTVKDIQATHLYTDASFNQ

SEQ ID NO: 16 - protein, 371 aa, Lacticaseibacillus casei
MTVVNGYEQSDAEKKLNILNLPSLADEAKKIIPTGGFGYIVGGSEDEWTLAENTKAFNHAQIVPKALSNIDSPDLSTSFLGIDLKTPVMMAPTAAQGLAHSQGEKDTARGVAAVGGLMAQSTYSSTSIADTAAAGNGAPQLFQLYMSKDWDFNKSLLDEAKKAGVKGIILTVDATVDGYREEDIINNFQFPIPMPNLEKYSEGDGKGKGIGEIYASAAQKIGEDDVRRIAEYTDLPVIVKGIQSPEDALRAIGAGAAAVYVSNHGGRQLNGGPASFDVLPAIAKAVNKRVPIIFDSGIRRGSHAFKALAAGADLVAFGRPVIYGLALGGAQGVQSVFEQIDHELEIIMQLAGTKTIEDVKHAPLTHFNYAD

SEQ ID NO: 17 - protein, 364 aa, Companilactobacillus farciminis
MAKYEASTEEKHIDIVNIASLEQRVKDHMSNEKGAFGYIRGGSEDEWTMKQNTEAFNKKRIMPRVLQGIDHADLSTKFWDIDLKTPIIEAPSAAHGLAHAKGEVDTAKGVAAAGSIFSMSTYGSTSIEDGAAAAPDAPQFFQLYMSKDDKFNEFLIKKAVKAGVKAIILTVDSTLGGYREEDVINKFQFPLPMPNLAAFSDDDGEGKGISEIYAAAKQGIVPSDIQKIKDMSGLPVFVKGIQSPDDAELAIEFGADGIWVSNHGGRQLDGGPASFDVLPAIAQVVDKRVPVVFDSGVRRGEHVFKALASGADLVAVGRPIIYGLNLGGAQGVTDVIEHLNKELSITMQLAGTKTIHDVKNVDLL

SEQ ID NO: 18 - protein, 367 aa, Companilactobacillus ginsenosidimutans
MTEINGYTQSDNEKELHILNLPSLEAEAKKIIPTGGFGYISGGSEDEWTLNENTKAFNNVQIVPRVLSDIDDPQTDTSIFGIDLTMPIITSPSAAQGLAHSQAEKDTARGIAKAGTIMSQSTYASTSIADTAEAGNGAPQFFQLYMSKDWTFNESLLNEAKRVGAKAILLTADATVDGYRESDIINNFQFPLPMANLEKFSSGAGKGKGIGEIYAAAAQKISPDDVQRIKDITGLPVVVKGVQSPEDALIAIGAGADGVYVSNHGGRQLNGGPASFDVLADVAKAVNHKVPVIFDSGIRRGSHVFKALASGADIVALARPIIYGLALGGADGVYSVIEHLNNEFKTVMQLAGTKTIDDVKHAKLIKK

SEQ ID NO: 19 - protein, 398 aa, Salmonella bongori
MKSSHLIRVVASLAMLATSGLAYAEEYKASTDEKTIKMTNVASLEARVQARMDKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIADVAKVSGKSPFFFQLYMSKNNKFNEFILSQAVKHGAKAIILTVDSPVGGYREEDIKNDFQFPLGFANLEMFARKNDDGSKTGKGAGISEIYAQAKQAFTPEDIAYVHRISGLPVIIKGIQSPEDAEIAIQAGAAGIWVSNHGGRQLDSGPSSFDMLPAIAKVVNKRVPVIFDSGVRRGSHVFKALASGADIVAIGRPVLYGLNLGGAQGVASVIEQLNKELTINMMLGGARNIEQVKNTRLLSEKDL

SEQ ID NO: 20 - protein, 367 aa, Companilactobacillus heilongjiangensis
MSNKKYEASTAENHIDIINLASLEARVKDHMSNEKGAFGYISEGAEDEWTKKQNTEAFNKKQIMPRVIQGIDHADLSTQIFGIDLKSPIIQAPSAAHGLAHAKGETDTAKGVAAAGSIYSISTYASTSVEDAAAAAPDAPMFFQLYMSKDDKFNEFLIKKAVKAGAKAIIMTVDSTLGGYREADAINKFQFPLPMPNLAGYSAGDGEGKGIGEIYASAKQGIVPTDIQKIKDMSGLPVFVKGIQSPDDAALAIEFGADGIWISNHGGRQLDGGPASFDMLPYIAEVVNKRVPIVFDSGVRRGEHVFKALASGADLVALGRPIIYGLNLGGAQGVTDVIEHLNMELSITMQLAGTKTINDVKNTKLIG

SEQ ID NO: 21 - protein, 366 aa, Lactococcus cremoris
MTYQTSTENKAIEIVNIKSLEGKVKESMESAGNKGAFGYIRGGAEDEWTMGENTSAFNKKQIMPRVLRGIDSADLSTSLFGIDLSTPIIQAPVAAQGLAHEEGEVATAKAMAEVGSIFSISTYGSTSVEDAAKAAPGAPQFFQLYMSKDDKFNEFFLKKAVAAGVKAIILTADSTLGGYREEDIVNHFQFPLPMPNLAAFSESDGTGKGISEIYAAAKQGLVLEDIKKIKEITKLPVIVKGIQSPIDADDAIKAGADGIWVSNHGGRQLDGGPASIDVLPLVAKIVNHRVPLIFDSGVRRGEHVFKALAQGADIIAIGRPVLYGLNLGGARGVQSVFEHLNKELSITMQLAGTKNIDEVKRTALID

SEQ ID NO: 22 - protein, 364 aa, Dellaglioa algida
MGVTKLAYQASQEEHSIEITNVQALEGLVKARMEKGAFGYIAGGAEDEWTLRENGVAFDHKHIAPRVLKNIEKPDLSTTFMGIDLKIPMIAAPIAAQGLAHEQGEIDTAKGVAAVGSIMSLSTYSNKTISETCDGGTGGPQFFQLYMSKDEKFNRFLLDQAKKVGMKAIILTADSTLGGNREADSINHFTFPIPMANLAEYGEGEGQGIAAIYANAKQTLSLEDIKTISDYTDLPVFVKGIQSPLDIDDIIEAGAKGVWVSNHGGRQLDGGPASFDVLESIAKKVNKRVPIVFDSGIRRGSHIFKALASGADVVAIGRPMVYGLHLGGAQGVQSVFEHLSKELTIDMQLAGTKTVDDIKKTELI

SEQ ID NO: 23 - protein, 409 aa, Lactobacillus amylovorus
MTYYYKGFPQSDRDEKISMINVDELEERAKKVMPEGAYYYIASGAENEWTWRANTSAFNHYQIVPRALTDMDDPQTDTEFMGMKLKTPIMISPIACHGIAHKDAEVATQKGAAAAGALFSSSTYANKSVEDIATAAPEAPRFFQLYLSKDWDFNKMIFDAIKKAGYKGIFLTVDALVSGYREANLRTHFTYPVPLDFFTRYLGGKGEGQSVAQMYASSAQKIGPEDVARIKKESGLPVFVKGVMCAEDAYKAIGAGADGIYVTNHGGREVDGAPATIDVLPEIAKAVNHRVPIVFDSGVRRGSHVFKALALGADIVGIGRPYLYGLALGGPKGVESVINQLNTELKIDMQLTGCKTIDDVKRAKIDRIHYGLDTMPSNTDPSRIKPYPVTADNQIKSEEPDATSGASKH

SEQ ID NO: 24 - protein, 369 aa, Schleiferilactobacillus kimchicus
MAIINGYEQSENEKELDILNLPSLEAQAKEIIPKGGFGYIVGGSEDEWTLHANRSAFTHKQIVPKALSNIENPLTDTSVFGLNLKTPIMMAPAAAQGLAHVKGEVDTAKGVASVGGLMAQSTYSSTSIADTAAAGDGAPQFFQLYMSKDWTFNESLLDEAKRAGVKAIILTVDATVDGYREEDIVNHFQFPIPMANLTKFSEGDGAGKGIGEIYAAAAQKIGPDDVRRIIEYTDLPVIVKGIESAEDALYAVGAGAAGVYVSNHGGRQLNGGPASFDVLEDVAKAVHGRVPVIFDSGVRRGSDVFKALASGADLVAIGRPAIYGLALGGAQGVASVFNHLNDELKIIMQLAGTQTIEDVKHAPLLNIRY

SEQ ID NO: 25 - protein, 370 aa, Companilactobacillus kimchii
MGFLMKKKYEASTAENHVDILNIAGLEARVKDHMSNEKGAFGYISGGSEDEWTKKQNTESFNKRKIEPRVLQGIDHADLSTKLWDIDLKTPIIQAPSAAQGLAHAKGETDTAKGVAAAGSIFSISTYASTSIEDAAAAAPDAPQFFQLYMSKDDNFNEFLIKKAVAAGAKAIVLTVDSTLGGYREADVANKFQFPLPMPNLAGYSAGDGEGKGISEIYASAKQGIVPTDIQKIKNMSGLPVIVKGIQSPDDAELAIEFGADGIWVSNHGGRQLDGGPASFEVLPDIADAVDQRVPVIFDSGVRRGEHVFKALASGADLVAIGRPIIYGLNLGGAQGVTDVIEHLNMELSITMQLAGTKTINDVKNTELLW

SEQ ID NO: 26 - protein, 368 aa, Companilactobacillus tucceti
MMNKEKYETSTAENHVDIINLASLEDRVKDHMSNEKGAFGYIRGGAEDEWTLNENTEAFNDKEIMPRVLQGIDHADLSTKLWDIDLKTPIIQAPSAAHGLAHVKGETDTAKGVAAAGSIYSISTYASTSVEDASAAAPDSPMFFQLYMSKDDKFNEFLIKKAVKAGAKAIIMTVDSTLGGYRESDVINKFQFPLPMPNLAGYSAGDGEGKGIGEIYASAKQGIVPTDIQKIKDMSGLPVFVKGIQSPDDAELAIQFGADGIWVSNHGGRQLDGGPASFALLPYIAEVVDKRVPIVFDSGVRRGEHVFKALASGADLVALGRPIIYGLNLGGAQGVTDVIEHLNMELAITMQLAGTKTIDDVKNAELLD

SEQ ID NO: 27 - protein, 371 aa, Lacticaseibacillus nasuensis
MTVINGYEQSDREQKLDILNLPSLEEQAKAIIPTGGFGYIVGGAEDNWTLRQNTKAFAHAQIVPKALSNIENPDLSTSIFGIPLKTPVMMAPAAAQGLAHSQGEKDTAKGVAAVGGLMSQSTYSSTSIADTQAAAPDAPQFFQLYMSKDWTFNESLLDEAKKAGAKAIILTVDATVDGYREADIINNFQFPIPMANLTKFSEGDGKGKGIGEIYASAAQKISEADVRRIAEYTDLPVIVKGIESPEDALRAIGAGAAGIYVSNHGGRELNGGPAAFDVLPAIAKAVNHQVPIIFDSGVRRGSHVFKALAAGADLVAFARPVIYGLALGGALGVQSVFEEIDHELAITMQLAGTQTIADVKHAPLTHFNYED

SEQ ID NO: 28 - protein, 367 aa, Companilactobacillus nodensis
MTETNGYFQNDNEKELNIVNLPSLEAEAEKIIPKGGFGYIAGGSEDNWTLKANTEAFNHVQIVPHVLSDIEDPQTNTSIFGIDVKTPIMMTATAAQGLAHAKGEMDTAKGIAKAGALMEQSTYSSTSIADTMAAGNGAPQFFQLYMSKDWTFNESLLKEAKQAGAKAIVLTADATVDGYRESDIINDFQFPTPMANLTKFSEGDGEGKGIGEIYAAAAQKISPKDIQRIKDIAGLPVIVKGVQSPEDALLAIGAGADVIQVSNHGGRQLNGGPASFDVLSDVAKAVNHRVPIIFDSGVRRGSHVFKALASGADMVAMGRPVIYGLALGGADGVYSVVEHLNDEFKTIMQLAGTKTIEDVKHAKLLKK

SEQ ID NO: 29 - protein, 369 aa, Lentilactobacillus sunkii
MTVVNGYKQNDTEQKLDILNLPELEEKAKQIIPTGGFGYIAGGSENNWTLKANRTAFTHKQIVPRALSNIEKPELDTNVFGIPLKTPIMMAPTAAQGLAHSQGEKDTAKGVAAVGGLMAQSTYSSTSIADTAAAGNGAPQFFQLYMSKDWDFNYSLLDEAKKAGVKGIILTVDATVDGYREDDIKNNFQFPIPMANLTKFSEGDGKGKGIGEIYASAAQKIGPDDVKKIADYTDLPVIVKGIESPEDALYAIGAGAAGVYVSNHGGRQLNGGPASFDVLEDVAKAVNGQVPIIFDSGVRRGSDVFKALASGADLVAMGRPVIYGLALGGAEGVQSVFEHLGDELKIIMQLAGTKTIADVKKANLLNIKY

SEQ ID NO: 30 - protein, 367 aa, Liquorilactobacillus hordei
MTIFNGYEQSDREQKLNILNLPSLEAEAKKIIPTGGFGYISGGSEDEWTLHENTSAFNHIQIIPRALTNVDQPSTNTKVFGLNLKTPIMMAPAAAQGLAHSRGEKATAEGLAEVGGLMAQSTYSSTSIADTAAAGNGAPQFFQLYMSKDWEFNYSLLDEAVKAGAKGIILTVDATVDGYRESDIINNFQFPIPMANLAKFSEGDGKGKGIMEIYAAAAQKISPDDVRRIVKYTKLPVIVKGIESAEDALLAIGAGAEGIYVSNHGGRQLNGGPASIDVLHDVAKAVNHQVPVIFDSGVRRGSHVFKALASGADLVALARPIIYGLALGGAKGVSSVINHLNDELKIDMQLAGTKTIEDVKKAKILRK

SEQ ID NO: 31 - protein, 369 aa, Schleiferilactobacillus perolens
MTVTNGYEQSDREQKLDFINVNDLEKEAEKIIPKGGFGYIRGGSEDEWTLRENTLAFNHAQIIPRALTDMENPSTDTEAFGLHFKTPLMMAPTAAQGLAHAKGETDTARGVAAAGALMAQSTYSSTSIADTAAAANGAPQFFQLYMSKDWDFNHHLLDEAKKAGIKGIILTLDAPVDGFREDDMRNHFQFPIPMANLTEYSEGDGAGKGIGEIYAAAAQKIGPKDIERIAEYTDLPVIAKGIMSPEDALKAIGAGAAGVYVSNHGGRQLNGGPASFDVLPSIAAAVNHQVPIIFDSGVRRGSHVFKALASGADLVAFGRPAIYGLALGGAEGVQGVFEHLNAELKIDMQLAGTKTIADVQHAKLAHFPA

SEQ ID NO: 32 - protein, 366 aa, Companilactobacillus paralimentarius
MKKKYEASTAENHVDILNIAGLEARVKDHMSNEKGAFGYISGGSEDEWTKKQNTESFNKRKISPRVLQGIDYADLSTKLWDIDLKTPIIQAPSAAQGLAHVKGETDTAKGVAAAGSIFSISTYASTSIEDAAAAAPDAPQFFQLYMSKDDKFNEFLIKKAVAAGAKAIVLTVDSTLGGYREADVANKFQFPLPMPNLAGYSAGDGEGKGISEIYASAKQGIVPTDIEKIKNMSGLPVIVKGIQSPDDAELAIDFGADGIWVSNHGGRQLDGGPASFEVLPDIADAVDQRVPVIFDSGVRRGEHVFKALASGADLVAIGRPIIYGLNLGGAQGVTDVIEHLNMELSITMQLAGTKTINDVKNTELLW

SEQ ID NO: 33 - protein, 367 aa, Liquorilactobacillus nagelii
MTTINGYEQSDREEKLDILNLPSLEAEAKKIIPKGGFGYISGGSEDEWTLHENTTAFNHVQIIPRALTDMEQPTTATSVFGINLKTPIMMAPAAAQGLAHSRGEEATAEGLAQAGGLMAQSTYSSTSIADTAAAGKGAPQFFQLYMSKDWDFNRSLLDEAVKAGAKGIILTVDATVDGYRESDIINNFQFPIPMANLAKFSEGDGKGKGIMEIYAAAAQKIAPADVRKIAEYTHLPVIVKGIESAEDALLAIGAGAQGIYVSNHGGRQLNGGPAAFDVLHEVAQAVDHRVPVIFDSGVRRGSHVFKALASGADLVALARPVIYGLALGGAQGVASVINHLNDELKIDMQLAGTKTIEDVKQAKLIRH

SEQ ID NO: 34 - protein, 367 aa, Companilactobacillus mindensis
MNKKKYEASTAENHVEIVNIASLEKRVQDHMSNEKGAFGYIRGGAEDEYTMKQNTEAFNKAKIMPRVIQGIDHADLSTKLWDIDLKTPIIQAPSAAQGLAHAKGETDTAKGVAAAGSIFSISTYASTSVEDAAAAAPDAPQFFQLYMSKDDKFNEFLIKKAVAAGVKAIVLTVDSTLGGYREADVINKFQFPLPMPNLAGYSAGDGEGKGISEIYASAKQGIVPADIQKIKDMSGLPVFVKGIQSPDDAELAIEFGADGIWVSNHGGRQLDGGPASFDVLPDIADRVDKRVPIVFDSGVRRGEHVFKALASGADLVAIGRPIIYGLNLGGAQGVTDVIDHLNMELSITMQLAGTKTINEVKNTDLYY

SEQ ID NO: 35 - protein, 371 aa, Lacticaseibacillus manihotivorans
MTVINGYEQSDRNEKLDILNLPSLEARAKKIIPQGGFGYIVGGAEDDWTLQQNTKAFTHAQIVPKALSNIENPDLSTNVFGIDLKTPVMMAPAAAQGLSHSLGEKATAQGVAAVGALMSQSTYSSTSIADTQAAAPDAPQFFQLYMSKDWDFNNALLDEAMKAGVKGIILTVDATVDGYREQDIINNFQFPIPMANLERFAAGDGKGKGIGEIYAAAAQKISEDDVRRIAEYTKLPVIVKGIESPEDAMRAIGAGAAGIYVSNHGGRELNGGPAAFDTLSAIAKAVNHQVPIIFDSGVRRGSHVFKALASGADLVAFARPVLYGLALGGALGVQSVFEQIDHELEIVMQLAGTKDIEAVKHAPLTHFHYEN

SEQ ID NO: 36 - protein, 406 aa, Lactobacillus psittaci
MTVFYKGFPQSDRNQAIKMVNVDELEERVKKVMPEAAYYYIASGSENEWTWRNNTAAFNHFQIIPRSLTDMDNPSTDTKFMGMDLKTPVMICPIACHGIAHKDAEVATAEGAKAAGALFSSSTYANRSVEDIAGATGDSPKFFQLYLSKDWDFNKMVLDAVKAAGYKGIMLTVDALVSGYREANLRTNFTYPVPLDFFTRYGKGEGMSVAQMYANSAQKIGPKDVEKIKELSDLPVFVKGIMNAEDAYLAMGAGADGIVVSNHGGREIDTAPATIDMLPEIAAAVNGRVPIILDSGVRRGSHVFKALALGADLVGIGRPFLYGLALGGAKGVESVINQINNEFKILMQLTGCKTVEDVKHADIRQINYTADNLPSNTDPAVRKPYPVTKDNQLEGTQDAVSGASKH

SEQ ID NO: 37 - protein, 369 aa, Lentilactobacillus diolivorans
MAVVNGYKQSENEKKLNILNLPELEQQAKEIIPTGGFGYISGGSEDEWTLRANRKAFTHKQIVPRALSNIEKPVLDTTVFGFPLKTPVMMAPTAAQGLAHAKGEVDTAKGVAAVGGLMAQSTYSSTSISDTAAAGDGAPQFFQLYMSKDWTFNESLLDEAKKAGVKGIILTVDATVDGYREADIINNFQFPIPMPNLTQYSEGNGQGKGIAEIYASAAQKIGPKDVERIASYTDLPVIVKGIESPEDALYAIGAGAAGVYVSNHGGRQLNGGPASFDVLADVAKAVNGKVPVIFDSGVRRGSDVFKAIASGADLVAIGRPAIYGLALGGAQGVQSVFKHLDQELEIIMQLAGTKTIDDIKKTNLLGINY

SEQ ID NO: 38 - protein, 369 aa, Schleiferilactobacillus paracollinoides
MVLTNGYEQNENENPLDILNLTELEGKAKAIIPTGGFGYISGGSEDEWTLRANRTAFQHRQIVPKALSNIENPSTDTTVFGIDLKTPIMMAPTAAQGLAHAKGEVDTAAGVAAAGGLMAQSTYSSTSIADTAAGGHGAPQFFQLYMSKDWSFNESLLDEAKKAGVKGIILTVDATVDGYREADIINNFQFPIPMANLTKFSEGDGKGKGIADIYAAAAQKIGPDDVRRIAEYTDLPVIVKGVESPEDALYAIGAGAAGIYVSNHGGRQLNGGPASFDVLESVAKAVNHRVPVIFDSGVRRGSDVFKALASGADLVAMGRPAIYGLALGGAQGVESVFTHLNDELKIIMQLAGTKTIADVKHAPLLDIKY

SEQ ID NO: 39 - protein, 371 aa, Lacticaseibacillus pantheris
MTLTNGYEQSDREEKLNILNLPSLEGEAKKIIPTGGFGYIFGGAEDEWTLKENTQAFNHAQIVPRALSNIDHPDLSTDVFGLHLKTPVMMAPTAAQGLAHAKGETDTAKGVAAVGGLMAQSTYSSTSIADTAAAGNGAPQFFQLYMSKDWTFNESLLDEAKKAGVKAIILTVDATVDGYREEDIINNFQFPIPMANLEKFASGDGKGQGIGEIYAAAAQKIGPDDVRRIAEYTDLPVIVKGIQSPEDALRAIGAGAAGIYVSNHGGRQLNGGPASFDVLADIAKAVNHQVPIIFDSGVRRGSHVFKALASGADLVAFGRPVIYGLALGGAEGVQSVFEAIDHELEITMQLAGTKTIEDVKHAPLTHFQYND

SEQ ID NO: 40 - protein, 410 aa, Lactobacillus kalixensis
MTSYFKGFPQSDRNEKIDMINVDELEERAKKVMPEGAYYYIASGSENEWTWRANTSAFNHYQIVPRALTGMDNPSTETEFMGMKLKTPIMISPIACHGIAHKDAEVATQEGAAAAGALFSSSTYANKSVEDIAAAAPDAPRFFQLYLSKDWDFNKMVFEAINKAGYKGIFLTVDALVSGYREANLRTKFTYPVPLDFFTRYLGGKGEGQSVAQMYASSAQNISPADVERIKKESGLPVFIKGVMCAEDAYKALGAGADGIYVTNHGGREVDGAPATIDVLPEIAKAVNHRVPIIFDSGVRRGSHVFKALAMGADLVGIGRPYLYGLALGGAKGVESVIDQLDDELKIDMQLTGCKTIDDVKHAKIDSIHYGRDDLPSNTDPSRIKPYPKTADNQIKQEAETDATSGASHH

SEQ ID NO: 41 - protein, 367 aa, Liquorilactobacillus ghanensis
MTTINGYEQSDREQKLNILNLPALEAEAKKIIPKGGFGYISGGSEDEWTLRENTKAFNHVQIVPRALTEMEHPTTETTVFGIKLKTPIMMAPAAAQGLAHSRGEEATAEGLAQAGGLMAQSTYSSTSIAATAAAGKGAPQFFQLYMSKDWDFNRSLLDEAVKAGAKGIILTVDATVDGYREADIINQFQFPIPMANLAKFSEGDGKGKGIMEIYAAAAQKIGPDDVRKIAEYTNLPVIVKGIESAEDALLAIGAGAQGIYVSNHGGRQLNGGPAAFDVLHEVAQAVDHQVPVIFDSGVRRGSHVFKALANGADLVALARPIIYGLALGGAQGVASVVNHLNDELKIDMQLAGTKTIADVKQAKLIRH

SEQ ID NO: 42 - protein, 409 aa, Lactobacillus kitasatonis
MTYYYKGFPQSDRDEKISMINVDELEERAKKVMPEGAYYYIASGAENEWTWRANTSAFNHYQIVPRALTDMDDPQTDTEFMGMKLKTPIMISPIACHGIAHKDAEVATQKGAAAAGALFSSSTYANKSVEDIAAAAPEAPRFFQLYLSKDWDFNKMVFDAIKKAGYKGIFLTVDALVSGYREANLRTHFTYPVPLDFFTRYLGGKGEGQSVAQMYASSAQKISPEDVARIKKESGLPVFVKGVMCAEDAYKAIGAGADGIYVTNHGGREVDGAPATIDVLPEIAKAVNHRVPIVFDSGVRRGSHVFKALALGADIVGIGRPYLYGLALGGPKGVESVIDQLDNELKIDMQLTGCKTIADVKHAKIDRIHYGLDTMPSNTDPSRMEPYPVTADNQIKSQEPDATSGASKH

SEQ ID NO: 43 - protein, 372 aa, Companilactobacillus nantensis
MGFLMMNKKKYEASTAENHVDIVNLASLEARVKDHMSNEKGAFGYIRGGAEDEWTLNENTEAFNDKEIMPRVLQGIDHADLSTKLWDIDLKTPIIQAPSAAHGLAHIKGETDTAKGVAAAGSIYSISTYASTSVEDAAAAAPDSPMFFQLYMSKDDKFNEFLIKKAVKAGAKAIILTVDSTLGGYRESDVINKFQFPLPMPNLAGYSAGDGEGKGIGEIYASAKQGIVPTDIQKIKDMSGLPVFVKGIQSPDDAELAIQFGADGIWVSNHGGRQLDGGPASFALLPYIAEVVDKRVPIVFDSGVRRGEHVFKALASGADLVALGRPIIYGLNLGGAQGVTDVIEHLNMELAITMQLAGTKTIDDVKNTELLD

SEQ ID NO: 44 - protein, 409 aa, Lactobacillus intestinalis
MTYYYKGFPQSTRDEVLHMVNVEELARKAKDVMPEAAYYYVASGAENEWTWRANTSAFNHYQIIPRALTNMSHPQLDTEFMGMKLKTPVMIAPIACHGIANKAAEVGTQKGAAAAGALFSSSTYANKSVEDIAAAAPNAPRYFQLYLSKDWKFNRMVFDAIKKTGYKGIWLTVDALVSGYREANLRTHFTYPVPLDFFTRYLGGKGEGQTVAQMYAASAQKIGPKDVQRIKDESGLPVFIKGITNAEDAYKAIGAGADGVYVTNHGGREVDGEPATVDDLPEVAKAVAHRVPVVFDSGVRRGSHVFKALALGADMVGIGRPFLYGLALGGAKGVKSVIDQYNKELLIDMELTGCKTIEDVKHAKIAHIRYTADNLPSDTDPTRMKPYPVTKYNQMKSDEADASSGASEA

SEQ ID NO: 45 - protein, 369 aa, Lentilactobacillus parafarraginis
MAIVNGYKQNENEKKLDILNLPELEEKAKQIIPTGGFGYIVGGSEDEWTLRANRQAFTHKQIVPRALSNIEKPELDTNVFGLPLKTPIMMAPTAAQGLAHVEGEKDTARGVAAAGGLMAQSTYSSTSISDTSAAGNGAPQFFQLYMSKDWTFNESLLDEAKKAGVKGIILTVDATVDGYREADIINNFQFPIPMANLTKFSEGDGKGKGIAEIYASAAQKIGPDDVKRIADYTDLPVIVKGIESPEDALYAIGAGAAGVYVSNHGGRQLNGGPASFDVLEDVAKAVNGRVPIIFDSGVRRGSDVFKALASGADLVALGRPAIYGLALGGAQGVQSVFEHLGDELKIIMQLAGTKTIADVKKTNLLNIKY

SEQ ID NO: 46 - protein, 369 aa, Paucilactobacillus vaccinostercus
MTITNGYKQNENEKELDILNLPALEQEAQKIIPTGGFGYIAGGSEDEWTLHANRTAFQHKQIVPKALSDIEDPQTDTRVFNLDLKTPIMMAPAAAQGLAHVKGEIDTAAGVAAAGGLMAQSTYSSTSIADTAEAGNGAPQFFQLYMSKDWNFNESLLDEAKKAGVKGIILTVDATVDGYREADIINHFQFPIPMANLTKFSEGDGKGKGIGEIYASAAQKIGPKDVARIIQYTDLPVIVKGIESAEDALYAIGAGAAGIYVSNHGGRQLNGGPASFDVLTDVASAVNHRVPIIFDSGVRRGSDVFKALAAGADLVAIGRPAIYGLALGGAQGVTSVFNHLNDELKIIMQLAGTKTIEDVKHAPLLDIKY

SEQ ID NO: 47 - protein, 371 aa, Lacticaseibacillus brantae
MTVVNGYEQSDNEKKLDILNLPALEDEAKKIIPTGGYGYIFGGSEDDWTLKQNTLAFEHAQIVPKALSDIEKPDLSTNVFGIDLKTPVMMAPTAAQGLAHAQGEDDTARGVAAVGGLMAQSTYSSTSIADTAAAGNGAPQFFQLYMSKDWSFNESLLDEAKKAGVKAIILTVDATVDGYREQDIINNFQFPIPMANLTKFSEGDGKGKGIADIYASAAQKIGPDDVRRIAEYTDLPVIVKGVQSPEDAMRAIGAGAAGIYVSNHGGRQLNGGPASFDVLESIAKAVNHQVPIIFDSGVRRGSHVFKALAAGADLVAFGRPVIYGLALGGAQGVQSVFEHIDHELEIIMQLAGTKTIEDVKHAPLLHIHYND

SEQ ID NO: 48 - protein, 369 aa, Schleiferilactobacillus collinoides
MVLTNGYEQNENEKALDILNLTDLEKQAKAIIPTGGFGYISGGSEDEWTLRANRTAFQHRQIVPKALSNIENPRTDTSVFGLDLKTPLMMAPAAAQGLAHAKGEVDTAAGVAAAGALMAQSTYSSTSIADTAAGGNGAPQFFQLYMSKDWAFNESLLDEAKKAGVKGIILTVDATVDGYREADIINNFQFPIPMANLTKFSEGDGKGKGIGEIYAAAAQKIGPDDVRRIADYTDLPVIVKGVESAEDALYAIGAGAAGIYVSNHGGRQLNGGPASFDVLESVAKGVNHRVPVIFDSGVRRGSDAFKAIALGADLVAIGRPAIYGLALGGAQGVESVFNHLNDELKIIMQLAGTQTIADIHGAPLLDIKY

SEQ ID NO: 49 - protein, 370 aa, Lapidilactobacillus dextrinicus
MMTIVNGYEQSDREEHINVLNLESLEARAEKIIPKGGFGYISGGSEDNWTLAQNRKAFTHKQVYPRALANIDQPDLSANVFGLDLKTPVMMPPLAAQGLAHAKGEMDTAKAFAAEGALMAQSTYSSASIADTAAAGNGAPQFFQLYMSKDWKFNEAVIDEAKKAGVKGIILTVDATVDGYREADIINHFQFPIPMANLEKFSQGAGKGQGIAEIYAAAAQKISADDVRRIAEYSDLPVIVKGIQTPEDAELAIGAGASGIYVSNHGGRQLNGGPGSFDTLSAIAQAVNGRVPVIFDSGVRRGSDVFKALAAGADLVAIGRPFVYALALGGALGVQDALQEINHEFATIMQLAGTKTIADVKKAKLADFNY

SEQ ID NO: 50 - protein, 371 aa, Lacticaseibacillus thailandensis
MTLTNGYEQSDREEQLHILNLPSLEAAAKKIIPTGGFGYIYGGAEDEWTLKANTQAFNHVQIVPRALANIDHPDLSTEIFGLHLDTPVMMAPAAAQGLAHAKGEVDTAKGVAAVGGLMAQSTYSSTSIADTAAAGDGAPQFFQLYMSKDWTFNASLLDEAKRAGVKAIILTVDATVDGYREEDIVNNFQFPIPMANLEKFANGDGKGQGIGEIYAAAAQKISPDDVRRIADYTDLPVIVKGIQSPADALRAIGAGAAGIYVSNHGGRQLNGGPASFDVLADIAKAVNHQVPIIFDSGVRRGSHVFKALAAGADLVALARPVIYGLALGGAQGVQSVFEALDHELAITMQLAGTKTIEDVKHAPLTHFRYDD

SEQ ID NO: 51 - protein, 369 aa, Lentilactobacillus senioris
MTVVNGYEQSDREEHINVLNLESLEKRAKEIIPTGGFGYIVGGSEDDWTLAQNRKAFTHKQIYPRTLANIDNPDMSTNVFGIDLKTPVMMAPLAAQGLAHSKGEVDTAKAFANEGALMAQSTYSSASIADTAAAGEGAPQFFQLYMSKDWKFNEAILDEAKKAGVKGIILTADATVDGYREADIINDFQFPIPMANLEKFSEGAGKGQGIAEIYAAAAQKISPDDVRRIAEYSDLPVIVKGIQTPEDAELAIGAGAAGIYVSNHGGRQLNGGPASFDALSAIADSVAGRVPIIFDSGVRRGSDVFKALAAGADLVALGRPFVYALALGGALGVQDAIQELNHEFATTMQLAGTKTIDEVKQSKLADFKY

SEQ ID NO: 52 - protein, 367 aa, Liquorilactobacillus cacaonum
MTIFNGYEQSDREQKLDILNLPSLEEEAKKIIPTGGFGYISGGSEDEWTLHENTSAFNHIQIIPRALTDVDQPSTDTEVFGLKFKTPIMMAPAAAQGLAHSRGEEATAEGLAEVGALMAQSTYSSTSIADTAAAGKGAPQFFQLYMSKDWEFNYSLLDEAVKAGAKGIILTVDATVDGYRESDIINNFQFPIPMANLAKFSEGDGKGKGIMEIYAAAAQKISPADVRRIAEYTKLPVIVKGIESAEDALLAIGAGAKGIYVSNHGGRQLNGGPASIDVLHEVAVAVNHQVPVIFDSGVRRGSHVFKALASGADLVALARPIIYGLALGGAQGVASVINHLNDELKIDMQLAGTKTIEDVKKAKVIRK

SEQ ID NO: 53 - protein, 412 aa, Lactobacillus taiwanensis
MTTYYKGFPQSTREEKLHMVNLNELENEAKYVIPEAAYYYIASGAENEWTWRNNTQAFNHFQIVPRALTGMQDPELNTEFLGMKLKTPVMICPIACHGIANAEAEIDTAKGAKAAGSLFGMSTYANKSVQEVQAAVGDSPRYMQLYLSKNWDFNKMVIEESVKAGFTGFFLTVDALVSGYREANLRTNFTYPVPLAFFNEWNGGKGEGQSVAQMYASSAQNIGPDDIRKIKEIADVPVIVKGIECAEDAMLAIGAGADGIVVSNHGGREVDGAPATIDVLPEIAKAVKSYDHRVPIILDGGVRRGSHVFKALALGADLVGIGRPFLYGLALGGAQGVQSVIEQLNKELLIDMQLTGCKTIEDIKHAKIDHIDYTADWGISSTSKSVMKPYPVTKENQLTGEAADAVSGASRH

SEQ ID NO: 54 - protein, 368 aa, Weissella confusa
MTYNASTAEEKLDIIDLPRLRALVESRTEAGAFGYVDGGSSDEQVLQDNETAFRHYQLIPRMLQNIAEPDMTTTLFDIPLGMPIIAAPIAAQGLMHEGGESVTVKGVGAAKSIFSASTYGNASVAAVAEASPETPKFFQLYMSRDDEFNQFLINQAVETGYKAIILTADSTLGGYREADIINNFEFPLPMDNLAAFSNAAGTGEGLSIAEIYARAKQDLALSDIKKIKDMASGLPVIVKGIQDPEDALAAIAGGADGIWVSNHGGRELNGAPASIDVLADIAKAVNHRVPIIFDSGIRWGEDIAKAIALGADVVALGRPMLWALNLGGAAGVQSAFEHLAEELKIVMQLTGSHTVAELKHAKIIEAKF

SEQ ID NO: 55 - protein, 369 aa, Pediococcus stilesii
MTMVNGYEQSDREEKIDILNLESLEGRAKEIIPTGGFGYIVGGSEDEWTLKKNRDAFHHRQIAPKALSGMENPDLSTNVFGIPLKTPVMMAPTAAQGLAHSQGEKDTAKGVAAVGGLMAQSTYSSTSIADTAAAADGAPQFFQLYMSKDWSFNESLLDEAKEAGVKAIILTVDATVDGYREADIINDFAFPIPMANLTKFSEGDGQGKGIEEIYASAAQNIKPEDVRRIAEYTDLPVIVKGIQTPEDAIRAIDAGAAGIYVSNHGGRQLNGGPASFDVLESISKAVNGQVPIIFDSGVRRGSDVFKALASGADLVAFGRPVIYGLALGGAQGVQAVFDHIDHELEIVMQLAGTKTIEDVKKTHLLDIKY

SEQ ID NO: 56 - protein, 366 aa, Companilactobacillus kimchiensis
MKKKYEASTAENHVEIVNIAGLEARVKDHMSNEKGAFGYIRGGAEDEYTMKENTAAFNKAKIMPRVLQGIDHADLSTNLWDIALKTPIIESPSAAQGLAHANGEKDTAKGVAAAGSIFSMSTYGSTSLEDGAAAAPDAPQFFQLYMSKDDKFNEFLINKAVKAGVKAIVLTVDSTLGGYREEDVINKFQFPLPMPNLAAYSEGDGEGKGIGEIYAAAKQGIVPSDIQKIKDMSGLPVIVKGIQSPDDADLAIEFGADGIWVSNHGGRQLDGAPASFDILPDIADRVDKRVPVIFDSGVRRGEHVFKALASGADLVAVGRPIIYGLNLGGAQGVTDVIEHLNMELSITMQLAGTKTINDVKNTDLYY

SEQ ID NO: 57 - protein, 371 aa, Lacticaseibacillus saniviri
MTVVNGYEQSDNEKKLDILNLPALEDEAKKIIPTGGYGYIFGGSEDDWTLRQNTKAFEHAQIVPKALSDIESPDLSTNVFGLDLKTPVMMAPTAAQGLAHAKGEADTARGVAAVGGLMAQSTYSSTSIADTAAAGNGAPQFFQLYMSKDWSFNESLLDEAKKAGVKAIILTVDATVDGYREQDIINNFQFPIPMANLTKFSEGDGKGKGIGEIYASAAQKIGPDDVRRIAEYTDLPVIVKGIQSPEDALRAIGAGAAGIYVSNHGGRQLNGGPASFDVLESIAKAVNHQVPIIFDSGVRRGSHVFKALAAGADLVAFGRPVIYGLALGGAEGVQSVFEHIDHELEIIMQLAGTKTIEDVKHAPLLHINYAD

SEQ ID NO: 58 - protein, 426 aa, Lactobacillus crispatus
MTHYYEGFPQSDRDEKIKMVNVDELEERVKKVMPEGAYYYIASGAENEWTWRNNTAAFNHFQIVPRALTEMADPQTDTDFMGMHLKTPIMIAPIACHGIAHKDAEVATQKGAAMAGALFSSSTYANKSVEEIAAAAPEAPRFFQLYLSKDWNFNQMVFDAIKKAGYQGIFLTVDALVSGYREANLRTNFTYPVPLDFFKRYLGAKGKGQSVAQMYASSAQKIGPEDVKRIKKESGLPVFVKGVMCAEDAYKAIGAGADGIYVTNHGGREVDGAPATIDVLPEIAQAVNHRVPIIFDSGVRRGSHIFKALALGADIVGIGRPYLYGLALGGAHGVASVIEQLNDELKIDMQLTGCKTIDDVKHAKLTHIAYTADNLPSNTDPSRRKEYPVTDENQVKQTDAVSGASKLEGKPAPEPETDTTTGASVR

SEQ ID NO: 59 - protein, 412 aa, Lactobacillus johnsonii
MTTYYKGFPQSTREEKLHMVNLDELENEAKYVMPEAAYYYVASGAENEWTWRNNTQAFNHFQIVPRALTGMQNPELNTEFLGMKLKTPVMICPIACHGIANAEAEIDTAKGAKAAGALFAMSTYANKSVQEVQSAVGDSPRFMQLYLSKNWDFNKMVIEESVKAGFTGFFLTVDALVSGYREANLRTNFTYPVPLAFFNEWNGGKGEGQSVAQMYASSAQNIGPDDIRKIKEIADVPVIVKGVECAEDAMLAIGAGADGIVVSNHGGREVDGAPATIDVLPEIAKAVRSCDHRVPIILDGGVRRGSHVFKALALGADLVGIGRPFLYGLALGGAQGVQSVIEQLNKELLIDMQLTGCKTIEDIKHAKIDHINYSADWGISSTSRSVMKPYPVTKENQLTGEAADAVSGASRH

SEQ ID NO: 60 - protein, 404 aa, Providencia stuartii
MLHRAKKQPIAVMSSAILALTLSVGAVQAADYQASTKEGPIKIINLDELEDQVAKNMEKGAFGYIRGGAEDELNLDKNTRSFDRKYIMPRVMQGIEIKDIDLSTQFLGINLKTPIIQAPMAAQGLAHQDGEIATAKGMAKAGSIFSLSTYGNKTIEEVAEVSGESPFFFQLYMSKNNAFNEFTLKRAKESGAKAIILTVDSPVGGYREDDIRNNFQFPLGFANLELFAKQNDDGSKTGKGAGISEIYAQAKQAFTPADIAYVKKLSGLPVIVKGIQSPEDADRVIKAGADAIWVSNHGGRQLDSGPASFDVLPSIAKVVNKRVPIVFDSGVRRGSHVFKALASGADVVAVGRPILYGLNLGGSEGVNSVIQQLNKELSINMMLGGAKNIESVKATKLYTDMDFQ

SEQ ID NO: 61 - protein, 410 aa, Myroides marinus
MKKIIMTLTAITLTTSVFAHTTTETVFQNKDKKTKGYQASTADKKLKIVNLYELEGQVKAEMDRGAFGYIVGGAEDQNNLKINTENFDKKYIMPRVLKGIKHEDIDLSTSLFGIPLKTPIIKAPMAAQGLSHVDGEIATAKGMIAAGSLFSLSTYGNKTIEEVAEGINGAPFFFQLYMSKNDEFNKFTLERAKKYGAKAIILTVDSPVGGYREEDIKTGFTFPLGMGNLELFAAQQADGNKTGKGAGITEIYAQAKQDFKPSDIKYVKDMTGLPVIIKGIQSPEDAEIAIQAGADAIWVSNHGGRQLDAGPSSFDVLPLVAKTVNKRVPIIFDSGVRRGSHIFKALASGADIVAIGRPILYALHLGGSQGVTSVIDQLNKELTINMFLGGAKNIKEIQNTKLYTDKDFQL

SEQ ID NO: 62 - protein, 366 aa, Lactococcus cremoris
MTYQTSTENKAIEIVNIKSLEGKVKESMESAGNKGAFGYIRGGAEDEWTMGENTSAFNKKQIMPRVLRGIDSADLSTSLFGIDLSTPIIQAPVAAQGLAHEEGEVATAKAMAEVGSIFSISTYGSTSVEDAAKAAPGAPQFFQLYMSKDDKFNEFLLKKALAAGVKAIILTADSTLGGYREEDIVNHFQFPLPMPNLAAFSESDGTGKGISEIYAAAKQGLVLEDIKKIKEITKLPVIVKGIQSPIDADDAIKAGADGIWVSNHGGRQLDGGPASIDVLPLVAKSVNHRVPIIFDSGVRRGGHVFKALAQGADIIAIGRPVLYGLNLGGAKGVQSVFEHLNKELSITMQLAGTKNIDEVKRTALID

SEQ ID NO: 63 - protein, 366 aa, Lactococcus cremoris
MKYQTSQENKKIEIINLQSLEARVKKSMEAAGNGGAFGYIRGGAEDEWTMRENTLAFNKKKIMPRVLRGIDHADLSTSLFGIPLTTPIIQAPVAAQGLAHEEGEVATAKALAKIGSIFSISTYGSTAVEEAAQAVPGAPQFFQLYMSKEDHFNEFLLAKAVAAGVKAIILTADSTLGGYREEDVVNHFQFPLPMPNLAAFSESDGKGRGISEIYAAAKQELVMEDIQKIKAFTHLPVIVKGIQSPIDAAEAIEAGADGIWVSNHGGRQLDGGPASIEVLPLIAEQVNHRVPIIFDSGIRRGSHVFKALALGADVVAIGRPVLYGLNLGGAQGVEAVFEHLNKELSITMQLAGTKTIDEVKHTPLMD

SEQ ID NO: 64 - protein, 366 aa, Enterococcus thailandicus
MTYHTSNEEHPIEIVNIASLEDRVKERMEAGAFGYIRGGSEDEWTMKENTSAFNTKKIMPRVLRGIDHADLHTSVFGIDLDTPIIQAPSAAQGLAHEKGEADTAKGVAAAGSIFSISTYANTTIKDAAAAAPNAPQFFQLYMSKDDGFNEFILTQAMEAGAKAIILTADSTLGGYREEDVINQFQFPLPMPNLAAYSEQSASGSGEGKGIAEIYAAAKQGLVPDDIKKIKEFTHLPVFVKGIQSPEDAEVAIAAGADGIWVSNHGGRQLDGGPASFEVLPMIAATVNKRVPIVFDSGVRRGEHVFKALASGADLVAIGRPVIYGLNLGGAEGVTSVFEHFNKELSITMQLAGTKTIEEVKNTTLLD

SEQ ID NO: 65 - protein, 371 aa, Lacticaseibacillus rhamnosus
MTVVNGYEQSDAEKKLDILNLPSLEGESKKIIPTGGFGYIVGGSEDEWTLAENTKAFNHAQIVPKALSNIDSPDLSTNFLGIDLKTPVMMAQTAAQGLAHSQGEKDTARGLAAVGGLMAQSTYSSTSIADTAAAGNGAPQLFQLYMSKDWDFNKSLLDEAKKAGVKGIILTVDATVDGYREEDIINNFQFPIPMPNLEKYSEGDGKGKGIGEIYASAAQKINEDDVRRIAEYTDLPVIVKGIQSPEDALRAIGAGAAAIYVSNHGGRQLNGGPASFDVLPAIAKAVNKQVPIIFDSGIRRGSHVFKALASGADLVAFGRPVIYGLALGGAQGVQSVFEQIDHELEIIMQLAGTKTIEDVKHAPLTHFNYAD

SEQ ID NO: 66 - protein, 369 aa, Pediococcus acidilactici
MTMINGYEQSDREEKIDILNLESLEKRAEEIIPAGGFGYIAGGSEDEWTLKQNRMAFHHRQIAPKALSGIEKPELNTEIFGIPLNTPVMMAPAAAQGLAHSQGEKDTARGLAAVGGLMAQSTYSSVSIAETAAAGGDAPQFFQLYMSKDWNFNESLLDEAKKANVKAIILTVDATVDGYREADIKNKFTFPLPMANLIKFSEGNGQGKGIEEIYASAAQNIRPEDVKRIADYTNLPVIVKGIQTPEDAIRAIDAGAAGIYVSNHGGRQLNGGPASFDVLEDIATAVNKQVPIIFDSGVRRGSDVFKALASGADLVALGRPVIYGLALGGAKGVQSVFEHLNHELEIVMQLAGTKTIEDVKNNSLLNIKY

SEQ ID NO: 67 - protein, 400 aa, Morganella psychrotolerans
MKKVKAGFFAATVLGLAMTMTTAYAAEYKASIKEGPLKIVNLNDLESQVKANMDKGAFGYIRGGAEDEKNMRDNTASFDRKYIMPRVMQGIELKDINISTSLLGIPLDTPVIQAPMAAQGLAHRDGEIATARGMAQAGSIFTLSTYGNKTIEEVAAVSDGHPFFFQLYMSKNDAFNEFTLKRAKESGAKAIILTVDSPVGGWREDDLRNNFQFPLGFANLELFAKQNNDGAKTGKGAGISEIYAQAKQAFTPSDIQYVKKMSGLPVIVKGIQSPEDADRVIEAGADAIWVSNHGGRQLDSGPASFDVLPAIAKTVNKRVPVVFDSGVRRGSHVFKALASGADIVAVGRPVLYGLNLGGAQGVNSVIQQLNKELRINMMLGGAKDINAVKQTKLYTDSELQ

SEQ ID NO: 68 - protein, 365 aa, Gilliamella apicola
MSYHTSNEEHAIEIVNIASLEALVKARMEPGAFGYIRGGAEDEWTMRENTLAFNRKKIIPRVLQGIDHANLSTKLWDISLKTPIIQAPSAAQGLAHEQGEKDTAKGVAAAGSIFCISTYANTSIEDAANAAPNVPYFFQLYMSKDDDFNRFIIDKAVKAGAKAIILTVDSTLGGYREEDIVNKFQFPLPMKNLSAYSQSNGNGDGSGKGISEIYAAAKQGIVPSDIQKIKAMANLPVIVKGIQSPEDASIAISAGADGIWISNHGGRQLDGAPASFEVLPSISAAVAKRVPIIFDSGIRRGEHVFKALASGADLVAIGRPILYGLNLGGAEGVKSVFDHLNKELSITMQLAGTKTIEDIKNTLLI

SEQ ID NO: 69 - protein, 365 aa, Gilliamella apicola
MSYHTSNEEHAIEIVNIASLEALVKARMEAGAFGYIRGGAEDEWTMRENTLSFNRKKIVPRVLQGVDHADLTTKLWDIPLKTPIIQSPSAAQGLAHEKGEMDTAKGVAAAGSIFSISTYANTSIEEAASAAPNVPYFFQLYMNKDDGFNRFIVDKAVKAGAKAIILTVDSTLGGYREEDIINKFQFPLPMKNLSAYSQSSGNGDGSGKGISEIYAAAKQGIVPSDIQKIKEMAKLPVIVKGIQSPEDAAIAISAGADGIWVSNHGGRQLDGGPASFDVLPNIAAVVAKRVPIIFDSGVRRGEHVFKALASGADLVAIGRPILYGLNLGGAEGVKSVFDHLNKELSITMQLAGTKTIEDIKNTPLI

SEQ ID NO: 70 - protein, 365 aa, Gilliamella apicola
MSYHTSNEEHAIEIVNLASLEALVKVRMEAGAFGYIRGGAEDEWTMRENTLAFNRKKIVPRVLQGIDHADLSTKLWDISLKTPIIQAPSAAQGLAHEQGEKDTAKGVAAAGSIFCISTYANTSIEDAANAAPNVPYFFQLYMSKDDDFNRFIIDKAVKAGAKAIILTVDSTLGGYREEDIVNKFQFPLPMKNLSAYSQSNGNGDGSGKGISEIYAAAKQGIVPSDIQKIKDMANLPIIVKGIQSPEDASIAISAGADGIWISNHGGRQLDGAPASFEVLPSISAAVAKRVPIIFDSGIRRGEHVFKALASGADLVAIGRPILYGLNLGGAEGVKSVFDHLNKELSITMQLAGTKTIEDIKNTLLI

SEQ ID NO: 71 - protein, 365 aa, Gilliamella apicola
MSYHTSNEEHAIEIVNIASLEALVKARMQAGAFGYIRGGAEDEWTMRENTLSFNRKKIIPRVLQGIDRADLSTKLWDISLKTPIIQAPSAAQGLAHEQGEKDTAKGVAAAGSIFCISTYANTSIEDAANAAPNVPYFFQLYMSKNDDFNRFIIDKAVKAGAKAIILTVDSTLGGYREEDIVNKFQFPLPMKNLSAYSQSNGNGDGSGKGISEIYAAAKQGIVPSDIQKIKAIANLPVIVKGIQSPEDASIAISAGADAIWISNHGGRQLDGAPASFEVLPNIAATVAKRVPIIFDSGVRRGEHVFKALASGADLVAIGRPILYGLNLGGAEGVKSVFDHLNKELSITMQLAGTKTIEDIKNTLLI

SEQ ID NO: 72 - protein, 409 aa, Lactobacillus apis
MTSYYNGFPQSDRDEAIDMINLDELEERAKEVMPEGAYYYIASGSENEWTWRNNIAAFNHFQIVPRALTNMDNPQLDTEFMGMKLKTPVMISPIACHGISHKDAEVATQKGAAAAGALFSSSTYANKSVEDIAAAAPDAPRFFQLYLSKDWDFNEMVFEAIKKAGYKGIFLTVDALVSGYREANLRTNFAYPVPLDFFTRYQGGKGEGQTVAQMYASSAQKIGPEDVKRIKEMSGLPVFVKGVVCAEDAYLAMGAGADGIYVTNHGGREVDCGPATIDMLPEIAKAVNHRVPIVFDSGVRRGSHVFKALALGADLVGVGRPYLYGLALGGAKGVQSVIEQLNKELLIDMQLTGCKTIEDVKHAKITHINYAADNLKSNTDPSRIKPYPVTAENQMKESAADAVSGASHH

SEQ ID NO: 73 - protein, 365 aa, Gilliamella bombicola
MSYHTSNEEHAIEIVNIASLEALVKARMEAGAFGYIRGGAEDEWTMRENTLSFNRKKIIPRVLQGIDHADLSTKLWDIPLKTPIIQAPSAAQGLAHEQGEKDTAKGVAAAGSIFCISTYANTSIEDAANAAPNVPYFFQLYMSKDDDFNRFIIDKAVKAGTKAIILTVDSTLGGYREEDIVNKFQFPLPMKNLSAYSQSNGNGDGSGKGISEIYAAAKQGIVPSDIQKIKDMANLPVIVKGIQSPEDASIAISAGADGIWISNHGGRQLDGAPASFEVLPSIAATVAKRVPIIFDSGVRRGEHVFKALASGADLVAIGRPILYGLNLGGAEGVKSVFDHLNKELSITMQLAGTKTIEDIKNTLLI

SEQ ID NO: 74 - protein, 398 aa, Shigella sp.
MKRQILKTTALAMALSVGVAQAAEYKASTAEGPIKIVNLKAMEAQVQANMEKGAFGYIRGGAEDENNLRSNTSAFDKKYIMPRSLQGIEFSDLDLTTEFLGIKLDTPIIQAPMAAQGLAHQQGEVATAKGMAKAGSIFSLSTYGNKTIKEVADAQPGYPFFFQLYMSKNDAFNEYILSQAKQYGAKGIIMTIDSSVGGYREDDVKNNFQFPLGFANLEAFAKISDDKSKTGKGAGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADVVAVGRPILYGLNLGGAEGVNSVIQHLNKELKINMMLGGAKTVKDIQATQLYTDASFNQ

SEQ ID NO: 75 - protein, 361 aa, Allisonella histaminiformans
MAYYTSNAEHPIDILNIKGLEARVKARMEPGAFGYIREGAEDEWTLRENTRSFEDKYIVPRVLRGISHADLSTSIFGIPLSTPVIEAPSAAHGLAHVKGEVDTAIGAAKAGTLFAMSTYGSTDLEEAAKAAPGAPQFFQIYMSKDDGFNEFLIRKAVRAGVKAIIMTVDSTLGGYREEDIATHFQFPLPMPNLAAYGNADGKGKGISEIYAAAKQDFVPSDIGKLKRLSGLPVIVKGIQSPEDALIALDAGADGIWVSNHGGRQLDGGPASFAVLPSIAKAVNKRAPIIFDSGVRRGNHVFKALASGADLVAIGRPVIYGLNLGGAEGVKSVFDHFNKELSITMQLAGTKTIEDVKKTELI

SEQ ID NO: 76 - protein, 367 aa, Propionibacterium cyclohexanicum
MTVTNGYEQSDREQAIKIINLDELEDQARQIIPQGGFGYISEGSEDEWTKARNRAAFNTVQIAPRVLHSVEAPSTSTSVFGVSIKTPVIMAPTAAQGLAHTRGEAATAEGVAAAGTIMSQSTYGTTSIAETAEASKGAPWFFQLYMSNDWEFNEALLDEAKSHGAAAVVLTVDSMQGGYREPDIRNEFQFPLPMANLAAFSETSGKGKGIFEIYAAAKQKITGADVRRVAEYVGLPVIVKGIQDPCDAALALGSGASGIWVSNHGGRQLNGGPGSFDVLPSIARAVNGRVPVIFDSGVRRGSQVFKALASGADLVAIGRPAIYALALGGAQGVRTVFEYLTHEFRIVMQLAGTQTVDDVKRAKLLHY

SEQ ID NO: 77 - protein, 409 aa, Lactobacillus bombicola
MTVYYNDFPQSDRDEYIKMINLDELEERAKKVMPEGAYYYIASGSENEWTWRNNTVAFNHFQIVPRALTNMADPQLDTDFMGMKLKTPIMISPIACHGIAHQDAEVATQKGAAAAGALFSSSTYANKSVEDISAAAPEAPRFFQLYLSKDWNFNQMVFDAIKKAGYQGIFLTVDALVSGFREANLRTQFAFPVPLDFFTRYQGTKGEGQTVAQMYASSAQKIGPEDIKRIKEMSGLPVFVKGVVCAEDAYLAIGAGADGIVVTNHGGREIDGGPATIDMLPEIAKAVDHRVPIVFDSGVRRGSHVFKALALGADLVGIGRPYLYGLALGGAKGVQSVIEQLNKELLIDMQLTGCKTIADIRHAKITNINYTADNLKSNTDPERIKPYPVTKENQLKAGGTDTVSGASHL

SEQ ID NO: 78 - protein, 402 aa, Providencia rettgeri
MLSFTRKNTFVTSTLFAVALTLGTAQAAEYKASDKEGPIKIVNLDELESQVAKSMDKGAFGYIRGGAEDELNLKKNTQHFDNKYIMPRVMQGIEISDIDLSTDFLGIKLKTPIIQAPMAAQGLAHKDGEIATAKGMAKAGSIFSLSTYGNKTIEEVAEVSGENPFFFQLYMSKNNAFNEFTLKRAKASGAKAIILTVDSPVGGYREDDIRNNFQFPLGFANLELFAKQNSDGSKTGKGAGISEIYAQAKQAFTPADIQYVKKLSGLPVIVKGIQSPEDADVVIKAGADAIWVSNHGGRQLDSGPASFDVLPSIAKVVNKRVPIVFDSGVRRGSHVFKALASGADVVAVGRPILYGLNLGGAEGVNSVIQQLNKELSINMMLGGAKNIEGVKATKLYTDKDFQ

SEQ ID NO: 79 - protein, 365 aa, Enterococcus canintestini
MYHTSNEEHPIDIVNIASLEGRVKERMEAGAFGYIRGGSEDEWTMKENTTSFMNKKIMPRILQGIDHADLSTKLWDIELKTPIIQAPSAAQGLAHEKGEADTAKGVAAAGSIFSISTYANTTIEDAAAAAPDAPQFFQLYMSKDDGFNEFILDKAVKAGAKAIILTADSTLGGYREEDVINQFQFPLPMPNLAAYSAQSASGDGEGKGIAEIYAAAKQGLVPEDIKKIKDLTHLPVFVKGIQSPEDADLAIKAGADGIWVSNHGGRQLDGGPASFEVLPAIAEVVDKRVPIVFDSGVRRGEHVFKALASGADLVAIGRPVIYGLNLGGAEGVKSVFDHLNKELSITMQLAGTKTIADVKKTKLID

SEQ ID NO: 80 - protein, 366 aa, Enterococcus canis
MAYHTSNEEHPIEIVNIASLEGRVKERMEAGAFGYIRGGSEDEWTMRENTAAFNKKTIMPRVLRGIDHADLSTKLWDIELATPIIQAPSAAQGLAHEKGEADTAKGVAAAGSIFSISTYANTTIEDAAKAAPEAPQFFQLYMSKDDGFNKFILDKAVQAGAKAIILTADSTLGGYREEDIINQFQFPLPMPNLAAYSAQSASGNGEGKGIAEIYAAAKQGLVPEDIKAIKDLTDLPVFVKGIQSPEDAEVAIAAGADGIWVSNHGGRQLDGGPASFDVLPKIAAVVNKRVPIVFDSGVRRGEHIFKALASGADLVAIGRPVIYALNLGGAEGVKSVFDHLNKELSITMQLAGTKTIEEVKNTVLFD

SEQ ID NO: 81 - protein, 368 aa, Enterococcus devriesei
MAYQTSNEEHPVEIVNIASLEGRVKERMDKGAFGYIRGGSEDEWTMKENTSSFNNKTIMPRVLRGVDHADLSTKLWDIELATPIIQAPSAAQGLAHEKGEADTAKGVAEAGSIFSISTYANTTVEDAAATAPEAPQFFQIYMSKDDGFNEFILDKAVKAGAKAIILTADSTLGGYREEDIVNNFQFPLPMPNLAAYSEQSASGDGEGKGIAEIYAAAKQGLVPEDIKKIKDLTNLPVLVKGIQSPEDAEVAIAAGADGIWISNHGGRQLDGGPASFDVLPLIAGVVNKRVPIIFDSGVRRGEHVFKALASGADLVAIGRPVIYGLNLGGAAGVKSVFDHLNKELSITMQLAGTRTIEEVKNTKLNDAK

SEQ ID NO: 82 - protein, 367 aa, Companilactobacillus allii
MTETNGYFQNDNEKEINVLNLQSLEAEAEKIIPKGGFGYIAGGSEDNWTLKENTEAFNHVQIVPHVLSNVEDPQTDTSIFGINVKTPIMMSPAAAQGLAHAKGEIDTAAGIAKAGALMSQSTYSSTSIADTMKAGNGAPQFFQLYMSKDWNFNENLLKEAKEAGAKAIILTSDATVDGYRESDVVNDFQFPIPMANLTKFSEGDGKGKGIGEIYAAAAQKIGPDDIKRIKDIAGLPVIVKGVQSPEDALLAIGAGADGIYVSNHGGRQLNGGPASFDVLSDVAKAVNHRVPIIFDSGIRRGSHVFKALASGADLVALARPIIYGLALGGADGVYSVVEHLNDEFKTTMQLAGTKTIEDVKNSKLLKK

SEQ ID NO: 83 - protein, 367 aa, Loigolactobacillus backii
MSNTYKQSTNEQAIEIVNLDELQERAKAIIPAGGFGYISSGSEDEWTLRANREAFNHKLIVPRSLTDMEKPLIDTSIFGIPLKTPVMMAPTAAQGLAHIEGEADTARGVAAVGGLMAQSTYSSRTITETTTAGNGAPQFFQLYMSKDWSFNNALLDQAKAAGIKAIILTVDATVGGYREADVRNKFSFPIPMANLENFSKDNGEGKGISEIYAAAAQKISPKDIARIANYTDLPVIVKGIQSPEDAELAISSGAAGVYVSNHGGRQLNGGPASFDVLASVAQVVNHRVPVIFDSGIRRGSHVFKALASGADLVAFGRPAIYGLALGGAQGVQSVFEHIDDELKIVMQLAGTQTIAAVKQTKLLDNHF

SEQ ID NO: 84 - protein, 397 aa, Proteus mirabilis
MKHTLLKTTAIAMALSVGVVQAAEYKASTAEGPIKIVNLKAMEAQVEANMDKGAFGYIRGGAEDENNLRANTRAFDKKYIMPRSLQGIEFSDINLKTEFLGIKLDTPIIQAPMAAQGLAHQQGEVATAKGMAKAGSIFSLSTYGNKTIKEVAQAQPGYPFFFQLYMSKNDAFNQYILSQAKQYGAKGIIMTIDSSVGGYREDDVKNNFQFPLGFANLEAFAKISDDKSKTGKGSGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADVVAVGRPILYGLNLGGAEGVNSVIQQLNKELRINMMLGGARNVKEIQATHLYTDADFK

SEQ ID NO: 85 - protein, 369 aa, Lentilactobacillus curieae
MTVVNGYEQSDREEKLNILNLPSLEAEAKKIIPTGGFGYIVGGSEDEWTLNHNSEAFNHVQIVPRALTDMENPSTATKVFGLDLKTPIMMAPTAAQGLAHSRGEEATAEGVAAAGALMAQSTYSSTSIADTAAAGKGAPQFFQLYMSKDWDFNESLLDEAKKAGVKAIILTVDATVDGYREADIINNFQFPIPMANLTKFSEGDGEGKGIEEIYASAAQKINADDVKRIADYTDLPVIVKGIESPEDALLAIGAGAKGIYVSNHGGRQLNGGPASFDVLEDVAKAVNHQVPIIFDSGIRRGSHVFKALASGADLVAMGRPVIYGLALGGAQGVQSVFEELNHELEIDMQLAGTKDIEAVKHAKLVKVHY

SEQ ID NO: 86 - protein, 368 aa, Weissella confusa
MAYQGSTKEEALNIIDLPRLRAAVQRDTEAGAFGYVDGGSSDEQVLHDNETAFRHYQLIPRMLQNISAPDLSTTLIDIPLSMPVIAAPIAAHGLMHENGERVTVKGVGAAGTIFSLSTYGNSRIADVAASSPDTPKFFQLYMSRDDEFNQYLLDEAVQNGYKAIILTADATLGGYREADIINNFAFPLPMENLAAFSNAAGSGEGLGISEIYARAKQDLALSDITKVKQMANGLPVFVKGIQDPDDALAAIAAGADGIWVSNHGGRELNGAPASIDTLAAVAKAVNHRVPVVFDSGIRRGEDVAKAIALGADVVALGRPMLWGLNQGGAAGVQSVYEHLAEELKIVMQLTGSHTVAELQHAKIIDAKF

SEQ ID NO: 87 - protein, 412 aa, Lactobacillus gasseri
MTTYYKGFPQSTREEKLHMVNLDELENEAKYVIPEAAYYYIASGAENEWTWRNNTQAFNHFQIVPRALTGMQDPELNTEFLGMKLKTPVMICPIACHGIANAEAEIDTAKGAKAAGALFGMSTYANKSVQDVQNAVGDSPRFMQLYLSKNWDFNKMVIEESVKAGFTGFFLTVDALVSGYREANLRTNFTYPVPLAFFNEWTGGKGEGQSVAEMYASSAQNIGPDDIRKIKDIADVPVIVKGVECAEDAMLAIGAGADGIVVSNHGGREVDGAPATIDVLPEIAKAVKSCDRPVPIILDGGVRRGSHVFKALALGADLVGIGRPFLYGLALGGAQGVQSVIEQLNKELLIDMQLTGCKTIDDIKHAKIDHLDYTADWGISSTSKSVMKPYPVTKENQLTGEAADAVSGASRH

SEQ ID NO: 88 - protein, 369 aa, Pediococcus pentosaceus
MTMINGYEQSDREEKIDILNLESLEERAEKIIPTGGFGYISGGSEDEWTLRQNRTAFQHRQIAPKALSGIEKPELNTEIFRIPLNTPVMMAPAAAQGLAHSQGEKDTARGLAAVGGLMAQSTYSSVSIADTAAAGEGAPQFFQLYMSKDWNFNESLLDEAKKAHVKAIILTVDATVDGYREADIKNKFAFPLPMANLTKFSEGDGQGKGIEEIYASAAQNIRPEDVKRIADYTQLPVIVKGIQTPEDAIRAIDAGAAGIYVSNHGGRQLNGGPGSFDVLEDIATSVNKQVPIIFDSGVRRGSDVFKALASGADIVALGRPVIYGLALGGAKGVQSVFEHIDHELEIVMQLAGTKTIDDIKNNPLLNIKY

SEQ ID NO: 89 - protein, 410 aa, Lactobacillus gallinarum
MTVYYKGFPQSDREEYLHMINVDELEQRVKKIMPEGAYYYIASGSENEWTWRNNTAAFNHWQIIPRALTDMENPSTATEFMGMKLKTPVMISPIACHGIAHTMAEVDTQKGAAAAGALFASSTYANKSVEDIAKAAPDAPRFFQLYLSKDWKFNEMVFDAIKKTGYKGIYLTVDALVSGYREANLRTKFTYPVPLDFFTRYLDGKGEGQSVAQMYASSAQKIGPEDVRRIKEETGLPVIVKGVESVEDAYKAMGAGADGIYVTNHGGRQLDGGPAAIDVLPAIAKAVNHQVPIIFDSGVRRGSHVFKALALGADMVGIGRPYLYGLALGGAPGVQSVIDQLDKELLIDMQLTGCKTIEDVKHAKIAHINYTADNLDSITSPDRMKPYPKTADNQIKTNGASDATSGASEH

SEQ ID NO: 90 - protein, 397 aa, Proteus mirabilis
MKHTLLKTTAIAMALSVGVVQATEYKASTAEHPIKIVNLDALENQVKENMDKGAFGYIRGGAEDENNLRSNTNAFNKKYIMPRALQGIEFSDLNLKTEFLGIKLDTPIIQAPMAAQGLAHQQGEVATAKGMAKAGSIFSLSTYGNKTIKEVAQAQPGYPFFFQLYMSKNDAFNQYILSQAKQYGAKGIILTVDSPVGGYREDDIKNSFQFPLGFANLEAFAKISDDKSKTGKGSGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADVVAVGRPILYGLNLGGAEGVNSVIEQLNKELRINMMLGGARNVKEIQATHLYTDADFK

SEQ ID NO: 91 - protein, 369 aa, Schleiferilactobacillus silagei
MVITNGYKQNENERLIDIVNLTQLEAEAKQIIPAGGFGYITSGSEDEFTLQANRKAFQHRQIVPRSLSNIEKPQTDTNVFGIDLKTPIIMAPAAAQGLAHVKGEADTAKGVAKAGALMTESTYSSASIADVAAGGGGAPQFFQLYMSKNWDFNRSILDEAKKAGVKAIILTVDATVGGYREADIVNHFQFPIPMANLVKFSQGDGEGKGISEIYAAAAQKIGPKDVQRIIDYTDLPVIVKGIESPEDALYAIGAGAAGVYVSNHGGRQLNGGPASFDVLESVAKAVNHQVPVLFDSGVRRGSDVFKALALGADLVGIGRPAFFGLALGGADGVFSVFEHLNNELKIVMQLAGTKTVADIKHAKLLNIQY

SEQ ID NO: 92 - protein, 369 aa, Secundilactobacillus silagincola
MVMTNGYEQNENEKTLDILNLTQLEEDAKKIIPTGGFGYIVSGSEDEWTLNANRKAFQHRQIVPKALSNIENPQTDTTVFGLDLKTPIMMAPAAAQGLAHAKGEVDTAKGVAKAGGLMAQSTYSSTSIADTAAGGDGAPQFFQLYMSKDWDFNKSLLDEAKQAGVKAIILTVDATVGGYREADIINHFQFPIPMANLIKFSEGDGEGKGISEIYASAAQKIGPDDVKRIIDYTDLPVIVKGIESPEDALYAIGAGAAGIYVSNHGGRQLNGGPASFDVLESVTKAVNHQVPVIFDSGVRRGSDVFKAIALGADLVAIGRPAIYGLALGGADGVYSVFEHLNNELKIVMQLAGTKTIEDIKHAELLHIQY

SEQ ID NO: 93 - protein, 369 aa, Secundilactobacillus mixtipabuli
MVMTNGYEQNENEKTLDILNLTQLEADAKKIIPTGGFGYIVSGSEDEWTLQANRKAFQHRQIVPKALSNIEDPQTDTNVFGLGLKTPIMMAPAAAQGLAHAKGEVDTAKGVAKAGGLMAQSTYSSTSIADTAAGGEGAPQFFQLYMSKDWDFNRSLLDEAKQAGVKAIILTVDATVGGYREADIINHFQFPIPMANLIKFSEGDGEGKGISEIYASAAQKIGPDDVKRIIDYTDLPVIVKGIESPEDALYAIGAGAAGVYVSNHGGRQLNGGPASFDVLESVAKAVNHQVPVIFDSGVRRGSDVFKAIALGADLVGIGRPAIYGLALGGADGVYSVFEHLNNELKIVMQLAGTKTIEDIKHAELLNVQY

SEQ ID NO: 94 - protein, 368 aa, Enterococcus sp.
MTYQASNEEHPIDIVNIASLEQRVKERMEKGAFGYIRGGSEDEWTMKENTTSFNNKTIMPRVLRGIDRADLRTKLWDMDLATPIIQAPSAAQGLAHENGEADTAKGVADAGSIFSISTYANTTIEDAAAAAPNAPQFFQLYMSKDDGFNEFILDKAVKAGAKAIILTADSTLGGYREDDVINHFQFPLPMPNLAAYSAKSDSGDGKGKGIAEIYAAAKQGLVPEDIKKIKDLTNLPVFVKGIQSPEDAEVAIAAGADGIWISNHGGRQLDGGPASFNVLPLIAGVVNKRVPIVFDSGVRRGEHVFKALASGADLVAIGRPVIYGLNLGGAEGVKSVFDHLNKELSITMQLAGTKTIEEVKNTTLHDAK

SEQ ID NO: 95 - protein, 362 aa, Latilactobacillus sakei
MTYPTSDAEHPVDVLNLVSLEGRVKERMEAGAFGYIRGGSEDEWTMAENTSAFNQKKIMPRVLQGVEHADLHTKLWDIDLKTPIIQAPSAAQGLAHEKGEVDTAKGMAAAGSIFSISTYANTLVEDAAAAAPDAPQFFQLYMSKDDQFNEFLLQKAVNAGVKGIVLTVDSTLGGYRETDIETKFQFPLPMPNLAAYSNSDGAGKGISETYATAKQGIVPEDIQKIKEITHLPVIIKGIQSPVDAELTVQAGADGIWVSNHGGRQLDGGSASFEVLPLVAQQVAKRVPIIFDSGVRRGEHVFKALASGADLVAIGRPIIYGLNLGGAQGVTSVIDHMNHELSITMQLAGTKTIDAVKETTLLD

SEQ ID NO: 96 - protein, 369 aa, Lactococcus reticulitermitis
MSYITSTQERHIDILDIPSLEPKVGANMAKGAFGYLSGAAEDELVLKANPLAFNHKLIAPRVLQDIENPDLTTEFLGLKLSAPIIAVPIAAHGLVHEKAELDTAQGVADAGTIFSLSTYGNATVDDVAKVVPEAPKFFQLYMSKDDNFNRWILDKAVKGGYKAIILTADSTLGGYRESDIVNNFTFPNVMRNLEEWSKLSSDGETGSGEGIAAIYAKAKQALSLKDIAFIKDYTHLPVFVKGVQSPKDVDPLIAAGVDGIWVSNHGGRQLDGGPASFDVLADIAKVVNKRVPIVFDSGVRRGQHIFKALASGADVVGIGRPMLWGLNLGGRQGVTDVFDHFKKELMITMQLAGTHNVAEIKATELIDAK

SEQ ID NO: 97 - protein, 403 aa, Acinetobacter sp.
MKSKHHKIVLSISTLAIAIATSFSNAAETSYKASSVNQPIKIVNIASLEAQAKAQMQPGAFGYIRGGAEDENNLRSNTSAFDKKYIMPRIMQAIELKDINLNTKFLGIDLKTPIIQAPMAAQGLANVDGELSTAKGMAKAGSIFSLSTYGNKTIEEVAQVSGKNPFFFQLYMSKNDKFNEFTLKRAKEHGAKAIILTVDSPVGGYREDDIKNNFQFPLGFANLELFAQQNKDGSKTGKGAGISEIYAQAKQAFTPSDIQYVKKLTGLPVIVKGIQSPEDADMAIRAGADAIWVSNHGGRQLDSGPSSFDVLPGIAKVVNKRVPIVFDSGVRRGSHVFKALASGADVVAIGRPILYGLQLGGAEGVNSVMEQFNKELRISMMLGGAKDIEAVKKTKLYTDKDFE

SEQ ID NO: 98 - protein, 366 aa, Enterococcus sp.
MTYQASNEEHPIEIVNIASLEGRVKERMDKGAFGYIRGGSEDEWTMKENTSSFNNKTIMPRVLRGIDHADLSTKLWDIDLATPIIQAPSAAQGLAHEKGEADTAKGVAAAGSIFSISTYANTTIEDAAAAAPDAPQFFQLYMSKDDGFNEFILEKAVKAGAKAIILTADSTLGGYREEDIINKFQFPLPMPNLAAYSAKSASGDGEGKGIAEIYAAAKQGLVPEDIKKIKDLTNLPVFVKGIQSPEDAEVAIAAGADGIWISNHGGRQLDGGPASFDVLPLIAGVVNKRVPIVFDSGVRRGEHVFKALASGADLVAIGRPVIYGLNLGGTEGVKSVFDHLNKELSITMQLAGTKNIDEVKNTRLND

SEQ ID NO: 99 - protein, 369 aa, Lactococcus raffinolactis
MSYITSTQERHIDILDIPALETKVAANMEKGAFGYLSGAAEDELVLKANPLAFNHKLIAPRVLRDIENPDLTTEFLGLKLSAPIIAVPIAAHGLVHEHAELDTAQGVADAGTIFSLSTYGNATVDDVAKIAPESPKFFQLYMSKDDNFNRWILDKAVKGGYKAIILTADSTLGGYREADIVNNFTFPNVMRNLEEWSKLSSEGETGSGEGIAAIYAKAKQALSLKDIAFIKDYTKLPVFVKGVQSSKDVEPLIEAGVDGLWVSNHGGRQLDGGPASFDVLADIAKVVNKRVPIVFDSGVRRGQHIFKALASGADVVGIGRPMLWGLNLGGSQGVTDVFDHFKKELMITMQLAGTHNVAEIKATELLDAK

SEQ ID NO: 100 - protein, 373 aa, Lactococcus raffinolactis
MSYITSTQERHIDILDIPALETKVAANMEKGAFGYLSGAAEDELVLKANPLAFNHKLIAPRVLQDIENPDLTTEFLGLKLSAPIIAVPIAAHGLVHEHAELDTAQGVADAGTIFSLSTYGNATVDDVAKIAPESPKFFQLYMSKDDNFNRWILDKAVKGGYKAIILTADSTLCGYREADIVNNFTFPNVMRNLEEWSKLSSDGETGSGEGIAAIYAKAKQALSLKDIAFIKDYTKLPVFVKGVQSSKDGEPLIEAGVDGIWVSNHGGRQLDGGPASFDVLADIAKVVNKRVPIVFDSGVRRGQHIFKALAIGADVVGIGRPMLWGLNLGGSQGVTDVFNHFKKELMITMQLAGTHNVAEIKATELLDAKLTIG

SEQ ID NO: 101 - protein, 398 aa, Proteus mirabilis
MKSKILKTTAIAMALSVGVAQAAEYKASTAEGPIKIVNLKAMEAQVQANMDKGAFGYIRGGAEDENNLRSNTTAFDKKYIMPRSLQGIEFSDLDLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIILTVDSPVGGYREDDIKNSFQFPLGFANLEAFAKISDDKSKTGKGAGISEIYAQAKQAFTPADIQYVKKMSGLPVIVKGIESPEDADMAIKAGADAIWVSNHGGRQLDSAPATIDVLPAIAKVVNKRVPIVFDSGVRRGSHVFKALASGADIVAVGRPILYGLNLGGAEGVNSVIQHLNKELKINMMLGGAKTVKDIQATQLYTDASFNQ

SEQ ID NO: 102 - protein, 362 aa, Latilactobacillus sakei
MTYHTSDAEHPIDVLNLASLEGRVKERMEADAFGYIRGGSEDEWTMAENTSAFNQKKIMPRVLQGVEHADLHTKLWDIDLKTPIIQAPSAAQGLAHEKGEVDTAKGMAAAGSIFSISTYANTLVEDAAAAAPDAPQFFQLYMSKDDQFNEFLLQKAVKSGVKGIILTVDSTLGGYRETDIETKFQFPLPMPNLAAYSNSDGAGKGISEIYAAAKQGIVPEDIQKIKEITHLPVIVKGIQSPVDAELAVQAGADGIWVSNHGGRQLDGGPASFEVLPLVAQQVAKRVPIIFDSGVRRGEHVFKALASGADLVAIGRPIIYGLNLGGAQGVTSVIDHLNHELSITMQLAGTKTIDAVKETTLLD

SEQ ID NO: 103 - protein, 366 aa, Companilactobacillus alimentarius
MKKKYEASTEEKHLEIVNIASLEKRVKDHMSNEKGAFGYIRGGSEDEWTMKQNTEAFSKKKIMPRVLQGIDHADLSTKLWDIDLKTPIIEAPSAAHGLAHAKGEVDTAKGVADAGSIFSMSTYGSTSVQDAAAAAPDAPQFFQLYMSKDDKFNEFLIKEAVKAGVKAIVLTVDSTLGGYREEDIINKFQFPLPMPNLAAFSEGDGEGKGISEIYAAAKQGIVPSDIQKIKDMSGLPVIVKGIQSPDDAELAIDFGADGIWISNHGGRQLDGGPASFDVLPTIASAVDKRVPVIFDSGVRRGEHVFKALASGADLVAVGRPIIYGLNLGGAQGVTDVINHLNMELSITMQLAGTKTINDVKNNQLLD

SEQ ID NO: 104 - protein, 410 aa, Lactobacillus crispatus
MTVYYKGFPQSDRDEYLHMINLQELEEKVKQVMPEAAYYYIASGAENEWTWRNNTAAFNHYQIVPRALTNMQDPQTDTEFMGMKLKTPVMISPIACHGIAHKDAEVATQKGAAAAGALFASSTYANKSVEDIAAAAPNAPRFFQLYLSKDWKFNRMVFDAIKKTGYKGIYLTVDALVSGYREANLRTHFTYPVPLDFFTRYIGAKGEGQSVAQMYASSAQKIGPKDVQRIKDETGLPVIVKGIECPEDAFKAIGAGADGVYVTNHGGREVDGGPATIDVLPSIAKAVAHRVPIIFDSGVRRGSHVFKALALGADMVGIGRPYLYGLALGGAKGVESVIDQLDKELKIDMQLTGCKTIEDVKHAKINHISYTADNLPSNTSPSRMKPYPVTADNQIKKDSAADASSGASEH

SEQ ID NO: 105 - protein, 366 aa, Companilactobacillus nuruki
MNKKYEASTAENHVDIVNIASLEARVKDHMSNEKGAFGYIAGGSEDEWTKKQNTESFNHKSIMPRVIQGIDHADLSTKLWDIDLKTPIIQAPSAAQGLAHAKGETDTAKGVAAAGSIFSISTYASTSVEDAAAAAPDAPQFFQLYMSKDDKFNEFLIKKAVAAGAKAIILTVDSTLGGYREADVVNKFQFPLPMPNLAGYSAGDGEGKGISEIYASAKQGIVPSDIQKIKDMSGLPVFVKGIQSPDDAELAIDFGADGIWVSNHGGRQLDGGPASFDVLPYIAEVVDKRVPIVFDSGVRRGEHVFKALASGADLVAIGRPIIYGLNLGGAQGVTDVIDHLNMELSITMQLAGTKTINDVKNTELLD

SEQ ID NO: 106 - protein, 368 aa, Enterococcus avium
MTYQASNEEHPIEIVNIASLEGRVKERMEKGAFGYIRGGSEDEWTMKENTSSFNKKTIMPRVLRGIDHADLSTKLWNIDLLTPIIQAPSAAQGLAHEKGEADTAKGVAAAGSIFSISTYANTTIEDAAAAAPNAPQFFQLYMSKDDGFNEFILEKAVKAGAKAIILTADSTLGGYREEDIVNKFQFPLPMPNLAAYSAKSASGDGEGKGIAEIYAAAKQGLVPEDIKKIKDLTSLPVFVKGIQSPEDAEVAIAAGADGIWVSNHGGRQLDGGPASFDVLPLIAGVVNKRVPIVFDSGVRRGEHVFKALASGADLVAIGRPVIYGLNLGGAEGVKSVFDHLNKELSITMQLAGTKTINEVKNTRLHDAK

SEQ ID NO: 107 - protein, 366 aa, Companilactobacillus formosensis
MKKKYEASTAENHVDIVNIAGLEARVKDHMSNEKGAFGYIRGGSEDEWTMDQNTKAFNSRKIVPRVLQGIDHADLSTSLWDIALKTPIIQAPSAAQGLAHANGEKDTAKGVADAGSIFSISTYASTSVEDAAAAAPDAPQFFQLYMSKDDKFNEFLIKKAVAAGVKAIVLTVDSTLGDYREADVINKFQFPLPMPNLVGYSAGDGEGKGISEIYASAKQGIVPTDIQKIKDMSGLPVIVKGIQSADDAELAVDFGADGIWVSNHGSRQLDGGPASFDVLPAIAGTVDKRVPIIFDSGVRRGEHVFKALASGADLVAIGRPIIYGLNLGGAQGVTDVINHLNMELSITMQLAGTKTIADVKNTQLIY

SEQ ID NO: 108 - protein, 400 aa, Morganella morganii
MQRSKAGVFAVAALGLAMTLSTAYAAEYKASTKEGPVKIVNLNDLESQVKANMDKGAFGYIRGGAEDEKNMRDNTAGFDRKYIMPRVMQGIELKDIDLSTSFLGIPLATPVIQAPMAAQGLAHRDGEIATAKGMAKAGSVFSLSTYGNKTIEEVAAVSDGHPFFFQLYMSKNNAFNEFTLKRAKESGAKAIILTVDSPVGGWREDDLRNNFQFPLGFANLELFAAQNNDGSKTGKGAGISEIYAQAKQAFTPDDIQYVKNLSGLPVIVKGIQSPEDADRVIEAGADAIWVSNHGGRQLDSGPASFDVLPSIAKVVNKRVPIVFDSGVRRGSHVFKALASGADVVAVGRPVLYGLNLGGAEGVNSVIQQLNKELRINMMLGGTKDINAVKQTRLYSDRDFQ

SEQ ID NO: 109 - protein, 369 aa, Levilactobacillus bambusae
MTEVNGYEQSDREQHIDVLNLESLEPEAEKIIPTGGFGYISGGSEDDWTLAANREAFTHKQIYPRVLSDMDNPDLSTTVFGVNVKTPIMMSPLAAQGLANSQGEKDTARAFAAEGAFMGQSTYSSTSIADTAAAGNGGPQFFQLYMSKDWDFNEALLDEAKKAGVKGIILTADATVDGYREDDIVNDFQFPIPMANLIQFSEASGQGKGIKEIYAAAAQRISPDDVKRIADYSGLPVIVKGIQDPNDADLAIGAGAAGIYVSNHGGRQLNGGPASFDVLPAIAEAVNGRVPVIFDSGVRRGSDVFKALASGADLVAMGRPFVYALALGGAMGVQDALQEINEEFKTVMQLAGTKTIEDVKHAQLADFRY

SEQ ID NO: 110 - protein, 417 aa, Lactobacillus rodentium
MTVYYKGFPQSTREKKLNLVNLNQLEDEVKYVMPEAAYYYIASGSENEWTWRNNTQAFNHFQIVPRALTDRSNPSTDTTFMGMDLKTPVMISPIACNGISHAEAEVDVAKGAKAAGALYSMSTYANKSVEDVQKAVGESPRFFQLYLSKDWDFNRMVITDALKAGFSGFFLTVDATVSGYREANLRTHFTYPVPLAFFNKWSGGKGEGQSVAQMYASSAQKIGPEDIKKIKEIAGKDVPVFVKGVECAEDAFLAIGAGADGIVVSNHGGRQLDGAPATIDVLPEIAKEVHHSDHRIPIILDSGVRRGSHVFKALALGADLVGIGRPYLYGLALGGALGVESVIEQLNKELLIDMQLTGCKTIEDVKHAKIDHISYSADWGVSSTSPSVRKPYPVTKENQLEKDADSTDAESGASEHE

SEQ ID NO: 111 - protein, 401 aa, Providencia huaxiensis
MLNFKKNTVVTSTLLAVALTLGTAQAAEYKASDKEGPIKIVNLDELESQVAKNMEKGAFGYIRGGAEDELNLNKNTQHFDKKYIMPRVMQGIEISDIDLSTDFLGIKLKTPIIQAPMAAQGLAHKEGEIATAKGMAKAGSIFSLSTYGNKTIEEVAEVSGNNPFFFQLYMSKNEAFNEFTLKRAKESGAKAIILTVDSPVGGYREDDIRNNFQFPLGFANLELFAQQNSDGSKTGKGAGISEIYAQAKQAFTPADIQYVKKLSGLPVIVKGIQSPEDADVVIKAGADAIWVSNHGGRQLDSGPASFDVLPSIAKVVNKRVPIVFDSGVRRGSHVFKALASGADVVAVGRPILYGLNLGGAEGVNSVIQQLNKELSINMMLGGAKNIEGVKATQLYTDKDFQ

SEQ ID NO: 112 - protein, 402 aa, Pseudochrobactrum asaccharolyticum
MTKFMSKKSVLSLSAIALALSLGVAHAADYKASTAEGPVKITNLNELEGQVKANMDKGAFGYIRGGAENEQNLRSNTSVFDKKYIMPRVMQGIELSEIDLKTNYLGIDLKTPIIQAPMAAQGLAHQDGEIATAKGMAKAGSIFSLSTYGNKTIEEVAAVSDGNPFFFQLYMSKNEAFNEFTLKRAKDSGAKAIILTVDSPVGGYREDDIKNDFQFPLGFANLELFAKQNDDGSKTGKGAGISEIYAQAKQAFTPSDIKYVKDMTGLPVIVKGIQSPEDADVVIKAGADSIWVSNHGGRQLDSGPASFDVLPSIAKVVNKRVPIVFDSGVRRGSHVFKALASGADVVAVGRPILYGLNLGGAEGVNSVIQQLNKELTINMMLGGAKNIEAVKKTKLYSDKDFE

SEQ ID NO: 113 - protein, 366 aa, Enterococcus durans
MAYQASTEEHSIDIVNIASLEGKVKERMEAGAFGYIRGGSEDEWTMKENTTSFNTKKIMPRVLRGIDSADLSTSIFGIDLKTPIIQAPSAAQGLAHEKGEANTAKGVAAAGSIFSISTYANTTIQDAAAAAPGAPQFFQLYMSKDDGFNEFILNKAVEAGAKAIILTADSTLGGYREEDIINHFQFPLPMPNLAAYSEQSASGNGEGKGISEIYAAAKQGLTPEDIKKIKDLTNLPVIVKGIQSPEDAEVAISAGADGIWVSNHGGRQLDGGPASFEVLPKVAEVVNKRVPVIFDSGVRRGEHVFKALASGADLVAIGRPVIYGLNLGGAQGVTSVFDHLNKELSITMQLAGTRTVNEIKNTKLLD

SEQ ID NO: 114 - protein, 401 aa, Salmonella enterica subsp. indica
MMKTNDLIRVVVSLAMLATSGLTYAEEYKASADEKAIKMTNVASLEARVQARMEKGAFGYIRGGAEDENNLRSNTESFDKKYIMPRVLQGIELKEIDLSTQLLGIPLKTPIIQAPMAAQGLAHVSGELATAKGMAQVGSIFSLSTYGNKTIEEVANVSGESPFFFQLYMSKNNQFNEFILAQAVKHGAKAIILTVDSPVGGYREEDIKNNFQFPLGFANLEMFARENDDGSKTGKGVGISEIYAQAKQAFTPEDIAYVHRVSGLPVIVKGIQSPEDAETAIQAGAAGIWVSNHGGRQLDSGPSSFDMLPAIAKVVNKRVPVIFDSGVRRGSHVFKALASGADIVAVGRPILYGLNLGGAQGVASVIEQLNKELTINMMLGGARNIEQVKTTRLLTENELPQ

SEQ ID NO: 115 - protein, 367 aa, Companilactobacillus zhachilii
MNKKKYEASTAENHVDIVNLASLEGRVKDHMSNEKGAFGYIRGGAEDEWTLNENTEAFNDKEIMPRVLQGIDHADLSTKLWDIDLKTPIIQAPSAAQGLAHIKGETDTAKGVAAAGSIYSISTYASTSVEDAAAAAPDSPMFFQLYMSKDDKFNEFLIKKAVKAGAKAIIMTVDSTLGGYREADVINKFQFPLPMPNLAGYSAGDGEGKGISEIYASAKQGIVPTDIQKIKDMSGLPVFVKGIQSPDDAELAIQFGADGIWVSNHGGRQLDGGPASFALLPYIAEVVDKRVPIVFDSGVRRGEHVFKALASGADLVALGRPIIYGLNLGGAQGVTDVIEHLNKELAITMQLAGTKTIDDVKNTELLD

SEQ ID NO: 116 - protein, 366 aa, Lactococcus allomyrinae
MAYITSSDEEKVEIVNIKSLETRVKERMEAQGNKGAFGYIRGGSEDEWTMAENTTAFNHKKIVPRVLRGVDSADLSTSVLGINLKTPIIQAPVAAQGLAHMKGEVDTAKAMAEVGSLFSISTYGSTSVEEAAAVAPGAPQFFQLYMSKDDHFNEFLLKKAVEAGVKAIILTADSTLGGYREEDVINHFQFPLPMPNLAAFSESDGTGKGIGEIYAAAKQGLVLEDIQKIKAITGLPVLVKGIQSPDDALLAIEAGADGIWVSNHGGRQLDGGPASFDVLPAIAKAVNKRVPVIFDSGVRRGEHVFKALASGADLVAVGRPVLYGLNLGGALGVQSVFEHLNKELSITMQLAGTKTIDEIKNTALID

SEQ ID NO: 117 - protein, 368 aa, Acidaminococcus sp.
MTYYTSMEEHPIDILNLPSLEERVKANMERGAFGYIRGGSEDEWTLRENTRAFDDLQIIPRVLQGLSGADLSTSIFGISLKTPVIEAPSAAHGLAHVKGEIDTAIGTAAAGSLFSLSTYGSTDLRDVAAAAPGAPQFFQLYMSKDDGFNAYLVKKAVKAGVKAIILTVDSTLGGYREEDVRNHFQFPLPMPNLAAYSSQDGVGKGIAEIYAAAKADFVPSDIDKIKTLSGLPVLVKGIQSPEDAEAAIKAGADGIWVSNHGGRQLNGGPASITVLPSIASVVRRRVPIVFDSGVRRGSHVFKALASGADLVALGRPLIYGLNLGGAEGVKSVFDQINHELSIVMQLAGTKDIEAIKRTPLLHKAAPLV

SEQ ID NO: 118 - protein, 400 aa, Morganella sp.
MKKVKTGLFTATVLGLAMTLTTAYAAEYKASIKEGPLKIVNLNDLESQVKANMDKGAFGYIRGGAEDEKNMRDNTASFDRKYIMPRVMQGIELKDINISTSFLGIPLDTPVIQAPMAAQGLAHRDGEIATAKGMAKAGSIFTLSTYGNKTIEEVAAVSDGHPFFFQLYMSKNDAFNEFTLKRAKESGAKAIILTVDSPVGGWREDDLRNNFQFPLGFANLELFAKQNNDGAKTGKGAGISEIYAQAKQAFTPSDIQYVKKMSGLPVIVKGIQSPEDADRVIEAGADAIWVSNHGGRQLDSGPASFDVLPAIAKTVNKRVPVVFDSGVRRGSHVFKALASGADIVAVGRPVLYGLNLGGAQGVDSVIQQLNKELRINMMLGGAKDINAVKQTKLYTDNDFQ

SEQ ID NO: 119 - protein, 365 aa, Enterococcus pseudoavium
MAYQTSNEEHPIEIVNIASLEGRVKERMDKGAFGYIRGGSEDEWTMKENTTSFNNKTIMPRVLRGVDQADLSTKLWDIELATPIIQAPSAAQGLAHEKGEADTAKGVAAAGSIFSISTYANTTIEDAAAAAPEAPQFFQLYMSKDDGFNEFILEKAVKAGAKAIILTADSTLGGYREEDIINNFQFPLPMPNLAAYSEKSASGDGEGKGIAEIYAAAKQGLVPEDIKKIKDLTNLPVFVKGIQSPEDAEVAIAAGADGIWVSNHGGRQLDGGPASFDVLPLIAAVVNKRVPIVFDSGVRRGEHVFKALASGADLVAIGRPVIYGLNLGGAEGVKSVFDHLNKELSITMQLAGTKTLDEIKNTKLM

SEQ ID NO: 120 - protein, 361 aa, Intestinibaculum porci
MAYYTSNAEHPIDIVNIASLENRVKARMDRGAFGYIREGAEDEWTLRENTRAFNDLRIAPRVLQGIDHVDLSTSIFGIDLKTPIIEAPSAAHGLAHVKGEVDTAIGAAKAGTLFAMSTYGSTPVEEAAAAAPEAPQFFQLYMSKDDGFNEFLIKKAVKAGVKAIIMTVDSTLGGYREEDIVSHFQFPLPMPNLAAYSASDGVGKGISEIYAAAKQDFVPSDIAKVKDMSGLPVFVKGIQSPEDAIVAIQAGADGIWVSNHGGRQLDGGPASISVLPAIAKAVNHQVPVIFDSGVRRGQHVFKALASGADLVAIGRPVIYGLNLGGAEGVKSVFDHLNMELSITMQLAGTKTIEEIKKTTLL

SEQ ID NO: 121 - protein, 409 aa, Lactobacillus sp.
MTSYYNGFPQSDRDEAINMINLDELEERAKNVMPEGAYYYIASGSENEWTWRNNTTAFNHFQIIPRALTNMTDPQLDTEFMGMKLKTPVMISPIACHGIAHKDAEVATQIGAAAAGALFSSSTYANKSVEDIAAAAPEAPRFFQLYLSKDWDFNKMVFDAVKKAGYKGIFLTVDALVSGFREANLRTKFAYPVPLDFFTRYQGAKGEGQTVAQMYASSAQKIGPEDVKRIKEMSGLPVFVKGVMCAEDAYLAMGAGADGIVVTNHGGREVDCGPATIDMLPEIAKAVNHRVPIIFDSGVRRGSHVFKALALGADLVGIGRPYLYGLALGGAKGVQSVIEQLNKELLIDMQLTGCKTIEDVKRARITHINYTADNLRSNTDPTRIKPYPVTKENQMKVDDSDTVSGASHS

SEQ ID NO: 122 - protein, 409 aa, Lactobacillus sp.
MTAYYNDFPQSDRDEYIKMINLDELEERAKKVMPEGAYYYIASGSENEWTWRNNTVAFNHFQIVPRALTNMADPQLDTDFMGMKLKTPIMISPIACHGIAHQDAEVATQKGAAAAGALFSSSTYANKSVEDIAAAAPEAPRFFQLYLSKDWNFNQMVFDAIKKAGYQGIFLTVDALVSGFREANLRTQFAFPVPLDFFTRYQGTKGEGQTVAQMYASSAQKIGPEDVKRIKEMSGLPVFIKGVVCAEDAYLAMGAGADGIVVTNHGGREIDGGPATIDMLPEIAKAVDHRVPIVFDSGVRRGSHVFKALALGADLVGIGRPYLYGLALGGAKGVQSVIEQLNKELLIDMQLTGCKTVADIQHAKITNINYTADNLSSNTDPERIKPYPVTKENQLKAGGMDTVSGASHS

SEQ ID NO: 123 - protein, 369 aa, Lactobacillus sp.
MVVVNGYKQNENEKKLNVLNLDQLEKQAKEIIPTGGFGYISGGSEDEWTLRENRRAFTHKQIVPRALTNIEKPELETNVFGIPLKTPLFMVPAAAQGLAHAKGEVDTAKGVAAVGGLMAQSTYSSTSIADTAASGNGAPQFFQLYMSKDWDFNEALLDEAKRAGVKGIILTVDATVDGYREADIINNFQFPIPMANLTKYSEGDGQGKGIAEIYASAAQKIGPDDVARIANYTDLPVIVKGIESPEDALYAIGAGASGIYVSNHGGRQLNGGPASFDVLEDVAKAVNGKVPVIFDSGVHRGSDVFKALASGADLVGIGRPVIYGLALGGAQGVQSVFEHLDHELEIIMQLAGTKTISDVKNAKLLNIRY

SEQ ID NO: 124 - protein, 369 aa, Lentilactobacillus curieae
MTVINGYEQSDREEKLNVLNLPSLEAEAKKIIPTGGFGYISGGSEDEWTLNHNSEAFNHVQIVPRALTNMENPSTATSIFGLDLKTPIMMAPAAAQGLAHSRGEVATAEGVSAAGALMGQSTYSSTSIADTAIAGHGAPQFFQLYMSKDWSFNESLLDEAKRSGVKAIILTVDATVDGYREADIINKFQFPIPMANLTKFSEGDGQGKGIEEIYASAAQKISAEDVKRIADYTDLPVIVKGIQSPEDALLAIGAGAQGIYVSNHGGRQLNGGPASFDVLADIAKAVNHRVPIIFDSGVRRGSHVFKALASGADIVALARPIIYGLALGGAQGVQSVIEELNHELEIDMQLAGTKDIEAVKHAKLIKINY

SEQ ID NO: 125 - protein, 397 aa, Proteus hauseri
MKNKILKTTAIAMALSIGVAQAAEYKASTAEGPIKIVNLNAMENQVKGNMEKGAFGYIRGGAEDENNLRANTKAFDKKYIMPRALQGIEFSDLDLKTEFLGIKLDTPIIQAPMAAQGLAHQQGEVATAKGMAKAGSIFSLSTYGNKTIKEVADAKPGYPFFFQLYMSKNDAFNEYILSQAKQYGAKGIIMTIDSSVGGYREDDVKNNFQFPLGFANLEAFAKISDDKSKTGKGAGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADVVAIGRPILYGLNLGGAEGVNSVIQQLNKELKINMMLGGTKTVKDIQATQLYTEADFK

SEQ ID NO: 126 - protein, 367 aa, Enterococcus asini
MSYQTSNEEKDIEIVNVAGLEEEVRPRMDAGAFGYIRGGAETEWTMKENTAAFNKRQITPRVLQGISSADLSTKLWDIDLKTPIIEAPSAAQGLAHEKGELDTAKGVAAAGSIFSISTYANTTIEAAAQAAPGAPQFFQLYMSKDDGFNEFIIKKAVAAGAKAIVLTVDSTLGGYREADVINHFQFPLPMPNLAAYSAQSASGDGEGKGIAEIYAAAKQDFVPEDIQKIKDLSGLPVIVKGIQSPLDAEVAIQAGADGIWVSNHGGRQLDGGPASFDVLPGIAKVVAKRVPVIFDSGVRRGEHVFKALASGADLVAIGRPVIYGLNLGGAQGVKAVFDHLNKELAITMQLAGTPTIDAVKKTPLVQF

SEQ ID NO: 127 - protein, 408 aa, Lactobacillus xujianguonis
MTYYYNGFPQSERDQKLNMVNVDDLEEKVKQIMPEGAYYYIASGSENEWTWRNNTAAFNHYQIVPRALTDMSDPQTDTEFFGMKLKTPVMISPIACHGIAHKDAEVATQKGAAAAGALFSSSTYANKSVEDIAAAAPDAPRFFQLYLSKDWNFNKMVFDAIKKAGYKGIFLTVDALVSGYREANLRTNFTYPVPLDFFTRYMGAKGEGQSVAQMYAASAQKIGPEDVRRIKEETGLPVIVKGVMCAEDAFKAIGAGADGVYVTNHGGREVDGAPATIDVLPEIAKAVDHRVPIIFDSGVRRGSHVFKALALGADMVGIGRPYLYGLALGGAKGVESVIEQYNKELKIDMQLTGCKTIEDVKHAKIDHIPYTADNLKSNTDPSRMKPYPKTEENQIKGSADATSGASHH

SEQ ID NO: 128 - protein, 368 aa, Enterococcus avium
MTYQASNEEHPIEIVNIASLEGRVKERMEKGAFGYIRGGSEDEWTMKENTSSFNKKTIMPRVLRGIDHADLSTKLWDMDLATPIIQAPSAAQGLAHKKGEADTAKGVAAAGSIFSISTYANTTIEDVAAAAPSAPQFFQLYMSKDDGFNAFILEKAVKAGAKAIILTADSTLGGYREEDIVNKFQFPLPMPNLAAYSAKSASGDGEGKGIAEIYAAAKQGLVPEDIKKIKDLTSLPVFVKGIQSPEDAEVAIAAGADGIWVSNHGGRQLDGGPASFDVLPLIAGVVNKRVPIVFDSGVRRGEHVFKALASGADLVAIGRPVIYGLNLGGAEGVKSVFDHLNKELSITMQLAGTKTINEVKNTRLHDAK

SEQ ID NO: 129 - protein, 412 aa, Lactobacillus johnsonii
MTTYYKGFPQSTREEKLHMVNLDELENEAKYVMPEAAYYYVASGAENEWTWRNNTQAFNHFQIVPRALTGMQDPELNTEFLGMKLKTPVMICPIACHGIANAEAEIDTAKGAKVAGALFAMSTYANKSVQEVQNAVGDSPRFMQLYLSKNWDFNKMVIEESVKAGFSGFFLTVDALVSGYREANLRTNFTYPVPLAFFNEWNGGKGEGQSVAQMYASSAQNIGPDDIRRIKEIADVPVIVKGVECAEDAMLAIGAGADGIVVSNHGGREVDGAPATIDVLPEIAKAVKSCDHRVPIILDGGVRRGSHVFKALALGADLVGIGRPFLYGLALGGAQGVQSVIEQLNKELLIDMQLTGCKTIEDIKKAKIDHINYGADWGISSTSRSVMKPYPVTKENQLTGEAADAVSGASRH

SEQ ID NO: 130 - protein, 365 aa, Enterococcus florum
MTYHTSNEEHPIDIVNIASLEELVKQRMDKGAFGYFRGGSEDEWTMKENTFAFTKKSIMPRVLRGIDQPDLRTTLWDIDLATPIIQAPSAAQGLAHEKGEVDTAKGVAAAGSIFSISTYANTTIEHAAAAAPEAPQFFQLYMSKDDGFNRFILEKAVDAGAKAIILTADSTLGGYREEDVINHFQFPLPMPNLAAYSEQSNQSDGEGKGIAEIYAAAKQALVPEDIKKIKELTNLPVFVKGIQSPEDAEVAISAGADGIWISNHGGRQLDGGPASFDVLPLVAGVVNKRVPIVFDSGVRRGEHVFKALASGADLVAIGRPVIYGLNLGGAEGVKSVFDHLNKELAITMQLAGTKTVEEIKNTNLL

SEQ ID NO: 131 - protein, 369 aa, Weissella cryptocerci
MAYHTSTEERPIDIVNIPALEPIVKAKMNKGAFGYLAGGAESEITLRENVTSFEHKKILPRVLRNVEKPDMSTELFGIHIDAPIIAAPIAAHGLVHEQAEKDTVQGVGAAGSIFSLSTYGNATVDEVAQASPDTAKFFQLYMSKDDDFNHWILDMAVNDGYKAIILTADSTLGGYRESDIMNNFAFPLPMKNLAAWAAKTAGPDSGEGEGIAAIYAKAKQKLSLADIKAIKDYTHLPVIVKGVQSPLDIEDLLNAGADGIWVSNHGGRQIDGAPGSFDTLAEIAAVVAKRVPVIFDSGVRRGQHIFKALASGADIVAIGRPMLWGLSLGGVQGVTDVYNHFKKELTIDMQLAGTQTIADVKNTVLADAK

SEQ ID NO: 132 - protein, 367 aa, Lactiplantibacillus paraplantarum
MTVINGYEQSDREQKLAILNLPSLEAAAKKIIPTGGFGYISGGSEDEWTLKQNTAAFNHVQIVPRALTDMEKPSTQTQVFGIDLKTPIMMAPAAAQGLAHARGEAATAEGMAQVGALMAQSTYSSTSIADTAAAGKGAPQFFQLYMSKDWDFNQSLLDEAVKAGAKAIILTVDATVDGYREADIINNFQFPIPMANLTKFSEGDGKGKGIMEIYAAAAQKISPADVRRITEYTNLPVIVKGIQSPEDALLAIGAGAQGIYVSNHGGRQLNGGPASFDVLHAIAQAVSGRVPIIFDSSVRRGSHVFKSLANGADLVALARPVIYGLALGGAQGVASVISHLNEELLVDMQLAGTKTIEDVKHAKLLRK

SEQ ID NO: 133 - protein, 403 aa, Pseudomonas sp.
MKTPPLSKMALASGLLTAVLSLNVAQAQEYRASDAEKAIKIVNIGALEAEVKASMDKGAFGYIRGGAEDENNMRSNTEHFDKKYIMPRIMQGIERTDIDLSTELLGIKLKTPIIQAPMAAQGLAHKEGEIATARGMAKAGSIFSLSTYGNKTIEEVATASKNNPFFFQLYMSKNEKFNEFTLKKARQFGAKAIILTVDSPVGGHREDDIRNNFQFPLGFANLELFAKQNSDGSKTGKGSGISEIYAQAKQAFTPADIAYVQKLSGLPVIVKGVQSPEDADMAIKAGAAAIWVSNHGGRQLDSGPSSFDVLPGIAKVVNKRVPIIFDSGVRRGSHVFKALASGADIVAVGRPILYGLHLGGSEGVNSVIEHLNKELSINMMLGGAKNIEAVKSTKLYTDKDFEG

SEQ ID NO: 134 - protein, 369 aa, Lentilactobacillus buchneri
MTVVNGYKQNDNEEKINILNLEESEERAKQIIPTGGFGYIVGGSENNWTLKANRKAFTHKQIVPRALSNIENPSLDTNVFGIPLKTPIMMAPTAAQGLAHSQGEKDTAKGVAAVGGLMAQSTYSSVSISDTAAAGNGAPQFFQLYMSKDWDFNYSLLDEAKKAGVKGIILTVDATVDGYREDDIKNNFQFPIPMANLTKFSEGDGKGKGIAEIYAAAAQKIGPDDVKKIADYTDLPVIVKGIESPEDALYAIGAGAAGVYVSNHGGRQLNGGPASFDVLEDVAKAVNGRVPIIFDSGVRRGSDAFKALASGADLVAMGRPVIYGLALGGAEGVQAVFEHLGDELKTTMQLAGTKTIADVKKTHLLSINY

SEQ ID NO: 135 - protein, 408 aa, Lactobacillus intestinalis
MTYYYNGFPQSEHDEKLNLINVDQLEEDAKRVIPEGAYYYIASGAENEWTWRNNTAAFNHYQIVPRALTDMEDPQLDTEFMGIKLKTPVMIAPIACHGIAHKDAEVATQKGAAAAGALFSSSTYANKSVEDIAAAAPDAPRFFQLYLSKDWNFNKMVFDAVKKAGYKAIMLTVDALVSGYREANLRTKFTYPVPLDFFTRYLGAKGEGQSVAQMYAASAQKIGPKDVARIKEESGLPVFIKGITCVEDAYKALGAGADGIYVTNHGGREIDCSPATIDVLPDIAKAVNHRVPIIFDSGVRRGSHVFKALALGADLVAIGRPYLYGLALGGAKGVESVIEQYNKELKIDMQLTGCKTIDDVKKAKIMHINYTADNLPSNTDPSRMKPYPVTSENQPQEDLDATSGASHH

SEQ ID NO: 136 - protein, 400 aa, Citrobacter sp.
MKVNKKILAIASVAMMISSGLAQAEAYKASTEEHPIKMVNIDSLEDQVKARMEKGAFGYIRGGAEDENNLRSNTESFDKKFIVPRVLQGIELKNINLSTSLLGIPLKTPIIQAPMAAQGLAHEAGELATARGMAQVGSIFSLSTYGNKTIEDVSRASEGNPFFFQLYMSKNNKFNEFILNEAVKHGAKAIILTVDSPVGGYREDDIKNNFQFPLGFANLEMFAKQSDDGSKTGKGSGISEIYAQAKQAFTPEDIKYVHQISGLPVIVKGIQSPEDAEVAIKAGAAAIWVSNHGGRQLDSGPSSFDVLPSIAKVVNKRVPIIFDSGVRRGSHVFKAIASGADIVAIGRPVLYGLNLGGAQGVASVIEQLNKELVINMMLGGTKDIEQVKKTKLLTLRDLEE

SEQ ID NO: 137 - protein, 365 aa, Companilactobacillus suantsaicola
MKKYEASTEEKHIEIVNIAGLEKRVRDHMSNEKGAFGYIRGGSEDEWTMKQNTEAFNKKKIMPRVLQGINHADLSTKLWDIDLKTPIIEAPSAAHGLAHAKGEVDTAKGVADAGTIFSMSTYGSTSLEDGAAAAPGAPQFFQLYMSKDDKFNEFLIKKAVKAGVKAIVLTVDSTLGGYREEDVISKFQFPLPMPNLAAYSNGDGEGKGISEIYAAAKQGIVPSDIQKIKDLSGLPVFVKGIQSPDDAELAIDFGADGIWISNHGGRQLDGGPASFDVLPAIASVVDKRVPIVFDSGVRRGEHVFKALASGADLVAIGRPIIYGLNLGGAQGVTDVIEHLNMELSITMQLAGTKTINDVKNTQLLD

SEQ ID NO: 138 - protein, 368 aa, Weissella confusa
MAYQGSTKEEALNIIDLPRLRAAVQRDTEAGAFGYVDGGSSDEQVLHDNETAFRHYQLIPRMLQNISAPDLSTTLLDIPLSMPVIAAPIAAHGLMHENGERVTVKGVGAAGTIFSLSTYGNSRIADVAASSPETPKFFQLYMSRDDEFNQYLLDEAVKNGYKAIILTADATLGGYREADIINNFAFPLPMENLAAFSNAAGSGEGLGIAEIYARAKQDLALSDITKVKQMANGLPVFVKGIQDPDDALAAIAAGADGIWVSNHGGRELNGAPASIDTLAAVAKAVNHRVPLVFDSGIRRGEDVAKAIALGADVVALGRPMLWGLNQGGAAGVQSVYEHLAEELKIVMQLTGSHTVAELQRAKIIDAKF

SEQ ID NO: 139 - protein, 369 aa, Schleiferilactobacillus harbinensis
MTVTNGYEQSDREQKLDFINLNDLEKEAEAIIPKGGFGYIRGGSEDEWTLRENTAAFNHAQIVPRALTDMENPATETDAFGLHFKTPLMMAPTAAQGLAHSQGEKDTARGVAAAGALMAQSTYSSTSIADTAAAGNGAPQFFQLYMSKDWDFNHHLLDEAKKAGIKGIILTLDAPVDGYREDDLRNHFQFPIPMANLTEYSEGDGSGKGIAEIYAAAAQKIGPKDIERIAAYTDLPVIAKGIMSPEDALKAIGAGAAGVYVSNHGGRQLNGGPASFDVLPSIAAAVNHQVPIIFDSGVRRGSHVFKALAAGADLVAFGRPAIYGLALGGAQGVQSVFEHLNDELKIDMQLAGTKTIADVKHAKLTHFPA

SEQ ID NO: 140 - protein, 366 aa, Lactococcus sp.
MAYITSSDEEKVEIVNIKSLETRVKERMEAQGNKGAFGYIRGGSEDEWTMVENTKAFNHKKIVPRVLRGVDSADLSTSIFGINLKTPIIQAPVAAQGLAHMKGEVDTAKAMAEVGSLFSISTYGSTSVEEAAAVAPGAPQFFQLYMSKDDHFNEFLLKKAVEAGVKAIILTADSTLGGYREEDVINHFQFPLPMPNLAAFSESDGTGKGIGEIYAAAKQGLVLEDIQKIKAITGLPVLVKGIQSPDDALLAIEAGADGIWVSNHGGRQLDGGPASFDVLPAIAKVVNKRVPVVFDSGVRRGEHVFKALASGADLVAVGRPVLYGLNLGGALGVQSVFEHLNKELSITMQLAGTKTIDEVKNTALID

SEQ ID NO: 141 - protein, 410 aa, Lactobacillus sp.
MTHYYEGFPQSDRNQYLHMVNLDQLEQDVKKVMPEGAYYYIASGAENEWTWRNNTAAFNHYQIVPRALTDMQDPVTDTEFMGMKLKTPVMISPIACHGIAHMDAEVATQKGAAAAGALFSSSTYANKSVADIAKAAPNAPRFFQLYLSKDWKFNEMVFDEIKKAGYKGIFLTVDALVSGYREANIRTHFTYPVPLAFFQEYMGAKGKGQSVAQMYASSAQKIGPEDVRRIKEATGLPVIVKGVECAEDAFKAIGAGADGIYVTNHGGREVDGAPATIDVLPSIAKAVNHRVPIVFDGGVRRGSHVFKALALGADIVGFGRPYLYALALGGAKGVESAINQLNKELKIDMQLTGCKTIDDVKHAKIDQFRYSADNLDSNTSFDRMKPYPKTSENQIKTGSQADATSGASEH

SEQ ID NO: 142 - protein, 408 aa, Lactobacillus jensenii
MTVYYKGFPQSDRNEAIKMVNVDELEDRVRKVMPEAAYYYIASGSENEWTWRNNTTAFNHFQIVPRSLTNMDSPSTATQFMGMDLKTPIMICPIACHGIAHKDAEVATAQGAKAAGALFSSSTYANKSVEDIAAATGDSPKFFQLYLSKDWDFNKMVFDAVKSAGYKGIMLTVDALVSGYREANLRTNFTFPVPLDFFTRYVGAEGEGMSVAQMYANSAQKIGPADVAKIKEMSGLPVFVKGIMNAEDAYMAIGAGADGIVVSNHGGREIDTAPATIDMLPEITAAVNGRVPIILDSGVRRGSHVFKALALGADLVGIGRPFLYGLALGGAKGVESVINQINNEFKILMQLTGCKTVEDVKHADIRQINYTADNLPSNTDPSVRRAYPVTKENQMEGTQDAATGASKH

SEQ ID NO: 143 - protein, 409 aa, Lactobacillus ultunensis
MTYYYKGFPQSDRDEKISMINVDELEERAKKVMPEGAYYYIASGAENEWTWRANTSAFNHYQIVPRALTDMQDPQTDTQFMGMKLKTPIMISPIACHGIAHKDAEVATQKGVAAAGALFSSSTYANKSVEDIAAVAPEAPRFFQLYLSKDWDFNKMVFDAIKKADYKGIFLTVDALVSGYREANLRTKFTYPVPLDFFTRYLGAKGEGQSVAQMYAASAQKIGPEDVARIKKESGLSVFVKGVMCAEDAYKAIGAGADGIYVTNHGGREIDGSPATIDVLPEIAKAVNHRVPIVFDSGVRRGSHVFKALALGADLVGIGRPYLYGLALGGPKGVESVIDQLNTELKIDMQLTGCKTIEDIKHAKISRIHYGLDAMPSNTDPSRMKPYPTTAENQIKSTNTDAVTGASQH

SEQ ID NO: 144 - protein, 366 aa, Enterococcus italicus
MDYTTSKEEHPIKIVNLSSLEKAVKEDMDKGAFGYIRGGSEDEWTLRENTQAFSKKKIIPRVLQGIDHADLSTELFGIPLKTPIIQAPSAAQGLAHVKGEVDTAIGVAKAGSIFAISTYANTKIEDAAAAAPDAPQFFQLYMSKDDAFNQFLLDKAVQSGAKAIILTVDSTLGGYREEDIVNDFTFPLPMPNLVAFSEASGAGEGKGKGISEIYVAAKQAIVPEDIQKIKKMSGLPVIVKGIQSAEDGEVAIQFGADGIWVSNHGGRQLDGAPASFDVLPQIAQVVRKRVPIIFDSGVRRGEHVFKALASGADVVALGRPIIYGLFLGGAEGVTSVFDHLNKELAITMQLAGTKTIEDVKQTTLVD

SEQ ID NO: 145 - protein, 410 aa, Lactobacillus pasteurii
MTYYYNGFPQSDRNEKLDMVNVEELEERVKDVMPEGAYYYIASGSENEWTWRNNTAAFNHYQIVPRALTDMDNPSTETEFMGMKLKTPIMISPIACHGIAHKDAEIATQQGAAAAGALFSSSTYANKSVEEIAAAAPEAPRFFQLYLSKDWEFNRMVFDAIKKAGYKGIFLTVDALISGYREANLRTNFTYPVPLDFFTRYLGGKGEGQSVAQMYASSAQKIGPADVRRIKEESGLPVFVKGVMCAEDAYLAMGAGADGIYVTNHGGREVDTAPATIDVLPEIAAAVDHRVPIIFDSGVRRGSHIFKALAMGADLVGIGRPYLYGLALGGAKGVESVIDELNEELIINMQLTGCKTIEDVKHAKISNIRYTADNLPSNTDPTRREAYPKTAENQIKVEADADATSGASKH

SEQ ID NO: 146 - protein, 412 aa, Lactobacillus hominis
MTVYYKGFPQSTREEKLKMTNLLELPEKVKKIMPEGAYYYIASGAENEWTWRNNTQAFNHFQIVPRALTNMQDPQLDTTFMGMNLKTPVMICPIACHGIANAEAEIDTAKGAKAAGALFGMSTYCNKSVQDVQKAVGKSHRFMQLYLSKDWDFNKMVIEESEKAGFEGFYLTVDALVSGYREANIRTNFTYPVPLAFFQEWTGGKGEGQSVAQMYANSAQKIGPDDVRKIKELTDLPLIVKGVKCAEDAYKALGAGADGIEVSNHGGREVDGGPATIDVLPGIAREVRHAGRKVPIIFDGGVRRGSHVFKALALGADLVGIGRPFLYGLALGGAQGVQSVIEQLNKELLIDMQLTGCKTIEDVKHAKIDHFTYSADWGISSTSKSVMPPYPVTEDNQLKGEAADAVSGASEH

SEQ ID NO: 147 - protein, 410 aa, Lactobacillus gigeriorum
MTHYYNGFPQSDRDEKLDMVNVDELEERVKQVMPEGAYYYIASGSENEWTWRNNTAAFNHYQIIPRALTNMDNPSTETEFMGMKLKTPIMISPIACHGIAHMDAEVATQKGAAAAGALFASSTYANKSVEDIAAAAPDAPRFFQLYLSKDWEFNRMVFDAIKKAGYKGIFLTVDALISGYREANLRTNFTYPVPLDFFTRYLGGKGEGQSVAQMYASSAQKIGPDDVRRIKEESGLPVIVKGVMCAEDAYLAIGAGADGVYVTNHGGREVDTAPATIDVLPEVVKAVNHRVPVIFDSGVRRGSHIFKALAMGADLVGIGRPYLYGLALGGAKGVQSVIEELNEELIINMQLTGCKTIEDVKHARIDHIRYTADNLPSNTDPTRRAAYPKTAENQIKVEADTDATSGASHH

SEQ ID NO: 148 - protein, 367 aa, Liquorilactobacillus mali
MTTINGYEQSDREQKLDILNLSSLEEKAKKIIPAGGFGYISGGSEDEWTLHENTSAFNHIQIIPRALTNVEQPTTATEVFGLKLKTPIMMAPAAAQGLAHSRGEKATAEGLTKVGGLMAQSTYSSTSIADTAAAGNGTPQFFQLYMSKDWDFNYSLLDEAVKAGAKGIILTVDATVDGYRESDIINNFQFPIPMANLAKFSEGDGKGKGIMEIYAAAAQKIGPDDVRRIAEYTNLPVIVKGIESAEDALLAIGAGAKGIYVSNHGGRQLNGGPASIDVLHEVAQAVNHRVPVIFDSGVRRGSHVFKALASGADLVALARPIIYGLALGGAQGVASVISHLNDELKIDMQLAGTKTIEDVKKAKVIRK

SEQ ID NO: 149 - protein, 409 aa, Lactobacillus equicursoris
MTRYIKGFPQSEADEDLKFVNVDELEERAKEVMPEGAYYYVASGSEYEWTWRNNTNAYNHYQIVPRSLTGMDNPSTETEFMGMKLKTPIMISPIACHGISHADAEVATQKGAALAGAMFSSSTYGNKPVEEIAAAAPDAPRMFQLYLSKNWDFNQMVFDAIKAAGYKAILLTVDALVSGYREANLRTDFAFPVPLDFFTRFQGAKGEGQTVAQMYASSAQNIGPDDIKRIKEMSGLPVIVKGINCAEDVEVALTAGADGVYVTNHGGREIDGAPATIDVLPEVVKAVNGRVPVIFDGGVRRGSHIFKALALGADLVGIGRPYLYGLALGGPHGVASVINELNDELKIDMQLTGCKTIEDVKHARLTHFEYAGDTRPSSTDPRVRKPYPVTAVNQIKKDGTDAVSGASHH

SEQ ID NO: 150 - protein, 365 aa, Lactococcus garvieae
MTYVTSKDEQKVEIVNIASLEARVKNRMEEQGNKGAFGYMRGGSEDEWTMRENTASFNTKTIVPRILRGIDSADLSTSIFGINLKTPFIQAPVAAQGLAHVAGEVDTAKAMEEVGSLFSISTYGSTSVEDAAKAVNGAPQFFQLYMSKDDQFNQFLLEKAVKAGVKAIILTADSTLGGYREEDVINHFQFPLPMPNLAAFSETDGVGKGIFEIYAAAKQGLVLSDIQKIKKWTNLPVIVKGIQDPTDAMEAIAAGADGIWVSNHGGRQLDGGPASFTVLPNIAKVVNKRVPIIFDSGVRRGAHVFKALASGADLVAVGRPVLYGLNLGGKEGVKSVFEHLNKELSITMQLAGAKDIEAVKKTELL

SEQ ID NO: 151 - protein, 408 aa, Lactobacillus sp.
MTYYYNGFPQSEHDEKLNLINVDQLEEDAKRVIPEGAYYYIASGAENEWTWRNNTAAFNHYQIVPRALTDMEDPQLDTEFMGMKLKTPVMIAPIACHGITHKDAEVATQKGAAAAGALFSSSTYANKSVEDIAAAAPDAPRFFQLYLSKDWNFNKMVFDAVKKAGYKAIMLTVDALISGYREANLRTEFTYPVPLDFFNRYLGAKGEGQSVAQMYAASAQKIGPKDVARIKEESGLPVLIKGITCVEDAYKALGAGADGIYVTNHGGREIDGSPATIDVLPDIAKAVNHRVPIIFDSGVRRGSHVFKALALGADLVAIGRPYLYGLALGGAKGVESVIEQYNKELKIDMQLTGCKTIDDVKKAKIMHINYTADNLPSNTDPSRMKPYPVTSENQPQEDLDATSGASHH

SEQ ID NO: 152 - protein, 368 aa, Enterococcus malodoratus
MTYQTSNEEHPIEIVNIASLEGRVKERMDKGAFGYIRGGSEDEWTMKENTSSFNNKTITPRVLRGIDHADLNTKLWDIDLATPIIQAPSAAQGLAHEKGEADTAKGVADAGSIFSISTYANTTIEDAAAVAPNAPQFFQLYMSKDDGFNEFILEKAVKAGAKAIILTADSTLGGYREEDIINNFQFPLPMPNLAAYSAKSASGNGEGKGIAEIYAAAKQGLVPEDIKKIKDLTNLPVLVKGIQSPEDAEVAIAAGADGIWISNHGGRQLDGGPASFDVLPLIAGVVNKRVPIIFDSGIRRGEHVFKALASGADLVAIGRPVIYGLNLGGAEGVKSVFDHLNKELAITMQLAGTKTIDEVKNTRLNDVK

SEQ ID NO: 153 - protein, 365 aa, Enterococcus pallens
MTYQASNEEHPIEIVNIASLEARVKERMEKGAFGYIRGGSEDEWTMKENTASFNKKSIMPRVLRGIDHADLRTKLWDMELKTPIIQAPSAAQGLAHEKGESDTAKGVAAVGSIFSISTYANTTIEDAAEAAPGAPQFFQLYMSKDDGFNEFILAKAVQAGAKAVILTADSTLGGYREEDIINHFEFPLPMPNLAAYSEKDAAGNGEGKGIAEIYAAAKQGLVPEDIKKIKDITNLPVFVKGIQSPEDAEVAISAGADGIWISNHGGRQLDGGPASFDVLPLIAEVVNKRVPIVFDSGVRRGEHVFKALASGADLVAIGRPVIYGLNLGGAEGVTSVFEHLNKELSITMQLAGTKTIEEVKNTKLI

SEQ ID NO: 154 - protein, 366 aa, Enterococcus phoeniculicola
MAYQASTEEHPIEIVNIASLENRVKENMERGAFGYIRGGSEDEWTMAENTHAFKKKKIIPRVLRGIDHVSLKTTLWDIELDTPIIQAPSAAQGLAHEKGETNTAKGVADAGSIFSISTYANTTIEDAAKAAPNAPQFFQIYMSKDDGFNQFILTKAMEAGAKAIILTVDSTLGGYREEDVVNKFQFPLPMPNLAAYSEQSASGDGEGKGISEIYAAAKQGLVPEDIRKIKAFTKLPVIVKGIQAPEDAEVAISAGADGIWVSNHGGRQLDGGPASFDMLPLIASIVNKRVPIIFDSGIRRGEHVFKALASGADLVAIGRPIIYGLNLGGAQGVKSVFDHLNKELSITMQLAGTKTIEDVKNTTLMD

SEQ ID NO: 155 - protein, 368 aa, Enterococcus avium
MTYQASNEEHPIEIVNIASLEGRVKERMEKGAFGYIRGGSEDEWTMKENTSSFNKKTIMPRVLRGIDHADLSTKLWDMDLATPIIQAPSAAQGLAHEKGEADTAKGVADAGSIFSISTYANTTIEDAAAAAPNAPQFFQLYMSKDDGFNAFILEKAVKAGAKAIILTADSTLGGYREEDIVNKFQFPLPMPNLAAYSAKSASGDGEGKGIAEIYAAAKQGLVPEDIKKIKDLTSLPVFVKGIQSPEDAEVAIAAGADGIWVSNHGGRQLDGGPASFDVLPLITGVVNKRVPIVFDSGVRRGEHVFKALASGADLVAIGRPVIYGLNLGGAEGVKSVFDHLNKELSITMQLAGTKTINEVKNTRLHDTK

SEQ ID NO: 156 - protein, 365 aa, Enterococcus dispar
MYHTSNEEHPIDIVNIASLEGRVKERMEAGAFGYIRGGSEDEWTMKENTASFMNKKIMPRILQGIDHADLSTKLWDIELQTPIIEAPSAAQGLAHEKGEADTAKGVAAAGSIFSISTYANTTVEDAAAAAPDAPQFFQLYMSKDDGFNEFILDKAVKAGAKAIILTADSTLGGYREEDVINQFQFPLPMPNLAAYSAQSASGNGEGKGIAEIYAAAKQGLVPEDIKKIKDLTHLPVLVKGIQSPEDADLAIKAGADGIWVSNHGGRQLDGGPASFDVLPAIAEVVDKRVPIVFDSGVRRGEHVFKALASGADLVAIGRPVIYGLNLGGAEGVKSVFDHFNKELSITMQLAGTKTIADVKKTKLID

SEQ ID NO: 157 - protein, 369 aa, Lentilactobacillus otakiensis
MTVVNGYKQNDNEEKINILNLEELEERAKQIIPTGGFGYIAGGSENNWTLKANRQAFTHKQIVPRALSNIEDPQLDTNVFGIPLKTPIMMAPTAAQGLAHSQGEKDTAKGVAAVGGLMAQSTYSSVSIADTAAAGNGAPQFFQLYMSKDWDFNYSLLDEAKKAGVKGIILTVDATVDGYREDDIKNNFQFPIPMANLTKFSEGDGKGKGIGEIYAAAAQKIGPDDVKKIADYTDLPVIVKGIESPEDALYAIGAGAAGVYVSNHGGRQLNGGPASFDVLEDVAKAVNGRVPIIFDSGVRRGSDAFKALASGADLVAMGRPVIYGLALGGAEGVQAVFEHLGDELKTIMQLAGTKTIDDVKKTDLLNIKY

SEQ ID NO: 158 - protein, 398 aa, Salmonella bongori
MKSSHLIRVVASLAMLATSGLAYAEEYKASTDEKTIKMTNVASLEARVQARMDKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEEVANVSGENPFFFQLYMSKNNKFNEFILAQAVKHGAKAIILTVDSPVGGYREEDIKNNFQFPLGFANLEMFARKNDDGSKTGKGAGISEIYAQAKQAFTPEDIAYVHRISGLPVIVKGIQSPEDAEIAIQAGAAGIWVSNHGGRQLDSGPSSFDMLPAIAKVVNKRVPVIFDSGVRRGSHVFKALASGADIVAIGRPVLYGLNLGGAQGVASVIEQLNKELTINMMLGGARNIEQVKNTRLLSEKDL

SEQ ID NO: 159 - protein, 369 aa, Schleiferilactobacillus shenzhenensis
MTMTNGYEQSDREQKLDFINVNDLAKEAQAIIPKGGYGYIRGGSEDEWTLRENTLAFNHAQIIPRALTDMENPATDTEVFGLKLKTPLMMAPLAAQGLAHAKGETDTARGVAAVGGLMAQSTYSSTSIADTAAAGNGAPQFFQLYMSKDWDFNHHLLDEAKKAGVKGIILTLDAPVDGFREDDLRNHFQFPIPMANLTEYSEGDGSGKGIAEIYAAAAQKIGPKDIERIAEYTDLPVIAKGIMSPDDAMKAIGAGAAGIYVSNHGGRQLNGGPASFDVLPSIAQAVNHKVPIIFDSGVRRGSHIFKALAAGADLVALGRPVVYGLALGGAQGVQSVFEHLADELKIDMQLAGTKTIADVKNTRLAHFPA

SEQ ID NO: 160 - protein, 398 aa, Proteus hauseri
MKRQILKATAIAMALSVGVAQAAEYKASTAEGPIKIVNLKAMEAQVQANMEKGAFGYIRGGAEDENNLRANTRAFDKKYIMPRSLQGIEFSDINLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIIMTIDSSVGGYREDDVKNNFQFPLGFANLEAFAKISDDKSKTGKGAGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADVVAVGRPILYGLNLGGSEGVNSVIQHLNKELRINMMLGGAKTVKDIQATPLYTDASFNQ

SEQ ID NO: 161 - protein, 369 aa, Lentilactobacillus farraginis
MAIVNGYKQNENEKALDILNLDQLEAQAKEIIPTGGFGYIAGGSEDEWTLQANRRAFTHKQIVPRALTNIEKPELDTNVFGLPLKTPIFMVPAAAQGLAHAKGEVDTAKGLAAVGGLMAQSTYSSTSIADTASAGAGAPQFFQLYMSKDWEFNKSLLDEAKKAGVKGIILTVDATVDGYREADIVNNFQFPIPMANLTKYSEGDGQGKGIAEIYASAAQKIGPDDVARIADYTDLPVIVKGIESPEDALYAIGAGAAGIYVSNHGGRQLNGGPASFDVLADVARAVNGKVPIIFDSGVRRGSDVFKALASGADLVGIGRPVIYGLALGGAQGVQSVFEHLDHELEIIMQLAGTKTIADVKKAKLLNIHY

SEQ ID NO: 162 - protein, 366 aa, Lactococcus lactis
MTYKTSTENKALEIVNVKSLEGKVKQSMEAAGNKGAFGYIRGGSEDEWTLNENTSAFNKKQIMPRVLRGVDSADLSTSLFGIKLKTPIIQAPVAAQGLAHEEGEVATAKAMAEVGSIFSISTYGSTSVEDAAKAAPDAPQFFQLYMSKDDRFNEFLLKKAVSAGVKAIILTADSTLGGYREEDIVNHFQFPLPMPNLAAFSESDGTGKGISEIYAAAKQGLVLEDIQKIKKITNLPVIVKGVQSPIDADDAINAGADGIWVSNHGGRQLDGGPASIDVLPLIAKSVNHRVPIVFDSGVRRGEHVFKALAQGADVVAVGRPVLYGLNLGGAKGVQSVFEHLNKELSITMQLAGTKNIEEIKHTSLID

SEQ ID NO: 163 - protein, 400 aa, Salmonella enterica
MKTHHLIRVVASLAMLATSGLAYAEEYQSQHGRKAIKMTNVASLEARVQARMEKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEEVANVSGKNPFFFQLYMSKNNQFNEFILAQAVKHGAKAIILTVDSPVGGYREEDIKNNFQFPLSFANLEMFARKNDDGSKTGKGAGISEIYAQAKQAFTPEDIAYVHRISGLPVIVKGIQSPEDAEIAIQAGAAGIWVSNHGGRQLDSGPSSFDMLPAIAKVVNKRVPVIFDSGVRRGSHVFKALASGADIVAVGRPVLYGLNLGGAQGVASVIEQLNKELTINMMLGGARNIEQVKTTRLLTEKDLPQ

SEQ ID NO: 164 - protein, 408 aa, Lactobacillus jensenii
MTVYYKGFPQSDRNEAIKMVNVDELEDRVRKVMPEAAYYYIASGSENEWTWRNNTAAFNHFQIVPRSLTNMDNPSTETQFMGMDLKTPIMICPIACHGIAHKDAEVATAQGAKAAGALFSSSTYANRSVEDIATATGDSPKFFQLYLSKDWDFNKMVFDAVKSAGYKGIMLTVDALVSGYREANLRTNFTFPVPLDFFTRYVGAEGEGMSVAQMYANSAQKIGPADVAKIKEMSGLPVFVKGVMNAEDAYMAIGAGADGIVVSNHGGREIDTAPATIDMLPEIAAAVNGRVPIILDSGVRRGSHVFKALALGADLVGIGRPFLYGLALGGAKGVESVINQINNEFKILMQLTGCKTVEDVKHADIRQINYTADNLPSNTDPSVRRAYPVTKENQMEGTQDAATGASKH

SEQ ID NO: 165 - protein, 365 aa, Lactococcus garvieae
MSYVTSNDEQKVEIVNIASLEGRVKERMEAQGNKGAFGYIRGGAEDEWTMRENTASFNTKTISPRVLRGIDSANLSTNIFGIELKTPIIQAPVAAQGLAHMEGEVDTAKAMEEVGSLFSISTYGSTSVEDAAAAVNSAPQFFQLYMSKDDQFNQFLLEKAVKSGVKAIILTADSTLGGYREEDVINHFQFPLPMPNLAAFSESDGVGKGIFEIYAEAKQGLVLSDIQKIKNWTNLPVIVKGIQDPVDAMEAIAAGADGIWVSNHGGRQLDGGPASFTVLPRIAQVVNKRVPIIFDSGVRRGEHVFKALASGADLVAIGRPVLYGLNLGGKEGVKSVFEHLNKELSITMQLAGAKDIEAIKNTDLL

SEQ ID NO: 166 - protein, 410 aa, Ligilactobacillus equi
MTAYYNGFPQSDKDEKITMVNVDELEEKVKEVMPEPAYYYIASGSENEWTWRNNTAAFNHFQIVPRSLTDMDNPSTATEFMGMKLKTPIMIAPIACHGIAHKDAEIATAQGAKAAGALFSSSTYANKSVEEIAAATGDAPRFFQLYLSKDWEFNQMVFDAVKAAGYKGIFLTVDALVSGYREANLRTNFTFPVPLDFFTRYLGGKGEGQSVAQMYASSAQKIGPADVARIKEMSGLPVFIKGVMNADDAYLALGAGADGIVVSNHGGREIDGAPATIDMLPEIARAVNKRVPIVFDSGVRRGSHVFKALALGADLVGIGRPYLYGLALGGASGVQSVIEQINSELEIDMQLTGCKTIEDVKHADVRNLSYGLDNLPSSTSPKVRKPYPVTAENQMKVEAETDATSGASSH

SEQ ID NO: 167 - protein, 402 aa, Brucellaceae
MKTFLSKKYAVSLSAIALALSLGVAHAADYKASTAEGPVKITNLGELEAQVKANMDKGAFGYIRGGAENEQNLRSNTSVFDKKYIMPRVMQGIELSDIDLKTNYLGIDLKTPVIQAPMAAQGLAHQDGEIATAKGMAKAGSIFSLSTYGNKTIEEVAAVSDGNPFFFQLYMSKNEAFNEFTLKRAKDSGAKAIILTVDSPVGGYREDDIKNNFQFPLGFANLELFAKQNDDGSKTGKGAGISEIYAQAKQAFTPADIKYVKDMTGLPVIVKGIQSPEDADVVIKAGADSIWVSNHGGRQLDSGPASFDVLPSIAKVVNKRVPIVFDSGVRRGSHVFKALASGADVVAVGRPILYGLNLGGSEGVNSVIQQLNKELTINMMLGGVKNIEEVKKTKLYSDKDFE

SEQ ID NO: 168 - protein, 366 aa, Enterococcus faecalis
MAYQASNEEHPINIVNIASLEGRVKERMEAGAFGYIRGGSEDEWTMRENTSSFNTKKIMPRVLRGIDSADLRTSVFGIDLDTPIIQAPSAAQGLAHEKGEAATAKGVADAGSIFSISTYANTTIKDAAAAAPDAPQFFQLYMSKDDGFNEFILNKAVEAGAKAVILTADSTLGGYREEDIINNFQFPLPMPNLAAYSEQSASGNGAGKGIAEIYAAAKQELTPADIRKIKELTNLPVLVKGIQSPEDAEVAISAGADGIWVSNHGGRQLDGGPASFEVLPKIAEVVSKRVPVIFDSGVRRGEHVFKALASGADLVAIGRPVIYGLNLGGAAGVTSVFEHLNKELAITMQLAGTKTIDEVKNTKLLD

SEQ ID NO: 169 - protein, 369 aa, Agrilactobacillus composti
MTITNGYEQSDREEKLDIINLNELEAQAKAIIPQGGFGYISGGSEDEWTLRENTNAFEHAQIVPRALTDMDNPETDTTVFGLNLKTPIMMAPTAAQGLAHAKGEVDTAKGVAAAGALMAQSTYSSTSIADTAAAGNGAPQFFQLYMSKDWDFNNHLLDQAKAAGVKAIILTVDATIDGYREADLRNKFQFPIPMANLTEYSEGDGAGKGIAEIYAAAAQKISAADVKRIADYTDLPVIVKGIESPEDALFAIGAGAKGIYVSNHGGRQLNGGPASFDVLADVAKAVDHRVPIIFDSGVRRGSHVFKALASGADLVAMGRPVIYGLALGGAQGVQSVFEELNGELKIDMQLAGTKTIEDVKKAKLLHIHY

SEQ ID NO: 170 - protein, 366 aa, Lactococcus
MTYKTSTENKALEIVNVKSLEGKVKQSMEAAGNKGAFGYIRGGSEDEWTLNENTSAFNKKQIMPRVLRGVDSADLSTSLFGIKLKTPIIQAPVAAQGLAHAEGEVATAKAMAEVGSIFSISTYGSTSVEDAAKTAPGAPQFFQLYMSKDDKFNEFLLKKAVDAGVKAIILTADSTLGGYREEDIVNHFQFPLPMPNLAAFSESDGTGKGISEIYAAAKQGLVLEDIQKIKKITNLPVIVKGVQSPIDADDAINAGADGIWVSNHGGRQLDGGPASIDVLPLIAKSVNHRVPVIFDSGVRRGEHVFKALAQGADVVAVGRPVLYGLNLGGAKGVQSVFEHLNKELSITMQLAGTKNIEEIKHTSLID

SEQ ID NO: 171 - protein, 367 aa, Enterococcus massiliensis
MTYQASKEEHPIEIVNIASLEKRAQETMEAGAFGYIRGGSEDEWTMAENTSSFNKKTIMPRVLRGIDSADLHTKLWDIQLETPIIQAPSAAQGLAHTNGEADTAKGVAAAGSIFSISTYANTTIEDAAAAAPNAPQFFQLYMSKDDGFNKFILDKAVKAGAKAIVLTVDSTLGGYREEDVINHFQFPLPMPNLAAYSEQSESGDGAGKGIAEIYAAAKQAIEPEDIQKIKTLTNLPVVVKGIQSPEDAEVAIAAGADGIWVSNHGGRQLDGGPASFDVLPEIAAVVNKRVPIVFDSGVRRGEHVFKALASGADLVAIGRPVIYGLNLGGAEGVKSVFDHLNKELSITMQLAGTKTIEEVKNTKLFSY

SEQ ID NO: 172 - protein, 410 aa, Lactobacillus acetotolerans
MTTYYNGFPQSDRNETIHMVNVDELEERAKKVMPEGAYYYIASGAENEWTWRNNIASFNHFQIVPRALTNMDNPQTDTEFMGMKLKTPIMISPIACHGIAHKDAEVATQEGAAAAGALFSSSTYANKSVEDIASAAPNAPRFFQLYLSKNWDFNKMVFDAVKKAGYKGIFLTVDALVSGYREANLRTKFAYPVPLDFFTRYQGAKGEGQTVAQMYAASAQKIGPKDVQRIKKMSGLPVFVKGVMCAEDAFKAIGAGADGIYVTNHGGREVDSGPATIDMLPSIAKAVNHRVPIVFDSGVRRGSHVFKALALGADMVGIGRPYLYGLALGGAKGVKSVIDQLDMELKIDMQLTGCKTIEDVKHAKIEHINYAADNLKSNTDPSRFKGYPVTKDNQIKEDKSSDAVSGASQA

SEQ ID NO: 173 - protein, 368 aa, Companilactobacillus farciminis
MGIFMAKYEASTEEKHIDIVNIASLEQRVKDHMSNEKGAFGYIRGGSEDEWTMKQNTEAFNKKRIMPRVLQGIDHADLSTKLWDIDLKTPIIEAPSAAHGLAHAKGEVDTAKGVAAAGSIFSMSTYGSTAIEDGAAAAPDAPQFFQLYMSKDDKFNEFLIKKAVKAGVKAIILTVDSTLGGYREADVINKFQFPLPMPNLAAFSDGDGEGKGISEIYAAAKQGIVPSDIQKIKDMSGLPVFVKGIQSPDDAELAIEFGADGIWVSNHGGRQLDGGPASFDVLPSIAQVVDKRVPIVFDSGVRRGEHVFKALASGADLVAVGRPIIYGLNLGGAQGVTDVIEHLNKELSITMQLAGTKTIHDVKNVDLL

SEQ ID NO: 174 - protein, 368 aa, Companilactobacillus futsaii
MGIFMAKYEASTEEKHIDIVNIASLEQRVKDHMSNEKGAFGYIRGGSEDEWTMKQNTEAFNKKRIMPRVLQGIDHADLSTKLWDIDLKTPIIEAPSAAHGLAHAKGEVDTAKGVAKAGSIFSMSTYGSTSIEDGAAAAPDAPQFFQLYMSKDDKFNEFLIKKAVKAGVKAIILTVDSTLGGYREADVINKFQFPLPMPNLAAFSDGDGEGKGISEIYAAAKQGIVPSDIQKIKDMSGLPVFVKGIQSPDDAQLAIEFGADGIWVSNHGGRQLDGGPASFDVLPAIAEAVDKRVPIVFDSGVRRGEHVFKALASGADLVAIGRPIIYGLNLGGAQGVTDVIEHLNKELSITMQLAGTKTIHDVKNVDLL

SEQ ID NO: 175 - protein, 369 aa, Secundilactobacillus odoratitofui
MVMTNGYEQNENEQALDILNLPQLEADAKKIIPTGGFGYISGGSEDEWTLRANRTAFQHRQIVPKALSNIENPQTDTTVFGLNLKTPIMMAPAAAQGLAHAKGEVDTAKGVAKVGGLMAQSTYSSTSIADTAAAGEGAPQFFQLYMSKDWEFNKSLLDEAKKAGVKAIILTVDATVDGYREADIINNFQFPIPMANLTKFSEGDGKGKGIGEIYAAAAQKIGPADLQQIIDYTDLPVIVKGVESAEDALYAIGAGAAGIYVSNHGGRQLNGGPASFDVLESVAKAVNHQVPVIFDSGVRRGSDVFKAIALGADLVAIGRPAIYGLALGGAEGVASVFTHLNDELKIIMQLAGTKTIDDIKQAELLNLNY

SEQ ID NO: 176 - protein, 369 aa, Lentilactobacillus kefiri
MTVVNGYKQNDNEQKLDILNLEDLEEKAKQIIPTGGFGYIAGGSENNWTLKANRKAFTHKQIVPRALSNIEKPELDTNVFGIPLKTPIMMAPTAAQGLAHSQGEKDTAKGLAAVGGLMAQSTYSSTSIADTAAAGDGAPQFFQLYMSKDWDFNYSLLDEAKKAGVKGIILTVDATVDGYREDDIKNNFQFPIPMANLTKFSEGDGKGKGIGEIYASAAQKIGPDDVKKIADYTDLPVIVKGIESPEDALYAIGAGAAGVYVSNHGGRQLNGGPASFDVLEDVAKAVNGQVPVIFDSGVRRGSDAFKALASGADLVAMGRPAIYGLALGGAQGVQSVFEHLGDELKIIMQLAGTKTIADVKKTNLINIKY

SEQ ID NO: 177 - protein, 371 aa, Lacticaseibacillus camelliae
MTLTNGYEQSDREQKLNIVNLPSLEAEAKKIIPTGGFGYIAGGSEDDWTLRQNTAAFGHLQIVPKALSNIEQPSLATHVFGLDLKTPIMMAPTAAQGLAHSQGEKDTARGVAAVGGLMAQSTYSSTSIADTAAAGNGAPQFFQLYMSKDWTFNESLLDEAVKAGVKAILLTVDATVDGYREADIVNNFQFPIPMANLTKFSEGDGKGKGIGEIYASAAQKISEDDVRRIADYTHLPVIVKGIQSPEDALRAIGAGAAGIYVSNHGGRQLNGGPASIDVLPAIAKAVNHQVPIIFDSGVRRGSHVFKALAAGADLVAMGRPVIYGLALGGAQGVQSVFEELNHELEITMQLAGTKTIEDVKHAPLTHFNYAE

SEQ ID NO: 178 - protein, 362 aa, Latilactobacillus graminis
MAYHTSDAEAPVDILNLDSLEGRVKERMEAGAFGYIRGGSEDEWTMAANTSAFNSKKIMPRVLQGIDHANLHTKLWDIDLKTPIIQAPSAAQGLAHEKGEVDTAKGVAAAGSIFSISTYANTLVEDAAAAAPDAPQFFQLYMSKDDQFNEFLLKKAVKAGVKAIILTVDSTLGGYREADIENHFQFPLPMPNLAAYSASDGEGKGISEIYAAAKQGLVPADIQKIKEITNLPVLVKGIQSPVDAEIAIQAGADGIWVSNHGGRQLDGGPASFEVLPLIAQQVAKRVPIVFDSGIRRGEHVFKALASGADLVAIGRPIIYGLNLGGAQGVKSVFDHLNEELSITMQLAGTKTIDEIKETTLLD

SEQ ID NO: 179 - protein, 369 aa, Lentilactobacillus parabuchneri
MTVVNGYKQNDNEQKIDILNLEELEERAKQIIPTGGFGYIVGGSENNWTLKANRKAFTHKQIVPRALSNIEDPQLDTNVFGIPLKTPIMMAPTAAQGLAHSQGEKDTAKGVAAVGGLMAQSTYSSASISDTAAAGDGAPQFFQLYMSKDWDFNYSLLDEAKKAGVKGIILTVDATVDGYREDDIKNNFQFPIPMANLTKFSEGDGKGKGIAEIYAAAAQKIGPDDVKKIADYTDLPVIVKGIESPEDALYAIGAGASGVYVSNHGGRQLNGGPASFDVLEDVAKAVNGRVPIIFDSGVRRGSDAFKALASGADLVAMGRPVIYGLALGGAQGVQSVFEHLGDELKITMQLAGTKTIADVKKTHLLNIQY

SEQ ID NO: 180 - protein, 397 aa, Proteus mirabilis
MKHTLLKTTAIAMALTVGVAQAAEYKASTAEHPIKIVNLDALENQVKENMDKGAFGYIRGGAEDENNLRSNTSAFNKKYIMPRALQGIEFSDLNLKTEFLGIKLDTPIIQAPMAAQGLAHQQGEVATAKGMAKAGSIFSLSTYGNKTIKEVAQAQPGYPFFFQLYMSKNDAFNQYILSQAKQYGAKGIILTVDSPVGGYREDDIKNSFQFPLGFANLEAFAKISDDKSKTGKGSGISEIYAQAKQAFTPADIQYVKKMSGLPVIVKGIGSPEDADIAIKAGADAIWVSNHGGRQLDSAPATIDMLPAIAKVVNKRVPIVFDSGVRRGSHVFKALASGADVVAVGRPILYGLNLGGAEGVNSVIQQLNKELRINMMLGGARNVKEIQATQLYTDADFK

SEQ ID NO: 181 - protein, 365 aa, Gilliamella apicola
MSYHTSNEEHAIEIVNIASLEALVKARMELGAFGYIRGGAEDEWTMRENTLAFNRKKIIPRVLQGIDHADLSTKLWDISLKTPIIQAPSAAQGLAHEEGEKDTAKGVAAAGSIFCISTYANTSIEDAANAAPNVPYFFQLYMSKDDDFNQFIIDKAVKAGAKAIILTVDSTLGGYREEDIVNKFQFPLPMKNLSAYSQSNGNGDGSGKGISEIYAAAKQGIVPSDIQKIKDMANLPIIVKGIQSPEDASIAISAGTDGIWISNHGGRQLDGAPASFEVLPSISAAVAKRVPIIFDSGIRRGEHVFKALASGADLVAIGRPILYGLNLGGAEGVKSVFDHLNKELSITMQLAGTKTIEDIKNTLLI

SEQ ID NO: 182 - protein, 368 aa, Weissella confusa
MAYQGSTKEEALNIIELPRLRAAVQRDREAGAFGYVDGGSSDEQVLHANETAFRHYQLIPRMLQNISAPDLSTTLLDIPLSMPVIAAPIAAHGLMHENGERVTVKGVGAAGTIFSLATYGNSRIADVAASSPNTPKYFQLYMSRDDEFNQYLLDEAVQNGYKAIILTADATLGGYREADIINNFAFPLPMENLAAFSNAAGSGEGLGISEIYARPKQDLALSDITKVKQMANGLPVFVKGIQYPDDALAAIAAGADGIWVSNHGGRELNGAPASIDTLAAVAKAVNHRVPVVFDSGIRRGEDVAKAIALGADVVALGRPMLWGLNQGGAAGVQSVYEHLAEELKTVMQLTGSHTVAELQHAKIIDAKF

SEQ ID NO: 183 - protein, 365 aa, Enterococcus hermanniensis
MVYQTSNEEHPIEIVNIASLEKRVKERMDKGAFGYIRGGSEDEWTMKENTSSFNNKTIMPRVLRGVDHADLTTKLWNIDLSTPIIQAPSAAQGLAHEKGEADTAKGVAAAGSIFSISTYANTTIEAAAASAPEAPQFFQLYMSKDDRFNEFILNKAVHAGAKAIILTADSTLGGYREEDIINDFQFPLPMPNLAAYSEQSASGDGAGKGIAEIYAAAKQGLIPEDIKKIKELTDLPVFVKGIQAPEDAEVAIAAGADGIWVSNHGGRQLDGGPASFDVLPLIAAVVNKRVPIVFDSGVRRGEHVFKALASGADLVAIGRPVIYGLNLGGAEGVTSVFEHLNKELAITMQLAGTKTIEEIKNVTLK

SEQ ID NO: 184 - protein, 398 aa, Proteus
MKNKLLKTTAIAMALSVGVAQAAEYKASTAEGPIKIVNLKAMEAQVQANMDKGAFGYIRGGAEDENNLRSNTTAFDKKYIMPRSLQGIEFSDLDLKTEFLGIKLDTPIIQAPMAAQGLAHQQGEVATAKGMAKAGSIFSLSTYGNKTIKEVADAKPGYPFFFQLYMSKNDAFNEYILSQAKQYGAKGIIMTIDSSVGGYREDDVKNNFQFPLGFANLEAFAKISDDKSKTGKGAGISEIYAQAKQAFTPADIQYVKKMSGLPVIVKGIQSPEDADVAIKAGADAIWVSNHGGRQLDSGPASIDVLPSIAKVVNKRVPIVFDSGVRRGSHVFKALASGADVVAVGRPILYGLNLGGAEGVNSVIQHLNKELKINMMLGGAKTVKDIQATQLYTDASFNQ

SEQ ID NO: 185 - protein, 402 aa, Brucella grignonensis
MISLHGRKYAASLSAVVLAFSLGVAHASDYKASTAEGPIKIVNLNDLEAQVQNNMEKGAFGYIRGGAENEQNLRSNTSAFDKKYIMPRVMQGIELSNIDLSTSFLGLKLKTPVIQAPMAAQGLAHQDGEIATAKGMAKAGSIFSLSTYGNKTIEEVAEVSGDNPFFFQLYMSKNNAFNEFTLKRAKDSGAKAIILTVDSPVGGYREDDIKNSFQFPLGFANLELFAKQKDDGSKTGKGAGISEIYAQAKQAFTPTDIKYVKDLTNLPVIVKGIQSPDDADTVIKAGADAIWVSNHGGRQLDSGPASFEVLPSIAKVVNKRVPIVFDSGVRRGSHVFKALASGADIVAVGRPILYGLNLGGAEGVNSVVQQLNKELTINMMLGGAQNIEAVKKTRLYTDKDFE

SEQ ID NO: 186 - protein, 412 aa, Lactobacillus johnsonii
MTTYYKGFPQSTREEKLHMVNLDELEKEAKYVIPEAAYYYIASGAENEWTWRNNTQAFNHFQIVPRALTGMQDPELNTEFLGMKLKTPVMICPIACHGIANAEAEVDTAKGAKVAGALFGMSTYANKSVQDVQSAVGDSPRFMQLYLSKNWDFNKMVIEESVKAGFTGFFLTVDALVSGYREANLRTNFTYPVPLAFFNEWTGGKGEGQSVAQMYASSAQNIGPDDIRKIKEIADVPVIVKGVECAEDAVLAIGAGADGIVVSNHGGREVDGAPATIDVLPEIAKAVKSCDHPVPIILDGGVRRGSHVFKALALGADLVGIGRPFLYGLALGGAQGVQSVIDQLNKELLIDMQLTGCKTIEDIKHAKIDHINYSADWGISSTSRSVMKPYPVTKENQLTGEAADAVSGASRH

SEQ ID NO: 187 - protein, 398 aa, Proteus alimentorum
MKRQILKATAIAMALSVGVAQAAEYKASTAEGPIKIVNLKAMEAQVQANMEKGAFGYIRGGAEDENNLRSNTTAFDKKYIMPRSLQGIEFSDLDLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIIMTIDSSVGGYREDDVKNNFQFPLGFANLEAFAKISDDKSKTGKGAGISEIYAQAKQAFTPADIQYVKKMSGLPVIVKGIESPEDADTAIKAGADAIWVSNHGGRQLDSAPATIDVLPAIAKVVNKRVPIVFDSGVRRGSHVFKALASGADIVAVGRPILYGLNLGGAEGVNSVIQHLNKELKINMMLGGAKTVKDIQATQLYTDASFNK

SEQ ID NO: 188 - protein, 362 aa, Lactobacillus terrae
MSYKASNAEEHIKIVNLKSLEKEVKDRMEPGAFGYIRGGSEDEWTMDQNTESFNSKKIVPRVFQGIDHADLSTKLWDVDLKTPIIQAPSAAQGLSHEKGEINTAIGMAKAGSIFSISTYANTLIKDAAAAAPDAPQFFQLYMSKDDGFNEFIIKKAIEAKVKAIVLTVDSTLGGYREEDVINDFEFPLPMPNLAEYSQTDGTGKGIGEIYASAKQNFVPEDIQKIKDLSGLPVIVKGIQSPDDAEAAIQAGADGIWVSNHGGRQLDGGPASFEVLPLIAEKVAKRVPVIFDSGIRRGEHVFKALASGADLVAIGRPIIYGLNLGGSQGVYDVMEHINMELSITMQLAGTKTVEDVKNNKLMD

SEQ ID NO: 189 - protein, 411 aa, Lactobacillus panisapium
MTSYYNGFPQSDRDEAINMINLDELEERAKEVMPEGAYYYIASGSENEWTWRNNTAAFNHFQIVPRALTNMDSPQLDTEFMGMKLKTPVMISPIACHGIAHKDAEVATQKGAAAAGALFTSSTYANKSVEDIAAAAPNAPRFFQLYLSKDWDFNKMVFDAVKKAGYSGILLTVDALVSGFREANLRTKFAYPVPLDFFTRYQGAKGEGQTVAQMYASSAQKIGPEDIKRIKEMSGLPVFVKGVVCAEDAFLAMGAGADGIYVTNHGGREIDCGPATIDMLPEIAKAVDHRVPIIFDSGVRRGSHVFKALALGADLVGIGRPYLYGLALGGAKGVQSVIEQLNQELLIDMQLTGCKTIADIKHARLTHFNYTADNLKSNTDPSRITPYPVTNDNQLKEEDSATDAVSGASQE

SEQ ID NO: 190 - protein, 362 aa, Lactimicrobium massiliense
MAYYTSNAEHPIEIVNIAGLEERVKARMDRGAFGYIREGAEDEWTLRENTRAFNDIRIVPRVLQGIDHADLSTSIFGIQLKTPIIEAPSAAHGLAHVKGEVDTAIGAAKAGTLFSMSTYGSTSVEEAAAAAPGAPQFFQLYMSKDDQFNEFLIRKAVKAGVKAIIMTVDSTLGGYREEDVVSHFQFPLPMPNLAAYSSSDGVGKGISEIYAAAKQDFVPEDVAKVKEMSGLPVLVKGIQSPQDALTAIQAGADGIWVSNHGGRQLDGGPASIAVLPSIAEAVSHRVPIIFDSGVRRGQHVFKALASGADLVAVGRPVIYGLNLGGAEGVKSVFDHLNMELSITMQLAGTKTIEDVKKAKLLR

SEQ ID NO: 191 - protein, 398 aa, Proteus vulgaris
MKNKLLKTTAIAMALSVGVAQAAEYKASTAEGPIKIVNLKAMEAQVQANMEKGAFGYIRGGAEDENNLRSNTTAFDKKYIMPRSLQGIEFSDINLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIIMTIDSSVGGYREDDVKNNFQFPLGFANLEAFAKISNDKSKTGKGVGISEIYAQAKQAFTPADIQYVKKMSGLPVIVKGIESPEDADTAIKAGADAIWVSNHGGRQLDSAPATIDMLPAIAKVVNKRVPIVFDSGVRRGSHVFKALASGADVVAVGRPILYGLNLGGAEGVNSVIQHLNKELRINMMLGGAKTVKDIQATQLYTDASFNK

SEQ ID NO: 192 - protein, 364 aa, Fusobacterium ulcerans
MSTKYFQSENEKEINIINFTILEEEAKKIIPAGGYGYINGGAEDEWTLRMNTEAFNHKQIVPRSLTDVEKPDLTTTIYGEKISMPIFMTTVASHGLAHKDGEIATAKGTAAAGTIMGISTYSTKSLDEIMTASTGPKWFQLYMSKDDKFNEYMIGKAVANGAKAVILTVDATLGGYREADLKNNFIFPLPMGNLESLGEGLGQSISEIFANAKQKIGIKDIEKIVAMTDLPVIVKGIESPEDALLAIGAGAKGIYVSNHGGRQLDGGPASFDVLESIAKAVNKKVPIIFDSGVRRGQHVFKALASGADLVGIGRPAIYGLAVGGSKGVTSVFKHFAKELKIVMQLAGCQTVEDIKKAKLLSIKY

SEQ ID NO: 193 - protein, 366 aa, Companilactobacillus musae
MKKKYEASTAENHVDIVNIAGLEARVKDHMSNEKGAFGYIRGGSEDEWTMKQNTEAFNKKKIMPRVIQGIDHADLSTKLWDIDLKTPIIQAPSAAQGLAHAKGEKDTAKGVADAGSIFSISTYGSTSVEDAAEAAPDAPQFFQLYMSKDDKFNEFLIKKAVKAGVKAIILTVDSTLGGYREADIVNKFQFPLPMPNLAGYAAGDGEGKGISEIYASAKQGIIPSDIQKIKDMSGLPVFVKGIQSPDDADLAIEFGADGIWVSNHGGRQLDGGPASFDVLPYIAEVVDKRVPIVFDSGVRRGEHVFKALASGADLVAIGRPIIYGLNLGGAQGVTDVIDHLNMELSITMQLAGTKTINDVKNTELLD

SEQ ID NO: 194 - protein, 411 aa, Myroides sp.
MKKTIVTLIAVTLMTSTYAHVPTTETTTQGKEKKGKGYQASTAEKKLKIVNLHDLEGQVKLEMDKGAFGYIVGGAEDQNNLKINTENFDKKYIMPRVLKGIKHEDIDLSTSLFGISLKTPIIQAPMAAQGLSHVDGEVATAKGMIAAGSLFSLSTYGNKTIEEVAEGINGAPFFFQLYMSKNDDFNKFTLERAKKHGAKAIILTVDSPVGGYREEDIKTGFTFPLVMGNLELFAAQQTDGNKTGKGAGITEIYAQAKQDFKPTDIKYVKDMTGLPVIVKGIQSPEDAEIAIQAGADAIWVSNHGGRQLDAGPSSFDVLPLVAKTVNKRVPIIFDSGVRRGSHIFKALASGADIVAIGRPILYALHLGGSQGVTSVIDQLNKELIINMFLGGAKNIQEIQNTKLYTDKDLSI

SEQ ID NO: 195 - protein, 369 aa, Lentilactobacillus raoultii
MAIVNGYKQNENENSLDILNLDQLEKQAKEIIPTGGFGYISGGSEDEWTLRANRQAFTHKQIVPRALTNIEKPELDTNVFGLPLKTPVFMVPAAAQGLAHVKGEVDTAKGVAAVGGLMAQSTYSSTSIADTAAAGNGAPQFFQLYMSDDWDFNEALLDEAKAAGVKGIILTVDATVDGYREADIINNFQFPIPMANLTKYSEGDGQGKGIAEIYASAAQKIGPDDIARIADYTDLPVIVKGIESPEDALYAIGAGAAGIYVSNHGGRQLNGGPASFDVLEDVAKAVNGKVPIIFDSGVRRGSDVFKALASGADLVGIGRPVIYGLALGGAEGVQSVFEHLDHELEIIMQLAGTKTIEDVKKAKLLNLHY

SEQ ID NO: 196 - protein, 402 aa, Hyphomicrobiales bacterium
MTKFISKKYAVSLSAVALALSLGVANAADYKASTAEGPVEITNLNELEGQVKANMDKGAFGYIRGGAENEQNLRSNTSVFDKKYIMPRVMQGIELSDIDLKTNYLGIDLKTPIIQAPMAAQGLAHQDGEIATAKGMAKAGSIFSLSTYGNKTIEEVAAVSDGNPFFFQLYMSKNEAFNEFTLKRAKDSGAKAIILTVDSPVGGYREDDIKNDFQFPLGFANLELFAKQNDDGSKTGKGAGISEIYAQAKQAFTPADIKYVKDMTGLPVIVKGIQSPEDADVVIKAGADSIWVSNHGGRQLDSGPASFDVLPSIAKVVNKRVPIVFDSGVRRGSHVFKALASGADVVAVGRPILYGLNLGGAEGVNSVIQQLNKELTINMMLGGAKNIEAVKKTKLYSDKDFE

SEQ ID NO: 197 - protein, 367 aa, Enterococcus mediterraneensis
MSYQASKEEHPIEIVNIASLEKAVKERMEAGAFGYIRGGAEDEWTMTENTHSFNKKQIVPRVLRGIDSADLSTKLWDIELKTPIIQAPSAAQGLAHENGEVDTAKGVAEVGSIFSISTYANTTIEDAAAAAPDAPQFFQLYMSKDDKFNEFILEKAVKAGAKAIILTVDSTLGGYREEDIINKFQFPLPMPNLAAYSAQTESGDGEGKGIAEIYAAAKQAIVPEDIQKIKELTSLPVFVKGIQSSEDAEVAIAAGADGIWVSNHGGRQLDGAPASFDLLPSIAEVVNKRVPIVFDSGIRRGEHVFKALASGADLVAIGRPVIYGLNLGGAEGVKSVFDHLNKELSITMQLAGTKTIEEVKNTKLVSF

SEQ ID NO: 198 - protein, 369 aa, Lacticaseibacillus songhuajiangensis
MTVINGYEQSDREQKLNIVNLPSLEAEAKKIIPTGGFGYIAGGSEDDWTLSANTKAFNHAQIVPKALSNIEQPDLSTSIFGIDLKTPVMMAPTAAQGLAHSRGEMATAEGVAAAGALMAQSTYSSTSIADTAAAGKGAPQFFQLYMSKDWDFNRSLLDEAKKAGVKAILLTVDATVDGYREADIVNNFQFPIPMANLTKFSEGDGQGKGIGEIYASAAQKINEDDVRRIAEYTDLPVIVKGIQSPEDALRAIGAGAAGIYVSNHGGRQLNGGPASFDVLPTIAKAVNKQVPIIFDSGVRRGSHVFKALAAGADLVAFGRPVIYGLALGGAQGVQSVFEEIDHELEITMQLAGTKTIEDVKRAPLTHFNY

SEQ ID NO: 199 - protein, 371 aa, Lacticaseibacillus suibinensis
MTMTNGYEQSDNEKKLEIVNLPSLEAEAKKIIPTGGFGYIAGGSEDDWTLKQNTAAFGHVQIVPKALSNIEQPSTATKVFGLDLKTPIMMAPTAAQGLAHAQGEKDTAKGVAAVGGLMAQSTYSSTSIADTAVAGDGAPQFFQLYMSKDWSFNESLLDEAVKAGVKGIILTVDATVDGYREADIINNFQFPIPMANLEKFSEGDGKGKGIGEIYAAAAQKINEDDVRRIAEYTSLPVIVKGIESPEDAMRAIGAGAAGIYVSNHGGRQLNGGPASIDVLPAIAKAVNHQVPIIFDSGVRRGSHVFKALASGADLVAMGRPVIYGLALGGAQGVQSVFEELNHELEITMQLAGTKTIEDVKHAPLTHFNYAD

SEQ ID NO: 200 - protein, 371 aa, Lacticaseibacillus jixianensis
MTLTNGYEQSDREQKLNIVNLPSLEAEAKKIIPTGGFGYIAGGSEDNWTLRQNTAAFGHLQIVPKALSNIEQPSTATRVFGIDLKTPIMMAPTAAQGLAHSQGEKDTARGVAAVGGLMAQSTYSSTSIADTAAAGNGAPQFFQLYMSKDWTFNESLLDEAVKAGAKAIILTVDATVDGYREADIINNFQFPIPMANLTKFSEGDGKGKGIGEIYASAAQKISEADVRRIADYTHLPVIVKGIESPEDALRAIGAGAAGVYVSNHGGRQLNGGPAAIDVLPAIAKAVNHQVPIIFDSGVRRGSHVFKALAAGADLVAMGRPVIYGLALGGAQGVQSVFEELNHELEITMQLAGTKTIEDVKHAPLTHFNYAD

SEQ ID NO: 201 - protein, 367 aa, Companilactobacillus jidongensis
MTETNGYFQNDNEKEIKIVNLPSLEAEAEKIIPKGGFGYIAGGSEDNWTLKANTEAFNHVQIVPHVLSDIENPQTNTSIFGIDVKTPIMMTATAAQGLAHAKGEMDTAKGIAKAGALMEQSTYSSTSIADTMAAGNGAPQFFQLYMSKDWTFNESLLKEAKQAGAKAIVLTADATVDGYRESDIINDFQFPIPMANLTKFSEGDGEGKGIGEIYAAAAQKISSKDIQRIKDIAGLPVIVKGVQSPEDALLAIGAGADGIQVSNHGGRQLNGGPASFDVLSDVAKSVNHKVPIIFDSGVRRGSHVFKALASGADMVALGRPVIYGLALGGADGVYSVVEHLNDEFETIMQLAGTKTIEDVKHAQLLKK

SEQ ID NO: 202 - protein, 367 aa, Companilactobacillus baiquanensis
MTEVNGYIQSDREEKIDVLNLASLEKRAEKIIPAGGFGYISGGSEDNWTLKENTEAFNHAQIVPRVLSDIDDPQTKTSIFGIDLDTPIMMSPAAAQGLAHSQGEKDTAKGMAKAGALMAQSTYSSTSISDAAKAGDGAPQFFQLYMSKDWEFNKSLLEEAKKAGVKAIVLTSDATVDGYRESDIVNDFQFPIPMANLTKFSEGDGKGKGIGEIYAAAAQKISPKDIKRIKDIVDLPVIVKGVQSPEDALLAIGAGADGIYVSNHGGRQLNGGPASFDVLADIAKAVNHKVPIIFDSGVRRGSHVFKALASGADMVALARPIIYGLALGGADGVYSVVEHLNDEFKTIMQLAGTKTIDDVKHAKLLHK

SEQ ID NO: 203 - protein, 367 aa, Companilactobacillus keshanensis
MTEINGYIQSDREEKIDVLNLASLEKRAEEIIPTGGFGYISGGSEDNWTLKENTEAFNHVQIVPRVLSDIDDPQTKTSILGINLETPIMMSPAAAQGLAHSQGEKDTAKGMAKAGALMGQSTYSSTSIADTAAAGNGSPQFFQLYMSKDWDFNKELLNEAKKAGAKAILLTADATVDGYRESDVINDFQFPIPMANLTKFSEGDGKGKGIGEIYAAAAQKIRPDDIQRIKDISGLPVIVKGIQSPEDALLAIGGGTDGIYVSNHGGRQLNGGPASFDVLADVAKAVNHKVPIIFDSGVRRGSHVFKALASGADIVALARPMIYGLALGGADGVYSVVEHLNDELKTIMQLAGTKTIDDVKHAKLLHK

SEQ ID NO: 204 - protein, 371 aa, Lacticaseibacillus yichunensis
MTIVNGYEQSDNENKLDILNLPSLEAEAKKIIPAGGFGYIVGGSEDEWTLAENTKAFSHAQIVPKVLSNIEQPDLSTSIFGISLKTPVMMAPTAAQGLAHSQGEKDTARGVAAVGGLMAQSTYSSTSIADTAAAGEGAPQFFQLYMSKDWTFNESLLDEAKKAGAKAILLTSDATVDGYREEDIVNNFQFPIPMANLTKFSEGDGKGKGIGEIYASAAQKISEDDVRRIAAYTDLPVIVKGVQSPEDALRAIGAGAAGIYVSNHGGRQLNGGPASFDVLPAIAKAVNHQVPIIFDSGVRRGSHVFKALAAGADLVAIGRPAIYGLALGGALGVQSVFEQLDHELTITMQLAGTKTIEDVKRAPLTHFNYAD

SEQ ID NO: 205 - protein, 371 aa, Lacticaseibacillus daqingensis
MTIVNGYEQSDNEKQLAIVNLPSLEKQAQAIIPTGGFGYIVGGAEDDWTLAQNTKAFTHAQIVPKALSEIEAPSTATTVFGLDLKTPIMMAPVAAQGLAHAKGETDTAKGVAAVGGLMSQSTYASTSIADTAAAGAGAPQFFQLYMSKDWDFNYSLLDEAKKAGVKAILLTVDATVDGYREQDIINNFQFPIPMANLQKFSDGDGKGKGIGEIYAAAAQKIGPDDVRRIAEYTDLPVIVKGIESPEDALRAIGAGAAGIYVSNHGGRQLNGGPASFDVLPAIAKAVNHQVPIIFDSGVRRGSHVFKALASGADLVAIGRPVVYGLALGGAQGVQSVFEALNHELEITMQLAGTKTIEDVKHAPLTHFSYTD

SEQ ID NO: 206 - protein, 366 aa, Companilactobacillus zhongbaensis
MTEINGYTQSDNEKELDILNLPSLEAEAKKIIPTGGFGYISGGSEDEWTLSENTKAFNHVQIVPRVLSNIEDPQTDTEIFGIKVKTPIMMSPAAAQGLAHSQGEKDTAKGMAKAGALMSQSTYSSTSIADTAKAGAGSPQFFQLYMSKDWEFNESLLTEAMNAGAKAIILTADATVDGYRESDIINNFQFPIPMANLEKFSTGDGKGKGIGEIYAAAAQKISPADVKRIKDFTNLPVIVKGVQSPEDALLAIGAGADGIYVSNHGGRQLNGGPASFDVLKSVADAVNHQVPIIFDSGVRRGSHVFKALANGADMVALARPIIYGLALGGADGVYSVVEHLNDEFKTIMQLAGTKTIEDVKKAKLLR

SEQ ID NO: 207 - protein, 367 aa, Companilactobacillus kedongensis
MTEINGYIQSDREGKIDVLNLTSLEKRAEEIIPAGGFGYISGGSEDNWTLKENTEAFNHVQIVPRVLSDIDDPQTKTSIFGINVETPIMMSPAAAQGLAHSQGEKDTAKGMAKAGALMGQSTYSSTAIADTAAAGNGSPQFFQLYMSKDWDFNKELLNEAKKAGAKAIILTADATVDGYRESDVINDFQFPIPMANLTKFSEGDGKGKGIGEIYAAAAQKISPDDIQRIKDISGLPVIVKGIQSPEDALLAIGGGADGIYVSNHGGRQLNGGPASFDVLADVAKAVNHKVPIIFDSGVRRGSHVFKALASGADIVALARPMIYGLALGGADGVYSVVEHLNDELKTIMQLAGTKTIDDVKHAKLLHK

SEQ ID NO: 208 - protein, 371 aa, Lacticaseibacillus baoqingensis
MTEVNGYQQSDNEKKLTILNLPALEAEAKKIIPTGGFGYIVGGAEDDWTLRQNTLAFQHAQIVPKALSNIESPDLSTQIFGIDLKTPVMMAPAAAQGLAHSQGEKATAKGVAAVGGLMSQSTYSSTSIADTAAAAPGAPQFFQLYMSKDWDFNYSLLDEAVKAGVKGIILTVDATVDGYREQDIINNFQFPIPMANLEKFSAGDGKGKGIGEIYASAAQKIGEDDVRRIAEYTKLPVIVKGIESPEDALRAIGAGASGIYVSNHGGRELNGGPAAFDVLPSIAKAVNHQVPIIFDSGVRRGSHVFKALAAGADLVAFARPVLYGLALGGAQGVQSVFEQIDHEFEIVMQLAGTKTIADVKQAPLTHFNYQD

SEQ ID NO: 209 - protein, 368 aa, Companilactobacillus hulinensis
MTETNGYFQNDNEKMIDVLNLPSLEARAEKIIPKGGFGYIAGGSEDNWTLKANTEAFNHVQIIPHVLSDIEDPQTNTSFFGINVKTPIMMTATAAQGLAHAKGEIDTAKGIAKAGTLMEQSTYSSTSIADTMAAGNGAPQFFQLYMSKDWTFNESLLKEAKKAGAKAIVLTADATVDGYRESDIINDFQFPIPMANLTKFSEGDGEGKGIGEIYAAAAQKISPADVQRIKDIAGLPVIVKGVQSPEDALLAIGAGADGIQVSNHGGRQLNGGPASFDVLTDVAKAVNHRVPIIFDSGVRRGSHVFKALASGADMVAMGRPVIYGLALGGADGVYSVVEHLNDELKTIMQLAGTKTIEDVKHAKLLKTN

SEQ ID NO: 210 - protein, 367 aa, Loigolactobacillus iwatensis
MSNTYKQSTNEQAIEIVNLDELQERAKAIIPAGGFGYISSGSEDEWTLRANREAFNHKLIVPRALTNMEKPLIDTSVFGISLKTPVMMAPTAAQGLAHVEGEADTARGVAAVGGLMAQSTYSSRTITETMAAGNGAPQFFQLYMSKDWSFNNALLDQTKAAGIKAIILTVDATVGGYREADVRNKFSFPIPMANLEDFSKDSGEGKGISEIYAAAAQKISPKDVARITDYTDLPVIVKGVQSPEDAELAINSGAAGIYVSNHGGRQLNGGPASFDVLASVAQVVNHRVPVIFDSGIRRGSHVFKALASGADLVAFGRPAIYGLALGGAKGVQSVFEHINDELKIVMQLAGTQTIAAVKQTKLLDNHF

SEQ ID NO: 211 - protein, 369 aa, Lacticaseibacillus hulanensis
MTIVNGYEQSDREQKLDILNLPSLENAAKKIIPAGGFGYISGGSEDNWTLGANTSAFNHAQIVPKALSNIENPDTSTSIFGIDLKTPVMMAPAAAQGLAHSRGEVSTAEGVAAAGALMAQSTYSSTSIAATAAAAGGAPQFFQLYMSKDWDFNYALLDEAKKAGVKAIILTVDATVDGYRESDIINDFQFPIPMANLQKFAEGDGKGKGIGEIYASAAQKINEDDVRRIAEYTKLPVIVKGIQSPEDALRAIGAGAAGIYVSNHGGRQLNGGPASFDVLPAIAKAVNKRVPIIFDSGVRRGSHVFKALAAGADLVAFARPAIYGLALGGSQGVQSVFEHITHELTITMQLAGTKTIADVKQAPLTHFNY

SEQ ID NO: 212 - protein, 368 aa, Weissella cibaria
MTYQGSTKEEKLTIIDLPRLREAVKRDTEAGAFGYVDGGSSDEQVLRDNEQAFRHYQLIPRMLQDISAPNLSTALFDIPLAMPVIAAPIAAHGLMHQDGEQVTVKGVGAAGSIFSLSTYGNSRIADVASAAPDTPKFFQLYMSRDDDFNQYLLDEAVNNGYKAIILTADATLGGYREADIINNFTFPLPMENLAAFSNAAGSGEGLGIADIYARAKQNLSLRDINKVKDMAHGLPVIVKGIQDPDDALAANGAGADGIWVSNHGGRELNGAPASIDTLAAITKAVNRRVPVIFDSGIRRGEDVAKALALGADVVALGRPMLWGLNQGGAAGVQSVYEHLATELKIVMQLTGAHTVAELQHAKIIDAKF

SEQ ID NO: 213 - protein, 366 aa, Paucilactobacillus kaifaensis
MSYQASNDERKLKVVNLASLESQVKPRMEAGAFGYIRGGAEDEWTMQQNTEAFKHKKILPHILKGIDNANLHTSIFGIDLDTPVIEAPSAAQGLAHEKGEVDTAKGVAAAGSIFSISTYANTKIEDAAAAAPNAPQFFQLYMSKDDGFNKFILDKAVKAGAKAIILTADSTLGGYREADVLNDFQFPLPMPNLAAYSDKTGSGDGKGKGIAEIYAAAKQGLVLEDIQRVKQWTNLPVIVKGIQAAEDAEASILAGADAIWVSNHGGRQLDGGPASFDVLPAVAKTVAKRVPIIFDSGVRRGEHVFKALASGADLVAIGRPVIYGLNLGGAEGVKSVFDHINMELSITMQLAGTKTIDAVKNTTLLD

SEQ ID NO: 214 - protein, 365 aa, Enterococcus pingfangensis
MAYQTSNEEHPIDIVNIASLEGRVKERMDKGAFGYIRGGSEDEWTMKENTTSFNHKTIMPRVLRGVDHADLSTKLWGIDLDTPIIQAPSAAQGLAHEKGEADTAKGVAAAGSIFSISTYANTTVEDAAAAAPNAPQFFQLYMSKDDGFNEFILEKAVKAGAKAIILTADSTLGGYREEDVINNFQFPLPMPNLAAYSEKSASGNGEGKGIGEIYAAAKQGLVPEDIKKIKDLTNLPVFVKGIQSPEDAEVAIAAGADGIWVSNHGGRQLDGGPASFDVLPMIAGVVNKRVPIVFDSGVRRGEHIFKALASGADLVAIGRPVIYGLNLGGAEGVKSVFDHLNKELSITMQLAGTKTIEEVKNTKLG

SEQ ID NO: 215 - protein, 367 aa, Companilactobacillus huachuanensis
MSNKKYEASTAENHVEIVNLASLEARVKDHMSNEKGAFGYIRGGAEDEWTLKQNTEAFNNKEIMPRVIQGIDHADLSTKLWDIDLKTPIIQAPSAAHGLAHVKGETDTAKGVAAAGSIFSISTYASTSVEDAAAAAPDSPLFFQLYMSKDDKFNEFLIKKAVKAGAKAIVLTVDSTLGGYRESDIINKFQFPLPMPNLAGYSAGDGEGKGISEIYASAKQGIVPTDIQKIKDMSGLPVLVKGIQSPDDAELAIEFGADGIWVSNHGGRQLDGGPASFEMLPYIAEVVDKRVPIVFDSGVRRGEHVFKALASGADLVALGRPIIYGLNLGGAQGVTDVIEHLNMELSITMQLAGTKTINDVKNTKLLG

SEQ ID NO: 216 - protein, 369 aa, Lactococcus raffinolactis
MSYITSTQERHIDILDIPALEAKVAANMEKGAFGYLSGAAEDELVLKANPLAFNHKLIAPRVLQDIENPDLTTEFLGLKLSAPIIAVPIAAHGLVHEHAELDTAQGVADAGTIFSLSTYGNATVDDVAKIAPESPKFFQLYMSKDDNFNRWILDKAVKGGYKAIILTADSTLGGYREADIVNNFTFPNVMHNLEEWSKLSSDGETGSGEGIAAIYAKAKQALSLKDITFIKDYTKLPVFVKGVQSPKDVEPLIEAGVDGIWVSNHGGRQLDGGPASFDVLADIAKVVNKRVPIVFDSGVRRGQHIFKALASGADVVGIGRPMLWGLNLGGSQGVTDVFDHFKKELMITMQLAGTHNVAEIKATELLDAK

SEQ ID NO: 217 - protein, 373 aa, Rikenellaceae bacterium
MKGDYLANDLVRELRIIDLTELEREAARVIGKGAMGYIRGGAGDEWTMRRNTACFEERPILPRVLASLAKPDTKCRILGIDLPFPIIMAPVAAQGLAHVSAEAGTARGTAEAGTIMCVSTYAGMTLEEIALAGNGASQWFQFYPSKDAGFNRHLLDQALAGGYRAVVLTADATVGGNREADLRNRFVFPLKMANLEQYGSGQGRSIEQIYADAMQQIGPAEVERIAAYTRLPVIVKGIQAPEDALRAIDAGAAGVQVSNHGGRQLDGGPGSFEALPAVARAVNGRVPVIFDSGIRRGQHIFKALASGADVVAIGRPAIYGLALGGWMGVRSVFEFFRHELEMVMQLAGTPDIEAVKKTRLFDPRADAQAGDPA

SEQ ID NO: 218 - protein, 365 aa, Lactococcus
MTYLTSTDEHAVEIVNLKSLEALVKERMVAQGNRGAFGYLRGAAEDEWTLRENTESFNRKTIVPRVLRGIDSADLSTSIFGIPLKTPIIQAPVAAQGLAHMEGEVDTAKGMEKVGSLFSISTYGSTTVEEAAEAVNGAPQFFQLYMSKDDDFNQYLLNKAVKAGVKAIILTADSTLGGYREEDVVSKFQFPIPMKNLESYSVTDGVGKGIFEIFGAAKQGLVLDDIQKIKNWTNLPVLVKGIQSPIDALEAITAGADGIWVSNHGGRQLDGGPASFDVLPAIAAVVKKRVPIVFDSGVRRGEHVFKALASGADLVAIGRPIIWGLNLGGADGVASVVEHLNHELSITMQLAGAKDIESVKTTELL

SEQ ID NO: 219 - protein, 400 aa, Morganella psychrotolerans
MKKVKAGLFTATVLGLAMTLTTAYAAEYKASIKEGPLKIVNLNDLESQVKANMDKGAFGYIRGGAEDEKNMRDNTASFDRKYIMPRVMQGIELKDINITTSFLGIPLDTPVIQAPMAAQGLAHRDGEIATAKGMAKAGSIFTLSTYGNKTIEEVAAVSDGHPFFFQLYMSKNDAFNEFTLKRAKESGAKAIILTVDSPVGGWREDDLRNNFQFPLGFANLELFAKQNNDGAKTGKGAGISEIYAQAKQAFTPSDIQYVKKMSGLPVIVKGIQSPEDADRVIEAGADAIWVSNHGGRQLDSGPASFDVLPAIAKTVNKRVPVVFDSGVRRGSHVFKALASGADIVAVGRPVLYGLNLGGAQGVNSVIQQLNKELRINMMLGGAKDINAVKQTRLYTDSDLQ

SEQ ID NO: 220 - protein, 366 aa, Companilactobacillus halodurans
MKKKYEASTAENHIDIVNIASLEGRVKDHMSNEKGAFGYIRGGSEDEWTMRQNTEAFNDKEIMPRVLQGIDHADLSTKLWDIDLKTPIIQAPSAAQGLANAKGEVDTAKGVADAGSIFSISTYGSTAVEDAAKAAPDAPQFFQLYMSKDDKFNEFLLKKAVAAGVKAIILTVDSTLGGYREADIINKFQFPLPMPNLAAYSDSDGEGKGISEIYAAAKQGIVPSDIQKIKDITHLPVIVKGIQSPYDAQLAIEFGADGIWVSNHGGRQLDGGPASFEVLPYIAEIVDKRVPVIFDSGVRRGEHVFKALASGADLVAIGRPILYGLNLGGAQGVTDVIEHLNKELSITMQLAGTKSINEVKNTDLID

SEQ ID NO: 221 - protein, 404 aa, Providencia
MLQRVKKHPIAMMSSAIVALALSVGAAQAVDYQASIKEGPIKIINLDELEDQVAKNMEKGAFGYIRGGAEDELNLDKNTRSFDRKYIMPRVMQGIEIKDIDLSTQFLGIDLKTPIIQAPMAAQGLAHQDGEIATARGMAQAGSIFSLSTYGNKTIEEVAEVSGESPFFFQLYMSKNNAFNEFTLKRAKESGAKAIILTVDSPVGGYREDDIRNNFQFPLGFANLELFAKQNDDGSKTGKGAGISEIYAQAKQAFTPADIAYVKKLSGLPVIVKGIQSPEDADRVIKAGADAIWVSNHGGRQLDSGPASFDVLPSIAKVVNKRVPIVFDSGVRRGSHVFKALASGADVVAIGRPILYGLNLGGAEGVNSVIQQLNKELSINMMLGGTKNIESVKATTLYTDKDFQ

SEQ ID NO: 222 - protein, 369 aa, Lactobacillaceae
MTTVNGYEQSDREQKLDILNLPALEADAKKIIPVGGFGYIAGGSEDNWTLAENTKAFEHAQIVPKALSNIENPDLSTSIFGIDLKTPVMMAPTAAQGLAHAKGEVDTAKGVAAAGALMAQSTYSSTSIADTAAAAPGAPQFFQLYMSKDWDFNYSLLDEAKKAGVKAILLTVDATVDGYRESDIINNFSFPIPMANLTKFSEGDGKGKGIGEIYASAAQKISEDDVRRIAEYTDLPVIVKGIESPEDALRAIGAGAAGIYVSNHGGRQLNGGPASFDVLPAIAKAVNKQVPIIFDSGVRRGSHVFKALASGADLVAFGRPAIYGLALGGAAGVQSVFEHIGHELAITMQLAGTKTIEDVKNAPLTHFHY

SEQ ID NO: 223 - protein, 369 aa, Secundilactobacillus folii
MVMTNGYEQQENEKILNILNLTELEAEAKKIIPTGGFGYISSGSEDEWTLKANRTAFTHRQIVPKSLSDMEDPQTNTNVFGLDLKTPIFMAPTAAQGLAHAKGEVDTAKGVAKAGGLMAQSTYSSTSIADTAAGGEGAPQFFQLYMSKDWDFNKSLLDEAKQAGMKAIILTVDATVGGYREADIINQFQFPIPMANLIKFSEGDGEGKGISEIYAAAAQKIGPADVKRIIDYSDLPVIVKGIESPEDAAYAIGAGAAGVYVSNHGGRQLNGGPASFDVLESVAKAVNGQVPVLFDSGVRRGSDVFKAIALGADLVGIGRPAIYGLALGGADGVFSVFEHLNNELKIIMQLAGTKTVDDIKKTELLNIHY

SEQ ID NO: 224 - protein, 369 aa, Pediococcus acidilactici
MTMINGYEQSDREEKIDILNLESLEERAEEIIPAGGFGYIAGGSEDEWTLKQNRMAFHHRQIAPKALSGIEKPELNTEIFGIPLSTPIMMAPAAAQGLAHSQGEKDTARGLAAVGGLMAQSTYSSVSIAETATAGGDAPQFFQLYMSKDWNFNESLLDEAKKANVKAIILTVDATVDGYREADIKNKFTFPLPMANLIKFSEGNGQGKGIEEIYASAAQNIRPEDVKRIADYTNLPVIVKGIQTPEDAIRAIDAGAAGIYVSNHGGRQLNGGPASFDVLEDIATAVNKQVPIIFDSGVRRGSDVFKALASGADLVALGRPAIYGLALGGAKGVQSVFEHLNHELEIVMQLAGTKTIEDVKNTSLLNIKY

SEQ ID NO: 225 - protein, 365 aa, Enterococcus sp.
MTYQASNEEHELEIVNIASLEAQVKDRMEKGAFGYIRGGSEDEWTMKENTVSFNKKAIMPRVLRGIDAADLSTKLWDIDLKTPIIQAPSAAQGLAHEKGETDTAKGMAEAGSIFSISTYANTTIEDAAAAAPNAPQFFQLYMSKDDGFNEFILDKAVKAGAKAIILTVDSTLGGYREEDVVNHFQFPLPMPNLAAYSERSASGNGEGKGIAEIYAAAKQGLLPADIKKIKDLTNLPVFVKGIQSPEDAEVAIAAGADGIWVSNHGGRQLDGGPASFDVLPMIANVVNKRVPVVFDSGVRRGEHVFKALASGADLVAVGRPIIYGLNLGGAAGVKSVMDHLNKELSITMQLAGTKTIEEVKNTKLL

SEQ ID NO: 226 - protein, 365 aa, Gilliamella sp.
MSYHTSNEEHAIEIVNIASLEALVKARMEAGAFGYIRGGAEDEWTMRENTLSFNRKKIVPRVLQGIDHADLSTKLWDISLKTPIIQAPSAAQGLAHEQGEKDTAKGVAAAGSIFCISTYANTSIEDAANAAPNVPYFFQLYMSKDDDFNRFIIDKAVKAGAKAIILTVDSTLGGYREEDIVNKFQFPLPMKNLSAYSQSNGNGDGSGKGISEIYAAAKQGIVPSDIQKIKAMANLPVIVKGIQSPEDASIAISAGADAIWISNHGGRQLDGAPASFEVLPSIAATIAKRVPIIFDSGVRRGEHVFKALASGADLVAIGRPILYGLNLGGAEGVKSVFDHLNKELSITMQLAGTKTIEDIKNTLLI

SEQ ID NO: 227 - protein, 365 aa, Enterococcus sp.
MYQASNEEHPIEIVNIASLEGKVKADMEVGAFGYIRGGSEDEWTMRENTASFNQKKIMPRVLRGIDHADLSTKLWDIDLKTPIIQAPSAAQGLAHEKGEADTAIGVAATGSIFSISTYANTTIEAAAAAAPGAPQFFQLYMSKDDGFNEFILEKAVKAGAKAIILTADSTLGGYREEDIINHFQFPLPMPNLAAYSEQSASGDGEGKGIAEIYAAAKQGLVPEDIKKIKDLTQLPVIVKGIQSPEDAEVAIAAGADGIWVSNHGGRQLDGGPASFEVLPAIASVVNKRVPVIFDSGVRRGEHVFKALASGADLVAIGRPIIYGLNLGGAQGVASVLEHLNKELSITMQLAGTKTIEEVKNTQLLD

SEQ ID NO: 228 - protein, 362 aa, Sharpea azabuensis
MAYYTSNEEHPIDIVNIASLEERVKARMDRGAFGYIREGAEDEWTLKENTRAFNDISIAPRVLQGMSHADLSTSIFGIDLKTPIIEAPSAAHGLAHVRGEVDTAIGAAKAGTLFAMSTYGSTPVEEAAAAVDGAPQFFQLYMSKDDKFNEFLINKAVKAGVKAIIMTVDSTLGGYREEDIVSHFQFPLPMPNLAAYSNSDGVGKGISEVYAAAKQDFVPSDIAKVKEMSGLPVIVKGIQSPEDAAVAIQAGADGIWVSNHGGRQLDGGPASIKVLPSIANVVNHQVPVIFDSGVRRGQHVFKALASGADLVAIGRPVIYGLNLGGAEGVKSVFDHLNMELSITMQLAGTETIEDIKKTILLK

SEQ ID NO: 229 - protein, 366 aa, Enterococcus
MSYQASTEEHPIEIVNIASLEGRVKERMEAGAFGYIRGGSEDEWTMKENTSSFNTKKIMPRVLRGIDSADLHTSVFGIDLKTPIIQAPSAAQGLAHEKGEADTAKGVAAAGSIFSISTYANTTIKDAADAAPGAPQFFQLYMSKDDGFNEFILKKAVEAGAKAIILTADSTLGGYREEDVINNFQFPLPMPNLAAYSEQSASGNGEGKGIAEIYAAAKQGLTPEDIKKIKDITNLPVIVKGIQSPEDAEVAIAAGADGIWVSNHGGRQLDGGPASFEVLPKIAEVVNKRVPVIFDSGVRRGEHVFKALASGADLVAIGRPVIYGLNLGGAEGVTSVFEHLNKELSITMQLAGTKTIDEVKNTKLMD

SEQ ID NO: 230 - protein, 398 aa, Proteus sp.
MKRKILKNTAIAMALSVSVAQAAEYKASTAEGPIKIVNLKTMEVQVQANMDKGAFSYIRGGAEDENNLRSNTTAFDKKYIMPRSLQGIEFSDLDLKTEFLGIKLDTPIIQAPMAAQGLAHQQGEVATAKGMAKAGSIFSLSTYGNKTIKEVADAQPGYPFFFQLYMSKNDAFNEYILSQAKQYGAKGIILTVDSPVGGYREDDIKNSFQFPLGFANLEAFAKISDDKSKTGKGAGISEIYAQAKQAFIPADIQYVKKMSGLPVIVKGIESPEDADTAIKAGADAIWVSNHGGRQLDSAPSTIDVLPAIAKVVNKRVPIVFDSGVRRGSHVFKALASGADVVAVGRPILYGLNLGGAEGVNSVIQHLNKELKINMMLGGAKTVKDIQATQLYTDTSFNQ

SEQ ID NO: 231 - protein, 369 aa, Lactobacillus sp.
MVMTNGYEQNENEKVLDILNLNQLEADAKKIIPTGGFGYIVSGSEDEWTLQANRKAFQHRQIVPKALSNIEDPQTDTTVFGLDLKTPIMMAPAAAQGLAHAKGEVDTAKGVAKAGGLMAQSTYSSTSIADTAAGGEGAPQFFQLYMSKDWDFNRSLLDEAKKAGVKAIILTVDATVGGYREADIINHFQFPIPMANLIKFSEGDGEGKGISEIYASAAQKIGPDDVKRIIDYTDLPVIVKGIESPEDALYAIGAGAAGIYVSNHGGRQLNGGPASFDVLESVTKAVNHQVPVIFDSGVRRGSDVFKAIALGADLVGIGRPAIYGLALGGADGVFSVFEHLNNELKIVMQLAGTKTIDDIKHAELLHIQY

SEQ ID NO: 232 - protein, 398 aa, Salmonella bongori
MKSSHLIQALASLAMLATSGFTYAEEYKASTDEKAIKMTNVASLEERVQARMEKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEEVANVSGKNPFFFQLYMSKNNQFNEFILAQAVKHGAKAIILTVDSPVGGYREEDIKNNFQFPLGFANLEMFARKNDDGSKTGKGAGISEIYAQAKQAFTPEDIAYVHRISGLPVIVKGIQSPEDAEIAIQAGAAGIWVSNHGGRQLDSGPSSFDMLPAIAKVVNKRVPVIFDSGVRRGSHVFKALASGADIVAIGRPVLYGLNLGGAQGVASVIEQLNKELTINMMLGGAKNIEQVKNTRLLSEKDL

SEQ ID NO: 233 - protein, 364 aa, Megasphaera cerevisiae
MTYHTSNEEHAIDIVNVASLETKVRDRMEKGAFGYIRGGAEDEWTMRENTRSFQSKKIYPRVLKGIDHADLRTSLWDLALDTPIIQAPSAAQGLAHEKGEVDTAIGVARSGSIFSISTYANTSITDAAAAAPGAPQFFQLYMSKDDGFNSFIVGEAIQAGAKAIILTVDSTLGGYREEDIVNKFQFPLPMPNLAAYSTANGAGTGKGIAEIYANAKQGIVLSDIVKIKNMSQLPVFVKGIQAPADAEAAIAAGADGIWVSNHGGRQLDGGPASFECLPKVAEAVHKRVPIVFDSGVRRGEHVFKALASGADLVAIGRPVIYGLNLGGAAGVQSVFEHLNKELSITMQLAGTKTIDDVKNTTLVD

SEQ ID NO: 234 - protein, 369 aa, Lentilactobacillus kribbianus
MTVINGYEQSDREEHINVLNLASLEKRAQAIIPTGGFGYIVGGSEDDWTLAQNRKAFTHKQIYPRTLANIDNPDLSTNVFGIDLQTPVMMAPLAAQGLAHSKGEVATAKAFADEGALMAQSTYSSASIADTAAAGAGAPQFFQLYMSKDWKFNEAILDEAKKAGVKGIILTADATVDGYREADIINNFQFPIPMANLEKFSQGAGKGQGIAEIYAAAAQKISPDDVRRIAEYSGLPVIVKGIQTPEDAELAIGAGAAGVYVSNHGGRQLNGGPASFDALSAIATAVAGRVPIIFDSGVRRGSDVFKSLAAGADLVALGRPFVYALALGGSLGVQDAIQELNHEFATTMQLAGTKTIAEVKQAKLADFNY

SEQ ID NO: 235 - protein, 368 aa, Lentilactobacillus kribbianus
MTMVNGYEQSDREKHIKVVDLESLEAEAQKIIPTGAFGYISGGSEDEWTLRQNRKSFTHKQIYPRVLTDVEKPEISTNFMGIDLKTPIMMPPLAAQGLANSQSEKDTARAFAAAGGLMALSTYGSATIADFAKAGNGAPQFFQPYMSKNDDFNKSLLDEAKRNGMKAIILTADSTLGGYREADVINNFQFPIPMANLEKLSQGAGKGQGIAAIYAAAAQKIGPDEIKKIADYTDLPVIVKGIQNPVDAELAIDAGAAAIYVSNHGGRQLNGGPGSFDVLPEIAVAVAKRVPIIFDSGIRHGSDVFKALATGADLVAIGRPFVYALALGGQLGVEDALAEINREFQIVMQLAGTQTIEDVKNTKLADLS

SEQ ID NO: 236 - protein, 368 aa, Lactococcus hodotermopsidis
MTYITSTQERHIDIVDIPSLEKLVQANMAKGAFGYLAGAAEDELVLKANPLAFNHKHIVPRVLQDIQDPDLTTEFLGLKLSSPIIGVPIAAHGLVHEKAELATAQGVAKAGTIFSLSTYGNATVDEVAAVAPDSPKFFQLYMSKDDDFNRWILDKAIKGGYKAIILTADSTLGGYRESDIINNFTFPNVMRNLEEWSKLSTDSENGAGEGIAAIYAKAKQALSLRDITFIKDYTHLPVFVKGIQSAHDVEPLINAGVDGIWVSNHGGRQLDAGPASFDVLADIAKAVNKRVPIIFDSGVRRGQHVFKALANGADIVGIGRPMLWGLNLGGSQGVTDVFEHFKKELRINMQLAGAHTIAEVKETKLIDA

SEQ ID NO: 237 - protein, 366 aa, Lactococcus insecticola
MAYTTSTDEQKVAIVNIKSLEGKVKARMEAQGNKGAFGYIRGGAEDEWTMSENTRAFNDKKIVPRVLRGVDSADLSTSIFGIDLKTPIIQAPVAAQGLAHMAGEVDTAKAMAEVGSLFSISTYGSTSVEDAAAAAPGAPQFFQLYMSKDDQFNEFLLKKAVAAGVKAIILTADSTLGGYREEDVINGFEFPLPMPNLAAFSESDGVGKGIGEIYAAAKQGLVLEDIKKIKDITGLPVLVKGVQSAEDASAAIDAGADGIWVSNHGGRQLDGGPASIETLPAIASAVAKRVPIVFDSGVRRGEHVFKAIAQGADLVAVGRPVLYGLNLGGAQGVQSVIEHLNKELSITMQLAGTKNIAEIKTTDLID

SEQ ID NO: 238 - protein, 365 aa, Enterococcus saigonensis
MYHTSNEEHPIDIVNIASLEGRVKERMEAGAFGYIRGGSEDEWTMKENTTSFMNKKIMPRILQGIDHADLSTKLWDIDLKTPIIQAPSAAQGLAHENGEADTAKGVAAAGSIFSISTYANTTIEDAAAAAPDAPQFFQLYMSKDDGFNEFILDKAVKAGAKAIILTADSTLGGYREEDIINQFQFPLPMPNLAAYSEQSASGNGEGKGIAEIYAAAKQGLVPEDIKKIKDLTHLPVFVKGIQSPEDADLAIKAGADGIWVSNHGGRQLDGGPASFEVLPAIAEVVDKRVPIVFDSGVRRGEHVFKALASGADLVAIGRPVIYGLNLGGAEGVKSVFDHLNKELSITMQLAGTKTISDVKKAKLID

SEQ ID NO: 239 - protein, 364 aa, Fusobacteriaceae bacterium
MSTKYFQSENEKEINIINFRMLEEEAKKIIPAGGYGYISGGAEDEWTLKMNTEAFNHKQIVPRSLTDVEKPDLTTTIYGEKISMPIFMTTVASHGLAHRDGEIATAKGAAAAETIMGISTYSTKSLDEIMTASTGPKWFQLYMSKDDNFNRYMIEKAVANGAKAVILTVDATLGGYREADLKNNFIFPLPMGNLESLGAGLGQSISEIFANAKQKIGIKDIEEIVSLTNLPVIVKGIESPEDALLAIGAGAKGIYVSNHGGRQLDGGPASFDVLESIAKAVNRKVPIIFDSGVRRGQHVFKALASGADLVGIGRPAIYGLAVGGSKGVTSVFKHFAKELKIVMQLAGCKTIEDIKKAKLLSIKY

SEQ ID NO: 240 - protein, 371 aa, Lacticaseibacillus absianus
MTTVNGYEQSDNEQRLAILNLPSLEAKAKAIIPTGGFGYIVGGAEDDWTLRQNTKAFTHAQIVPKALSDIEAPSTATTVFGLDLKTPIMMAPVAAQGLAHAKGETDTAKGVAAVGALMSQSTYSSTSIADTAAAGAGAPQFFQLYMSKDWAFNYSLLDEAKKAGVKAILLTVDATVDGYREQDIINDFQFPIPMANLQKFSEGDGAGKGIGEIYAAAAQKIGPDDVRRIAEYTDLPVIVKGIQSPEDAMRAIGAGAAGLYVSNHGGRQLNGGPASFDVLPAIAKAVNHRVPIIFDSGIRRGSHVFKALASGADLVAIGRPVLYGLALGGAEGVQSVFEALNHELEITMQLAGTKTIEDVKHAPLTHFQYAD

SEQ ID NO: 241 - protein, 398 aa, Salmonella enterica
MKSSHLIQALASLAMLATSGFTYAEEYKASTDEKAIKMTNVASLEERVQARMEKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEDVAKVSGKNPFFFQLYMSKNNEFNEFILSQAVKHGAKAIILTVDSPVGGYREEDIKNDFQFPLGFANLEMFASKNDDGSKTGKGSGISEIYAQAKQAFTPVDIAYVHRISGLPVIVKGIQSPEDAEIAIQAGAAGIWVSNHGGRQLDSGPSSFDMLPAIAKVVNKRVPVIFDSGVRRGSHVFKALASGADIVAIGRPVLYGLNLGGAQGVASVIEQLNKELTINMMLGGARNIEQVKNTRLLSEKDL

SEQ ID NO: 242 - protein, 398 aa, Salmonella enterica
MKSSHLIQALASLAMLATSGLAYAEEYKASTDEKTIKMTNVASLEARVQARMEKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEDVAKVSGKSPFFFQLYMSKNNKFNEFILSQAVKHGAKAIILTVDSPVGGYREEDIKNDFQFPLGFANLEMFASKNDDGSKTGKGSGISEIYAQAKQAFTPEDIAYVHRISGLPVIIKGIQSPEDAEIAIQAGAAGIWVSNHGGRQLDSGPSSFDMLPAIAKVVNKRVPVIFDSGVCRGSHVFKALASGADIVAIGRPVLYGLNLGGAQGVASVIEQLNKELTINMMLGGARNIEQVKNTRLLSEKDL

SEQ ID NO: 243 - protein, 400 aa, Salmonella enterica
MKKNHLIRVVASLAMLVTSGFTYAEEYKASTDEKAIKMTNVASLEARVQARMEKGAFGYIRGGAEDENNLRSNTESFDKKYIMPRVLQGIELKEIDLSTQLLGIPLKTPVIQAPMAAQGLAHASGELATAKGMAQVGSIFSLSTYGNKTIEEVANVSGKNLFFFQLYMSKNNQFNEFILAQAVKHGAKAIILTVDSPVGGYREEDIKNNFQFPLGFANLEMFARKNDDGSKTGKGAGISEIYAQAKQAFTPEDIAYVHSVSGLPVIVKGIQTPEDADIAIQAGAAGIWVSNHGGRQLDSGPSSFDMLPAIAKVVNKRVPIIFDSGVRRGSHVFKALASGADIVAVGRPILYGLNLGGSQGVASVIEQLNKELTINMMLGGARNIEQVKMTRLLTEKDLQQ

SEQ ID NO: 244 - protein, 400 aa, Salmonella enterica
MKTNHLIRAVASLAMLATSSFTYAEEYKASTDEKAVKMTNVASLEARVQARMEKGAFGYIRGGAEDENNLRSNTESFDKKYIVPRVMQGIELKEIDLSTQLLGIPLKTPVIQAPMAAQGLAHASGELATAKGMAQVGSIFSLSTYGNKTIEEVAKASGKNPFFFQLYMSKNNQFNEFILAQAVKHGAKAIILTVDSPVGGHREEDIKNDFQFPLGFANLEMFARQNDDGSKTGKGSGISEIYAQAKQAFTPEDIAYVHRVSGLPVIVKGIQSPEDAEIAIQAGAAGIWVSNHGGRQLDSGPSSFDMLPAIAKVVNKRVPVIFDSGVRRGSHIFKALASGADIVAVGRPILYGLNLGGAQGVASVIEQLNKELTINMMLGGARNIEQVKTTRLLTEKELPQ

SEQ ID NO: 245 - protein, 404 aa, Salmonella enterica
MKTHHLIRVVVASLAMLAMLATSGLAYAEEYKASTDEKAIKMTNVASLEARVQAKMEKGAFGYIRGGAEDENNLRSNTESFDKKYIMPRVLQGIELKEIDLSTQLLGIPLKTPIIQAPMAAQGLAHASGELATAKGMAQVGSIFSLSTYGNKTIEEVANVSGKNPFFFQLYMSKNNQFNEFILAQAVKHGAKAIILTVDSPVGGYREEDIKNNFQFPLGFANLEMFARKNDDGSKTGKGAGISEIYAQAKQAFTPEDIAYVHRISGLPVIVKGIQSPEDAEIAIQAGAAGIWVSNHGGRQLDSGPSSFDMLPAIAKVVNKRVPVIFDSGVRRGSHVFKALASGADIVAVGRPVLYGLNLGGAQGVASVIEQLNKELTINMMLGGARNIEQVKTTRLLTEKDLPQ

SEQ ID NO: 246 - protein, 368 aa, Pseudoclavibacter sp.
MTVTNGYEQSDREESIDILNLDELEERARQIIPKGGFGYISEGSEDEWTKARNREAFNAVQIVPRVLTEVDAPSTATSVFGIDVKTPVIMAPAAAQGLAHARGEAATAEGIAAAGSLMVQSTYGTTTIAQTATAAPGAPWFFQLYMSTDWDFNHALIDEAKKYGAAAIVLTVDSMQGGYREPDIANKFQFPLPMANLEQFSSRGSKGKGIAEIYAAAAQKITPTDVEKIIDYSGLPVIVKGIQDPDDANLALGAGAAGIWVSNHGGRQLNGGPGSFDVLPSIARAVNGRVPVVFDSGVRRGSHVFKALASGADLVATARPFIYALALGGAQGVQAAVEHLTHEFRIVMQLAGTNTVDDVKRAKLLRVS

SEQ ID NO: 247 - protein, 365 aa, Enterococcus asini
MAYQTSNEEHPIDIVNIASLEGRVKERMDKGAFGYIRGGSEDEWTMKENTTSFNHKTIMPRVLRGVDHADLSTKLWDIDLDTPIIQAPSAAQGLAHEKGEADTAKGVAAAGSIFSISTYANTTVEDAAAAAPNAPQFFQLYMSKDDGFNEFILEKAVKAGAKAIILTADSTLGGYREEDVINNFQFPLPMPNLAAYSEKSASGDGEGKGIGEIYAAAKQGLVPEDIKKIKYLTNLPVFVKGIQSPEDAEVAIAAGADGIWISNHGGRQLDGGPASFDVLPMIAGVVNKRVPIVFDSGVRRGEHVFKALASGADLVAIGRPVIYGLNLGGAEGVKSVFDHLNKELSITMQLAGTKTIDEVKNTKLS

SEQ ID NO: 248 - protein, 396 aa, Alcaligenaceae bacterium
MTPRVVFAQTSSDSASSTSAATGGYQASTDEGAIKIVNLEELEALVKPRMEEGAFGYIRSGAENEINLRANTTSFNRKYIMPRILQGLESTDINLSTQLLGIDLKTPIIQAPMAAQGLAHVEGEIATARGVAEAGSIFSLSTYGNKTIEDVAEAGGNNPFFFQIYMSKNDAFNEFTLKRAKDSGAKAIILTVDSPVGGYREEDIRTKFTFPLGMPNLELFAAQNDDGTKTGQGSGITEIYAQAKQAFTPEDIAYIKDLTGLPVLVKGVQAPEDATVAIKAGADGIWVSNHGGRQFDSGPASFDVLPHIAKVVNKRVPIIFDSGVRRGSHVFKALASGADIVAVGRPILYGLNLGGGAGVHSIIEHLNKELRINMMLAGARDIEAIKKTPLYTDEDF

SEQ ID NO: 249 - protein, 365 aa, Enterococcus faecalis
MYYASNEEHPIEVINVASLEDKVKNHMDKGAFGYIRGGAEDEWTMRQNTRSFMNKKIIPRVLQGVDHADLSTNLWNIDLKTPIIQAPTAAQGLAHKKGEVNTAKAMAEVGSLFSISTYANTSIEDAASAAPGSPQFFQLYMSKDDNFNEFILEKAVKVGAKAIILTADSTLGGYREEDIINNFRFPLPMPNLASYSKQSDSGDGKGKGIAEIYAAAKQGLVPEDIKKIKDISNLPVFVKGIQSSDDAEVAINAGADGIWVSNHGGRQLDGGPASFEVLPDIARVVNKRVPIIFDSGVRRGEHIFKALASGADLVAIGRPVLYGLNLGGASGVVSVLEHLNKELSITMQLAGTKTIDEIKTTILSE

SEQ ID NO: 250 - protein, 18 aa, synthetic construct
  REGION 1..18: note = tag
GSSHHHHHHGLEVLFQGP